(19) United States
(12) Patent Application Publication
(10) Pub. No.: US 2024/0239784 A1
(43) Pub. Date: Jul. 18, 2024

(54) INDOLE COMPOUNDS AND METHODS OF USE

(71) Applicant: GENZYME CORPORATION, Cambridge, MA (US)

(72) Inventors: Junkai Liao, Andover, MA (US); Mark Munson, Acton, MA (US); Sukanthini Thurairatnam, Cambridge, MA (US); Bradford Hirth, Cambridge, MA (US); Zhongli Gao, Waltham, MA (US); Gregory Hurlbut, Boxborough, MA (US); David Borcherding, Cambridge, MA (US); Matthieu Barrague, Cambridge, MA (US); Timothy Alan Gillespy, Cambridge, MA (US); Alexandre Gross, Cambridge, MA (US); Thaddeus Nieduzak, Cambridge, MA (US); Andrew Good, Cambridge, MA (US); Roy Vaz, Cambridge, MA (US); Jinyu Liu, Cambridge, MA (US); Yi Li, Cambridge, MA (US); Markus Metz, Cambridge, MA (US); Anatoly Ruvinsky, Cambridge, MA (US); Michael Kothe, Cambridge, MA (US)

(21) Appl. No.: 18/593,643

(22) Filed: Mar. 1, 2024

Related U.S. Application Data
(63) Continuation of application No. PCT/US2022/075875, filed on Sep. 2, 2022.
(60) Provisional application No. 63/240,758, filed on Sep. 3, 2021.

(51) Int. Cl. C07D 413/14; C07D 401/14; C07D 403/10; C07D 403/12; C07D 403/14; C07D 413/12; C07D 471/04

(52) U.S. Cl. CPC C07D 413/14; C07D 401/14; C07D 403/10; C07D 403/12; C07D 403/14; C07D 413/12; C07D 471/04

(57) ABSTRACT

The invention relates to heterocyclic compounds, pharmaceutically acceptable salts thereof, and pharmaceutical preparations thereof. Also described herein are compositions and the use of such compounds in methods of treating diseases and conditions mediated by deficient CFTR activity, in particular cystic fibrosis.

### CROSS-REFERENCE TO RELATED APPLICATIONS

This application is a continuation of International Application No. PCT/US2022/075875, filed Sep. 2, 2022, which claims priority to U.S. Provisional Application No. 63/240,758, filed Sep. 3, 2021, the contents of which are incorporated herein by reference.

### BACKGROUND

Cystic fibrosis (CF), an autosomal recessive disorder, is caused by functional deficiency of the cAMP-activated plasma membrane chloride channel, cystic fibrosis transmembrane conductance regulator (CFTR), which results in pulmonary and other complications. The gene encoding CFTR has been identified and sequenced (See Gregory, R. J. et al. (1990) Nature 347:382-386; Rich, D. P. et al. (1990) Nature 347:358-362), (Riordan, J. R. et al. (1989) Science 245:1066-1073). CFTR, a member of the ATP binding cassette (ABC) superfamily is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate and thiocyanate into and out of the cell. CFTR may have a regulatory role over other electrolyte channels, including the epithelial sodium channel ENaC.

In cystic fibrosis patients, the absence or dysfunction of CFTR leads to exocrine gland dysfunction and a multisystem disease, characterized by pancreatic insufficiency and malabsorption, as well as abnormal mucociliary clearance in the lung, mucostasis, chronic lung infection and inflammation, decreased lung function and ultimately respiratory failure. While more than 1,900 mutations have been identified in the CFTR gene, a detailed understanding of how each CFTR mutation may impact channel function is known for only a few. (Derichs, European Respiratory Review, 22:127, 58-65 (2013)). The most frequent CFTR mutation is the in-frame deletion of phenylalanine at residue 508 (ΔF508) in the first nucleotide binding domain (NBD1). Over 80% of cystic fibrosis patients have the deletion at residue 508 in at least one allele. The loss of this key phenylalanine renders NBD1 conformationally unstable at physiological temperature and compromises the integrity of the interdomain interface between NDB1 and CFTR's second transmembrane domain (ICL4). The ΔF508 mutation causes production of misfolded CFTR protein which, rather than traffic to the plasma membrane, is instead retained in the endoplasmic reticulum and targeted for degradation by the ubiquitin-proteasome system.

The loss of a functional CFTR channel at the plasma membrane disrupts ionic homeostasis and airway surface hydration leading to reduced lung function. Reduced periciliary liquid volume and increased mucus viscosity impede mucociliary clearance resulting in chronic infection and inflammation. In the lung, the loss of CFTR-function leads to numerous physiological effects downstream of altered anion conductance that result in the dysfunction of additional organs such as the pancreas, intestine and gall bladder.

By studying the mechanistic aspects of CFTR misfolding and corrections, small molecules have been identified as CF modulators, that can act as stabilizers.

Despite the identification of compounds that modulate CFTR, there is no cure for this fatal disease and identification of new compounds and new methods of therapy are needed as well as new methods for treating or lessening the severity of cystic fibrosis and other CFTR mediated conditions and diseases in a patient.

### SUMMARY

Disclosed herein are compounds of Formula (I):

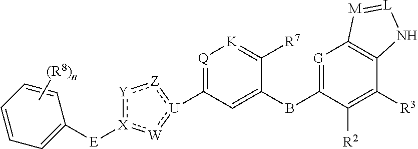

wherein
U is C;
W is N or NR^{a}, wherein R^{a} is H;
X is N or C;
Y is CR^{b}, wherein R^{b} is H, alkyl or carboxy, N, or NR^{c}, where R^{c} is alkyl;
Z is O, S, N or NR^{d}, where R^{d} is H;
B is —O—, —C(O)—, —S—, —S(O)—, —S(O)₂—, or —C(R¹⁰R¹¹)—, wherein R¹⁰ and R¹¹ are independently selected from H, hydroxy, and —(CH₂)pJ, wherein p=1-3, and J is —C(O)NH₂, —COOH, or —CN;
G is N or CR¹, wherein R¹ is H, halo, alkyl, alkenyl, amido, sulfinyl, sulfonyl and sulfonamido;
R² is H or halo;
R³ is H or halo;
L is N or CH;
M is N or CR⁹, wherein R⁹ is H, —CH₂COOH, —CH₂OH, or R⁹ taken together with R¹ and any intervening atoms forms an N-containing 6- or 7-membered heterocycle, optionally substituted with oxo;
Q is N or CR^{4c};
R^{4c} is selected from H or halo;
K is N or CH;
R⁷ is H or amino;
E is —S(O)—, —S(O)₂—, or —C(R⁵R⁶)—, wherein
R⁵ is selected from H, alkyl, hydroxy, alkoxy, carboxy, and amino;
R⁶ is H or alkyl;
R⁸ is halo, haloalkyl, or unsubstituted alkyl; and
n=0-1;
wherein each alkyl, alkenyl, amino, amido, and alkoxy is independently unsubstituted or substituted with one or more substituents selected from halo, hydroxy, amino, amido, alkyl, alkoxy, carboxy, heterocyclyl, and sulfonyl; wherein the alkyl substituent is optionally further substituted with one or more substituents selected from halo, hydroxy, amino, amido, carboxy, and sulfonamido.

Disclosed herein are methods for treating a disease or condition mediated by deficient CFTR activity. Such diseases and conditions include, but are not limited to, cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome. In some embodiments, the disease is cystic fibrosis.

In certain embodiments, the present invention provides a pharmaceutical composition suitable for use in a subject in the treatment or prevention of disease and conditions associate with deficient CFTR activity, comprising any of the compounds described herein (e.g., a compound of the invention, such as a compound of formula (I)), and one or more pharmaceutically acceptable carriers or excipients. In certain embodiments, the pharmaceutical preparations may be for use in treating or preventing a condition or disease as described herein.

Provided herein are combination therapies of compounds of formula (I) with CFTR-active agents that can enhance the therapeutic benefit beyond the ability of the primary therapy alone.

### DETAILED DESCRIPTION

The present invention provides compounds of Formula (I):

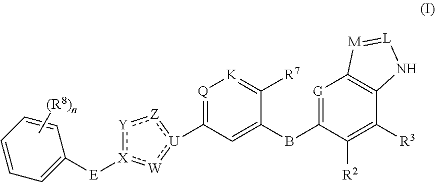

wherein
U is C;
W is N or NR^{a}, wherein R^{a} is H;
X is N or C;
Y is CR^{b}, wherein R^{b} is H, alkyl or carboxy, N, or NR^{c}, where R^{c} is alkyl;
Z is O, S, N or NR^{d}, where R^{d} is H;
B is —O—, —C(O)—, —S—, —S(O)—, —S(O)₂—, or —C(R¹⁰R¹¹)—, wherein R¹⁰ and R¹¹ are independently selected from H, hydroxy, and —(CH₂)pJ, wherein p=1-3, and J is —C(O)NH₂, —COOH, or —CN;
G is N or CR¹, wherein R¹ is H, halo, alkyl, alkenyl, amido, sulfinyl, sulfonyl and sulfonamido;
R² is H or halo;
R³ is H or halo;
L is N or CH;
M is N or CR⁹, wherein R⁹ is H, —CH₂COOH, —CH₂OH, or R⁹ taken together with R¹ and any intervening atoms forms an N-containing 6- or 7-membered heterocycle, optionally substituted with oxo;
Q is N or CR^{4c};
R^{4c} is selected from H or halo;
K is N or CH;
R⁷ is H or amino;
E is —S(O)—, —S(O)₂—, or —C(R⁵R⁶)—, wherein
R⁵ is selected from H, alkyl, hydroxy, alkoxy, carboxy, and amino;
R⁶ is H or alkyl;
R⁸ is halo, haloalkyl, or unsubstituted alkyl; and
n=0-1;
wherein each alkyl, alkenyl, amino, amido, and alkoxy is independently unsubstituted or substituted with one or more substituents selected from halo, hydroxy, amino, amido, alkyl, alkoxy, carboxy, heterocyclyl, and sulfonyl; wherein the alkyl substituent is optionally further substituted with one or more substituents selected from halo, hydroxy, amino, amido, carboxy, and sulfonamido.

### Variables W, X, Y, and Z of Formula (I)

Below are exemplary embodiments of variables U, W, X, Y, and Z of the disclosed compound of Formula (I). The values for the remaining variables are as described above and below.

In some embodiments, W is NH; X is C; Y is CH; and Z is N.

In some embodiments, W is NH; X is C; Y is N; and Z is N.

In some embodiments, W is N; X is C; Y is NH; and Z is N.

In some embodiments, W is N; X is C; Y is CH; and Z is NH.

In some embodiments, W is N; X is C; Y is CR^{b}; and Z is NH.

In some embodiments, W is N; X is C; Y is C; and Z is N.

In some embodiments, W is N; X is C; Y is N; and Z is NH.

In some embodiments, W is N; X is N; Y is CH; and Z is N.

In some embodiments, W is N; X is C; Y is CH; and Z is O.

In some embodiments, W is N; X is C; Y is CH; and Z is S.

Variables R¹, R², R³, L, M, and G of Formula (I)

Below are exemplary embodiments of variables R¹, R², R³, M, and G of the disclosed compound of Formula (I). The values for the remaining variables are as described above and below.

In some embodiments, L is CH; G is N; M is CH; R² is H; R³ is H; R^{4c} is H.

In some embodiments, G is N; M is CCH₂OH; R² is H; R³ is H; R^{4c} is H.

In some embodiments, G is CR¹, and R¹ is selected from —COOH, —CH₂OH, —CH₂COOH, —CH₂NH₂, —CH₂NHMe, —CH₂NHEt, —CH₂NHiPr, —CH₂NHtBu, —CH₂NHCOMe, —CH₂CONH—SO₂Me, —CH₂CONH—CF₃, —CH₂CONH—SO₂-cyclopropyl, —SOMe, and —SO₂Me; R² is H; R³ is H; and R^{4c} is H.

In some embodiments, L is N; M is CH; G is CR¹; R² is H; R³ is H; and R^{4c} is H.

In some embodiments, R¹ is selected from —CH₂CH₂COOH, —CH₂CH₂CONH₂, —CH₂CH₂CONHSO₂Me, and —CH₂CH₂CONHSO₂cyclopropyl; R² is H; R³ is H; and R^{4c} is H.

In some embodiments, R¹ is selected from C(O)NHR¹⁵; R² is H; R³ is H; R^{4c} is H; and R¹⁵ is selected from H, —CH₂CH₂OH, —CH₂CH₂OMe, —CH₂CH₂NMe₂, —CH₂CH₂OCH₂CH₂OMe, —CH₂CH(OH)CH₂OH, —CH₂CH(OH)CH₂NEt₂, —CH₂CONHMe, —CH₂CMe₂OH, —CH₂CH(OH)CF₃, —CH₂CH₂SO₂Me, —CH₂-(3-oxetanyl)—CH₂OH, —CH₂-4-(2,2-dimethyl-1,3-dioxolanyl), —CH₂-thiazolyl, —OMe, —OCH₂CH₂OMe and

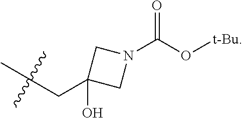 [SMILES: *CC1(O)CN(C(=O)OC(C)(C)C)C1]

In some embodiments, R¹ is selected from —CH(OH)CH₃, —COMe, —CONH₂, —CH₂OH, —SO₂Me, —SOCH₂CH₂OMe, —SO₂CH₂CH₂OMe, —SO₂CH₂CH₂NHMe

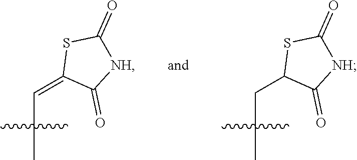 [SMILES: */C=C1/SC(=O)NC1=O ; "and" ; *CC1SC(=O)NC1=O]

R² is F; R³ is H; and R^{4c} is H.

In some embodiments, R¹ is selected from Me, —CH₂COOH, —CH₂CH₂COOH, —CH₂CH₂SO₂NH₂, —SO₂Me, —SO₂NHMe,

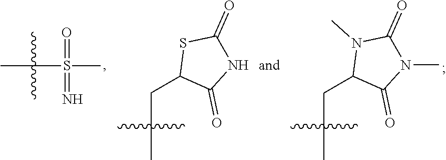

R² is F; R³ is H; and R^{4c} is F.

In some embodiments, L is CH; M is N; G is CH; R² is H; R³ is H; R^{4c} is H.

In some embodiments, L is CH; M is CH; G is CH; R² is H; R³ is H; R^{4c} is H.

In some embodiments, M is CR⁹, where R⁹ is selected from —CH₂COOH and —CH₂OH.

In some embodiments, M is CR⁹, where R⁹ taken together with R¹ and any intervening atoms forms an N-containing 6-membered heterocycle.

In some embodiments, M is CR⁹, where R⁹ taken together with R¹ and any intervening atoms forms an N-containing 7-membered heterocycle.

In some embodiments, M is CR⁹, where R⁹ taken together with R¹ and any intervening atoms forms an N-containing 6-membered heterocycle substituted with carboxyl.

In some embodiments, M is CR⁹, where R⁹ taken together with R¹ and any intervening atoms forms an N-containing 7-membered heterocycle substituted with carboxyl.

In some embodiments, M is CCH₂COOH; R¹ is Me; R² is F; R³ is H; and R^{4c} is F.

In some embodiments, M is CCH₂OH; R¹ is Me; R² is H; R³ is H; and G is N.

In some embodiments, R¹ is F; R² is F; R³ is H; and R^{4c} is H.

In some embodiments, R¹ is F; R² is H; R³ is H; and R^{4c} is H.

In some embodiments, R¹ is F; R² is F; R³ is H; and R^{4c} is F.

In some embodiments, R¹ is H; R² is F; R³ is H; and R^{4c} is H.

In some embodiments, R¹ is H; R² is H; R³ is F; and R^{4c} is H.

In some embodiments, R¹ is H; R² is H; R³ is H; and R^{4c} is H.

### Variables B, Q, K, and R⁷ of Formula (I)

Below are exemplary embodiments of variables B, Q, K, and R⁷ of the disclosed compound of Formula (I). The values for the remaining variables are as described above and below.

In some embodiments B is —O—; Q is CR^{4c}; K is CH; R^{4c} is H; R⁷ is H.

In some embodiments B is —O—; Q is CR^{4c}; K is N; R^{4c} is H; R⁷ is H.

In some embodiments B is —O—; Q is N; R⁷ is H.

In some embodiments B is —O—; Q is N; R⁷ is NH₂.

In some embodiments B is —O—; Q is CR^{4c}; R^{4c} is F; R⁷ is H.

In some embodiments B is selected from —C(O)—, —S—, —S(O)—, —S(O)₂—. In some embodiments, B is —C(R¹⁰R¹¹)—, where R¹⁰ and R¹¹ are independently selected from H, hydroxy, and —(CH₂)pJ.

In some embodiments p=1.

In some embodiments, J is —C(O)NH₂, —COOH, or —CN.

### Variables E, and R⁸ of Formula (I)

Below are exemplary embodiments of variables E and R⁸ of the disclosed compound of Formula (I). The values for the remaining variables are as described above and below.

In some embodiments, E is selected from —S(O)— and —S(O)₂—.

In some embodiments, E is —C(R⁵R⁶)—, where R⁵ is selected from H and hydroxy; R⁶ is H.

In some embodiments, E is —C(R⁵R⁶)—, where R⁵ is selected from H and hydroxy; R⁶ is alkyl.

In some embodiments, E is —C(R⁵R⁶)—, wherein R⁵ is selected from alkoxy, carboxy, and amino; R⁶ is H.

In some embodiments, E is —C(R⁵R⁶)—, wherein R⁵ is selected from alkoxy, carboxy, and amino; R⁶ is alkyl.

In some embodiments, R⁵ and R⁶ are H, R⁸ is halo.

In some embodiments, R⁸ is selected from halo, haloalkyl, or unsubstituted alkyl.

In some embodiments, R⁵ is selected from H, hydroxy, methyl, NH₂, CF₃, —COOH, —CH₂CH₂COOH, —OCH₂OH, —OCH₂COOH, —OCH₂CH₂NH₂, and —OCH₂CH(OH)CH₂OH; and R⁶ is selected from H and CH₃.

In some embodiments, R⁵ and R⁶ are each H.

In some embodiments, R⁵ is hydroxy and R⁶ is H.

In some embodiments, R⁵ is hydroxy or methyl, and R⁶ is methyl.

In some embodiments, R⁵ is amino or methyl, and R⁶ is H.

### Compounds of Formula IA

In some embodiments, the compound of Formula (I) has the structure of compound (IA):

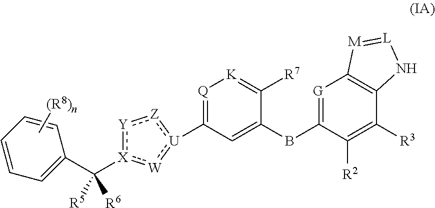

wherein
U is C;
W is N or NR^{a}, wherein R^{a} is H;
X is N or C;
Y is CR^{b}, wherein R^{b} is H, alkyl or carboxy, N, or NR^{c}, where R^{c} is alkyl;
Z is O, S, N or NR^{d}, where R^{d} is H;
B is —O—, —C(O)—, —S—, —S(O)—, —S(O)₂—, or —C(R¹⁰R¹¹)—, wherein R¹⁰ and R¹¹ are independently selected from H, hydroxy, and —(CH₂)pJ, wherein p=1-3, and J is —C(O)NH₂, —COOH, or —CN;
G is N or CR¹, wherein R¹ is H, halo, alkyl, alkenyl, amido, sulfinyl, sulfonyl and sulfonamido;
R² is H or halo;
R³ is H or halo;
L is N or CH;
M is N or CR⁹, wherein R⁹ is H, —CH₂COOH, —CH₂OH, or R⁹ taken together with R¹ and any intervening atoms forms an N-containing 6- or 7-membered heterocycle, optionally substituted with oxo;
Q is N or CR^{4c};
R^{4c} is selected from H or halo;
K is N or CH;
R⁷ is H or amino;
R⁵ is selected from H, alkyl, hydroxy, alkoxy, carboxy, and amino;
R⁶ is H or alkyl;
R⁸ is halo, haloalkyl, or unsubstituted alkyl; and
n=0-1;
wherein each alkyl, alkenyl, amino, amido, and alkoxy is independently unsubstituted or substituted with one or more substituents selected from halo, hydroxy, amino, amido, alkyl, alkoxy, carboxy, heterocyclyl, and sulfonyl; wherein the alkyl substituent is optionally further substituted with one or more substituents selected from halo, hydroxy, amino, amido, carboxy, and sulfonamido.

### Variables W, X, Y, and Z of Formula (IA)

Below are exemplary embodiments of variables U, W, X, Y, and Z of the disclosed compound of Formula (IA). The values for the remaining variables are as described above and below.

In some embodiments, W is NH; X is C; Y is CH; and Z is N.

In some embodiments, W is NH; X is C; Y is N; and Z is N.

In some embodiments, W is N; X is C; Y is NH; and Z is N.

In some embodiments, W is N; X is C; Y is CH; and Z is NH.

In some embodiments, W is N; X is C; Y is CR^{b}; and Z is NH.

In some embodiments, W is N; X is C; Y is C; and Z is N.

In some embodiments, W is N; X is C; Y is N; and Z is NH.

In some embodiments, W is N; X is N; Y is CH; and Z is N.

In some embodiments, W is N; X is C; Y is CH; and Z is O.

In some embodiments, W is N; X is C; Y is CH; and Z is S.

Variables R¹, R², R³, L, M, and G of Formula (IA)

Below are exemplary embodiments of variables R¹, R², R³, M, and G of the disclosed compound of Formula (IA). The values for the remaining variables are as described above and below.

In some embodiments, L is CH; G is N; M is CH; R² is H; R³ is H; R^{4c} is H.

In some embodiments, G is N; M is CCH₂OH; R² is H; R³ is H; R^{4c} is H.

In some embodiments, G is CR¹, and R¹ is selected from —COOH, —CH₂OH, —CH₂COOH, —CH₂NH₂, —CH₂NHMe, —CH₂NHEt, —CH₂NHiPr, —CH₂NHtBu, —CH₂NHCOMe, —CH₂CONH—SO₂Me, —CH₂CONH—CF₃, —CH₂CONH—SO₂-cyclopropyl, —SOMe, and —SO₂Me; R² is H; R³ is H; and R^{4c} is H.

In some embodiments, L is N; M is CH; G is CR¹; R² is H; R³ is H; and R^{4c} is H.

In some embodiments, R¹ is selected from —CH₂CH₂COOH, —CH₂CH₂CONH₂, —CH₂CH₂CONHSO₂Me, and —CH₂CH₂CONHSO₂cyclopropyl; R² is H; R³ is H; and R^{4c} is H.

In some embodiments, R¹ is selected from C(O)NHR¹⁵; R² is H; R³ is H; R^{4c} is H; and R¹⁵ is selected from H, —CH₂CH₂OH, —CH₂CH₂OMe, —CH₂CH₂NMe₂, —CH₂CH₂OCH₂CH₂OMe, —CH₂CH(OH)CH₂OH, —CH₂CH(OH)CH₂NEt₂, —CH₂CONHMe, —CH₂CMe₂OH, —CH₂CH(OH)CF₃, —CH₂CH₂SO₂Me, —CH₂-(3-oxetanyl)—CH₂OH, —CH₂-4-(2,2-dimethyl-1,3-dioxolanyl), —CH₂-thiazolyl, —OMe, —OCH₂CH₂OMe and

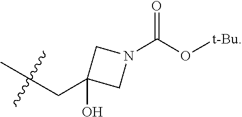 [SMILES: *CC1(O)CN(C(=O)OC(C)(C)C)C1]

In some embodiments, R¹ is selected from —CH(OH)CH₃, —COMe, —CONH₂, —CH₂OH, —SO₂Me, —SOCH₂CH₂OMe, —SO₂CH₂CH₂OMe, —SO₂CH₂CH₂NHMe

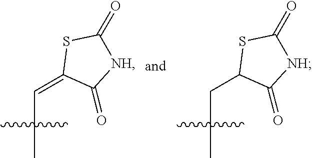 [SMILES: */C=C1/SC(=O)NC1=O ; *CC1SC(=O)NC1=O]

R² is F; R³ is H; and R^{4c} is H.

In some embodiments, R¹ is selected from Me, —CH₂COOH, —CH₂CH₂COOH, —CH₂CH₂SO₂NH₂, —SO₂Me, —SO₂NHMe,

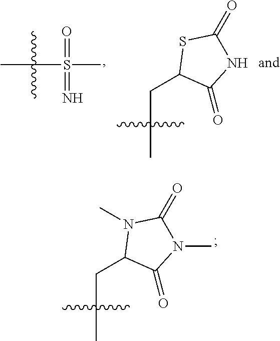

R² is F; R³ is H; and R^{4c} is F.

In some embodiments, L is CH; M is N; G is CH; R² is H; R³ is H; R^{4c} is H.

In some embodiments, L is CH; M is CH; G is CH; R² is H; R³ is H; R^{4c} is H.

In some embodiments, M is CR⁹, where R⁹ is selected from —CH₂COOH and —CH₂OH.

In some embodiments, M is CR⁹, where R⁹ taken together with R¹ and any intervening atoms forms an N-containing 6-membered heterocycle.

In some embodiments, M is CR⁹, where R⁹ taken together with R¹ and any intervening atoms forms an N-containing 7-membered heterocycle.

In some embodiments, M is CR⁹, where R⁹ taken together with R¹ and any intervening atoms forms an N-containing 6-membered heterocycle substituted with carboxyl.

In some embodiments, M is CR⁹, where R⁹ taken together with R¹ and any intervening atoms forms an N-containing 7-membered heterocycle substituted with carboxyl.

In some embodiments, M is C—CH₂COOH; R¹ is Me; R² is F; R³ is H; and R^{4c} is F.

In some embodiments, M is C—CH₂OH; R¹ is Me; R² is H; R³ is H; and G is N.

In some embodiments, R¹ is F; R² is F; R³ is H; and R^{4c} is H.

In some embodiments, R¹ is F; R² is H; R³ is H; and R^{4c} is H.

In some embodiments, R¹ is F; R² is F; R³ is H; and R^{4c} is F.

In some embodiments, R¹ is H; R² is F; R³ is H; and R^{4c} is H.

In some embodiments, R¹ is H; R² is H; R³ is F; and R^{4c} is H.

In some embodiments, R¹ is H; R² is H; R³ is H; and R^{4c} is H.

### Variables B, Q, K, and R⁷ of Formula (IA)

Below are exemplary embodiments of variables B, Q, K, and R⁷ of the disclosed compound of Formula (IA). The values for the remaining variables are as described above and below.

In some embodiments B is —O—; Q is CR^{4c}; K is CH; R^{4c} is H; R⁷ is H.

In some embodiments B is —O—; Q is CR^{4c}; K is N; R^{4c} is H; R⁷ is H.

In some embodiments B is —O—; Q is N; R⁷ is H.

In some embodiments B is —O—; Q is N; R⁷ is NH₂.

In some embodiments B is —O—; Q is CR^{4c}; R^{4c} is F; R⁷ is H.

In some embodiments B is selected from —C(O)—, —S—, —S(O)—, —S(O)₂—.

In some embodiments, B is —C(R¹⁰R¹¹)—, where R¹⁰ and R¹¹ are independently selected from H, hydroxy, and —(CH₂)pJ.

In some embodiments p=1.

In some embodiments, J is —C(O)NH₂, —COOH, or —CN.

Variables R⁵, R⁶, and R⁸ of Formula (IA)

Below are exemplary embodiments of variables R⁵, R⁶, and R⁸ of the disclosed compound of Formula (IA). The values for the remaining variables are as described above and below.

In some embodiments, R⁸ is halo.

In some embodiments, R⁸ is selected from haloalkyl and unsubstituted alkyl.

In some embodiments, R⁵ is selected from H, hydroxy, methyl, NH₂, CF₃, —COOH, —CH₂CH₂COOH, —OCH₂OH, —OCH₂COOH, —OCH₂CH₂NH₂, and —OCH₂CH(OH)CH₂OH; and R⁶ is selected from H and CH₃.

In some embodiments, R⁵ and R⁶ are each H.

In some embodiments, R⁵ is hydroxy and R⁶ is H.

In some embodiments, R⁵ is hydroxy or methyl, and R⁶ is methyl.

In some embodiments, R⁵ is amino or methyl, and R⁶ is H.

### Compounds of Formula IB

In some embodiments, the compound of Formula (I) has the structure of compound (IB):

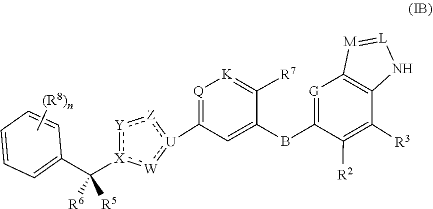

wherein
U is C;
W is N or NR^{a}, wherein R^{a} is H;
X is N or C;
Y is CR^{b}, wherein R^{b} is H, alkyl or carboxy, N, or NR^{c}, where R^{c} is alkyl;
Z is O, S, N or NR^{d}, where R^{d} is H;
B is —O—, —C(O)—, —S—, —S(O)—, —S(O)₂—, or —C(R¹⁰R¹¹)—, wherein R¹⁰ and R¹¹ are independently selected from H, hydroxy, and —(CH₂)pJ, wherein p=1-3, and J is —C(O)NH₂, —COOH, or —CN;
G is N or CR¹, wherein R¹ is H, halo, alkyl, alkenyl, amido, sulfinyl, sulfonyl and sulfonamido;
R² is H or halo;
R³ is H or halo;
L is N or CH;
M is N or CR⁹, wherein R⁹ is H, —CH₂COOH, —CH₂OH, or R⁹ taken together with R¹ and any intervening atoms forms an N-containing 6- or 7-membered heterocycle, optionally substituted with oxo;
Q is N or CR^{4c};
R^{4c} is selected from H or halo;
K is N or CH;
R⁷ is H or amino;
R⁵ is selected from H, alkyl, hydroxy, alkoxy, carboxy, and amino;
R⁶ is H or alkyl;
R⁸ is halo, haloalkyl, or unsubstituted alkyl; and
n=0-1;
wherein each alkyl, alkenyl, amino, amido, and alkoxy is independently unsubstituted or substituted with one or more substituents selected from halo, hydroxy, amino, amido, alkyl, alkoxy, carboxy, heterocyclyl, and sulfonyl; wherein the alkyl substituent is optionally further substituted with one or more substituents selected from halo, hydroxy, amino, amido, carboxy, and sulfonamido.

### Variables W, X, Y, and Z of Formula (IB)

Below are exemplary embodiments of variables U, W, X, Y, and Z of the disclosed compound of Formula (IB). The values for the remaining variables are as described above and below.

In some embodiments, W is NH; X is C; Y is CH; and Z is N.

In some embodiments, W is NH; X is C; Y is N; and Z is N.

In some embodiments, W is N; X is C; Y is NH; and Z is N.

In some embodiments, W is N; X is C; Y is CH; and Z is NH.

In some embodiments, W is N; X is C; Y is CR^{b}; and Z is NH.

In some embodiments, W is N; X is C; Y is C; and Z is N.

In some embodiments, W is N; X is C; Y is N; and Z is NH.

In some embodiments, W is N; X is N; Y is CH; and Z is N.

In some embodiments, W is N; X is C; Y is CH; and Z is O.

In some embodiments, W is N; X is C; Y is CH; and Z is S.

Variables R¹, R², R³, L, M, and G of Formula (IB)

Below are exemplary embodiments of variables R¹, R², R³, M, and G of the disclosed compound of Formula (IB). The values for the remaining variables are as described above and below.

In some embodiments, L is CH; G is N; M is CH; R² is H; R³ is H; R^{4c} is H.

In some embodiments, G is N; M is CCH₂OH; R² is H; R³ is H; R^{4c} is H.

In some embodiments, G is CR¹, and R¹ is selected from —COOH, —CH₂OH, —CH₂COOH, —CH₂NH₂, —CH₂NHMe, —CH₂NHEt, —CH₂NHiPr, —CH₂NHtBu, —CH₂NHCOMe, —CH₂CONH—SO₂Me, —CH₂CONH—CF₃, —CH₂CONH—SO₂-cyclopropyl, —SOMe, and —SO₂Me; R² is H; R³ is H; and R^{4c} is H.

In some embodiments, L is N; M is CH; G is CR¹; R² is H; R³ is H; and R^{4c} is H.

In some embodiments, R¹ is selected from —CH₂CH₂COOH, —CH₂CH₂CONH₂, —CH₂CH₂CONHSO₂Me, and —CH₂CH₂CONHSO₂cyclopropyl; R² is H; R³ is H; and R^{4c} is H.

In some embodiments, R¹ is selected from C(O)NHR¹⁵; R² is H; R³ is H; R^{4c} is H; and R¹⁵ is selected from H, —CH₂CH₂OH, —CH₂CH₂OMe, —CH₂CH₂NMe₂, —CH₂CH₂OCH₂CH₂OMe, —CH₂CH(OH)CH₂OH, —CH₂CH(OH)CH₂NEt₂, —CH₂CONHMe, —CH₂CMe₂OH, —CH₂CH(OH)CF₃, —CH₂CH₂SO₂Me, —CH₂-(3-oxetanyl)—CH₂OH, —CH₂-4-(2,2-dimethyl-1,3-dioxolanyl), —CH₂-thiazolyl, —OMe, —OCH₂CH₂OMe and

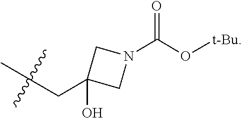 [SMILES: *CC1(O)CN(C(=O)OC(C)(C)C)C1]

In some embodiments, R¹ is selected from —CH(OH)CH₃, —COMe, —CONH₂, —CH₂OH, —SO₂Me, —SOCH₂CH₂OMe, —SO₂CH₂CH₂OMe, —SO₂CH₂CH₂NHMe

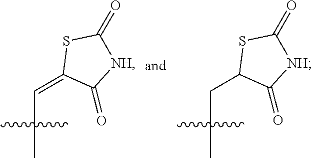 [SMILES: */C=C1/SC(=O)NC1=O ; *CC1SC(=O)NC1=O]

R² is F; R³ is H; and R^{4c} is H.

In some embodiments, R¹ is selected from Me, —CH₂COOH, —CH₂CH₂COOH, —CH₂CH₂SO₂NH₂, —SO₂Me, —SO₂NHMe,

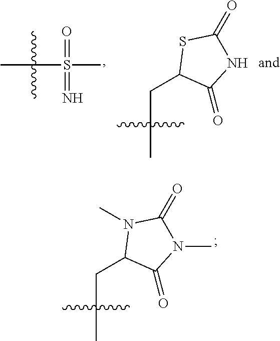

R² is F; R³ is H; and R^{4c} is F.

In some embodiments, L is CH; M is N; G is CH; R² is H; R³ is H; R^{4c} is H.

In some embodiments, L is CH; M is CH; G is CH; R² is H; R³ is H; R^{4c} is H.

In some embodiments, M is CR⁹, where R⁹ is selected from —CH₂COOH and —CH₂OH.

In some embodiments, M is CR⁹, where R⁹ taken together with R¹ and any intervening atoms forms an N-containing 6-membered heterocycle.

In some embodiments, M is CR⁹, where R⁹ taken together with R¹ and any intervening atoms forms an N-containing 7-membered heterocycle.

In some embodiments, M is CR⁹, where R⁹ taken together with R¹ and any intervening atoms forms an N-containing 6-membered heterocycle substituted with carboxyl.

In some embodiments, M is CR⁹, where R⁹ taken together with R¹ and any intervening atoms forms an N-containing 7-membered heterocycle substituted with carboxyl.

In some embodiments, M is C—CH₂COOH; R¹ is Me; R² is F; R³ is H; and R^{4c} is F.

In some embodiments, M is C—CH₂OH; R¹ is Me; R² is H; R³ is H; and G is N.

In some embodiments, R¹ is F; R² is F; R³ is H; and R^{4c} is H.

In some embodiments, R¹ is F; R² is H; R³ is H; and R^{4c} is H.

In some embodiments, R¹ is F; R² is F; R³ is H; and R^{4c} is F.

In some embodiments, R¹ is H; R² is F; R³ is H; and R^{4c} is H.

In some embodiments, R¹ is H; R² is H; R³ is F; and R^{4c} is H.

In some embodiments, R¹ is H; R² is H; R³ is H; and R^{4c} is H.

### Variables B, Q, K, and R⁷ of Formula (IB)

Below are exemplary embodiments of variables B, Q, K, and R⁷ of the disclosed compound of Formula (IB). The values for the remaining variables are as described above and below.

In some embodiments B is —O—; Q is CR^{4c}; K is CH; R^{4c} is H; R⁷ is H.

In some embodiments B is —O—; Q is CR^{4c}; K is N; R^{4c} is H; R⁷ is H.

In some embodiments B is —O—; Q is N; R⁷ is H.

In some embodiments B is —O—; Q is N; R⁷ is NH₂.

In some embodiments B is —O—; Q is CR^{4c}; R^{4c} is F; R⁷ is H.

In some embodiments B is selected from —C(O)—, —S—, —S(O)—, —S(O)₂—.

In some embodiments, B is —C(R¹⁰R¹¹)—, where R¹⁰ and R¹¹ are independently selected from H, hydroxy, and —(CH₂)pJ.

In some embodiments p=1.

In some embodiments, J is —C(O)NH₂, —COOH, or —CN.

Variables R⁵, R⁶, and R⁸ of Formula (IB)

Below are exemplary embodiments of variables R⁵, R⁶, and R⁸ of the disclosed compound of Formula (IB). The values for the remaining variables are as described above and below.

In some embodiments, R⁸ is halo.

In some embodiments, R⁸ is selected from haloalkyl and unsubstituted alkyl.

In some embodiments, R⁵ is selected from H, hydroxy, methyl, NH₂, CF₃, —COOH, —CH₂CH₂COOH, —OCH₂OH, —OCH₂COOH, —OCH₂CH₂NH₂, and —OCH₂CH(OH)CH₂OH; and R⁶ is selected from H and CH₃.

In some embodiments, R⁵ and R⁶ are each H.

In some embodiments, R⁵ is hydroxy and R⁶ is H.

In some embodiments, R⁵ is hydroxy or methyl, and R⁶ is methyl.

In some embodiments, R⁵ is amino or methyl, and R⁶ is H.

The present invention provides compounds of Formula (I′):

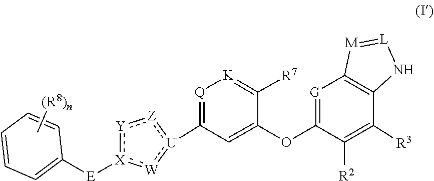

wherein
U is C;
W is N or NR^{a}, wherein R^{a} is H;
X is N or C;
Y is CR^{b}, wherein R^{b} is H, alkyl or carboxy, N, or NR^{c}, where R^{c} is alkyl;
Z is O, S, N or NR^{d}, where R^{d} is H;
G is N or CR¹, wherein R¹ is H, halo, alkyl, alkenyl, amido, sulfinyl, sulfonyl and sulfonamido;
R² is H or halo;
R³ is H or halo;
L is N or CH;
M is N or CR⁹, wherein R⁹ is H, —CH₂COOH, —CH₂OH, or R⁹ taken together with R¹ and any intervening atoms forms an N-containing 6- or 7-membered heterocycle, optionally substituted with oxo;
Q is N or CR^{4c};
R^{4c} is selected from H or halo;
K is N or CH;
R⁷ is H or amino;
E is —S(O)—, —S(O)₂—, or —C(R⁵R⁶)—, wherein
R⁵ is selected from H, alkyl, hydroxy, alkoxy, carboxy, and amino;
R⁶ is H or alkyl;
R⁸ is halo, haloalkyl, or unsubstituted alkyl; and
n=0-1;
wherein each alkyl, alkenyl, amino, amido, and alkoxy is independently unsubstituted or substituted with one or more substituents selected from halo, hydroxy, amino, amido, alkyl, alkoxy, carboxy, heterocyclyl, and sulfonyl; wherein the alkyl substituent is optionally further substituted with one or more substituents selected from halo, hydroxy, amino, amido, carboxy, and sulfonamido.

### Variables W, X, Y, and Z of Formula (I)

Below are exemplary embodiments of variables U, W, X, Y, and Z of the disclosed compound of Formula (I′). The values for the remaining variables are as described above and below.

In some embodiments, W is NH; X is C; Y is CH; and Z is N.

In some embodiments, W is NH; X is C; Y is N; and Z is N.

In some embodiments, W is N; X is C; Y is NH; and Z is N.

In some embodiments, W is N; X is C; Y is CH; and Z is NH.

In some embodiments, W is N; X is C; Y is CR^{b}; and Z is NH.

In some embodiments, W is N; X is C; Y is C; and Z is N.

In some embodiments, W is N; X is C; Y is N; and Z is NH.

In some embodiments, W is N; X is N; Y is CH; and Z is N.

In some embodiments, W is N; X is C; Y is CH; and Z is O.

In some embodiments, W is N; X is C; Y is CH; and Z is S.

Variables R¹, R², R³, L, M, and G of Formula (I)

Below are exemplary embodiments of variables R¹, R², R³, M, and G of the disclosed compound of Formula (I′). The values for the remaining variables are as described above and below.

In some embodiments, L is CH; G is N; M is CH; R² is H; R³ is H; R^{4c} is H.

In some embodiments, G is N; M is M is CCH₂OH; R² is H; R³ is H; R^{4c} is H.

In some embodiments, G is CR¹, and R¹ is selected from —COOH, —CH₂OH, —CH₂COOH, —CH₂NH₂, —CH₂NHMe, —CH₂NHEt, —CH₂NHiPr, —CH₂NHtBu, —CH₂NHCOMe, —CH₂CONH—SO₂Me, —CH₂CONH—CF₃, —CH₂CONH—SO₂-cyclopropyl, —SOMe, and —SO₂Me; R² is H; R³ is H; and R^{4c} is H.

In some embodiments, L is N; M is CH; G is CR¹; R² is H; R³ is H; and R^{4c} is H.

In some embodiments, R¹ is selected from —CH₂CH₂COOH, —CH₂CH₂CONH₂, —CH₂CH₂CONHSO₂Me, and —CH₂CH₂CONHSO₂cyclopropyl; R² is H; R³ is H; and R^{4c} is H.

In some embodiments, R¹ is selected from C(O)NHR¹⁵; R² is H; R³ is H; R^{4c} is H; and R¹⁵ is selected from H, —CH₂CH₂OH, —CH₂CH₂OMe, —CH₂CH₂NMe₂, —CH₂CH₂OCH₂CH₂OMe, —CH₂CH(OH)CH₂OH, —CH₂CH(OH)CH₂NEt₂, —CH₂CONHMe, —CH₂CMe₂OH, —CH₂CH(OH)CF₃, —CH₂CH₂SO₂Me, —CH₂-(3-oxetanyl)—CH₂OH, —CH₂-4-(2,2-dimethyl-1,3-dioxolanyl), —CH₂-thiazolyl, —OMe, —OCH₂CH₂OMe and

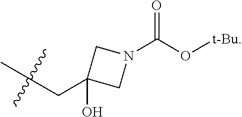 [SMILES: *CC1(O)CN(C(=O)OC(C)(C)C)C1]

In some embodiments, R¹ is selected from —CH(OH)CH₃, —COMe, —CONH₂, —CH₂OH, —SO₂Me, —SOCH₂CH₂OMe, —SO₂CH₂CH₂OMe, —SO₂CH₂CH₂NHMe

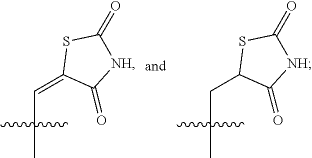 [SMILES: */C=C1/SC(=O)NC1=O ; *CC1SC(=O)NC1=O]

R² is F; R³ is H; and R^{4c} is H.

In some embodiments, R¹ is selected from Me, —CH₂COOH, —CH₂CH₂COOH, —CH₂CH₂SO₂NH₂, —SO₂Me, —SO₂NHMe, NH 0 and

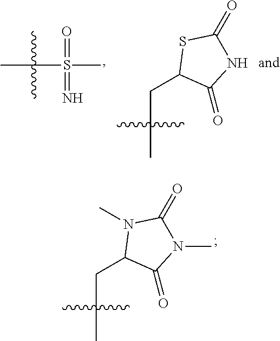

R² is F; R³ is H; and R^{4c} is F.

In some embodiments, L is CH; M is N; G is CH; R² is H; R³ is H; R^{4c} is H.

In some embodiments, L is CH; M is CH; G is CH; R² is H; R³ is H; R^{4c} is H.

In some embodiments, M is CR⁹, where R⁹ is selected from —CH₂COOH and —CH₂OH.

In some embodiments, M is CR⁹, where R⁹ taken together with R¹ and any intervening atoms forms an N-containing 6-membered heterocycle.

In some embodiments, M is CR⁹, where R⁹ taken together with R¹ and any intervening atoms forms an N-containing 7-membered heterocycle.

In some embodiments, M is CR⁹, where R⁹ taken together with R¹ and any intervening atoms forms an N-containing 6-membered heterocycle substituted with carboxyl.

In some embodiments, M is CR⁹, where R⁹ taken together with R¹ and any intervening atoms forms an N-containing 7-membered heterocycle substituted with carboxyl.

In some embodiments, M is C—CH₂COOH; R¹ is Me; R² is F; R³ is H; and R^{4c} is F.

In some embodiments, M is C—CH₂OH; R¹ is Me; R² is H; R³ is H; and G is N.

In some embodiments, R¹ is F; R² is F; R³ is H; and R^{4c} is H.

In some embodiments, R¹ is F; R² is H; R³ is H; and R^{4c} is H.

In some embodiments, R¹ is F; R² is F; R³ is H; and R^{4c} is F.

In some embodiments, R¹ is H; R² is F; R³ is H; and R^{4c} is H.

In some embodiments, R¹ is H; R² is H; R³ is F; and R^{4c} is H.

In some embodiments, R¹ is H; R² is H; R³ is H; and R^{4c} is H.

### Variables Q, K, and R⁷ of Formula (I′)

Below are exemplary embodiments of variables Q, K, and R⁷ of the disclosed compound of Formula (I′). The values for the remaining variables are as described above and below.

In some embodiments Q is CR^{4c}; K is CH; R^{4c} is H; R⁷ is H.

In some embodiments Q is CR^{4c}; K is N; R^{4c} is H; R⁷ is H.

In some embodiments Q is N; R⁷ is H.

In some embodiments Q is N; R⁷ is NH₂.

In some embodiments Q is CR^{4c}; R^{4c} is F; R⁷ is H.

Variables E, and R⁸ of Formula (I′) Below are exemplary embodiments of variables E and R⁸ of the disclosed compound of Formula (I′). The values for the remaining variables are as described above and below.

In some embodiments, E is selected from —S(O)— and —S(O)₂—.

In some embodiments, E is —C(R⁵R⁶)—, where R⁵ is selected from H and hydroxy; R⁶ is H.

In some embodiments, E is —C(R⁵R⁶)—, where R⁵ is selected from H and hydroxy; R⁶ is alkyl.

In some embodiments, E is —C(R⁵R⁶)—, wherein R⁵ is selected from alkoxy, carboxy, and amino; R⁶ is H.

In some embodiments, E is —C(R⁵R⁶)—, wherein R⁵ is selected from alkoxy, carboxy, and amino; R⁶ is alkyl.

In some embodiments, R⁵ and R⁶ are H, R⁸ is halo.

In some embodiments, R⁸ is selected from halo, haloalkyl, or unsubstituted alkyl.

In some embodiments, R⁵ is selected from H, hydroxy, methyl, NH₂, CF₃, —COOH, —CH₂CH₂COOH, —OCH₂OH, —OCH₂COOH, —OCH₂CH₂NH₂, and —OCH₂CH(OH)CH₂OH; and R⁶ is selected from H and CH₃.

In some embodiments, R⁵ and R⁶ are each H.

In some embodiments, R⁵ is hydroxy and R⁶ is H.

In some embodiments, R⁵ is hydroxy or methyl, and R⁶ is methyl.

In some embodiments, R⁵ is amino or methyl, and R⁶ is H.

### Compounds of Formula IA′

In some embodiments, the compound of Formula (I) has the structure of compound (IA′):

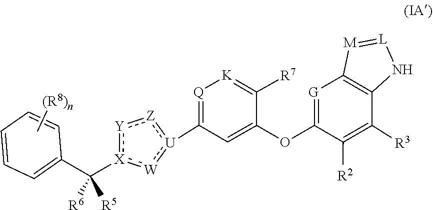

wherein
U is C;
W is N or NR^{a}, wherein R^{a} is H;
X is N or C;
Y is CR^{b}, wherein R^{b} is H, alkyl or carboxy, N, or NR^{c}, where R^{c} is alkyl;
Z is O, S, N or NR^{d}, where R^{d} is H;
G is N or CR¹, wherein R¹ is H, halo, alkyl, alkenyl, amido, sulfinyl, sulfonyl and sulfonamido;
R² is H or halo;
R³ is H or halo;
L is N or CH;
M is N or CR⁹, wherein R⁹ is H, —CH₂COOH, —CH₂OH, or R⁹ taken together with R¹ and any intervening atoms forms an N-containing 6- or 7-membered heterocycle, optionally substituted with oxo;
Q is N or CR^{4c};
R⁴, is selected from H or halo;
K is N or CH;
R⁷ is H or amino;
R⁵ is selected from H, alkyl, hydroxy, alkoxy, carboxy, and amino;
R⁶ is H or alkyl; and
n=0-1;
R⁸ is halo, haloalkyl, or unsubstituted alkyl;
wherein each alkyl, alkenyl, amino, amido, and alkoxy is independently unsubstituted or substituted with one or more substituents selected from halo, hydroxy, amino, amido, alkyl, alkoxy, carboxy, heterocyclyl, and sulfonyl; wherein the alkyl substituent is optionally further substituted with one or more substituents selected from halo, hydroxy, amino, amido, carboxy, and sulfonamido.

### Variables W, X, Y, and Z of Formula (IA′)

Below are exemplary embodiments of variables U, W, X, Y, and Z of the disclosed compound of Formula (IA′). The values for the remaining variables are as described above and below.

In some embodiments, W is NH; X is C; Y is CH; and Z is N.

In some embodiments, W is NH; X is C; Y is N; and Z is N.

In some embodiments, W is N; X is C; Y is NH; and Z is N.

In some embodiments, W is N; X is C; Y is CH; and Z is NH.

In some embodiments, W is N; X is C; Y is CR^{b}; and Z is NH.

In some embodiments, W is N; X is C; Y is C; and Z is N.

In some embodiments, W is N; X is C; Y is N; and Z is NH.

In some embodiments, W is N; X is N; Y is CH; and Z is N.

In some embodiments, W is N; X is C; Y is CH; and Z is O.

In some embodiments, W is N; X is C; Y is CH; and Z is S.

Variables R¹, R², R³, L, M, and G of Formula (IA′)

Below are exemplary embodiments of variables R¹, R², R³, M, and G of the disclosed compound of Formula (IA′). The values for the remaining variables are as described above and below.

In some embodiments, L is CH; G is N; M is CH; R² is H; R³ is H; R^{4c} is H. In some embodiments, G is N; M is CH—CCH₂OH; R² is H; R³ is H; R^{4c} is H.

In some embodiments, G is CR¹, and R¹ is selected from —COOH, —CH₂OH, —CH₂COOH, —CH₂NH₂, —CH₂NHMe, —CH₂NHEt, —CH₂NHiPr, —CH₂NHtBu, —CH₂NHCOMe, —CH₂CONH—SO₂Me, —CH₂CONH—CF₃, —CH₂CONH—SO₂-cyclopropyl, —SOMe, and —SO₂Me; R² is H; R³ is H; and R^{4c} is H.

In some embodiments, L is N; M is CH; G is CR¹; R² is H; R³ is H; and R^{4c} is H.

In some embodiments, R¹ is selected from —CH₂CH₂COOH, —CH₂CH₂CONH₂, —CH₂CH₂CONHSO₂Me, and —CH₂CH₂CONHSO₂cyclopropyl; R² is H; R³ is H; and R^{4c} is H.

In some embodiments, R¹ is selected from C(O)NHR¹⁵; R² is H; R³ is H; R^{4c} is H; and R¹⁵ is selected from H, —CH₂CH₂OH, —CH₂CH₂OMe, —CH₂CH₂NMe₂, —CH₂CH₂OCH₂CH₂OMe, —CH₂CH(OH)CH₂OH, —CH₂CH(OH)CH₂NEt₂, —CH₂CONHMe, —CH₂CMe₂OH, —CH₂CH(OH)CF₃, —CH₂CH₂SO₂Me, —CH₂-(3-oxetanyl)—CH₂OH, —CH₂-4-(2,2-dimethyl-1,3-dioxolanyl), —CH₂-thiazolyl, —OMe, —OCH₂CH₂OMe and

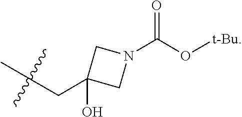 [SMILES: *CC1(O)CN(C(=O)OC(C)(C)C)C1]

In some embodiments, R¹ is selected from —CH(OH)CH₃, —COMe, —CONH₂, —CH₂OH, —SO₂Me, —SOCH₂CH₂OMe, —SO₂CH₂CH₂OMe, —SO₂CH₂CH₂NHMe

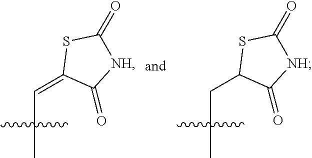 [SMILES: */C=C1/SC(=O)NC1=O ; *CC1SC(=O)NC1=O]

R² is F; R³ is H; and R^{4c} is H.

In some embodiments, R¹ is selected from Me, —CH₂COOH, —CH₂CH₂COOH, —CH₂CH₂SO₂NH₂, —SO₂Me, —SO₂NHMe,

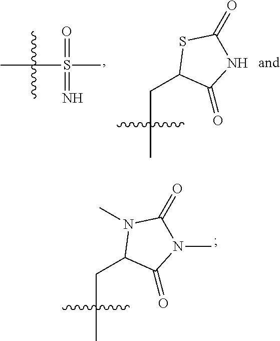

R² is F; R³ is H; and R^{4c} is F.

In some embodiments, L is CH; M is N; G is CH; R² is H; R³ is H; R^{4c} is H.

In some embodiments, L is CH; M is CH; G is CH; R² is H; R³ is H; R^{4c} is H.

In some embodiments, M is CR⁹, where R⁹ is selected from —CH₂COOH and —CH₂OH.

In some embodiments, M is CR⁹, where R⁹ taken together with R¹ and any intervening atoms forms an N-containing 6-membered heterocycle.

In some embodiments, M is CR⁹, where R⁹ taken together with R¹ and any intervening atoms forms an N-containing 7-membered heterocycle.

In some embodiments, M is CR⁹, where R⁹ taken together with R¹ and any intervening atoms forms an N-containing 6-membered heterocycle substituted with carboxyl.

In some embodiments, M is CR⁹, where R⁹ taken together with R¹ and any intervening atoms forms an N-containing 7-membered heterocycle substituted with carboxyl.

In some embodiments, M is C—CH₂COOH; R¹ is Me; R² is F; R³ is H; and R^{4c} is F.

In some embodiments, M is C—CH₂OH; R¹ is Me; R² is H; R³ is H; and G is N.

In some embodiments, R¹ is F; R² is F; R³ is H; and R^{4c} is H.

In some embodiments, R¹ is F; R² is H; R³ is H; and R^{4c} is H.

In some embodiments, R¹ is F; R² is F; R³ is H; and R^{4c} is F.

In some embodiments, R¹ is H; R² is F; R³ is H; and R^{4c} is H.

In some embodiments, R¹ is H; R² is H; R³ is F; and R^{4c} is H.

In some embodiments, R¹ is H; R² is H; R³ is H; and R^{4c} is H.

### Variables Q, K, and R⁷ of Formula (IA′)

Below are exemplary embodiments of variables Q, K, and R⁷ of the disclosed compound of Formula (IA′). The values for the remaining variables are as described above and below.

In some embodiments Q is CR^{4c}; K is CH; R^{4c} is H; R⁷ is H.

In some embodiments Q is CR^{4c}; K is N; R^{4c} is H; R⁷ is H.

In some embodiments Q is N; R⁷ is H.

In some embodiments Q is N; R⁷ is NH₂.

In some embodiments Q is CR^{4c}; R^{4c} is F; R⁷ is H.

Variables R⁵, R⁶, and R⁸ of Formula (IA′)

Below are exemplary embodiments of variables R⁵, R⁶, and R⁸ of the disclosed compound of Formula (IA′). The values for the remaining variables are as described above and below.

In some embodiments, R⁸ is halo.

In some embodiments, R⁸ is selected from haloalkyl and unsubstituted alkyl.

In some embodiments, R⁵ is selected from H, hydroxy, methyl, NH₂, CF₃, —COOH, —CH₂CH₂COOH, —OCH₂OH, —OCH₂COOH, —OCH₂CH₂NH₂, and —OCH₂CH(OH)CH₂OH; and R⁶ is selected from H and CH₃.

In some embodiments, R⁵ and R⁶ are each H.

In some embodiments, R⁵ is hydroxy and R⁶ is H.

In some embodiments, R⁵ is hydroxy or methyl, and R⁶ is methyl.

In some embodiments, R⁵ is amino or methyl, and R⁶ is H.

### Compounds of Formula IB′

In some embodiments, the compound of Formula (I) has the structure of compound (IB′):

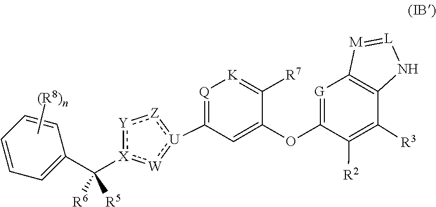

wherein
U is C;
W is N or NR^{a}, wherein R^{a} is H;
X is N or C;
Y is CR^{b}, wherein R^{b} is H, alkyl or carboxy, N, or NR^{c}, where R^{c} is alkyl;
Z is O, S, N or NR^{d}, where R^{d} is H;
G is N or CR¹, wherein R¹ is H, halo, alkyl, alkenyl, amido, sulfinyl, sulfonyl and sulfonamido;
R² is H or halo;
R³ is H or halo;
L is N or CH;
M is N or CR⁹, wherein R⁹ is H, —CH₂COOH, —CH₂OH, or R⁹ taken together with R¹ and any intervening atoms forms an N-containing 6- or 7-membered heterocycle, optionally substituted with oxo;
Q is N or CR^{4c};
R^{4c} is selected from H or halo;
K is N or CH;
R⁷ is H or amino;
R⁵ is selected from H, alkyl, hydroxy, alkoxy, carboxy, and amino;
R⁶ is H or alkyl;
R⁸ is halo, haloalkyl, or unsubstituted alkyl; and
n=0-1;
wherein each alkyl, alkenyl, amino, amido, and alkoxy is independently unsubstituted or substituted with one or more substituents selected from halo, hydroxy, amino, amido, alkyl, alkoxy, carboxy, heterocyclyl, and sulfonyl; wherein the alkyl substituent is optionally further substituted with one or more substituents selected from halo, hydroxy, amino, amido, carboxy, and sulfonamido.

### Variables W, X, Y, and Z of Formula (IB′)

Below are exemplary embodiments of variables U, W, X, Y, and Z of the disclosed compound of Formula (IB′). The values for the remaining variables are as described above and below.

In some embodiments, W is NH; X is C; Y is CH; and Z is N.

In some embodiments, W is NH; X is C; Y is N; and Z is N.

In some embodiments, W is N; X is C; Y is NH; and Z is N.

In some embodiments, W is N; X is C; Y is CH; and Z is NH.

In some embodiments, W is N; X is C; Y is CR^{b}; and Z is NH.

In some embodiments, W is N; X is C; Y is C; and Z is N.

In some embodiments, W is N; X is C; Y is N; and Z is NH.

In some embodiments, W is N; X is N; Y is CH; and Z is N.

In some embodiments, W is N; X is C; Y is CH; and Z is O.

In some embodiments, W is N; X is C; Y is CH; and Z is S.

Variables R¹, R², R³, L, M, and G of Formula (IB′)

Below are exemplary embodiments of variables R¹, R², R³, M, and G of the disclosed compound of Formula (IB′). The values for the remaining variables are as described above and below.

In some embodiments, L is CH; G is N; M is CH; R² is H; R³ is H; R^{4c} is H. In some embodiments, G is N; M is —CCH₂OH; R² is H; R³ is H; R^{4c} is H.

In some embodiments, G is CR¹, and R¹ is selected from —COOH, —CH₂OH, —CH₂COOH, —CH₂NH₂, —CH₂NHMe, —CH₂NHEt, —CH₂NHiPr, —CH₂NHtBu, —CH₂NHCOMe, —CH₂CONH—SO₂Me, —CH₂CONH—CF₃, —CH₂CONH—SO₂-cyclopropyl, —SOMe, and —SO₂Me; R² is H; R³ is H; and R^{4c} is H.

In some embodiments, L is N; M is CH; G is CR¹; R² is H; R³ is H; and R^{4c} is H.

In some embodiments, R¹ is selected from —CH₂CH₂COOH, —CH₂CH₂CONH₂, —CH₂CH₂CONHSO₂Me, and —CH₂CH₂CONHSO₂cyclopropyl; R² is H; R³ is H; and R^{4c} is H.

In some embodiments, R¹ is selected from C(O)NHR¹⁵; R² is H; R³ is H; R^{4c} is H; and R¹⁵ is selected from H, —CH₂CH₂OH, —CH₂CH₂OMe, —CH₂CH₂NMe₂, —CH₂CH₂OCH₂CH₂OMe, —CH₂CH(OH)CH₂OH, —CH₂CH(OH)CH₂NEt₂, —CH₂CONHMe, —CH₂CMe₂OH, —CH₂CH(OH)CF₃, —CH₂CH₂SO₂Me, —CH₂-(3-oxetanyl)—CH₂OH, —CH₂-4-(2,2-dimethyl-1,3-dioxolanyl), —CH₂-thiazolyl, —OMe, —OCH₂CH₂OMe and

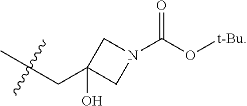 [SMILES: *CC1(O)CN(C(=O)OC(C)(C)C)C1]

In some embodiments, R¹ is selected from —CH(OH)CH₃, —COMe, —CONH₂, —CH₂OH, —SO₂Me, —SOCH₂CH₂OMe, —SO₂CH₂CH₂OMe, —SO₂CH₂CH₂NHMe

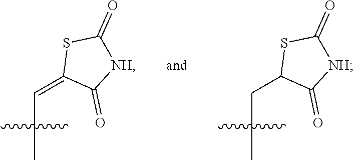 [SMILES: */C=C1/SC(=O)NC1=O ; *CC1SC(=O)NC1=O]

R² is F; R³ is H; and R^{4c} is H.

In some embodiments, R¹ is selected from Me, —CH₂COOH, —CH₂CH₂COOH, —(CH₂CH₂SO₂NH₂, —SO₂Me, —SO₂NHMe,

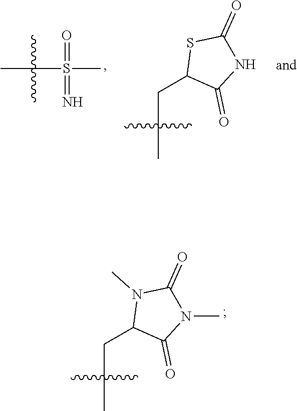

R² is F; R³ is H; and R^{4c} is F.

In some embodiments, L is CH; M is N; G is CH; R² is H; R³ is H; R^{4c} is H.

In some embodiments, L is CH; M is CH; G is CH; R² is H; R³ is H; R^{4c} is H.

In some embodiments, M is CR⁹, where R⁹ is selected from —CH₂COOH and —CH₂OH.

In some embodiments, M is CR⁹, where R⁹ taken together with R¹ and any intervening atoms forms an N-containing 6-membered heterocycle.

In some embodiments, M is CR⁹, where R⁹ taken together with R¹ and any intervening atoms forms an N-containing 7-membered heterocycle.

In some embodiments, M is CR⁹, where R⁹ taken together with R¹ and any intervening atoms forms an N-containing 6-membered heterocycle substituted with carboxyl.

In some embodiments, M is CR⁹, where R⁹ taken together with R¹ and any intervening atoms forms an N-containing 7-membered heterocycle substituted with carboxyl.

In some embodiments, M is C—CH₂COOH; R¹ is Me; R² is F; R³ is H; and R^{4c} is F.

In some embodiments, M is C—CH₂OH; R¹ is Me; R² is H; R³ is H; and G is N.

In some embodiments, R¹ is F; R² is F; R³ is H; and R^{4c} is H.

In some embodiments, R¹ is F; R² is H; R³ is H; and R^{4c} is H.

In some embodiments, R¹ is F; R² is F; R³ is H; and R^{4c} is F.

In some embodiments, R¹ is H; R² is F; R³ is H; and R^{4c} is H.

In some embodiments, R¹ is H; R² is H; R³ is F; and R^{4c} is H.

In some embodiments, R¹ is H; R² is H; R³ is H; and R^{4c} is H.

### Variables Q, K, and R⁷ of Formula (IB′)

Below are exemplary embodiments of variables Q, K, and R⁷ of the disclosed compound of Formula (IB′). The values for the remaining variables are as described above and below.

In some embodiments Q is CR^{4c}; K is CH; R^{4c} is H; R⁷ is H.

In some embodiments Q is CR^{4c}; K is N; R^{4c} is H; R⁷ is H.

In some embodiments Q is N; R⁷ is H.

In some embodiments Q is N; R⁷ is NH₂.

In some embodiments Q is CR^{4c}; R^{4c} is F; R⁷ is H.

Variables R⁵, R⁶, and R⁸ of Formula (IB′)

Below are exemplary embodiments of variables R⁵, R⁶, and R⁸ of the disclosed compound of Formula (IB′). The values for the remaining variables are as described above and below.

In some embodiments, R⁸ is halo.

In some embodiments, R⁸ is selected from haloalkyl and unsubstituted alkyl.

In some embodiments, R⁵ is selected from H, hydroxy, methyl, NH₂, CF₃, —COOH, —CH₂CH₂COOH, —OCH₂OH, —OCH₂COOH, —OCH₂CH₂NH₂, and —OCH₂CH(OH)CH₂OH; and R⁶ is selected from H and CH₃.

In some embodiments, R⁵ and R⁶ are each H.

In some embodiments, R⁵ is hydroxy and R⁶ is H.

In some embodiments, R⁵ is hydroxy or methyl, and R⁶ is methyl.

In some embodiments, R⁵ is amino or methyl, and R⁶ is H.

The present invention also provides compounds of Formula (II):

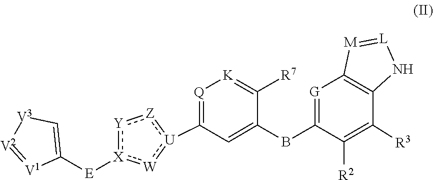

wherein
U is C;
W is N or NR^{a}, wherein R^{a} is H;
X is N or C;
Y is CR^{b}, wherein R^{b} is H, alkyl or carboxy, N, or NR^{c}, where R^{c} is alkyl;
Z is O, S, N or NR^{d}, where R^{d} is H; B is —O—, —C(O)—, —S—, —S(O)—, —S(O)₂—, or —C(R¹⁰R¹¹)—, wherein R¹⁰ and R¹¹ are independently selected from H, hydroxy, and —(CH₂)pJ, wherein p=1-3, and J is —C(O)NH₂, —COOH, or —CN;
G is N or CR¹, wherein R¹ is H, halo, alkyl, alkenyl, amido, sulfinyl, sulfonyl and sulfonamido;
R² is H or halo;
R³ is H or halo;
L is N or CH;
M is N or CR⁹, wherein R⁹ is H, —CH₂COOH, —CH₂OH, or R⁹ taken together with R¹ and any intervening atoms forms an N-containing 6- or 7-membered heterocycle, optionally substituted with oxo;
Q is N or CR^{4c};
R^{4c} is selected from H or halo;
K is N or CH;
R⁷ is H or amino;
E is —S(O)—, —S(O)₂—, or —C(R⁵R⁶)—, wherein
R⁵ is selected from H, alkyl, hydroxy, alkoxy, carboxy, and amino;
R⁶ is H or alkyl;
V¹ is N or CH;
V² is N or CH;
V³ is NR¹² or CHR¹²; and
R¹² is H, unsubstituted alkyl or haloalkyl;
wherein each alkyl, alkenyl, amino, amido, and alkoxy is independently unsubstituted or substituted with one or more substituents selected from halo, hydroxy, amino, amido, alkyl, alkoxy, carboxy, heterocyclyl, and sulfonyl; wherein the alkyl substituent is optionally further substituted with one or more substituents selected from halo, hydroxy, amino, amido, carboxy, and sulfonamido.

### Variables W, X, Y, and Z of Formula (II)

Below are exemplary embodiments of variables U, W, X, Y, and Z of the disclosed compound of Formula (II). The values for the remaining variables are as described above and below.

In some embodiments, W is NH; X is C; Y is CH; and Z is N.

In some embodiments, W is NH; X is C; Y is N; and Z is N.

In some embodiments, W is N; X is C; Y is NH; and Z is N.

In some embodiments, W is N; X is C; Y is CH; and Z is NH.

In some embodiments, W is N; X is C; Y is CR^{b}; and Z is NH.

In some embodiments, W is N; X is C; Y is C; and Z is N.

In some embodiments, W is N; X is C; Y is N; and Z is NH.

In some embodiments, W is N; X is N; Y is CH; and Z is N.

In some embodiments, W is N; X is C; Y is CH; and Z is O.

In some embodiments, W is N; X is C; Y is CH; and Z is S.

Variables R¹, R², R³, L, M, and G of Formula (II)

Below are exemplary embodiments of variables R¹, R², R³, M, and G of the disclosed compound of Formula (II). The values for the remaining variables are as described above and below.

In some embodiments, L is CH; G is N; M is CH; R² is H; R³ is H; R^{4c} is H.

In some embodiments, G is N; M is CCH₂OH; R² is H; R³ is H; R^{4c} is H.

In some embodiments, G is CR¹, and R¹ is selected from —COOH, —CH₂OH, —CH₂COOH, —CH₂NH₂, —CH₂NHMe, —CH₂NHEt, —CH₂NHiPr, —CH₂NHtBu, —CH₂NHCOMe, —CH₂CONH—SO₂Me, —CH₂CONH—CF₃, —CH₂CONH—SO₂-cyclopropyl, —SOMe, and —SO₂Me; R² is H; R³ is H; and R^{4c} is H.

In some embodiments, L is N; M is CH; G is CR¹; R² is H; R³ is H; and R^{4c} is H.

In some embodiments, R¹ is selected from —CH₂CH₂COOH, —CH₂CH₂CONH₂, —CH₂CH₂CONHSO₂Me, and —CH₂CH₂CONHSO₂cyclopropyl; R² is H; R³ is H; and R^{4c} is H.

In some embodiments, R¹ is selected from C(O)NHR¹⁵; R² is H; R³ is H; R^{4c} is H; and R¹⁵ is selected from H, —CH₂CH₂OH, —CH₂CH₂OMe, —CH₂CH₂NMe₂, —CH₂CH₂OCH₂CH₂OMe, —CH₂CH(OH)CH₂OH, —CH₂CH(OH)CH₂NEt₂, —CH₂CONHMe, —CH₂CMe₂OH, —CH₂CH(OH)CF₃, —CH₂CH₂SO₂Me, —CH₂-(3-oxetanyl)—CH₂OH, —CH₂-4-(2,2-dimethyl-1,3-dioxolanyl), —CH₂-thiazolyl, —OMe, —OCH₂CH₂OMe and

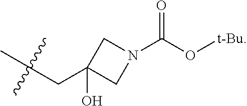 [SMILES: *CC1(O)CN(C(=O)OC(C)(C)C)C1]

In some embodiments, R¹ is selected from —CH(OH)CH₃, —COMe, —CONH₂, —CH₂OH, —SO₂Me, —SOCH₂CH₂OMe, —SO₂CH₂CH₂OMe, —SO₂CH₂CH₂NHMe

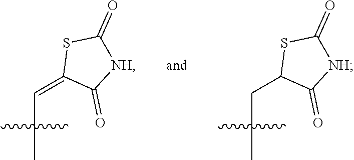 [SMILES: */C=C1/SC(=O)NC1=O ; *CC1SC(=O)NC1=O]

R² is F; R³ is H; and R^{4c} is H.

In some embodiments, R¹ is selected from Me, —CH₂COOH, —CH₂CH₂COOH, —CH₂CH₂SO₂NH₂, —SO₂Me, —SO₂NHMe,

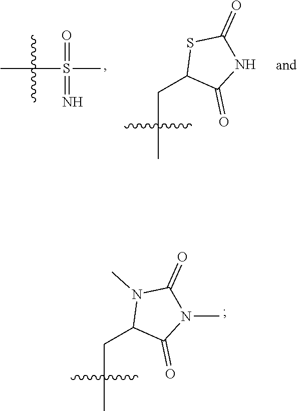

R² is F; R³ is H; and R^{4c} is F.

In some embodiments, L is CH; M is N; G is CH; R² is H; R³ is H; R^{4c} is H.

In some embodiments, L is CH; M is CH; G is CH; R² is H; R³ is H; R^{4c} is H.

In some embodiments, M is CR⁹, where R⁹ is selected from —CH₂COOH and —CH₂OH.

In some embodiments, M is CR⁹, where R⁹ taken together with R¹ and any intervening atoms forms an N-containing 6-membered heterocycle.

In some embodiments, M is CR⁹, where R⁹ taken together with R¹ and any intervening atoms forms an N-containing 7-membered heterocycle.

In some embodiments, M is CR⁹, where R⁹ taken together with R¹ and any intervening atoms forms an N-containing 6-membered heterocycle substituted with carboxyl.

In some embodiments, M is CR⁹, where R⁹ taken together with R¹ and any intervening atoms forms an N-containing 7-membered heterocycle substituted with carboxyl.

In some embodiments, M is CCH₂COOH; R¹ is Me; R² is F; R³ is H; and R^{4c} is F.

In some embodiments, M is CCH₂OH; R¹ is Me; R² is H; R³ is H; and G is N.

In some embodiments, R¹ is F; R² is F; R³ is H; and R^{4c} is H.

In some embodiments, R¹ is F; R² is H; R³ is H; and R^{4c} is H.

In some embodiments, R¹ is F; R² is F; R³ is H; and R^{4c} is F.

In some embodiments, R¹ is H; R² is F; R³ is H; and R^{4c} is H.

In some embodiments, R¹ is H; R² is H; R³ is F; and R^{4c} is H.

In some embodiments, R¹ is H; R² is H; R³ is H; and R^{4c} is H.

### Variables B, Q, K, and R⁷ of Formula (II)

Below are exemplary embodiments of variables B, Q, K, and R⁷ of the disclosed compound of Formula (I). The values for the remaining variables are as described above and below.

In some embodiments B is —O—; Q is CR^{4c}; K is CH; R^{4c} is H; R⁷ is H.

In some embodiments B is —O—; Q is CR^{4c}; K is N; R^{4c} is H; R⁷ is H.

In some embodiments B is —O—; Q is N; R⁷ is H.

In some embodiments B is —O—; Q is N; R⁷ is NH₂.

In some embodiments B is —O—; Q is CR^{4c}; R^{4c} is F; R⁷ is H.

In some embodiments B is selected from —C(O)—, —S—, —S(O)—, —S(O)₂—. In some embodiments, B is —C(R¹⁰R¹¹)—, where R¹⁰ and R¹¹ are independently selected from H, hydroxy, and —(CH₂)pJ.

In some embodiments p=1.

In some embodiments, J is —C(O)NH₂, —COOH, or —CN.

Variables E, V¹, V², and V³ of Formula I(II)

Below are exemplary embodiments of variables E and R⁸ of the disclosed compound of Formula (II). The values for the remaining variables are as described above and below.

In some embodiments, E is selected from —S(O)— and —S(O)₂—.

In some embodiments, E is —C(R⁵R⁶)—, where R⁵ is selected from H and hydroxy; R⁶ is H.

In some embodiments, E is —C(R⁵R⁶)—, where R⁵ is selected from H and hydroxy; R⁶ is alkyl.

In some embodiments, E is —C(R⁵R⁶)—, wherein R⁵ is selected from alkoxy, carboxy, and amino; R⁶ is H.

In some embodiments, E is —C(R⁵R⁶)—, wherein R⁵ is selected from alkoxy, carboxy, and amino; R⁶ is alkyl.

In some embodiments, R⁵ and R⁶ are H, R⁸ is halo.

In some embodiments, R⁸ is selected from halo, haloalkyl, or unsubstituted alkyl.

In some embodiments, R⁵ is selected from H, hydroxy, methyl, NH₂, CF₃, —COOH, —CH₂CH₂COOH, —OCH₂OH, —OCH₂COOH, —OCH₂CH₂NH₂, and —OCH₂CH(OH)CH₂OH; and R⁶ is selected from H and CH₃.

In some embodiments, R⁵ and R⁶ are each H.

In some embodiments, R⁵ is hydroxy and R⁶ is H.

In some embodiments, R⁵ is hydroxy or methyl, and R⁶ is methyl.

In some embodiments, R⁵ is amino or methyl, and R⁶ is H.

In some embodiments, V¹ is CH; V² is N; V³ is NR¹²; and R¹² is unsubstituted alkyl.

In some embodiments, V¹ is CH; V² is N; V³ is NR¹²; and R¹² is methyl.

In some embodiments, V¹ is CH; V² is N; V³ is NR¹²; R¹² is methyl; and R⁵ is hydroxy.

In some embodiments, V¹ is CH; V² is N; V³ is NR¹²; R¹² is methyl; R⁶ is H; and R⁵ is hydroxy.

In some embodiments, the compound of Formula (I) or Formula (II) is selected from the following compounds represented in Table 1 below:

TABLE 1

| | |
|---|---|
| 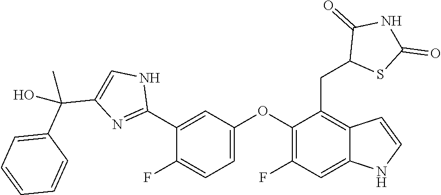 [SMILES: CC(O)(c1ccccc1)c1c[nH]c(-c2cc(Oc3c(F)cc4[nH]ccc4c3CC3SC(=O)NC3=O)ccc2F)n1] | 1 |
| 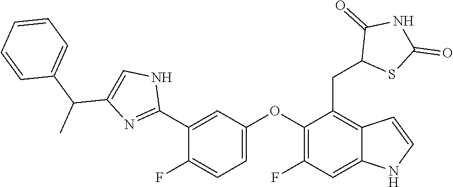 [SMILES: CC(c1ccccc1)c1c[nH]c(-c2cc(Oc3c(F)cc4[nH]ccc4c3CC3SC(=O)NC3=O)ccc2F)n1] | 2 |
| 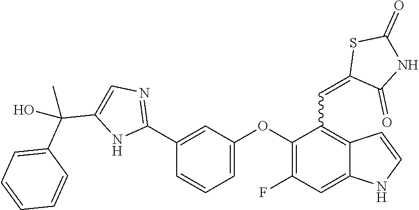 [SMILES: CC(O)(c1ccccc1)c1cnc(-c2cccc(Oc3c(F)cc4[nH]ccc4c3C=C3SC(=O)NC3=O)c2)[nH]1] | 3 |
| 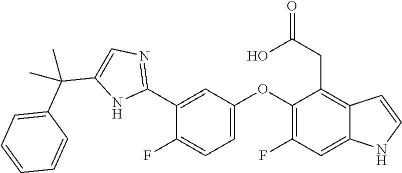 [SMILES: CC(C)(c1ccccc1)c1cnc(-c2cc(Oc3c(F)cc4[nH]ccc4c3CC(=O)O)ccc2F)[nH]1] | 4 |
| 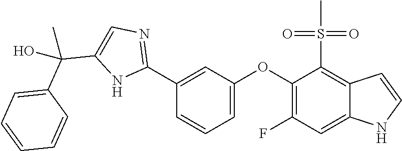 [SMILES: CC(O)(c1ccccc1)c1cnc(-c2cccc(Oc3c(F)cc4[nH]ccc4c3S(C)(=O)=O)c2)[nH]1] | 5 |
| 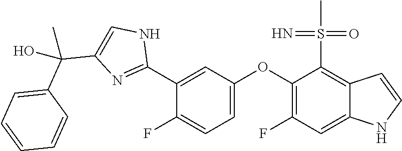 [SMILES: CC(O)(c1ccccc1)c1c[nH]c(-c2cc(Oc3c(F)cc4[nH]ccc4c3S(C)(=N)=O)ccc2F)n1] | 6 |
| 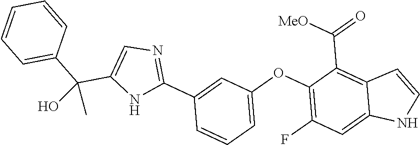 [SMILES: COC(=O)c1c(Oc2cccc(-c3ncc(C(C)(O)c4ccccc4)[nH]3)c2)c(F)cc2[nH]ccc12] | 7 |
| 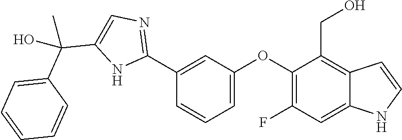 [SMILES: CC(O)(c1ccccc1)c1cnc(-c2cccc(Oc3c(F)cc4[nH]ccc4c3CO)c2)[nH]1] | 8 |
| 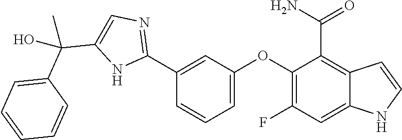 [SMILES: CC(O)(c1ccccc1)c1cnc(-c2cccc(Oc3c(F)cc4[nH]ccc4c3C(N)=O)c2)[nH]1] | 9 |
| 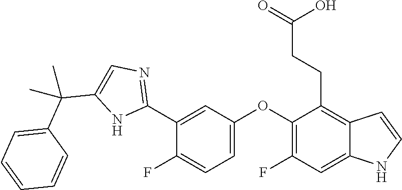 [SMILES: CC(C)(c1ccccc1)c1cnc(-c2cc(Oc3c(F)cc4[nH]ccc4c3CCC(=O)O)ccc2F)[nH]1] | 10 |
| 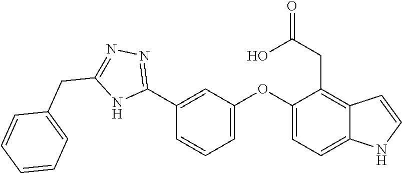 [SMILES: O=C(O)Cc1c(Oc2cccc(-c3nnc(Cc4ccccc4)[nH]3)c2)ccc2[nH]ccc12] | 11 |
| 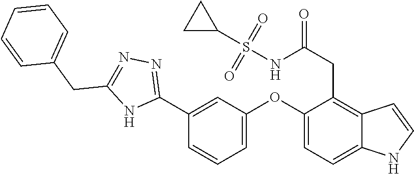 [SMILES: O=C(Cc1c(Oc2cccc(-c3nnc(Cc4ccccc4)[nH]3)c2)ccc2[nH]ccc12)NS(=O)(=O)C1CC1] | 12 |
| 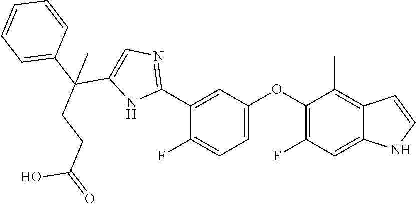 [SMILES: Cc1c(Oc2ccc(F)c(-c3ncc(C(C)(CCC(=O)O)c4ccccc4)[nH]3)c2)c(F)cc2[nH]ccc12] | 13 |
| 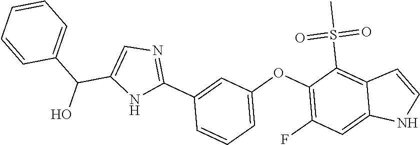 [SMILES: CS(=O)(=O)c1c(Oc2cccc(-c3ncc(C(O)c4ccccc4)[nH]3)c2)c(F)cc2[nH]ccc12] | 14 |
| 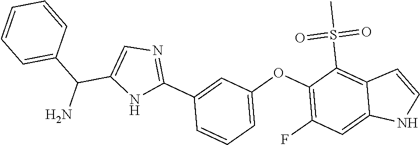 [SMILES: CS(=O)(=O)c1c(Oc2cccc(-c3ncc(C(N)c4ccccc4)[nH]3)c2)c(F)cc2[nH]ccc12] | 15 |
| 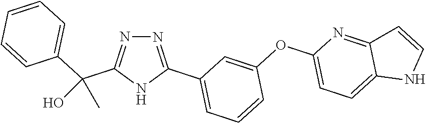 [SMILES: CC(O)(c1ccccc1)c1nnc(-c2cccc(Oc3ccc4[nH]ccc4n3)c2)[nH]1] | 16 |
| 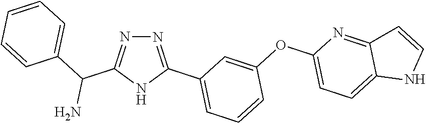 [SMILES: NC(c1ccccc1)c1nnc(-c2cccc(Oc3ccc4[nH]ccc4n3)c2)[nH]1] | 17 |
| 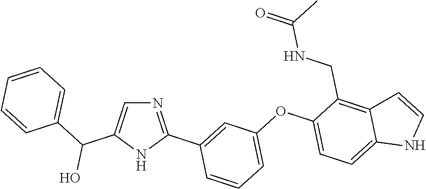 [SMILES: CC(=O)NCc1c(Oc2cccc(-c3ncc(C(O)c4ccccc4)[nH]3)c2)ccc2[nH]ccc12] | 18 |
| 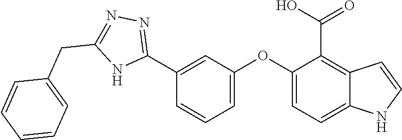 [SMILES: O=C(O)c1c(Oc2cccc(-c3nnc(Cc4ccccc4)[nH]3)c2)ccc2[nH]ccc12] | 19 |
| 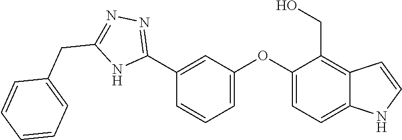 [SMILES: OCc1c(Oc2cccc(-c3nnc(Cc4ccccc4)[nH]3)c2)ccc2[nH]ccc12] | 20 |
| 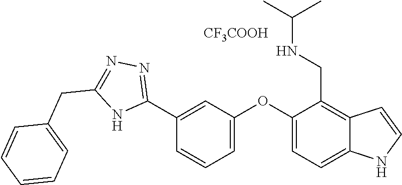 [SMILES: CC(C)NCc1c(Oc2cccc(-c3nnc(Cc4ccccc4)[nH]3)c2)ccc2[nH]ccc12 ; "CF3COOH"] | 21 |
| 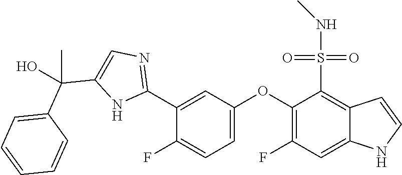 [SMILES: CNS(=O)(=O)c1c(Oc2ccc(F)c(-c3ncc(C(C)(O)c4ccccc4)[nH]3)c2)c(F)cc2[nH]ccc12] | 22 |
| 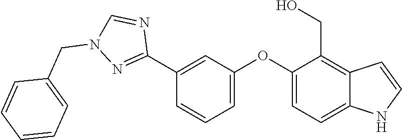 [SMILES: OCc1c(Oc2cccc(-c3ncn(Cc4ccccc4)n3)c2)ccc2[nH]ccc12] | 23 |
| 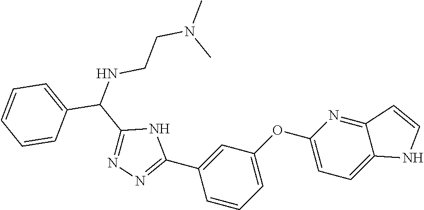 [SMILES: CN(C)CCNC(c1ccccc1)c1nnc(-c2cccc(Oc3ccc4[nH]ccc4n3)c2)[nH]1] | 24 |
| 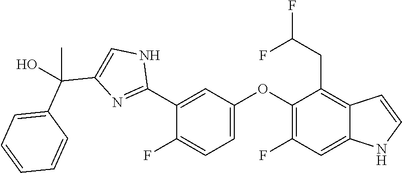 [SMILES: CC(O)(c1ccccc1)c1c[nH]c(-c2cc(Oc3c(F)cc4[nH]ccc4c3CC(F)F)ccc2F)n1] | 25 |
| 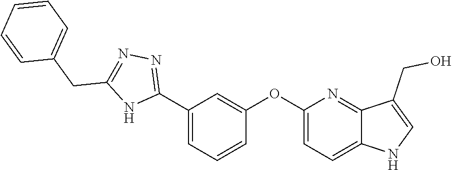 [SMILES: OCc1c[nH]c2ccc(Oc3cccc(-c4nnc(Cc5ccccc5)[nH]4)c3)nc12] | 26 |
| 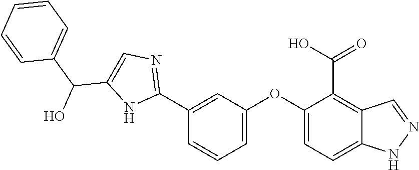 [SMILES: O=C(O)c1c(Oc2cccc(-c3ncc(C(O)c4ccccc4)[nH]3)c2)ccc2[nH]ncc12] | 27 |
| 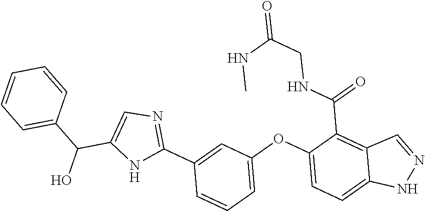 [SMILES: CNC(=O)CNC(=O)c1c(Oc2cccc(-c3ncc(C(O)c4ccccc4)[nH]3)c2)ccc2[nH]ncc12] | 28 |
| 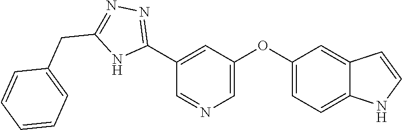 [SMILES: c1ccc(Cc2nnc(-c3cncc(Oc4ccc5[nH]ccc5c4)c3)[nH]2)cc1] | 29 |
| 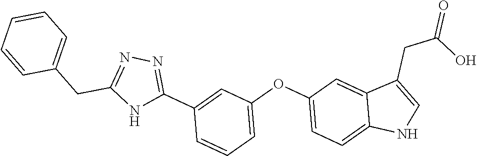 [SMILES: O=C(O)Cc1c[nH]c2ccc(Oc3cccc(-c4nnc(Cc5ccccc5)[nH]4)c3)cc12] | 30 |
| 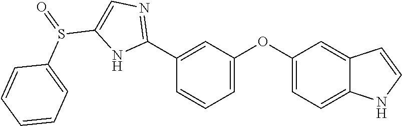 [SMILES: O=S(c1ccccc1)c1cnc(-c2cccc(Oc3ccc4[nH]ccc4c3)c2)[nH]1] | 31 |
| 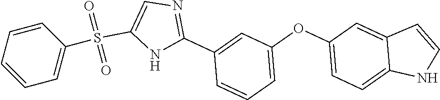 [SMILES: O=S(=O)(c1ccccc1)c1cnc(-c2cccc(Oc3ccc4[nH]ccc4c3)c2)[nH]1] | 32 |
| 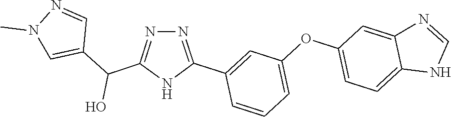 [SMILES: Cn1cc(C(O)c2nnc(-c3cccc(Oc4ccc5[nH]cnc5c4)c3)[nH]2)cn1] | 33 |
| 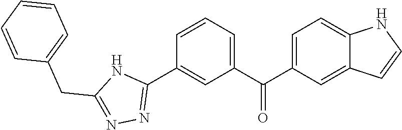 [SMILES: O=C(c1cccc(-c2nnc(Cc3ccccc3)[nH]2)c1)c1ccc2[nH]ccc2c1] | 34 |
| 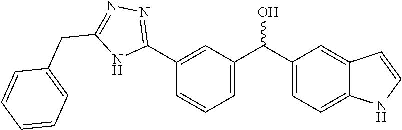 [SMILES: OC(c1cccc(-c2nnc(Cc3ccccc3)[nH]2)c1)c1ccc2[nH]ccc2c1] | 35A |
| 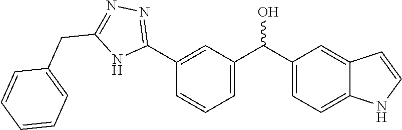 [SMILES: OC(c1cccc(-c2nnc(Cc3ccccc3)[nH]2)c1)c1ccc2[nH]ccc2c1] | 35B |
| 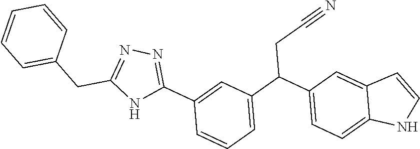 [SMILES: N#CCC(c1cccc(-c2nnc(Cc3ccccc3)[nH]2)c1)c1ccc2[nH]ccc2c1] | 36 |
| 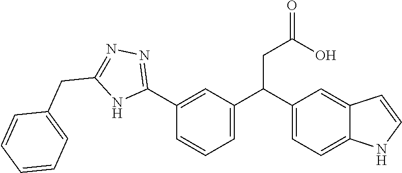 [SMILES: O=C(O)CC(c1cccc(-c2nnc(Cc3ccccc3)[nH]2)c1)c1ccc2[nH]ccc2c1] | 37 |
| 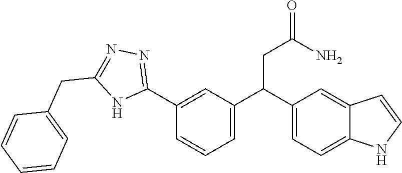 [SMILES: NC(=O)CC(c1cccc(-c2nnc(Cc3ccccc3)[nH]2)c1)c1ccc2[nH]ccc2c1] | 38 |
| 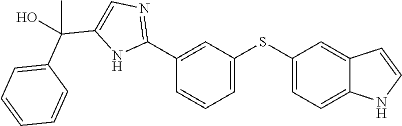 [SMILES: CC(O)(c1ccccc1)c1cnc(-c2cccc(Sc3ccc4[nH]ccc4c3)c2)[nH]1] | 39 |
| 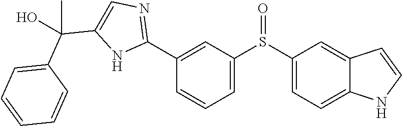 [SMILES: CC(O)(c1ccccc1)c1cnc(-c2cccc(S(=O)c3ccc4[nH]ccc4c3)c2)[nH]1] | 40 |
| 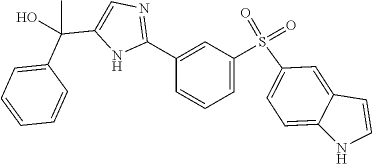 [SMILES: CC(O)(c1ccccc1)c1cnc(-c2cccc(S(=O)(=O)c3ccc4[nH]ccc4c3)c2)[nH]1] | 41 |
| 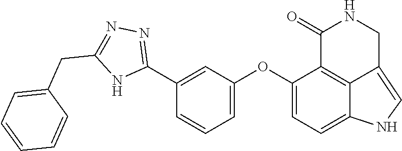 [SMILES: O=C1NCc2c[nH]c3ccc(Oc4cccc(-c5nnc(Cc6ccccc6)[nH]5)c4)c1c23] | 42 |
| 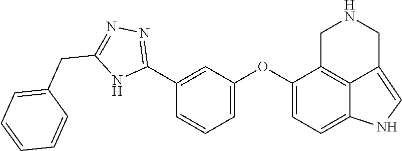 [SMILES: c1ccc(Cc2nnc(-c3cccc(Oc4ccc5[nH]cc6c5c4CNC6)c3)[nH]2)cc1] | 43 |
| 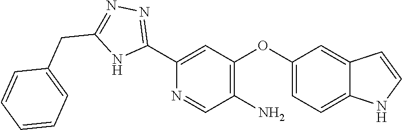 [SMILES: Nc1cnc(-c2nnc(Cc3ccccc3)[nH]2)cc1Oc1ccc2[nH]ccc2c1] | 44 |
| 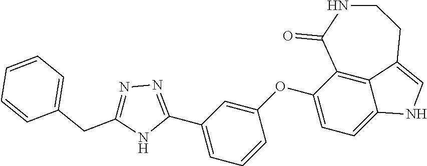 [SMILES: O=C1NCCc2c[nH]c3ccc(Oc4cccc(-c5nnc(Cc6ccccc6)[nH]5)c4)c1c23] | 45 |
| 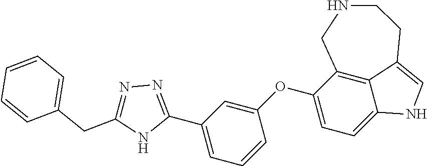 [SMILES: c1ccc(Cc2nnc(-c3cccc(Oc4ccc5[nH]cc6c5c4CNCC6)c3)[nH]2)cc1] | 46 |
| 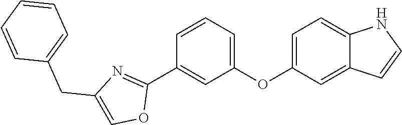 [SMILES: c1ccc(Cc2coc(-c3cccc(Oc4ccc5[nH]ccc5c4)c3)n2)cc1] | 47 |
| 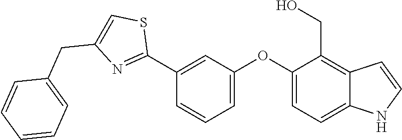 [SMILES: OCc1c(Oc2cccc(-c3nc(Cc4ccccc4)cs3)c2)ccc2[nH]ccc12] | 48 |
| 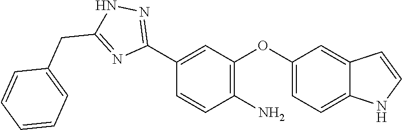 [SMILES: Nc1ccc(-c2n[nH]c(Cc3ccccc3)n2)cc1Oc1ccc2[nH]ccc2c1] | 49 |
| 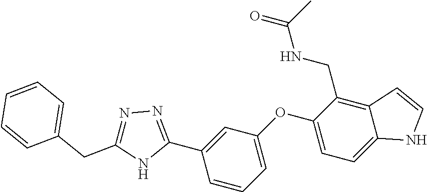 [SMILES: CC(=O)NCc1c(Oc2cccc(-c3nnc(Cc4ccccc4)[nH]3)c2)ccc2[nH]ccc12] | 50 |
| 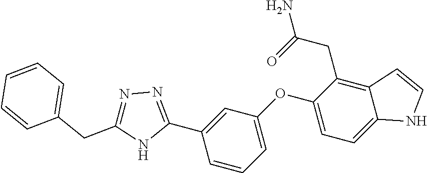 [SMILES: NC(=O)Cc1c(Oc2cccc(-c3nnc(Cc4ccccc4)[nH]3)c2)ccc2[nH]ccc12] | 51 |
| 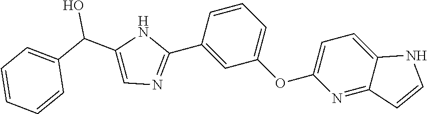 [SMILES: OC(c1ccccc1)c1cnc(-c2cccc(Oc3ccc4[nH]ccc4n3)c2)[nH]1] | 52 |
| 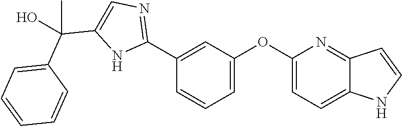 [SMILES: CC(O)(c1ccccc1)c1cnc(-c2cccc(Oc3ccc4[nH]ccc4n3)c2)[nH]1] | 53 |
| 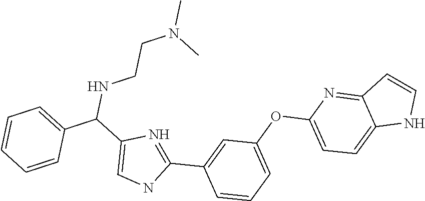 [SMILES: CN(C)CCNC(c1ccccc1)c1c[nH]c(-c2cccc(Oc3ccc4[nH]ccc4n3)c2)n1] | 54 |
| 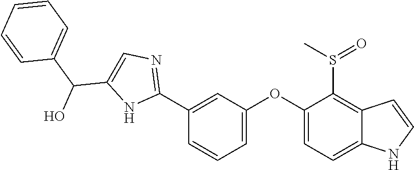 [SMILES: CS(=O)c1c(Oc2cccc(-c3ncc(C(O)c4ccccc4)[nH]3)c2)ccc2[nH]ccc12] | 55 |
| 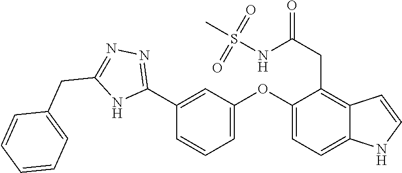 [SMILES: CS(=O)(=O)NC(=O)Cc1c(Oc2cccc(-c3nnc(Cc4ccccc4)[nH]3)c2)ccc2[nH]ccc12] | 56 |
| 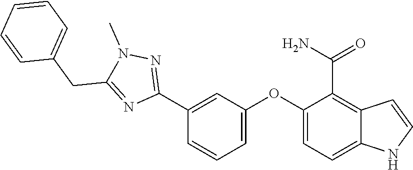 | 57 |
| 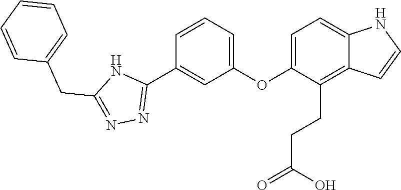 [SMILES: O=C(O)CCc1c(Oc2cccc(-c3nnc(Cc4ccccc4)[nH]3)c2)ccc2[nH]ccc12] | 58 |
| 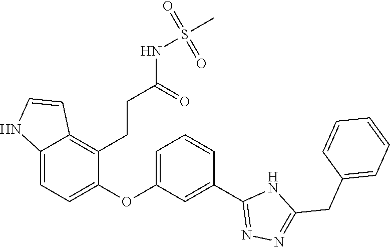 [SMILES: CS(=O)(=O)NC(=O)CCc1c(Oc2cccc(-c3nnc(Cc4ccccc4)[nH]3)c2)ccc2[nH]ccc12] | 59 |
| 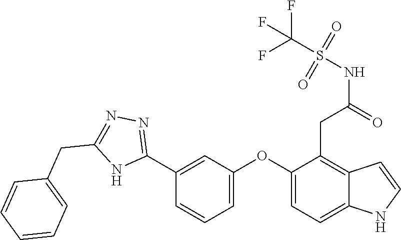 | 60 |
| 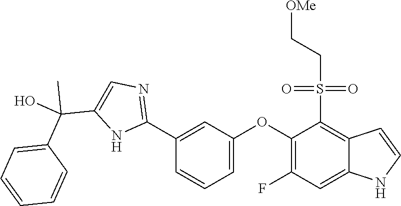 | 61 |
| 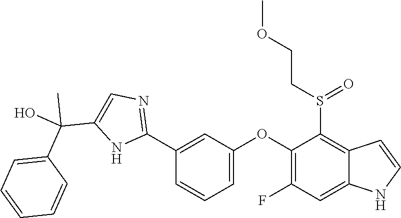 | 62 |
| 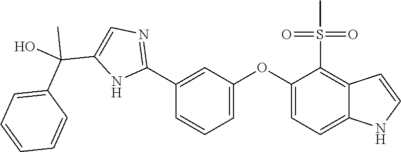 | 63 |
| 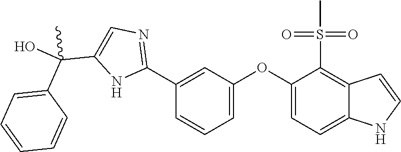 | 63A |
| 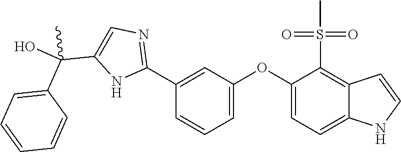 | 63B |
| 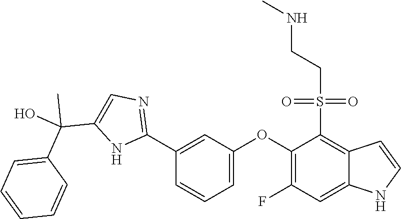 | 64 |
| 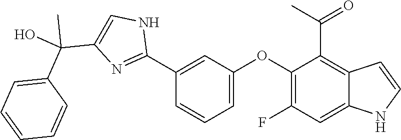 | 65 |
| 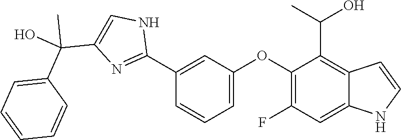 | 66 |
| 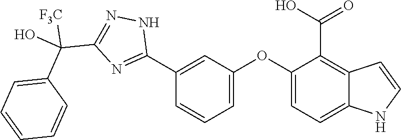 | 67 |
| 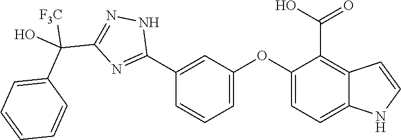 | 68 |
| 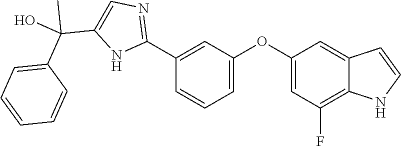 | 69 |
| 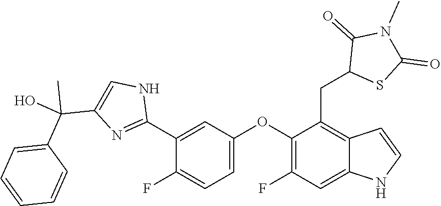 | 70 |
| 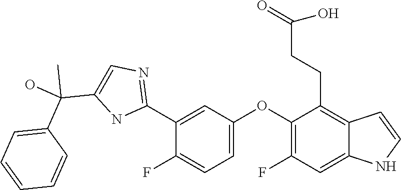 [SMILES: CC(O)(c1ccccc1)c1cnc(-c2cc(Oc3c(F)cc4[nH]ccc4c3CCC(=O)O)ccc2F)[nH]1] | 71 |
| 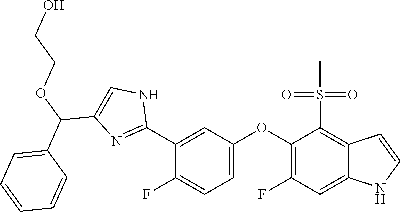 [SMILES: CS(=O)(=O)c1c(Oc2ccc(F)c(-c3nc(C(OCCO)c4ccccc4)c[nH]3)c2)c(F)cc2[nH]ccc12] | 72 |
| 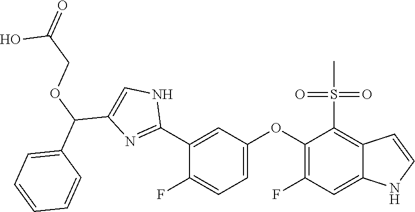 [SMILES: CS(=O)(=O)c1c(Oc2ccc(F)c(-c3nc(C(OCC(=O)O)c4ccccc4)c[nH]3)c2)c(F)cc2[nH]ccc12] | 73 |
| 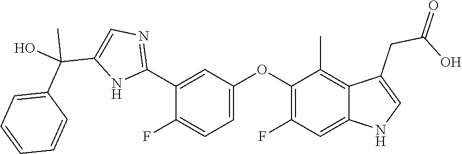 [SMILES: Cc1c(Oc2ccc(F)c(-c3ncc(C(C)(O)c4ccccc4)[nH]3)c2)c(F)cc2[nH]cc(CC(=O)O)c12] | 74 |
| 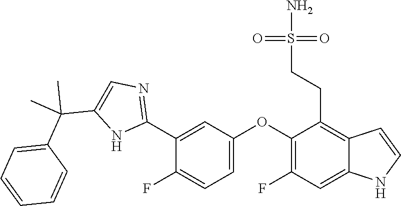 [SMILES: CC(C)(c1ccccc1)c1cnc(-c2cc(Oc3c(F)cc4[nH]ccc4c3CCS(N)(=O)=O)ccc2F)[nH]1] | 75 |
| 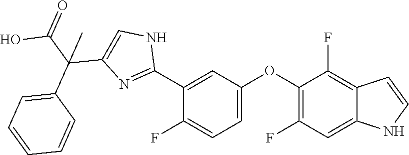 [SMILES: CC(C(=O)O)(c1ccccc1)c1c[nH]c(-c2cc(Oc3c(F)cc4[nH]ccc4c3F)ccc2F)n1] | 76 |
| 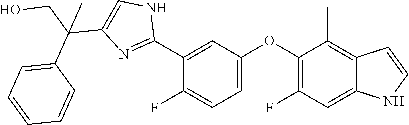 [SMILES: Cc1c(Oc2ccc(F)c(-c3nc(C(C)(CO)c4ccccc4)c[nH]3)c2)c(F)cc2[nH]ccc12] | 77 |
| 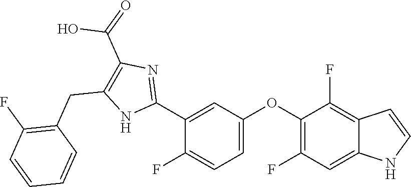 [SMILES: O=C(O)c1nc(-c2cc(Oc3c(F)cc4[nH]ccc4c3F)ccc2F)[nH]c1Cc1ccccc1F] | 78 |
| 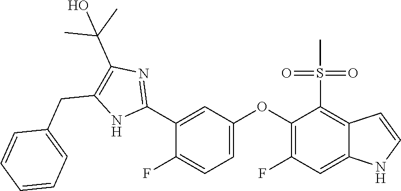 [SMILES: CC(C)(O)c1nc(-c2cc(Oc3c(F)cc4[nH]ccc4c3S(C)(=O)=O)ccc2F)[nH]c1Cc1ccccc1] | 79 |
| 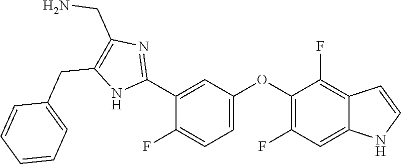 [SMILES: NCc1nc(-c2cc(Oc3c(F)cc4[nH]ccc4c3F)ccc2F)[nH]c1Cc1ccccc1] | 80 |
| 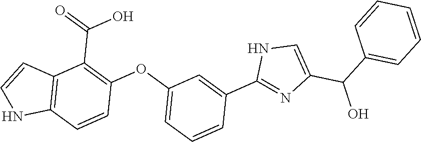 [SMILES: O=C(O)c1c(Oc2cccc(-c3nc(C(O)c4ccccc4)c[nH]3)c2)ccc2[nH]ccc12] | 81 |
| 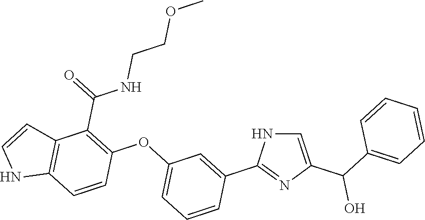 [SMILES: COCCNC(=O)c1c(Oc2cccc(-c3nc(C(O)c4ccccc4)c[nH]3)c2)ccc2[nH]ccc12] | 82 |
| 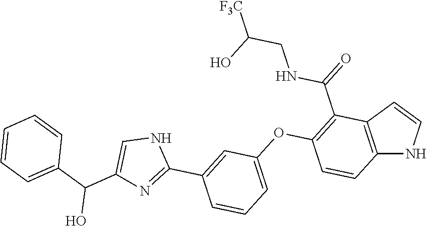 [SMILES: O=C(NCC(O)C(F)(F)F)c1c(Oc2cccc(-c3nc(C(O)c4ccccc4)c[nH]3)c2)ccc2[nH]ccc12] | 83 |

In certain embodiments, the present invention provides a pharmaceutical preparation suitable for use in a subject, comprising any of the compounds shown above (e.g., a compound of the invention, such as a compound of formula (I), and one or more pharmaceutically acceptable excipients. In certain embodiments, the pharmaceutical preparations may be for use in treating or preventing cystic fibrosis.

Any of the disclosed compounds may be used in the manufacture of medicaments for the treatment of any diseases or conditions disclosed herein.

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to a straight chained or branched aliphatic group containing at least one double bond. Typically, an alkenyl group has from 2 to about 20 carbon atoms, preferably from 2 to about 10, more preferably from 2-6 or 2-4. unless otherwise defined. The term “alkenyl” is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds.

Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10, more preferably from 1-6 or 1-4. unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “haloalkyl”, as used herein, refers to an alkyl group in which at least one hydrogen has been replaced with a halogen, such as fluoro, chloro, bromo, or iodo.

Exemplary haloalkyl groups include trifluoromethyl, difluoromethyl, fluoromethyl, 2-fluoroethyl, 2,2-difluoroethyl, and 2,2,2-trifluoroethyl.

The term “alkynyl”, as used herein, refers to a straight chained or branched aliphatic group containing at least one triple bond. Typically, an alkenyl group has from 2 to about 20 carbon atoms, preferably from 2 to about 10, more preferably from 2-6 or 2-4. unless otherwise defined. The term “alkynyl” is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

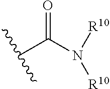

wherein each R¹⁰ independently represents a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

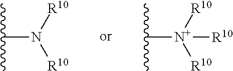

wherein each R¹⁰ independently represents a hydrogen or a hydrocarbyl group, or two R¹⁰ are taken together with the N atom to which they are attached complete a heterocycle having from 4 to 8 atoms in the ring structure. The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably, the ring is a 6- to 10-membered ring, such as a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

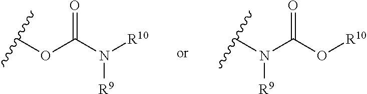

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring.

Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 9 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings.

A “cycloalkenyl” group is a cyclic hydrocarbon containing one or more double bonds. The cycloalkenyl ring may have 3 to 10 carbon atoms, such as 4 to 9 carbon atoms. As such, cycloalkenyl groups can be monocyclic or multicyclic. Individual rings of such multicyclic cycloalkenyl groups can have different connectivities, e.g., fused, bridged, spiro, etc. in addition to covalent bond substitution. Exemplary cycloalkenyl groups include cyclopropenyl, cyclobutenyl, cyclopentyl, cyclohexenyl, cycloheptenyl, 1,3-cyclohexadienyl, 1,4-cyclohexadienyl and 1,5-cyclooctadienyl.

Exemplary cycloalkyl groups include cyclopropyl, cyclobutyl, cyclopentyl, cyclohexyl, norbornanyl, bicyclo[3.2.1]octanyl, octahydro-pentalenyl, spiro[4.5]decanyl, cyclopropyl, and adamantyl.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical.

Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 3- to 10-membered rings, more preferably 5- to 9-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

Individual rings of such multicyclic heteroaryl groups can have different connectivities, e.g., fused, etc. in addition to covalent bond substitution. Exemplary heteroaryl groups include furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5,6,7,8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl and benzoxazinyl, etc. In general, the heteroaryl group typically is attached to the main structure via a carbon atom.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

Individual rings of such multicyclic heterocycloalkyl groups can have different connectivities, e.g., fused, bridged, spiro, etc. in addition to covalent bond substitution. Exemplary heterocycloalkyl groups include pyrrolidinyl, tetrahydrofuranyl, dihydrofuranyl, tetrahydropyranyl, pyranyl, thiopyranyl, azindinyl, azetidinyl, oxiranyl, methylenedioxyl, chromenyl, barbituryl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, piperizin-2-onyl, piperizin-3-onyl, chromanyl, 2-pyrrolinyl, 3-pyrrolinyl, imidazolidinyl, 2-imidazolidinyl, 1,4-dioxanyl, 8-azabicyclo[3.2.1]octanyl, 3-azabicyclo[3.2.1]octanyl, 3,8-diazabicyclo[3.2.1]octanyl, 2,5-diazabicyclo[2.2.1]heptanyl, 2,5-diazabicyclo[2.2.2]octanyl, octahydro-2H-pyrido[1,2-a]pyrazinyl, 3-azabicyclo[4.1.0]heptanyl, 3-azabicyclo[3.1.0]hexanyl 2-azaspiro[4.4]nonanyl, 7-oxa-1-aza-spiro[4.4]nonanyl, 7-azabicyclo[2.2.2]heptanyl, octahydro-1H-indolyl, etc. In general, the heterocycloalkyl group typically is attached to the main structure via a carbon atom or a nitrogen atom. The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms.

Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof. The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

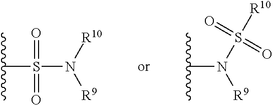

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

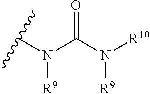

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

The term “protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The invention also includes various isomers and mixtures thereof. Certain of the compounds of the present invention may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. “R” and “S” represent the configuration of substituents around one or more chiral carbon atoms. When a chiral center is not defined as R or S, either a pure enantiomer or a mixture of both configurations is present.

“Racemate” or “racemic mixture” means a compound of equimolar quantities of two enantiomers, wherein such mixtures exhibit no optical activity; i.e., they do not rotate the plane of polarized light. In certain embodiments, compounds of the invention may be racemic.

In certain embodiments, compounds of the invention may be enriched in one enantiomer. For example, a compound of the invention may have greater than about 30% ee, about 40% ee, about 50% ee, about 60% ee, about 70% ee, about 80% ee, about 90% ee, or even about 95% or greater ee. In certain embodiments, compounds of the invention may have more than one stereocenter. In certain such embodiments, compounds of the invention may be enriched in one or more diastereomer. For example, a compound of the invention may have greater than about 30% de, about 40% de, about 50% de, about 60% de, about 70% de, about 80% de, about 90% de, or even about 95% or greater de.

In certain embodiments, the therapeutic preparation may be enriched to provide predominantly one enantiomer of a compound (e.g., of Formula (I)). An enantiomerically enriched mixture may comprise, for example, at least about 60 mol percent of one enantiomer, or more preferably at least about 75, about 90, about 95, or even about 99 mol percent. In certain embodiments, the compound enriched in one enantiomer is substantially free of the other enantiomer, wherein substantially free means that the substance in question makes up less than about 10%, or less than about 5%, or less than about 4%, or less than about 3%, or less than about 2%, or less than about 1% as compared to the amount of the other enantiomer, e.g., in the composition or compound mixture. For example, if a composition or compound mixture contains about 98 grams of a first enantiomer and about 2 grams of a second enantiomer, it would be said to contain about 98 mol percent of the first enantiomer and only about 2% of the second enantiomer.

In certain embodiments, the therapeutic preparation may be enriched to provide predominantly one diastereomer of a compound (e.g., of Formula (I)). A diastereomerically enriched mixture may comprise, for example, at least about 60 mol percent of one diastereomer, or more preferably at least about 75, about 90, about 95, or even about 99 mol percent.

The compounds of the invention may be prepared as individual isomers by either isomer specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least about 60%, about 70%, about 80%, about 90%, about 99% or about 99.9% by weight pure relative to the other stereoisomers. When a single enantiomer is named or depicted by structure, the depicted or named enantiomer is at least about 60%, about 70%, about 80%, about 90%, about 99% or about 99.9% by weight optically pure. Percent optical purity by weight is the ratio of the weight of the enantiomer that is present divided by the combined weight of the enantiomer that is present and the weight of its optical isomer.

In the pictorial representation of the compounds given through this application, a thickened tapered line ( [symbol] ) indicates a substituent which is above the plane of the ring to which the asymmetric carbon belongs and a dotted line ( [symbol] ) indicates a substituent which is below the plane of the ring to which the asymmetric carbon belongs.

As used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

An isotope-labelled form of a disclosed compound has one or more atoms of the compound replaced by an atom or atoms having an atomic mass or mass number different than that which usually occurs in greater natural abundance. Examples of isotopes which are readily commercially available and which can be incorporated into a disclosed compound by well-known methods include isotopes of hydrogen, carbon, nitrogen, oxygen, phosphorus, fluorine and chlorine, for example, 2H, 3H, 13C, 14C, 15N, 180, 170, 31P, 32P, 35S, 18F and 36Cl, respectively. An isotope-labelled compound provided herein can usually be prepared by carrying out the procedures disclosed herein, replacing a non-isotope-labelled reactant by an isotope-labelled reactant.

The concentration of such a heavier isotope, specifically deuterium, may be defined by the isotopic enrichment factor. The term “isotopic enrichment factor” as used herein means the ratio between the isotopic abundance and the natural abundance of a specified isotope. If a hydrogen atom in a compound of this invention is replaced with deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

An isotope-labelled compound as provided herein can be used in a number of beneficial ways. Compounds having 14C incorporated are suitable for medicament and/or substrate tissue distribution assays. Tritium (3H) and carbon-14 (14C), are preferred isotopes owing to simple preparation and excellent detectability. Heavier isotopes, for example deuterium (2H), has therapeutic advantages owing to the higher metabolic stability. Metabolism is affected by the primary kinetic isotope effect, in which the heavier isotope has a lower ground state energy and causes a reduction in the rate-limiting bond breakage. Slowing the metabolism can lead to an increased in vivo half-life or reduced dosage requirements or an improvement in therapeutic index.

For a further discussion, see S. L. Harbeson and R. D. Tung, Deuterium In Drug Discovery and Development, Ann. Rep. Med. Chem. 2011, 46, 403-417, Foster, A. B., “Deuterium Isotope Effects in Studies of Drug Metabolism,” Trends in Pharmacological Sciences, 5: 524-527 (1984) AND Foster, A. B., “Deuterium Isotope Effects in the Metabolism of Drugs and Xenobiotics: Implications for Drug Design,” Advances in Drug Research, 14: 1-40 (1985).

Metabolic stability can be affected by the compound's processing in different organs of the body. For example, compounds with poor pharmacokinetic profiles are susceptible to oxidative metabolism. In vitro liver microsomal assays currently available provide valuable information on the course of oxidative metabolism of this type, which in turn assists in the rational design of deuterated compounds as disclosed herein. Improvements can be measured in a number of assays known in the art, such as increases in the in vivo half-life (t½), concentration at maximum therapeutic effect (Cmax), area under the dose response curve (AUC), and bioavailability; and in terms of reduced clearance, dose and materials costs. Another effect of deuterated compounds can be diminishing or eliminating undesired toxic metabolites. For example, if a toxic metabolite arises through oxidative carbon-hydrogen (C—H) bond cleavage, the deuterated analogue will have a slower reaction time and slow the production of the unwanted metabolite, even if the particular oxidation is not a rate-determining step. See, e.g., Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33(10) 2927-2937, 1994, and Jarman et al. Carcinogenesis 16(4), 683-688, 1993.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” means to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition. Without being bound by any theory, in some embodiments, treating includes augmenting deficient CFTR activity. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, the term “prodrug” means a pharmacological derivative of a parent drug molecule that requires biotransformation, either spontaneous or enzymatic, within the organism to release the active drug. For example, prodrugs are variations or derivatives of the compounds of the invention that have groups cleavable under certain metabolic conditions, which when cleaved, become the compounds of the invention. Such prodrugs then are pharmaceutically active in vivo, when they undergo solvolysis under physiological conditions or undergo enzymatic degradation. Prodrug compounds herein may be called single, double, triple, etc., depending on the number of biotransformation steps required to release the active drug within the organism, and the number of functionalities present in a precursor-type form. Prodrug forms often offer advantages of solubility, tissue compatibility, or delayed release in the mammalian organism (See, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, C A, 1992). Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability.

As such, those of skill in the art will appreciate that certain of the presently disclosed compounds having free amino, amido, hydroxy or carboxylic groups can be converted into prodrugs. Prodrugs include compounds having an amino acid residue, or a polypeptide chain of two or more (e.g., two, three or four) amino acid residues which are covalently joined through peptide bonds to free amino, hydroxy or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrullinehomocysteine, homoserine, ornithine and methionine sulfone. Prodrugs also include compounds having a carbonate, carbamate, amide or alkyl ester moiety covalently bonded to any of the above substituents disclosed herein.

A “therapeutically effective amount”, as used herein refers to an amount that is sufficient to achieve a desired therapeutic effect. For example, a therapeutically effective amount can refer to an amount that is sufficient to improve at least one sign or symptom of cystic fibrosis.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein, “CFTR” means cystic fibrosis transmembrane conductance regulator. Loss of function mutations of CFTR are a cause of cystic fibrosis and lead to exocrine gland dysfunction and abnormal mucocilliary clearance. Mutations in the CFTR gene or protein may result in reduced activity of CFTR. The most common mutation is a specific mutation of the deletion of three nucleotides of the codon for phenylalanine at positon 508 (about 70% of cystic fibrosis patients) referred to as “ΔF508”. The ΔF508 mutation decreases the stability of the CFTR NBD1 domain and limits CFTR interdomain assembly. A patient can be ΔF508 homozygous or ΔF508 heterozygous (ΔF508/ΔF508). In particular mutations, the result is a gating mutation leading to a low probability of the ion channel in the open position. Such mutations include but are not limited to G551D, G178R, S549N, S549R, G551S, G970R, G1244E, 51251N, 51255P, and G1349D.

As used herein, the term “CFTR modulator” refers to a compound that increases the activity of CFTR. In certain aspects, a CFTR modulator is a CFTR corrector or a CFTR potentiator or a dual-acting compound having activities of a corrector and a potentiator. These dual acting agents are useful when the mutations result in absence or reduced amount of synthesized CFTR protein.

As used herein, the term “CFTR corrector” refers to a compound that increases the amount of functional CFTR protein to the cell surface and thus enhances ion transport. The CFTR correctors partially “rescue” misfolding of CFTR, thereby permitting its maturation and functional expression on the cell surface and may modify the folding environment and compounds that interact directly to modify folding and conformational maturation during synthesis. Examples of correctors include, but are not limited to, VX-809, VX-661, VX-152, VX-440, VX-983, and GLPG2222.

As used herein, the term “CFTR potentiator” refers to a compound that increases the ion channel activity of CFTR protein located at the cell surface, resulting in enhanced ion transport. CFTR potentiators repair the defective channel functions caused by mutations. Examples of potentiators include, but are not limited to, ivacaftor (VX770), deuterated ivacaftor (CPT 656), genistein and GLPG1837.

As used herein, the term “CTFR pharmacological chaperone” (PC) refers to compounds that stabilize the CTFR protein in its native state by binding directly to the protein.

As used herein, the term “CTFR proteostasis regulator” (PR) refers to compounds that enhance the protein folding efficiency within the cell. PRs can alter the activity of transcriptional, folding and/or membrane trafficking machinery, as well as impeding the degradation of partially folded, but functional, conformers at the endoplasmic reticulum (ER) or plasma membrane.

As used herein, “CFTR disease or condition” refers to a disease or condition associated with deficient CFTR activity, for example, cystic fibrosis, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), chronic sinusitis, dry eye disease, protein C deficiency, A.beta.-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

### Methods of Use

As discussed above, CTFR is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate and thiocyanate into and out of the cell. The most frequent CFTR mutation is the in-frame deletion of phenylalanine at residue 508 (ΔF508) in the first nucleotide binding domain (NBD1). The mutation has several deleterious effects on the production of CFTR in the ER, its correct folding, its movement to the plasma membrane and its normal function as an ion channel for the cell.

One such negative effect is that the NBD1 domain is partially or mis-folded which is recognized within the cell as an aberrant protein and tagged for disposal by ER-associated degradation (ERAD) via the ubiquitin-proteasome system (UPS). Should a partially or mis-folded CFTR protein emerge from the ER, the protein must travel to the plasma membrane through complex glycosylation in the Golgi compartment and be functionally inserted. In wild-type CFTR, only 20-40% of CTFR reaches the plasma membrane, indicating that CTFR has energetic instability of individual NBDs, a slow domain assembly, and relatively fast ERAD kinetics which all contribute to inefficient folding and sensitize CFTR to structural perturbations by mutations.

In wild-type CTFR, the NBD1 domain folds co-translationally while other domains fold post-translationally. Mutated ΔF508 CTFR has impaired NBD1 folding but its backbone structure and thermodynamic stability are similar to wild-type CTFR. With delayed folding kinetics, mutated ΔF508 CTFR NBD1 has an increased folding activation energy. Lack of proper folding results in hydrophobic residues being exposed to the surface of NBD1 which causes aggregation with other CTFR proteins. Thus, the aggregation temperature of mutated CTFR drops from 41° C. to 33° C. This level of instability creates a greater percentage of mis-folded mutant CFTR at physiological temperature (37° C. in humans). Mutant CFTR suffers from both kinetic and thermodynamic folding defects. CFTR stabilizers can address these folding defects, but complete energetic correction of mutant NBD1 folding has been shown to not result the CTFR biosynthetic processing, underscoring the need for interface stability as well.

The disclosed CFTR correctors can interact with the NBD domain to stabilize the correct folded position R, such that CTFR is not labeled for elimination from the cell. The preservation of correct folding enables CFTR to function as a chloride ion channel at wild-type levels. In some embodiments, disclosed CFTR correctors can enhance the performance of wild-type CTFR.

CFTR stabilizers can function in combination with other therapeutic agents such as CFTR correctors that promote Δ508 CFTR exit from the ER and accumulation in the plasma membrane. Increasing the amount of CFTR cell surface expression can result in improved chloride conductance following channel activation by both potentiators and a cAMP agonist. Thus, disclosed herein are combinations of CFTR stabilizers with CFTR correctors and potentiators, optionally with cAMP agonists or another therapeutic agent as described below.

Disclosed herein are methods of treating deficient CFTR activity in a cell, comprising contacting the cell with a compound of formula (I), or a pharmaceutically acceptable salt thereof. In certain embodiments, contacting the cell occurs in a subject in need thereof, thereby treating a disease or disorder mediated by deficient CFTR activity.

Also, disclosed herein are methods of treating a disease or a disorder mediated by deficient CFTR activity comprising administering a compound of Formula (I) or a pharmaceutically acceptable salt thereof. In some embodiments, the subject is a mammal, preferably a human. In some embodiments, the disease is associated with the regulation of fluid volumes across epithelial membranes, particularly an obstructive airway disease such as CF or COPD.

Such diseases and conditions include, but are not limited to, cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear plasy, Pick's disease, several polyglutamine neurological disorders, Huntington's, spinocerebullar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

Such diseases and conditions include, but are not limited to, cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome. In some embodiments, the disease is cystic fibrosis.

Provided herein are methods of treating cystic fibrosis, comprising administering to a subject in need thereof, a compound as disclosed herein or a pharmaceutically acceptable salt thereof. Also provided herein are methods of lessening the severity of cystic fibrosis, comprising administering to a subject in need thereof, a compound as disclosed herein or a pharmaceutically acceptable salt thereof. In some embodiments, the subject is a human. In some embodiments, the subject is at risk of developing cystic fibrosis, and administration is carried out prior to the onset of symptoms of cystic fibrosis in the subject.

Provided herein are compounds as disclosed herein for use in treating a disease or condition mediated by deficient CFTR activity. Also provided herein are uses of a compound as disclosed herein for the manufacture of a medicament for treating a disease or condition mediated by deficient CFTR activity.

Provided herein are kits for use in measuring the activity of CFTR or a fragment thereof in a biological sample in vitro or in vivo. The kit can contain: (i) a compound as disclosed herein, or a pharmaceutical composition comprising the disclosed compound, and (ii) instructions for: a) contacting the compound or composition with the biological sample; and b) measuring activity of said CFTR or a fragment thereof. In some embodiments, the biological sample is biopsied material obtained from a mammal or extracts thereof; blood, saliva, urine, feces, semen, tears, other body fluids, or extracts thereof. In some embodiments, the mammal is a human.

### Combination Treatments

As used herein, the term “combination therapy” means administering to a subject (e.g., human) two or more CFTR modulators, or a CFTR modulator and an agent such as antibiotics, ENaC inhibitors, GSNO (S-nitrosothiol s-nitroglutanthione) reductase inhibitors, and a CRISPR Cas correction therapy or system (as described in US 2007/0022507 and the like).

In certain embodiments, the method of treating or preventing a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

Additional therapeutic agents include, for example, ENaC inhibitors, mucolytic agents, modulators of mucus rheology, bronchodilators, antibiotics, anti-infective agents, anti-inflammatory agents, ion channel modulating agents, therapeutic agents used in gene or mRNA therapy, agents that reduce airway surface liquid and/or reduce airway surface PH, CFTR correctors, and CFTR potentiators, or other agents that modulate CFTR activity. Other therapeutics include liposomal composition components such as those described in WO2012/170889, hybrid oligonucleotides that facilitate RNA cleavage such as those described in WO2016/130943, and single stranded oligonucleotides that modulate gene expression as described in WO2016/130929.

In some embodiments, at least one additional therapeutic agent is selected from one or more CFTR modulators, one or more CFTR correctors and one or more CFTR potentiators.

Non-limiting examples of additional therapeutics include VX-770 (Ivacaftor), VX-809 (Lumacaftor, 3-(6-(I-(2,2-5 difluorobenzo[d][1,3]dioxol-5-yl)cyclopropanecarboxamido)-3-methylpyridin-2-yl) benzoic acid, VX-661 (Tezacaftor, I-(2,2-difluoro-1,3-benzodioxol-5-yl)-N—[I-[(2R)-2,3-dihydroxypropyl]-6-fluoro-2-(2-hydroxy-1,I-dimethylethyl)-IH-indol-5-yl]-cyclopropanecarboxamide), VX-983, VX-152, VX-440, VX-445, VX-659, VX-371, Orkambi, Ataluren (PTC 124) (3-[5-(2-fluorophenyl)-1,2,4-oxadiazol-3-yl]benzoic acid), PTI-130 (Proteostasis), PTI-801, PTI-808, PTI-428, N91115.74 (cavosonstat), QBW251 (Novartis) compounds described in WO2011113894, compounds N30 Pharmaceuticals (e.g., WO 2014/186704), deuterated ivacaftor (e.g., CTP-656 or VX-561), GLPG 2222, GLPG2451, GLPG3067, GLPG2851, GLPG2737, GLPG 1837 (N-(3-carbamoyl-5,5,7,7-tetramethyl-5,7-dihydro-4H-thieno[2,3-c]pyran-2-yl)-1H-pyrazole-5-carboxamide), GLPG 2665 (Galapagos), FDL 169 (Flatley Discovery lab), FDL 176, FDL438, FDL304, FD2052160, FD1881042, FD2027304, FD2035659, FD2033129, FD1860293, CFFT-Pot01, CFFT-Pot-02, P-1037, glycerol, phenylbutyrate, and the like.

Non-limiting examples of additional therapeutics include compounds disclosed in US Patent Application Nos. 62/944,141, 62/944,158 and 62/944,188, each of which is incorporated by reference in its entirety.

Non-limiting examples of anti-inflammatory agents are N6022 (3-(5-(4-(IH-imidazol-I-yl)10 phenyl)-I-(4-carbamoyl-2-methylphenyl)-′H-pyrrol-2-yl) propanoic acid), Ibuprofen, Lenabasum (anabasum), Acebilustat (CTX-4430), LAU-7b, POL6014, docosahexaenoic acid, alpha-1 anti-trypsin, sildenafil. Additional therapeutic agents also include, but are not limited to a mucolytic agent, a modifier of mucus rheology (such as hypertonic saline, mannitol, and oligosaccharide based therapy), a bronchodialator, an anti-infective (such as tazobactam, piperacillin, rifampin, meropenum, ceftazidime, aztreonam, tobramycin, fosfomycin, azithromycin, amitriptyline (?), vancomycin, gallium and colistin), an anti-infective agent, an anti-inflammatory agent, a CFTR modulator other than a compound of the present invention, and a nutritional agent. Additional therapeutic agents can include treatments for comorbid conditions of cyctic fibrosis, such as exocrine pancreatic insufficiency which can be treated with Pancrelipase or Liprotamase.

Examples of CFTR potentiators include, but are not limited to, Ivacaftor (VX-770), CTP-656, NVS-QBW251, FD1860293, GLPG451, GLPG1837, and N-(3-carbamoyl-5,5,7,7-tetramethyl-5,7-dihydro-4H-thieno[2,3-c]pyran-2-yl)-1H-pyrazole-5-carboxamide. Examples of potentiators are also disclosed in publications: WO2005120497, WO2008147952, WO2009076593, WO2010048573, WO2006002421, WO2008147952, WO2011072241, WO2011113894, WO2013038373, WO2013038378, WO2013038381, WO2013038386, WO2013038390, WO2014180562. WO2015018823, and U.S. patent application Ser. Nos. 14/271,080, 14/451,619 and 15/164,317.

Non-limiting examples of correctors include Lumacaftor (VX-809), 1-(2,2-difluoro-1,3-benzodioxol-5-yl)-N-{1-[(2R)-2,3-dihydroxypropyl]-6-fluor-2-(1-hydroxy-2-methylpropan-2-yl-1-H-indol-5-yl}cyclopropane arboxamide (VX-661), VX-983, GLPG2222, GLPG2665, GLPG2737, VX-152, VX-440, FDL169, FDL304, FD2052160, and FD2035659. Examples of correctors are also disclosed in US20160095858A1, and U.S. application Ser. Nos. 14/925,649 and 14/926,727.

In certain embodiments, the additional therapeutic agent is a CFTR amplifier, CFTR amplifiers enhance the effect of known CFTR modulators, such as potentiators and correctors Examples of CFTR amplifier include PTI130 and PTI428. Examples of amplifiers are also disclosed in publications: WO2015138909 and WO2015138934.

In certain embodiments, the additional therapeutic agent is an agent that reduces the activity of the epithelial sodium channel blocker (ENaC) either directly by blocking the channel or indirectly by modulation of proteases that lead to an increase in ENaC activity (e.g., serine proteases, channel-activating proteases). Exemplary of such agents include camostat (a trypsin-like protease inhibitor), QAU145, 552-02, GS-9411, INO-4995, Aerolytic, amiloride, AZD5634, and VX-371. Additional agents that reduce the activity of the epithelial sodium channel blocker (ENaC) can be found, for example, in PCT Publication No. WO2009074575 and WO2013043720; and U.S. Pat. No. 8,999,976.

In one embodiment, the ENaC inhibitor is VX-371.

In one embodiment, the ENaC inhibitor is SPX-101 (S18).

In certain embodiments, the combination of a compound of Formula (I), with a second therapeutic agent may have a synergistic effect in the treatment of cancer and other diseases or disorders mediated by adenosine. In other embodiments, the combination may have an additive effect.

### Pharmaceutical Compositions

The compositions and methods of the present invention may be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals.

A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the subject's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

In certain embodiments, the dosing follows a 3+3 design. The traditional 3+3 design requires no modeling of the dose-toxicity curve beyond the classical assumption for cytotoxic drugs that toxicity increases with dose. This rule-based design proceeds with cohorts of three patients; the first cohort is treated at a starting dose that is considered to be safe based on extrapolation from animal toxicological data, and the subsequent cohorts are treated at increasing dose levels that have been fixed in advance. In some embodiments, the three doses of a compound of formula (I) range from about 100 mg to about 1000 mg orally, such as about 200 mg to about 800 mg, such as about 400 mg to about 700 mg, such as about 100 mg to about 400 mg, such as about 500 mg to about 1000 mg, and further such as about 500 mg to about 600 mg. Dosing can be three times a day when taken with without food, or twice a day when taken with food. In certain embodiments, the three doses of a compound of formula (I) range from about 400 mg to about 800 mg, such as about 400 mg to about 700 mg, such as about 500 mg to about 800 mg, and further such as about 500 mg to about 600 mg twice a day. In certain preferred embodiments, a dose of greater than about 600 mg is dosed twice a day.

If none of the three patients in a cohort experiences a dose-limiting toxicity, another three patients will be treated at the next higher dose level. However, if one of the first three patients experiences a dose-limiting toxicity, three more patients will be treated at the same dose level. The dose escalation continues until at least two patients among a cohort of three to six patients experience dose-limiting toxicities (i.e., ≥about 33% of patients with a dose-limiting toxicity at that dose level). The recommended dose for phase II trials is conventionally defined as the dose level just below this toxic dose level.

In certain embodiments, the dosing schedule can be about 40 mg/m² to about 100 mg/m², such as about 50 mg/m² to about 80 mg/m², and further such as about 70 mg/m² to about 90 mg/m² by IV for 3 weeks of a 4 week cycle.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the invention with one or more additional therapeutic agent(s) (e.g., one or more additional chemotherapeutic agent(s)) provides improved efficacy relative to each individual administration of the compound of the invention (e.g., compound of formula I or Ia) or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

This invention includes the use of pharmaceutically acceptable salts of compounds of the invention in the compositions and methods of the present invention. A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, (3-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Although specific embodiments of the present disclosure will now be described with reference to the preparations and schemes, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

Unless defined otherwise, all technical and scientific terms used herein have the same meaning as commonly understood by one having ordinary skill in the art to which this disclosure belongs. Although other compounds or methods can be used in practice or testing, certain preferred methods are now described in the context of the following preparations and schemes.

A number of synthetic protocols were used to produce the compounds described herein. These synthetic protocols (see schemes below) have common intersections and can be used alternatively for synthesis of the compounds described herein.

### EXAMPLES

### General Synthetic Schemes for Compounds of Formula (I)

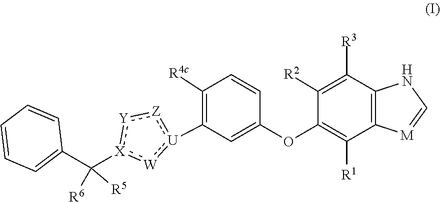

All the substituents and articles are defined as in the general description.

The compounds of the present disclosure can be better understood in connection with the following synthetic schemes and methods which illustrate a means by which the compounds of formula (I) can be prepared. The compounds of this disclosure can be prepared by a variety of synthetic procedures illustrated in Schemes 1 to 7.

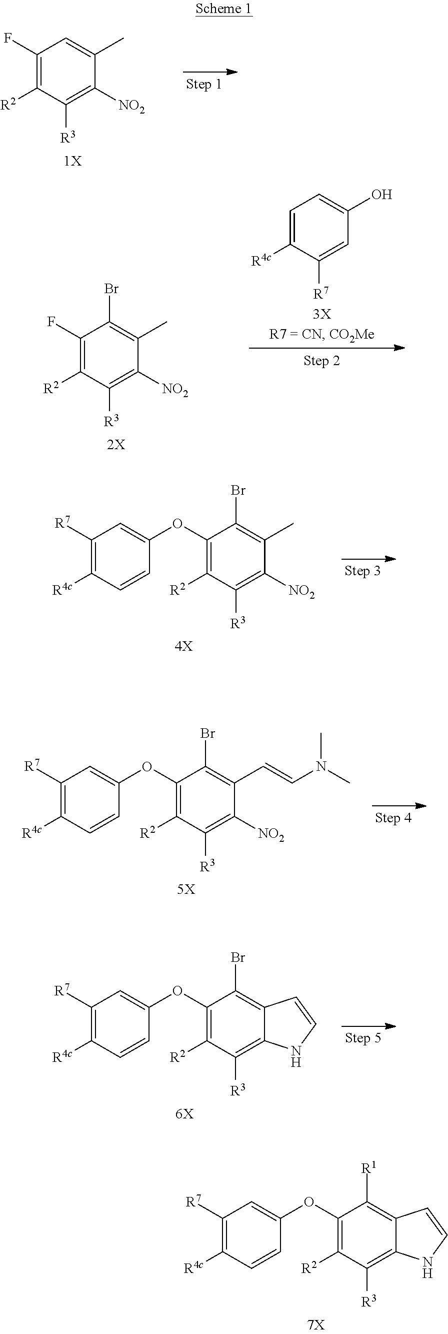

In Scheme 1, properly substituted methyl nitrobenzene (1X) is brominated (step 1) to form bromo compound 2. Treatment of 2X with phenol 3X (step 2) yields 4X. Reaction of 4X with DMF-DMA (step 3) affords 5X which can be reduced and then cyclized (step 4) to give indole 6X. The Br in indole 6X can then be converted into many different functional groups (R¹) in intermediate 7X.

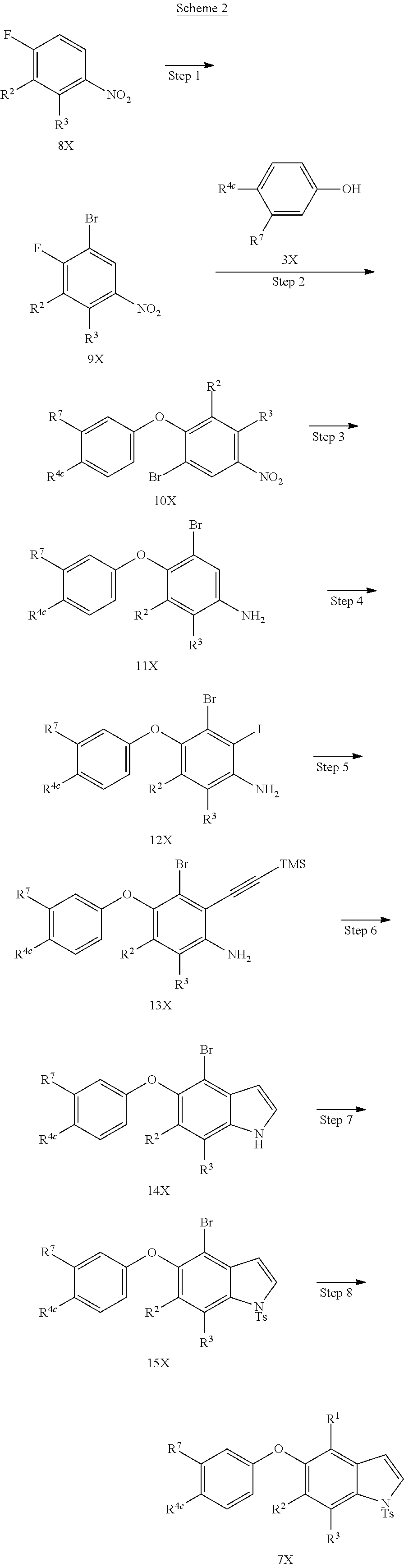

The intermediate 7X can also be prepared as illustrated in Scheme 2. Properly substituted nitrobenzene (8X) is brominated to form the bromo intermediate 9X, which is treated with substituted phenol 3X to yield intermediate 10X (step 2). Reduction of the nitro group into amine (step 3) gives 11X, which is then iodinated (step 4) to afford intermediate 12X. Intermediate 12X is further coupled with protected acetylene to form intermediate 13X (step 5) and then cyclized (step 6) to yield the key intermediate 14X. Proper protection (step 7) of indole (such as tosyl group in indole 15X) followed by conversion of the Br at 4-position of indole 15X to R¹ (step 8) yields different intermediates of general structure 7X.

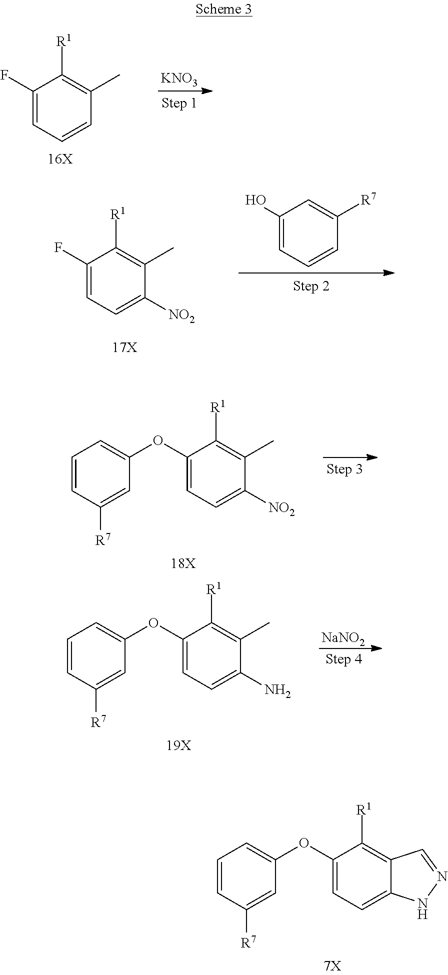

Scheme 3 illustrates the preparation of the intermediate 7X for synthesis of 1H-indazole analogues of formula (I). Properly substituted intermediate 16X is nitrated to give 17X and then treated with phenols to form 18X. Reduction of the nitro group yields 19X. Diazotization followed by cyclization gives the key intermediate of general structure 7X.

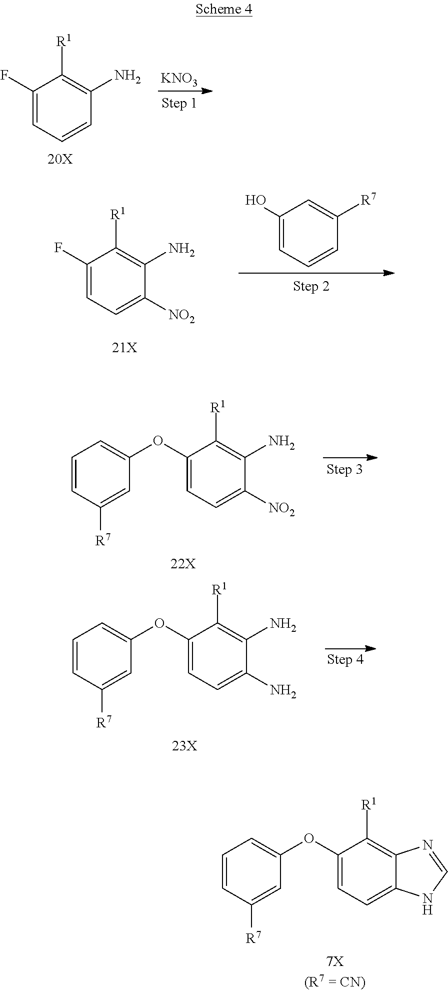

The benzimidazole analogues can be prepared as shown in scheme 4. Properly substituted amino compound 20X is nitrated to give 21X, which is then reacted with phenols (step 2) to form 22X. Reduction of the nitro group (step 3) yields the diamine 23X, which is then converted to the key benzimidazole intermediate 7X.

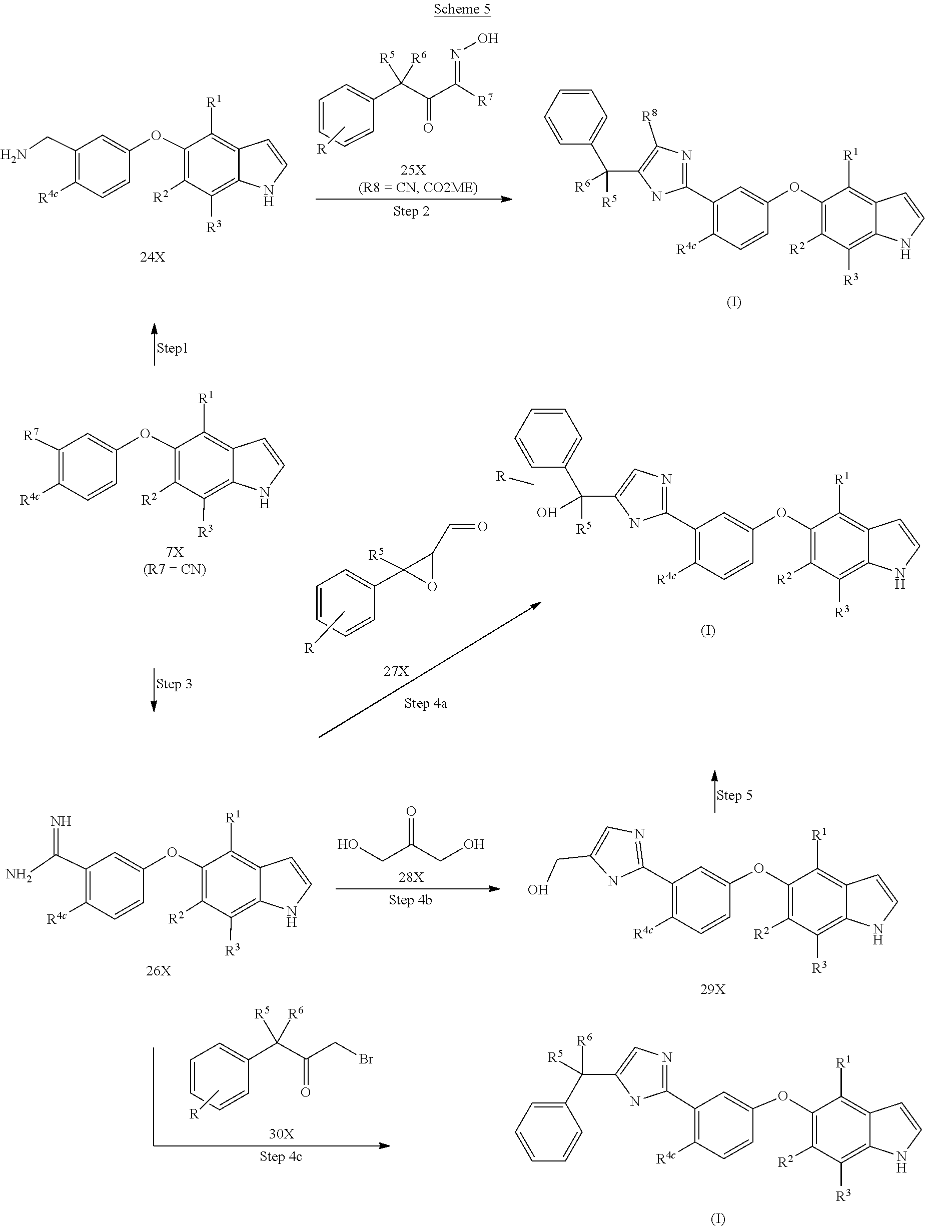

Scheme 5 illustrates the preparation of the compounds of formula (I) from various intermediates of general structure 7X. In step 1, intermediate 7X is reduced to give an amine 24X. Treatment of 24X with 25X (step 2) yields the imidazole compounds of formula (I).

Intermediate 7X can also be converted to amidine 26X, which can be cyclized with epoxide 27X (step 4a) to imidazole compounds of formula (I). Alternatively, 26X can be treated with 28X (step 4b) to give intermediate 29X, which is then further derivatized to compounds of formula (I). For example, oxidation of a hydroxy group followed by reaction with a Grignard reagent to give compounds of formula (I). Furthermore, Intermediate 7X can also be converted to amidine 26X, which can be easily cyclized with bromo ketone 30X (step 4c) to yield the compounds of formula (I).

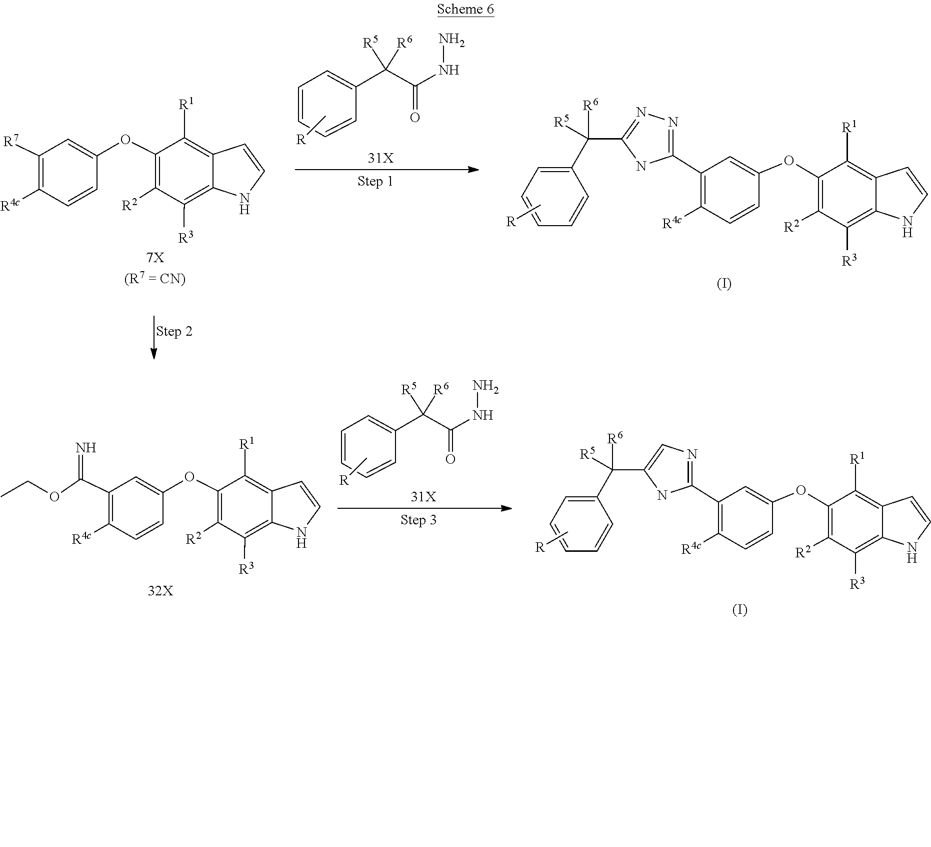

Scheme 6 illustrates an alternative route to derive the compounds of formula (I). Intermediate 7X can be treated with intermediate 31X directly to yield the compounds of formula (I). Alternatively, intermediate 7X is first converted to intermediate 32X (step 2) and then reacted with 31X to form the compounds of formula (I).

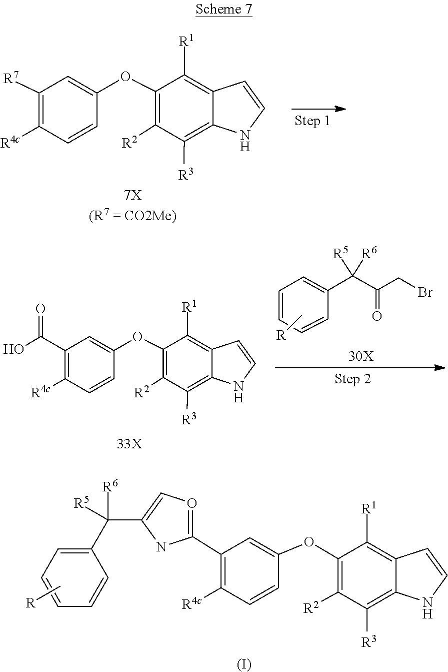

Scheme 7 illustrates a route for the synthesis of oxazole analogs of general formula (I). Intermediate 7X is hydrolyzed to an acid 33X, which is then treated with a bromo ketone 30X to give the compounds of formula (I).

### Analytical Procedures

1H NMR spectra were recorded with Bruker AC 400 MHz apparatus. Chemical shift (6) are quoted in parts per million (ppm) and coupling constants (J) in hertz (Hz).

LC-MS spectra were obtained with UPLC Acquity device of Waters for liquid chromatography part, coupling with mass spectrometer ZMD of Waters. This system was piloted by MassLynx v4.1 software. Detection was made in UV at 220 nm.

Operational conditions for liquid chromatography part are the following:
Column: Assentis Express C₁₈ 50×2.1 mm, 2.7μ supelco
Eluent: Way A: H₂O+0.02% TFA;
Way B: CH₃CN+0.014% TFA;
Gradient: To min: 2% B, T₁ min: 98% B, T_{1.3} min: 98% B, T_{1.33} min: 2% B, T_{1.5} min: following injection;
Flow: 1 mL/min;
Temperature: 55° C.
SQD: ESI+30V
UV: 220 nm
Injection: 0.2 μl.

### Preparation of Intermediates

### Intermediate 1

### 5-((4-Bromo-6-fluoro-1H-indol-5-yl)oxy)-2-fluorobenzonitrile

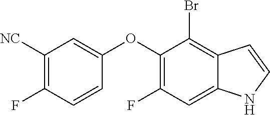 [SMILES: N#Cc1cc(Oc2c(F)cc3[nH]ccc3c2Br)ccc1F]

To a stirred solution of (E)-5-(2-bromo-3-(2-(dimethylamino)vinyl)-6-fluoro-4-nitrophenoxy)-2-fluorobenzonitrile (100 g, 236 mmol, Intermediate 1C) in acetic acid (800 mL) and toluene (800 mL) was added silica gel (42.5 g, 707 mmol). Iron powder (132 g, 2.4 mol) was added in small portions at 50° C. The suspension was stirred at 100° C. for 12 hours, cooled to room temperature, filtered through a pad of Celite and rinsed with ethyl acetate (5 L). The filtrate was diluted with water (10 L) and extracted with ethyl acetate (5 L×2). The combined organic layers were washed with water (10 L) and brine (10 L), dried over sodium sulfate, filtered and concentrated. The resulting dark brown oil was purified by column chromatography (SiO₂, 1-20% ethyl acetate in petroleum ether). The reaction and purification procedure was repeated twice on the same scale (total (E)-5-(2-bromo-3-(2-(dimethylamino)vinyl)-6-fluoro-4-nitrophenoxy)-2-fluorobenzonitrile used: 300 g, 707 mmol) and the three batches of product were combined to afford the title compound as a white solid (220 g, 81%). ¹H NMR (400 MHz CDCl₃) δ 8.41 (s, 1H), 7.25-7.26 (m, 1H), 7.15-7.18 (m, 3H), 6.95-7.09 (m, 1H), 6.55 (m, 1H) ppm.

### Intermediate 1A

### 3-Bromo-1,2-difluoro-4-methyl-5-nitrobenzene

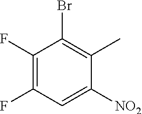

1,3-Dibromo-5,5-dimethylimidazolidine-2,4-dione (136 g, 476 mmol) and concentrated sulfuric acid (200 mL) were added to a solution of 1,2-difluoro-4-methyl-5-nitrobenzene (150 g, 866 mmol) in trifluoroacetic acid (800 mL) and the solution was stirred at 25° C. for 10 hours. Three batches of reaction mixtures (from a total of 450 g 1,2-difluoro-4-methyl-5-nitrobenzene) were combined, poured into ice water (5 L), stirred for 15 minutes and extracted with petroleum ether (4 L×2). The combined organic layers were washed with brine (5 L), dried over sodium sulfate, filtered and concentrated. The resulting crude product was purified by silica gel column chromatography (petroleum ether) to give the title compound as a yellow oil (417 g, 64%). ¹H NMR (400 MHz CDCl₃) δ 7.68 (m, 1H), 2.55 (s, 3H) ppm.

### Intermediate 1B

### 5-(2-Bromo-6-fluoro-3-methyl-4-nitrophenoxy)-2-fluorobenzonitrile

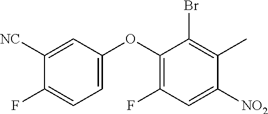 [SMILES: Cc1c([N+](=O)[O-])cc(F)c(Oc2ccc(F)c(C#N)c2)c1Br]

2-Fluoro-5-hydroxybenzonitrile (118 g, 861 mmol) and potassium carbonate (227 g, 1.6 mol) were added to a solution of 3-bromo-1,2-difluoro-4-methyl-5-nitrobenzene (207 g, 820 mmol) in DMF (1 L) and the suspension was stirred at 100° C. for one hour. Two batches of product from 3-bromo-1,2-difluoro-4-methyl-5-nitrobenzene (total of 414 g) were combined, cooled, poured into ice-water (7 L) and extracted with ethyl acetate (3 L×2). The combined organic layers were washed with water (5 L) and brine (3 L), dried over sodium sulfate, filtered and concentrated to give the title compound as a yellow solid (585 g, 97%), which was used without further purification. ¹H NMR (400 MHz CDCl₃) δ 7.68 (d, J=9.2 Hz, 1H), 7.10-7.14 (m, 2H), 7.01-7.02 (m, 1H), 2.58 (s, 3H) ppm.

### Intermediate 1C

### (E)-5-(2-Bromo-3-(2-(dimethylamino)vinyl)-6-fluoro-4-nitrophenoxy)-2-fluorobenzonitrile

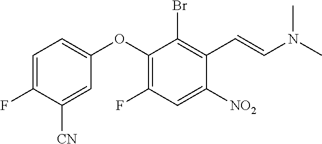 [SMILES: CN(C)/C=C/c1c([N+](=O)[O-])cc(F)c(Oc2ccc(F)c(C#N)c2)c1Br]

To a solution of 5-(2-bromo-6-fluoro-3-methyl-4-nitrophenoxy)-2-fluorobenzonitrile (124 g, 336 mmol) in DMF (1 L) was added dimethylformamid-dimethylacetal (178 ML, 160 g, 1.34 mol) and the solution was stirred at 100° C. for six hours. Six batches (from 744 g of 5-(2-bromo-6-fluoro-3-methyl-4-nitrophenoxy)-2-fluorobenzonitrile) were combined and poured into ice-water (20 L) and extracted with ethyl acetate (8 L×2). The combined organic extracts were washed with water (10 L) and brine (10 L), dried over sodium sulfate and concentrated to give the title compound as a black oil (786 g, 921%), which was used without further purification. ¹H NMR (400 MHz CDCl₃) δ7.38 (d, J=8.8 Hz, 1H), 7.19 (s, 1H), 7.10-7.12 (m, 2H), 6.44 (d, J=13.6 Hz, 1H), 4.96 (d, J=13.6 Hz, 1H), 2.80 (s, 6H) ppm.

The following intermediates were prepared utilizing the procedures described for Intermediate 1.

| No. | Structure | Name | ¹H NMR | MS m/z |
|---|---|---|---|---|
| 1-1 | 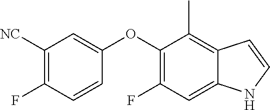 [SMILES: Cc1c(Oc2ccc(F)c(C#N)c2)c(F)cc2[nH]ccc12] | 2-fluoro-5-((6-fluoro-4- methyl-1H-indol-5- yl)oxy)benzonitrile | (400 MHz, CDCl₃) δ 8.28 (s, 1H), 7.27 (s, 1H), 7.20-7.07 (m, 3H), 6.98 (dd, J = 4. 8, 2.8 Hz, 1H), 6.58 (s, 1H), 2.39 (s, 3H) ppm. | 285 [M + H]⁺ |
| 1-2 | 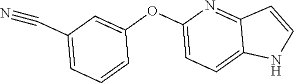 [SMILES: N#Cc1cccc(Oc2ccc3[nH]ccc3n2)c1] | 3-((1H-pyrrolo[3,2- b]pyridin-5- yl)oxy)benzonitrile | | 236 [M + H]⁺ |
| 1-3 | 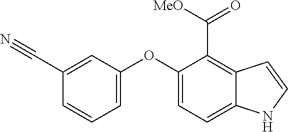 [SMILES: COC(=O)c1c(Oc2cccc(C#N)c2)ccc2[nH]ccc12] | methyl 5-(3- cyanophenoxy)-1H-indole- 4-carboxylate | (400 MHz, CD₃OD) δ 7.68 (d, J = 8.8 Hz, 1H), 7.43-7.47 (m, 2H), 7.35 (d, J = 7.6 Hz, 1H), 7.12- 7.15 (m, 2H), 6.92 (d, J = 8.8 Hz, 1H), 6.86 (d, J = 3.2 Hz, 1H), 3.79 (s, 3H) ppm. | 293 [M + H]⁺ |
| 1-4 | 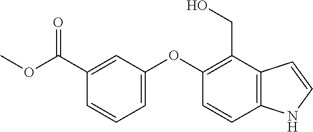 [SMILES: COC(=O)c1cccc(Oc2ccc3[nH]ccc3c2CO)c1] | methyl 3-((4- (hydroxymethyl)-1H-indol- 5-yl)oxy)benzoate | | 296 [M − H]⁻ |
| 1-5 | 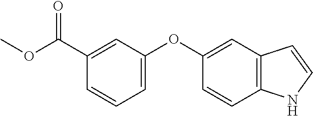 [SMILES: COC(=O)c1cccc(Oc2ccc3[nH]ccc3c2)c1] | methyl 3-((1H-indol-5- yl)oxy)benzoate | | 268 [M + H]⁺ |
| 1-6 | 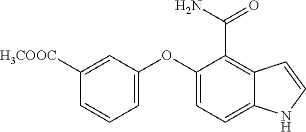 | methyl 3-((4-carbamoyl- 1H-indol-5- yl)oxy)benzoate | | 311 [M + H]⁺ |
| 1-7 | 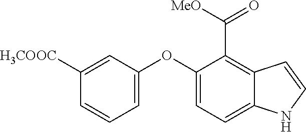 | methyl 5-(3- (methoxycarbonyl)phenox y)-1H-indole-4-carboxylate | | 326 [M + H]⁺ |
| 1-8 | 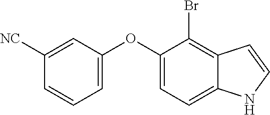 [SMILES: N#Cc1cccc(Oc2ccc3[nH]ccc3c2Br)c1] | 3-((4-bromo-1H-indol-5- yl)oxy)benzonitrile | | 313/315 [M + H]⁺ |
| 1-9 | 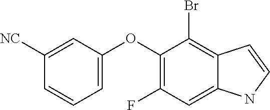 [SMILES: N#Cc1cccc(Oc2c(F)cc3[nH]ccc3c2Br)c1] | 3-((4-bromo-6-fluoro-1H- indol-5-yl)oxy)benzonitrile | | 331/333 [M + H]⁺ |

### Intermediate 2

### 2-Fluoro-5-((6-fluoro-4-vinyl-1H-indol-5-yl)oxy)benzonitrile

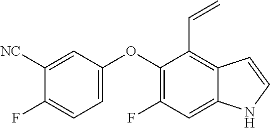 [SMILES: C=Cc1c(Oc2ccc(F)c(C#N)c2)c(F)cc2[nH]ccc12]

Pd(dppf)Cl₂ (0.7 g, 0.86 mmol) was added to a solution of 5-((4-Bromo-6-fluoro-1H-indol-5-yl)oxy)-2-fluorobenzonitrile (3 g, 8.59 mmol), 4,4,5,5-tetramethyl-2-vinyl-1,3,2-dioxaborolane (2.65 g, 17.2 mmol) and cesium carbonate (5.58 g, 17.2 mmol) in dioxane (60 mL) and water (15 mL). The reaction mixture was stirred overnight at 100° C. under nitrogen atmosphere and concentrated to remove solvent. The residue was dissolved in ethyl acetate (120 mL), washed with water and brine and dried over sodium sulfate. The resulting crude product was purified by silica gel column chromatography (0-30% ethyl acetate in petroleum ether) to give 2-fluoro-5-((6-fluoro-4-vinyl-1H-indol-5-yl)oxy)benzonitrile (2.1 g, 85%) as oil. MS m/z: 297 [M+H]⁺.

The following intermediates were prepared utilizing the procedures described for Intermediate 2.

| No. | Structure | Name | MS m/z |
|---|---|---|---|
| 2-1 | 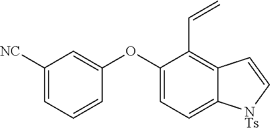 [SMILES: C=Cc1c(Oc2cccc(C#N)c2)ccc2c1ccn2S(=O)(=O)c1ccc(C)cc1] | 3-((1-tosy1-4-vinyl-1H-indol-5- yl)oxy)benzonitrile | 415 [M + H]⁺ |
| 2-2 | 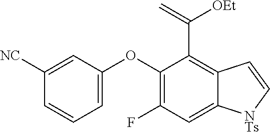 [SMILES: C=C(OCC)c1c(Oc2cccc(C#N)c2)c(F)cc2c1ccn2S(=O)(=O)c1ccc(C)cc1] | 3-((4-(1-ethoxyvinyl)-6-fluoro-1- tosyl-1H-indol-5- yl)oxy)benzonitrile | 477 [M + H]⁺ |

### Intermediate 3

### 3-Methyl-3-phenyloxirane-2-carbaldehyde

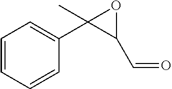

A solution of (3-methyl-3-phenyloxiran-2-yl)methanol (730 mg, 4.45 mmol), BAIB (1.58 g, 4.90 mmol) and TEMPO (69 mg, 0.44 mmol) in dichloromethane (5 mL) was stirred at room temperature for two hours. The mixture was concentrated to remove dichloromethane and the residue was purified by silica gel column chromatography (petroleum ether/ethyl acetate, v/v, 8/1) to give the title compound as a colorless oil (560 mg, 78%). ¹H NMR (400 MHz, CDCl₃) δ 9.62 (d, J=4.8 Hz, 1H), 7.39 (m, 5H), 3.36 (d, J=4.8 Hz, 1H), 1.87 (s, 3H) ppm.

### Intermediate 3A

### (3-Methyl-3-phenyloxiran-2-yl)methanol

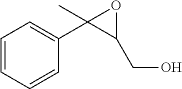

A suspension of (E)-3-phenylbut-2-en-1-ol (1.23 g, 8.31 mmol), m-CPBA (2.66 g, 11.6 mmol) and sodium bicarbonate (1.19 g, 14.1 mmol) in dichloromethane (80 mL) was stirred at room temperature for two hours. The reaction was quenched with aqueous sodium sulfite solution, stirred for 30 minutes and then extracted with dichloromethane (80 mL×2). The combined organic extracts were washed with brine, dried over sodium sulfate and concentrated. The resulting residue was purified by silica gel column chromatography (petroleum ether/ethyl acetate, v/v, 10/1 to 1/1) to give (3-methyl-3-phenyloxiran-2-yl)methanol as a light yellow syrup (730 mg, 47%). MS m/z: 165 [M+H]⁺. ¹H NMR (400 MHz, CDCl₃) δ 7.35 (m, 5H), 4.0-3.96 (dd, J=4.4, 12 Hz, 1H), 3.87-3.82 (dd, J=6.4, 12 Hz, 1H), 3.10 (q, J=6.8 Hz, 1H), 1.71 (s, 3H) ppm.

The following intermediate was prepared utilizing the procedures described for Intermediate 3.

| No. | Structure | Name | MS m/z |
|---|---|---|---|
| 3-1 | 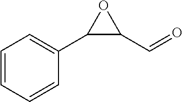 [SMILES: O=CC1OC1c1ccccc1] | 3-phenyloxirane-2- carbaldehyde | 149 [M + H]⁺ |

### Intermediate 4

### 1-Chloro-3-methyl-3-phenylbutan-2-one

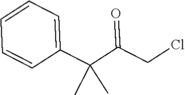

Oxalyl chloride (4.58 mL, 52.37 mmol) was added dropwise to a solution of 2-methyl-2-phenylpropanoic acid (4.3 g, 26 mmol) and DMF (41 μL, 523 μmol) in dichloromethane (50 mL) at 0° C. The reaction mixture was stirred for two hours and concentrated to remove solvent. The resulting chloride was co-evaporated with heptane twice, dissolved in acetonitrile (20 mL) and THF (20 mL) and then cooled to 0° C. To this solution was added trimethylsilyldiazomethane (26 mL, 52 mmol, 2M in hexane). The mixture was stirred for two hours and concentrated. The residue was dissolved in dichloromethane (20 mL), hydrogen chloride in dioxane (13.09 mL, 52.37 mmol, 4N) was added at 0° C. and stirred for 30 minutes. The solvent was evaporated and the residue was purified by silica gel column chromatography (0-10% ethyl acetate in heptane) to give a colorless oil. MS m/z: 195 [M−1]⁻.

The following intermediate was prepared utilizing the procedure described for Intermediate 4.

| No. | Structure | Name | ¹H NMR | MS m/z |
|---|---|---|---|---|
| 4-1 | 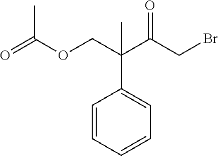 [SMILES: CC(=O)OCC(C)(C(=O)CBr)c1ccccc1] | 4-bromo-2-methyl-3- oxo-2-phenylbutyl acetate | (400 MHz, CDCl₃) δ 7.34-7.41 (m, 3H), 7.22-7.24 (m, 2H), 4.60 (d, 1H), 4.36 (d, 1H), 3.90 (dd, 2H), 2.01 (s, 3H), 1.67 (s, 3H) ppm. | 299 /301 [M + H]⁺ |
| 4-2 | 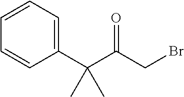 | 1-bromo-3-methyl-3- phenylbutan-2-one | | 241/243 [M + H]⁺ |

### Intermediate 5

### 5-((4-Bromo-6-fluoro-1-tosyl-1H-indol-5-yl)oxy)-2-fluorobenzonitrile

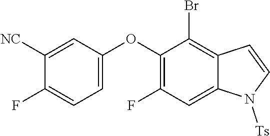 [SMILES: Cc1ccc(S(=O)(=O)n2ccc3c(Br)c(Oc4ccc(F)c(C#N)c4)c(F)cc32)cc1]

To a stirred solution of 5-((4-bromo-6-fluoro-1H-indol-5-yl)oxy)-2-fluorobenzonitrile (92 g, 262 mmol, Intermediate 1) in DMF (500 mL) was added, portion wise, a 60% dispersion of sodium hydride in mineral oil (12.7 g, 316 mmol, 60% purity) at 25° C. The reaction mixture was stirred for 30 minutes, 4-methylbenzenesulfonyl chloride (60.3 g, 316 mmol) was added and stirred for another 9.5 hours. The reaction was quenched with water (3 L) and extracted with ethyl acetate (2 L×2). The combined organic extracts were washed with brine (2 L), dried over sodium sulfate, filtered and concentrated to afford the title compound as a white solid (124 g, 94%). The product was used without further purification. ¹H NMR (400 MHz CDCl₃) δ 7.86 (d, J=10.4 Hz, 1H), 7.79 (d, J=8.0 Hz, 2H), 7.67 (d, J=2.8 Hz, 1H), 7.31 (d, J=8.0 Hz, 2H), 7.14 (d, J=6.8 Hz, 2H), 6.98 (s, 1H), 6.72 (d, J=2.8 Hz, 1H), 2.39 (s, 3H) ppm.

The following intermediates were prepared utilizing the procedures described for Intermediate 5.

| No. | Structure | Name | 1H NMR | MS m/z |
|---|---|---|---|---|
| 5-1 | 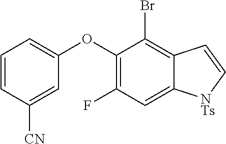 [SMILES: Cc1ccc(S(=O)(=O)n2ccc3c(Br)c(Oc4cccc(C#N)c4)c(F)cc32)cc1] | 3-((4-bromo-6- fluoro-1-tosy1-1H- indol-5- yl)oxy)benzonitrile | (400 MHz, CDCl₃) δ 7.87 (d, J = 10.0 Hz, 1H), 7.81 (d, J = 8.1 Hz, 2H), 7.68 (d, J = 3.6 Hz, 1H), 7.44-7.28 (m, 4H), 7.16 (d, J = 8.1 Hz, 1H), 7.05 (s, 1H), 6.73 (d, J = 3.0 Hz, 1H), 2.40 (s, 3H) ppm. | 507, 509 [M + Na]⁺ |
| 5-2 | 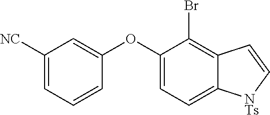 [SMILES: Cc1ccc(S(=O)(=O)n2ccc3c(Br)c(Oc4cccc(C#N)c4)ccc32)cc1] | 3-((4-bromo-1- tosyl-1H-indol-5- yl)oxy)benzonitrile | (400 MHz, CDCl₃) δ 7.97 (d, J = 8.8 Hz, 1H), 7.80 (d, J = 8.2 Hz, 2H), 7.70 (d, J = 3.6 Hz, 1H), 7.42-7.26 (m, 4H), 7.16-7.12 (m, 1H), 7.06-7.03 (m, 2H), 6.77 (d, J = 3.6 Hz, 1H), 2.39 (s, 3H) ppm. | 467, 469 [M + H]⁺ |
| 5-3 | 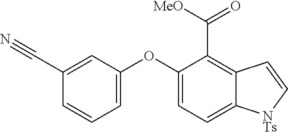 [SMILES: COC(=O)c1c(Oc2cccc(C#N)c2)ccc2c1ccn2S(=O)(=O)c1ccc(C)cc1] | methyl 5-(3- cyanophenoxy)-1- tosy1-1H-indole-4- carboxylate | | 447 [M + H]⁺ |

### Intermediate 6

### 2-Fluoro-5-((6-fluoro-4-(methylthio)-1H-indol-5-yl)oxy)benzonitrile

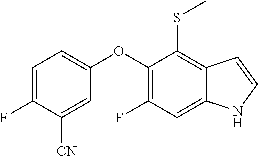 [SMILES: CSc1c(Oc2ccc(F)c(C#N)c2)c(F)cc2[nH]ccc12]

2-Fluoro-5-((6-fluoro-4-(methylthio)-1-tosyl-1H-indol-5-yl)oxy)benzonitrile (10 g, 21.2 mmol, Intermediate 6C) was added to a stirred solution of tetrabutylammonium chloride (1 M, 106 mL, 106 mmol) in THF. The reaction mixture was heated at 75° C. for two hours, cooled to room temperature, diluted with water (400 mL) and extracted with ethyl acetate (300 mL×2). The combined organic layers were washed with brine (400 mL), dried over sodium sulfate, filtered and concentrated. The resulting residue was purified by column chromatography (1-50% ethyl acetate in petroleum ether) to afford the title compound as white solid (3.65 g, 47%). MS m/z: 317 [M+H]⁺. ¹H NMR (400 MHz CDCl₃) δ 8.30 (s, 1H), 7.23-7.25 (m, 1H), 7.06-7.16 (m, 3H), 6.94-6.95 (m, 1H), 6.70-6.71 (m, 1H), 2.39 (s, 3H) ppm.

### Intermediate 6A

### 2-Fluoro-5-((6-fluoro-4-((4-methoxybenzyl)thio)-1-tosyl-1H-indol-5-yl)oxy)benzonitrile

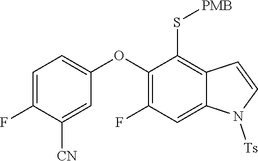

To a stirred solution of 5-((4-bromo-6-fluoro-1-tosyl-1H-indol-5-yl)oxy)-2-fluorobenzonitrile (57 g, 113 mmol, Intermediate 5) in dioxane (I L) were added (4-methoxyphenyl)methanethiol (17.5 g, 113 mmol, 16 mL), diisopropylethylamine (29.3 g, 226 mmol, 40 mL), Xantphos (6.6 g, 11.3 mmol) and Pd₂(dba)₃ (5.2 g, 5.7 mmol) under nitrogen atmosphere. The suspension was purged with nitrogen three times and then stirred under nitrogen at 100° C. for twelve hours. The mixture was cooled to room temperature, quenched with water (200 mL) and extracted with ethyl acetate (200 mL×2). The combined organic layers were washed with brine (300 mL), dried over sodium sulfate, filtered and concentrated. The resulting solid was purified by column chromatography over silica (2-50% ethyl acetate in petroleum ether) to afford the title compound (104 g, 80%) as a yellow solid. ¹H NMR (400 MHz CDCl₃) δ 7.76-7.84 (m, 3H), 7.58-7.59 (m, 1H), 7.31 (d, J=8.4 Hz, 2H), 7.06-7.08 (m, 2H), 6.89-6.91 (m, 3H), 7.75 (d, J=3.2 Hz, 1H), 6.62 (d, J=8.8 Hz, 2H), 3.96 (s, 2H), 3.74 (s, 3H), 2.40 (s, 3H) ppm.

### Intermediate 6B

### 2-Fluoro-5-((6-fluoro-4-mercapto-1-tosyl-1H-indol-5-yl)oxy)benzonitrile

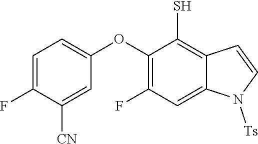 [SMILES: Cc1ccc(S(=O)(=O)n2ccc3c(S)c(Oc4ccc(F)c(C#N)c4)c(F)cc32)cc1]

To a stirred solution of 2-fluoro-5-((6-fluoro-4-((4-methoxybenzyl)thio)-1-tosyl-1H-indol-5-yl)oxy)benzonitrile (54.0 g, 93.6 mmol) in anisole (250 mL) was added, dropwise, trifluoroacetic acid (250 mL, 372 g, 3.26 mol). The reaction was stirred at 50° C. for 12 hours and then concentrated to afford the crude title compound as a brown solid (54.0 g, 126%; assuming quantitative conversion, the product was assigned a weight purity of 79%). The product was used without purification. MS n/z: 457 [M+H]⁺.

### Intermediate 6C

### 2-Fluoro-5-((6-fluoro-4-(methylthio)-1-tosyl-1H-indol-5-yl)oxy)benzonitrile

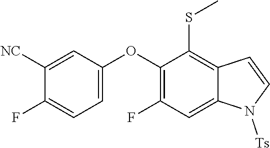 [SMILES: CSc1c(Oc2ccc(F)c(C#N)c2)c(F)cc2c1ccn2S(=O)(=O)c1ccc(C)cc1]

To a stirred solution of 2-fluoro-5-((6-fluoro-4-mercapto-1-tosyl-1H-indol-5-yl)oxy)benzonitrile (˜79 weight %, 54.0 g impure=42.7 g pure, 93.6 mmol) in THF (250 mL) was added PPh₃ (27.0 g, 103 mmol). The solution was stirred at room temperature for four hours, concentrated and redissolved in acetone (400 mL). To this stirred solution was added potassium carbonate (32.7 g, 237 mmol) and iodomethane (8.50 mL, 19.4 g, 137 mmol). The mixture was stirred for another six hours and then filtered and concentrated. The resulting oil was triturated with petroleum ether to afford the title compound as a yellow solid (44.0 g, 79%). ¹H NMR (400 MHz CDCl₃) δ 7.81-7.83 (m, 3H), 7.64 (d, J=3.6 Hz, 1H), 7.30 (d, J=8.0 Hz, 2H), 7.12 (dd, J=1.6, 1.6 Hz, 2H), 6.96-6.97 (m, 1H), 6.88 (d, J=3.6 Hz, 1H), 2.39 (s, 3H) ppm.

The following intermediates were prepared utilizing the procedures described for Intermediate 6.

| No. | Structure | Name | ¹H NMR | MS m/z |
|---|---|---|---|---|
| 6-1 | 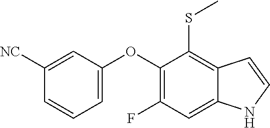 [SMILES: CSc1c(Oc2cccc(C#N)c2)c(F)cc2[nH]ccc12] | 3-((6-fluoro-4- (methylthio)-1H- indol-5- yl)oxy)benzonitrile | (400 MHz, CDCl₃) δ 8.36 (s, 1H), 7.39 (t, J = 8.0 Hz, 1H), 7.31 (dd, J = 6.1, 3.2 Hz, 2H), 7.21 (d, J = 9.9 Hz, 1H), 7.20-7.16 (m, 1H), 7.09 (s, 1H), 6.78 (t, J = 2.6 Hz, 1H), 2.46 (s, 3H) ppm. | 299 [M + H]⁺ |
| 6-2 | 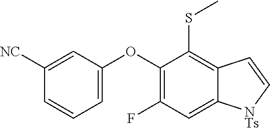 [SMILES: CSc1c(Oc2cccc(C#N)c2)c(F)cc2c1ccn2S(=O)(=O)c1ccc(C)cc1] | 3-((6-fluoro-4- (methylthio)-1-tosyl- 1H-indol-5- yl)oxy)benzonitrile | (400 MHz, CDCl₃) δ 7.84-7.80 (m, 3H), 7.65 (d, J = 3.6 Hz, 1H), 7.39 (t, J = 7.9 Hz, 1H), 7.34-7.30 (m, 3H), 7.14-7.12 (m, 1H), 7.05 (br s, 1H), 6.89 (d, J = 3.6 Hz, 1H), 2.40 (s, 3H), 2.39 (s, 3H) ppm. | 453 [M + H]⁺ |
| 6-3 | 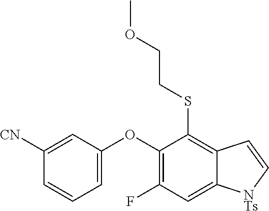 | 3-((6-fluoro-4-((2- methoxyethyl)thio)-1- tosy1-1H-indol-5- yl)oxy)benzonitrile | (400 MHz, CDCl₃) δ 7.86 (d, J = 10.2 Hz, 1H), 7.82 (d, J = 8.3 Hz, 2H), 7.65 (d, J = 3.7 Hz, 1H), 7.38 (t, J = 7.9 Hz, 1H), 7.36-7.29 (m, 3H), 7.12 (d, J = 7.7 Hz, 1H), 7.03 (s, 1H), 6.92 (d, J = 3.7 Hz, 1H), 3.38 (t, J = 6.5 Hz, 2H), 3.22 (s, 3H), 3.01 (t, J = 6.5 Hz, 2H), 2.40 (s, 3H) ppm. | 519 [M + Na]⁺ |
| 6-4 | 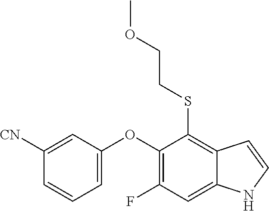 | 3-((6-fluoro-4-((2- methoxyethyl)thio)- 1H-indol-5- yl)oxy)benzonitrile | (400 MHz, CDCl₃) δ 8.37 (s, 1H), 7.38 (t, J = 7.9 Hz, 1H), 7.33-7.30 (m, 2H), 7.23 (s, 1H), 7.17 (d, J = 8.4 Hz, 1H), 7.08 (s, 1H), 6.80 (s, 1H), 3.43 (t, J = 6.7 Hz, 2H), 3.27 (s, 3H), 3.08 (t, J = 6.7 Hz, 2H) ppm. | 365 [M + Na]⁺ |
| 6-5 | 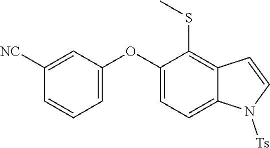 [SMILES: CSc1c(Oc2cccc(C#N)c2)ccc2c1ccn2S(=O)(=O)c1ccc(C)cc1] | 3-((4-(methylthio)-1- tosy1-1H-indol-5- yl)oxy)benzonitrile | (400 MHz, CDCl₃) δ 7.95 (d, J = 8.8 Hz, 1H), 7.81 (d, J = 8.4Hz, 2H), 7.68 (d, J = 3.6 Hz, 1H), 7.38 (t, J = 8.0 Hz, 1H), 7.32-7.26 (m, 3H), 7.12 (dd, J = 8.0, 1.6 Hz, 1H), 7.05 (s, 1H), 7.00 (d, J = 8.8 Hz, 1H), 6.93 (d, J = 3.6 Hz, 1H), 2.39 (s, 3H), 2.36 (s, 3H) ppm. | 457 [M + Na]⁺ |
| 6-6 | 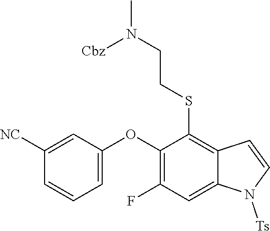 [SMILES: Cc1ccc(S(=O)(=O)n2ccc3c(SCCN(C)C(=O)OCc4ccccc4)c(Oc4cccc(C#N)c4)c(F)cc32)cc1] | benzyl (2-((5-(3- cyanophenoxy)-6- fluoro-1-tosyl-1H- indol-4- yl)thio)ethyl)(methyl) carbamate | | 652 [M + Na]⁺ |
| 6-7 | 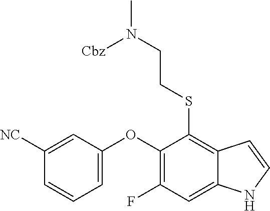 [SMILES: CN(CCSc1c(Oc2cccc(C#N)c2)c(F)cc2[nH]ccc12)C(=O)OCc1ccccc1] | benzyl (2-((5-(3- cyanophenoxy)-6- fluoro-1H-indol-4- yl)thio)ethyl)(methyl) carbamate | | 476 [M + H]⁺ |

### Intermediate 7

### 2-Fluoro-5-((6-fluoro-4-(S-methylsulfonimidoyl)-1H-indol-5-yl)oxy)benzonitrile

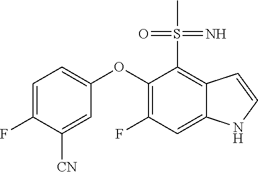 [SMILES: CS(=N)(=O)c1c(Oc2ccc(F)c(C#N)c2)c(F)cc2[nH]ccc12]

N-((5-(3-cyano-4-fluorophenoxy)-6-fluoro-1-tosyl-1H-indol-4-yl)(methyl)(oxo)-16-sulfaneylidene)-2,2,2-trifluoroacetamide (570 mg, 0.95 mol, Intermediate 7B) was dissolved in a 1M solution of tetrabutylammonium fluoride in THF (15 mL) and stirred at 70° C. for four hours. The mixture was diluted with ethyl acetate (50 mL), washed with water (30 mL×2) and brine (20 mL×3), dried over sodium sulfate, filtered and concentrated. The resulting crude product was purified by flash chromatography (0-70% ethyl acetate in petroleum ether) to afford the title compound (240 mg, 73%) as a solid. MS m/z: 348 [M+H]⁺. ¹H NMR (400 MHz, CDCl₃) δ 8.70 (s, 1H), 7.49 (d, J=9.8 Hz, 1H), 7.46-7.39 (m, 2H), 7.24-7.11 (m, 2H), 7.05 (dd, J=4.7, 3.1 Hz, 1H), 3.37 (s, 3H), 3.06 (s, 1H) ppm.

### Intermediate 7A

### 2-Fluoro-5-((6-fluoro-4-(methylsulfinyl)-1-tosyl-1H-indol-5-yl)oxy)benzonitrile

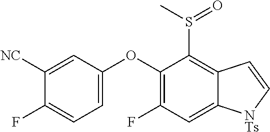 [SMILES: Cc1ccc(S(=O)(=O)n2ccc3c(S(C)=O)c(Oc4ccc(F)c(C#N)c4)c(F)cc32)cc1]

To a stirred solution of 2-fluoro-5-((6-fluoro-4-(methylthio)-1-tosyl-1H-indol-5-yl)oxy)benzonitrile (1.1 g, 2.34 mmol, Intermediate 6C) in THF (18 mL) was added acetic acid (6 mL) followed by H₂O₂ (30% in water, 2.66 g, 23.4 mmol) and the mixture was stirred at room temperature overnight and then at 40° C. for two days. The mixture was diluted with ethyl acetate (100 mL), washed with water (30 mL×2), saturated sodium bicarbonate solution (20 mL) and brine (20 mL×2), dried over sodium sulfate, filtered and concentrated. The resulting crude product was purified by flash chromatography (0-50% ethyl acetate in petroleum ether) to afford the title compound (803 mg, 70%) as a solid. MS m/z: 487 [M+H]⁺.

### Intermediate 7B

### N-((5-(3-cyano-4-fluorophenoxy)-6-fluoro-1-tosyl-1H-indol-4-yl)(methyl)(oxo)-16-sulfaneylidene)-2,2,2-trifluoroacetamide

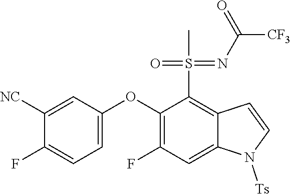 [SMILES: Cc1ccc(S(=O)(=O)n2ccc3c(S(C)(=O)=NC(=O)C(F)(F)F)c(Oc4ccc(F)c(C#N)c4)c(F)cc32)cc1]

To a stirred solution of 2-fluoro-5-((6-fluoro-4-(methylsulfinyl)-1-tosyl-1H-indol-5-yl)oxy)benzonitrile (833 mg, 1.71 mol) in dichloromethane (25 mL) was added CF₃CONH₂ (386 mg, 3.42 mmol), MgO (274 mg, 6.84 mmol), PhI(OAc)₄ (1.13 g, 2.57 mmol) and Rh₂(OAc)₄ (76 mg, 171 μmol). The mixture was stirred at room temperature over the weekend, filtered and rinsed with dichloromethane (10 mL×2). The filtrate was concentrated and the crude product was purified by flash chromatography (0-50% ethyl acetate in petroleum ether) to afford the title compound as a solid (380 mg, 35%) and recovered starting material 2-fluoro-5-((6-fluoro-4-(methylsulfinyl)-1-tosyl-1H-indol-5-yl)oxy)benzonitrile (380 mg, 46%). MS m/z: 598 [M+H]⁺. ¹H NMR (400 MHz, CDCl₃) δ 8.25 (d, J=10.3 Hz, 1H), 7.88-7.82 (m, 3H), 7.49 (dd, J=3.8, 0.5 Hz, 1H), 7.37 (d, J=8.1 Hz, 2H), 7.18-7.02 (m, 3H), 3.66 (s, 3H), 2.44 (s, 3H) ppm.

### Intermediate 8

### 3-((6-Fluoro-4-((2-methoxyethyl)thio)-1H-indol-5-yl)oxy)benzimidamide

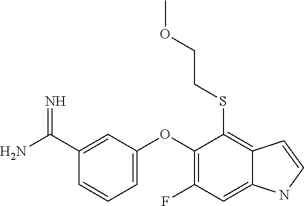 [SMILES: COCCSc1c(Oc2cccc(C(=N)N)c2)c(F)cc2[nH]ccc12]

To a stirred solution of 3-((6-fluoro-4-((2-methoxyethyl)thio)-1H-indol-5-yl)oxy)benzonitrile (235 mg, 0.687 mmol) in THF (5.5 mL) was added lithium bis(trimethylsilyl)amide (1M in THF, 5.5 mL) at 0° C. The reaction mixture was stirred at room temperature overnight, quenched with water (70 mL) and extracted with dichloromethane (50 mL×3). The combined organic extracts were washed with brine (30 mL), dried over sodium sulfate, filtered and concentrated to afford the crude title compound (280 mg) as an oil, which was used without further purification. MS m/z: 360 [M+H]⁺.

The following intermediates were prepared utilizing the procedures described for Intermediate 8.

| No. | Structure | Name | MS m/z |
|---|---|---|---|
| 8-1 | 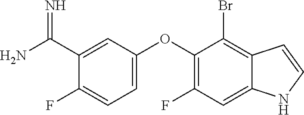 [SMILES: N=C(N)c1cc(Oc2c(F)cc3[nH]ccc3c2Br)ccc1F] | 5-((4-bromo-6-fluoro-1H-indol-5-yl)oxy)-2- fluorobenzimidamide | 366/368 [M + H]⁺ |
| 8-2 | 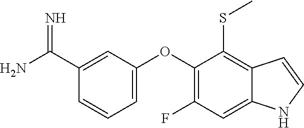 [SMILES: CSc1c(Oc2cccc(C(=N)N)c2)c(F)cc2[nH]ccc12] | 3-((6-fluoro-4-(methylthio)-1H-indol-5- yl)oxy)benzimidamide | 316 [M + H]⁺ |
| 8-3 | 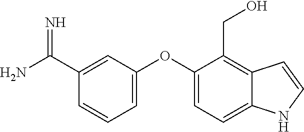 [SMILES: N=C(N)c1cccc(Oc2ccc3[nH]ccc3c2CO)c1] | 3-((4-(hydroxymethyl)-1H-indol-5- yl)oxy)benzimidamide | 282 [M + H]⁺ |
| 8-4 | 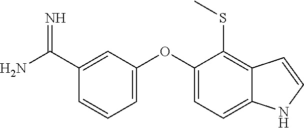 [SMILES: CSc1c(Oc2cccc(C(=N)N)c2)ccc2[nH]ccc12] | 3-((4-(methylthio)-1H-indol-5- yl)oxy)benzimidamide | 298 [M + H]⁺ |
| 8-5 | 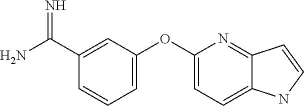 [SMILES: N=C(N)c1cccc(Oc2ccc3[nH]ccc3n2)c1] | 3-((1H-pyrrolo[3,2-b]pyridin-5- yl)oxy)benzimidamide | 253 [M+H]+ |
| 8-6 | 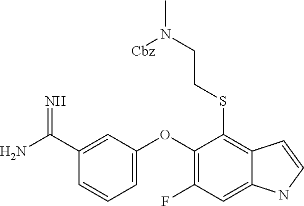 [SMILES: CN(CCSc1c(Oc2cccc(C(=N)N)c2)c(F)cc2[nH]ccc12)C(=O)OCc1ccccc1] | benzyl (2-((5-(3-carbamimidoylphenoxy)-6- fluoro-1H-indol-4- yl)thio)ethyl)(methyl)carbamate | 493 [M + H]⁺ |
| 8-7 | 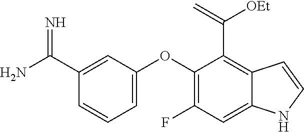 [SMILES: C=C(OCC)c1c(Oc2cccc(C(=N)N)c2)c(F)cc2[nH]ccc12] | 3-((4-(1-ethoxyvinyl)-6-fluoro-1H-indol-5- yl)oxy)benzimidamide | 340 [M + H]⁺ |
| 8-8 | 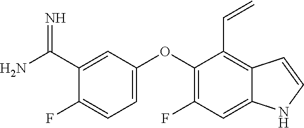 [SMILES: C=Cc1c(Oc2ccc(F)c(C(=N)N)c2)c(F)cc2[nH]ccc12] | 2-fluoro-5-((6-fluoro-4-vinyl-1H-indol-5- yl)oxy)benzimidamide | 314 [M + H]⁺ |
| 8-9 | 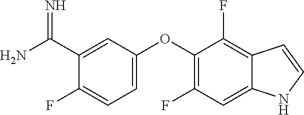 [SMILES: N=C(N)c1cc(Oc2c(F)cc3[nH]ccc3c2F)ccc1F] | 5-((4,6-difluoro-1H-indol-5-yl)oxy)-2- fluorobenzimidamide | 306 [M + H]⁺ |
| 8-10 | 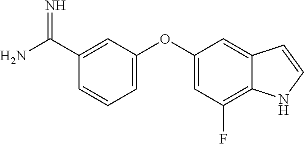 [SMILES: N=C(N)c1cccc(Oc2cc(F)c3[nH]ccc3c2)c1] | 3-((7-fluoro-1H-indol-5- yl)oxy)benzimidamide | 270 [M + H]⁺ |
| 8-11 | 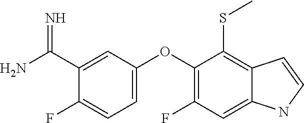 [SMILES: CSc1c(Oc2ccc(F)c(C(=N)N)c2)c(F)cc2[nH]ccc12] | 2-fluoro-5-((6-fluoro-4-(methylthio)-1H- indol-5-y1)oxy)benzimidamide | 334 [M + H]⁺ |
| 8-12 | 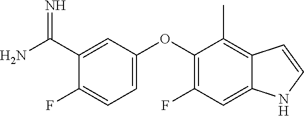 [SMILES: Cc1c(Oc2ccc(F)c(C(=N)N)c2)c(F)cc2[nH]ccc12] | 2-fluoro-5-((6-fluoro-4-methyl-1H-indol-5- yl)oxy)benzimidamide | 302 [M + H]⁺ |
| 8-13 | 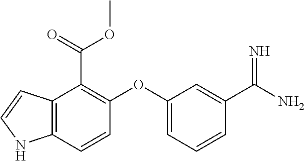 [SMILES: COC(=O)c1c(Oc2cccc(C(=N)N)c2)ccc2[nH]ccc12] | methyl 5-(3-carbamimidoylphenoxy)-1H- indole-4-carboxylate | 310 [M + H]⁺ |
| 8-14 | 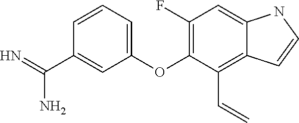 [SMILES: C=Cc1c(Oc2cccc(C(=N)N)c2)c(F)cc2[nH]ccc12] | 3-((6-fluoro-4-vinyl-1H-indol-5- yl)oxy)benzimidamide | 296 [M+H]+ |

### Intermediate 9

### 1,2-Difluoro-3,4-dimethyl-5-nitrobenzene

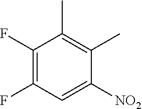

A mixture of 3-bromo-1,2-difluoro-4-methyl-5-nitrobenzene 0 g, 40 mmol, Intermediate 1A), MeB(OH)₂ (12 g, 200 mmol), Pd(dppf)Cl₂ (2.9 mg, 4.0 mmol) and sodium bicarbonate (10 g, 120 mmol) in 1,4-dioxane (160 mL) and water (40 mL) was stirred under nitrogen atmosphere at 80° C. for three days. The mixture was filtered and the filtrate was diluted with ethyl acetate (300 mL). The organic phase was washed with water (100 mL×2) and brine (50 mL), dried over sodium sulfate, filtered and concentrated under vacuum. The resulting residue was purified by silica gel column chromatography (petroleum ether/ethyl acetate, v/v, 12:1) to afford the title compound as a light yellow oil (6.4 g, 85%). ¹H NMR (400 MHz, CDCl₃) δ 7.59 (m, 1H), 2.42 (s, 3H), 2.32 (d, J=2.4 Hz, 3H) ppm.

### Intermediate 10

### 3-((4-Vinyl-1H-indol-5-yl)oxy)benzonitrile

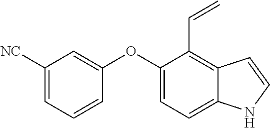 [SMILES: C=Cc1c(Oc2cccc(C#N)c2)ccc2[nH]ccc12]

A mixture of 3-((1-tosyl-4-vinyl-1H-indol-5-yl)oxy)benzonitrile (3 g, 7.25 mmol, Intermediate 8-1) and potassium carbonate (3 g, 21.74 mmol) in methanol (30 ml) was refluxed for 1.5 hours and then concentrated. The residue was diluted with water (30 ml) and extracted with ethyl acetate (30 mL×3). The combined organic extracts were dried over sodium sulfate, filtered and concentrated. The crude product was purified by silica gel column (10-20% ethyl acetate in petroleum ether) to afford the title compound as light yellow oil (1.4 g, 74%). MS m/z: 261 [M+H]⁺.

The following intermediates were prepared utilizing the procedures described for Intermediate 10.

| No. | Structure | Name | ¹H NMR | MS m/z |
|---|---|---|---|---|
| 10-1 | 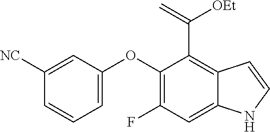 [SMILES: C=C(OCC)c1c(Oc2cccc(C#N)c2)c(F)cc2[nH]ccc12] | 3-((4-(1-ethoxyvinyl)- 6-fluoro-1H-indol-5- yl)oxy)benzonitrile | | 323 [M + H]⁺ |
| 10-2 | 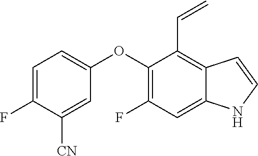 [SMILES: C=Cc1c(Oc2ccc(F)c(C#N)c2)c(F)cc2[nH]ccc12] | 2-fluoro-5-((6-fluoro- 4-vinyl-1H-indol-5- yl)oxy)benzonitrile | | 297 [M + H]⁺ |
| 10-3 | 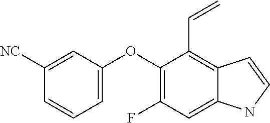 [SMILES: C=Cc1c(Oc2cccc(C#N)c2)c(F)cc2[nH]ccc12] | 3-((6-fluoro-4-vinyl- 1H-indol-5- yl)oxy)benzonitrile | (400 MHz, CDCl₃) δ 8.48 (br s, 1H), 7.37 (t, J = 8.0 Hz, 1H), 7.31-7.26 (m, 2H), 7.18 (d, J = 10.0Hz, 2H), 7.08 (s, 1H), 6.96 (dd, J = 18.0, 11.6 Hz, 1H), 6.82 (s, 1H), 6.01 (d, J = 18.0 Hz, 1H), 5.54 (d, J = 11.6 Hz, 1H) ppm. | 279 [M + H]⁺ |

### Intermediate 11

### 3-((4-(Hydroxymethyl)-1H-indol-5-yl)oxy)benzonitrile

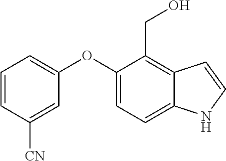 [SMILES: N#Cc1cccc(Oc2ccc3[nH]ccc3c2CO)c1]

To a stirred solution of 3-((4-formyl-1H-indol-5-yl)oxy)benzonitrile (2.32 g, 8.85 mmol, Intermediate 11A) in methanol (20 mL) was added NaBH₄ (1.01 g, 26.6 mmol). The resulting mixture was stirred at 25° C. for two hours, quenched with saturated ammonium chloride solution (100 mL) and extracted with ethyl acetate (50 mL×3). The combined organic extracts were washed with brine (50 mL), dried over sodium sulfate, filtered and concentrated in vacuo. The resulting residue was purified by silica gel column chromatography (petroleum ether/ethyl acetate, v/v, 2/1) to afford the title compound (2.16 g, 92%) as light yellow oil. MS m/z: 263 [M−H]⁻.

### Intermediate 11A

### 3-((4-Formyl-1H-indol-5-yl)oxy)benzonitrile

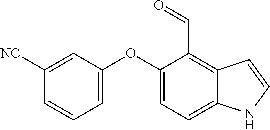 [SMILES: N#Cc1cccc(Oc2ccc3[nH]ccc3c2C=O)c1]

To a stirred solution of 3-((4-vinyl-1H-indol-5-yl)oxy)benzonitrile (2.80 g, 10.8 mmol, Intermediate 10) in a 3:1 mixture of THF and water (40 mL) were added OsO₄ (0.5 mL, 4% in water) and NaIO₄ (6.91 g, 32.3 mmol). The resulting mixture was stirred at 25° C. for two hours, quenched with saturated sodium thiosulfate solution (100 mL) and extracted with ethyl acetate (50 mL×3). The combined organic extracts were washed with brine (50 mL), dried over sodium sulfate, filtered and concentrated in vacuo to afford the crude title compound as brown oil (2.32 g), which was used without further purification. MS m/z: 285 [M+Na]⁺.

The following intermediates were prepared utilizing the procedures described for Intermediate 11.

| No. | Structure | Name | MS m/z |
|---|---|---|---|
| 11-1 | 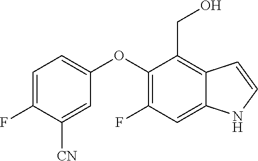 [SMILES: N#Cc1cc(Oc2c(F)cc3[nH]ccc3c2CO)ccc1F] | 2-fluoro-5-((6-fluoro-4-(hydroxymethyl)-1H- indol-5-yl)oxy)benzonitrile | 301 [M + H]⁺ |
| 11-2 | 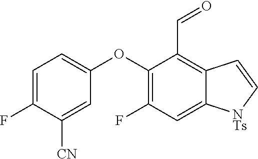 [SMILES: Cc1ccc(S(=O)(=O)n2ccc3c(C=O)c(Oc4ccc(F)c(C#N)c4)c(F)cc32)cc1] | 2-fluoro-5-((6-fluoro-4-formyl-1-tosyl-1H- indol-5-yl)oxy)benzonitrile | 453 [M + H]⁺ |

### Intermediate 12

### 3-((4-(Methylthio)-1H-indol-5-yl)oxy)benzonitrile

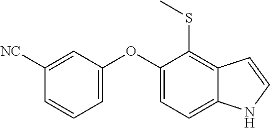 [SMILES: CSc1c(Oc2cccc(C#N)c2)ccc2[nH]ccc12]

To a stirred solution of 3-(3-methyl-2-(methylthio)-4-nitrophenoxy)benzonitrile (540 mg, 1.8 mmol, Intermediate 12B) in DMF (20 mL) was added DMF-DMA (2.14 g, 18.0 mmol). The reaction mixture was stirred at 120° C. overnight and concentrated in vacuo to afford the crude compound. To a stirred solution of the crude compound (1.0 g) in acetic acid (10 mL) was added zinc powder (1.0 g, 18.0 mmol). The resulting mixture was stirred at 90° C. overnight, cooled to room temperature, filtered through Celite and concentrated in vacuo. The resulting residue was dissolved in ethyl acetate (100 ml), washed with water (30 mL) and brine (20 mL), dried over sodium sulfate, filtered and concentrated in vacuo. The residue was purified by silica gel column chromatography (petroleum ether/ethyl acetate, v/v, 10/1 to 3/1) to afford the title compound as yellow sticky oil (260 mg, 52%). MS-ESI m/z: 281 [M+H]⁺.

### Intermediate 12A

### 3-(2-Bromo-3-methyl-4-nitrophenoxy)benzonitrile

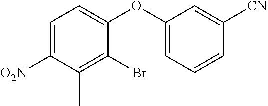 [SMILES: Cc1c([N+](=O)[O-])ccc(Oc2cccc(C#N)c2)c1Br]

To a stirred solution of 2-bromo-1-fluoro-3-methyl-4-nitrobenzene (2.34 g, 10.0 mmol) in DMF (30 mL) were added 3-hydroxybenzonitrile (1.43 g, 12.0 mmol) and potassium carbonate (2.76 g, 20.0 mmol). The reaction mixture was stirred at 90° C. for twenty hours, cooled to room temperature, quenched with water (150 mL) and extracted with ethyl acetate (2×200 mL). The combined organic phase was washed with brine (80 mL), dried over sodium sulfate, filtered and concentrated in vacuo. The resulting residue was purified by silica gel column chromatography (2-5% ethyl acetate in petroleum ether) to afford the title compound as a pale yellow solid (2.5 g, 75%). ¹H NMR (400 MHz, CD₃OD) δ 7.92 (d, J=9.2 Hz, 1H), 7.62-7.60 (m, 2H), 7.45-7.44 (m, 1H), 7.37-7.34 (m, 1H), 7.04 (d, J=8.8 Hz, 1H), 2.67 (s, 3H) ppm.

### Intermediate 12B

### 3-(3-Methyl-2-(methylthio)-4-nitrophenoxy)benzonitrile

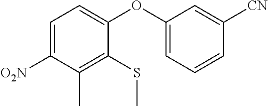 [SMILES: CSc1c(Oc2cccc(C#N)c2)ccc([N+](=O)[O-])c1C]

To a stirred solution of 3-(2-bromo-3-methyl-4-nitrophenoxy)benzonitrile (1.66 g, 5.0 mmol) in toluene (60 mL) were added sodium methanethiolate (700 mg, 10.0 mmol), Pd₂(dba)₃ (457 mg, 0.5 mmol) and Xantphos (433 mg, 0.75 mmol) at room temperature. The resulting mixture was stirred at 90° C. for 16 hours, water (30 mL) was added and extracted by ethyl acetate (2×100 mL). The combined organic layers were washed with brine (50 mL), dried over sodium sulfate, filtered and concentrated in vacuo to give the crude product which was purified by silica gel column chromatography (2-5% ethyl acetate in petroleum ether) to afford the title compound as a yellow solid (540 mg, 36%). ¹H NMR (400 MHz, CD₃OD) δ 7.80 (d, J=8.8 Hz, 1H), 7.50-7.47 (m, 2H), 7.25-7.21 (m, 2H), 6.85 (d, J=8.8 Hz, 1H), 2.78 (s, 3H), 2.37 (s, 3H) ppm.

### Intermediate 13

### (2-(3-((4-(Methylthio)-1H-indol-5-yl)oxy)phenyl)-1H-imidazol-5-yl)methanol

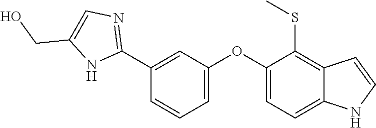 [SMILES: CSc1c(Oc2cccc(-c3ncc(CO)[nH]3)c2)ccc2[nH]ccc12]

To a stirred solution of 3-((4-(methylthio)-1H-indol-5-yl)oxy)benzimidamide (Intermediate 8-4, 180 mg, 0.6 mmol) in concentrated ammonium hydroxide solution (10 mL) were added 1,3-dihydroxypropan-2-one (270 mg, 3.0 mmol) and ammonium chloride (194 mg, 3.6 mmol) at room temperature. The reaction tube was sealed, the mixture was stirred at 90° C. for 16 hours, cooled to room temperature and extracted with dichoromethane (30 mL×2). The combined organic extracts were washed with brine (20 mL), dried over sodium sulfate, filtered and concentrated in vacuo. The resulting crude was purified by silica gel column chromatography (dichloromethane/methanol, v/v, 30/1 to 10/1) to afford the title compound as a yellow solid (100 mg, 48%). MS-ESI m/z: 352[M+H]⁺.

### Intermediate 14

### 2-(3-((4-(Methylthio)-1H-indol-5-yl)oxy)phenyl)-1H-imidazole-5-carbaldehyde

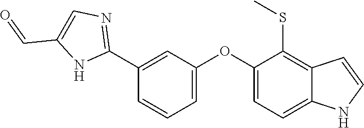 [SMILES: CSc1c(Oc2cccc(-c3ncc(C=O)[nH]3)c2)ccc2[nH]ccc12]

To a stirred solution of (2-(3-((4-(methylthio)-1H-indol-5-yl)oxy)phenyl)-1H-imidazol-5-yl)methanol (100 mg, 0.28 mmol) in DMF (5 mL) was added manganese dioxide (243 mg, 2.8 mmol). The resulting mixture was stirred at 50° C. for 20 hours, filtered through Celite and concentrated in vacuo to give a residue. The residue was purified by silica gel column chromatography (petroleum ether/ethyl acetate, v/v, 5/1 to 2/1) to afford the title compound (65 mg, 67%) as a yellow solid. MS-ESI m/z: 350 [M+H]⁺.

### Intermediate 15

### 5-((4,6-Difluoro-1H-indol-5-yl)oxy)-2-fluorobenzonitrile

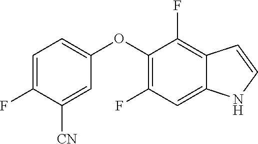 [SMILES: N#Cc1cc(Oc2c(F)cc3[nH]ccc3c2F)ccc1F]

### A. 5-(2,6-Difluoro-4-nitrophenoxy)-2-fluorobenzonitrile

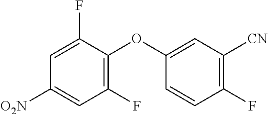 [SMILES: N#Cc1cc(Oc2c(F)cc([N+](=O)[O-])cc2F)ccc1F]

To a stirred solution of 1,2,3-trifluoro-5-nitrobenzene (1.10 g, 6.21 mmol) in DMF (5 mL) were added potassium carbonate (1.71 g, 12.4 mmol) and 2-fluoro-5-hydroxybenzonitrile (936 mg, 6.83 mmol). The resulting mixture was heated at 100° C. for two hours, cooled to room temperature and quenched with water (20 mL). The yellow precipitate was collected by filtration, washed with water and dried to afford the title compound (1.70 g, 94%). ¹H NMR (400 MHz, CDCl₃) δ 8.06-8.01 (m, 2H), 7.30-7.20 (m, 3H) ppm.

### B. 5-(4-Amino-2,6-difluorophenoxy)-2-fluorobenzonitrile

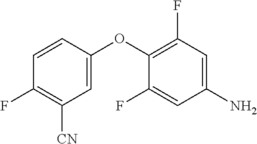 [SMILES: N#Cc1cc(Oc2c(F)cc(N)cc2F)ccc1F]

To a suspension of 5-(2,6-difluoro-4-nitrophenoxy)-2-fluorobenzonitrile (1.70 g, 5.78 mmol) in ethanol (30 mL) were added a solution of ammonium chloride (2.45 g, 46.2 mmol) in water (10 mL) and iron power (1.48 g, 23.1 mmol). The reaction mixture was heated at reflux for four hours, the insoluble material was removed by filtration and the filtrate was concentrated. The resulting residue was dissolved in ethyl acetate (100 mL), washed with water (30 mL×3) and brine (30 mL), dried over sodium sulfate, filtered and concentrated to afford the title compound as a yellow solid (1.60 g, 98%). MS m/z: 263 [M−1]⁻.

### C. 5-(4-Amino-2,6-difluoro-3-iodophenoxy)-2-fluorobenzonitrile

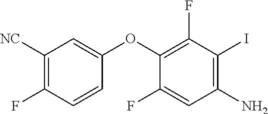 [SMILES: N#Cc1cc(Oc2c(F)cc(N)c(I)c2F)ccc1F]

A solution of 5-(4-amino-2,6-difluorophenoxy)-2-fluorobenzonitrile (1.60 g, 6.06 mmol) and NIS (1.36 g, 6.06 mmol) in acetic acid was stirred at 30° C. for one hour and then concentrated under reduced pressure. The resulting residue was purified by silica gel column chromatography (20% ethyl acetate in petroleum ether) to afford the title compound (2.08 g, 87%) as a yellow solid. MS m/z: 389 [M−1]⁻.

### D. 5-(4-Amino-2,6-difluoro-3-((trimethylsilyl)ethynyl)phenoxy)-2-fluorobenzonitrile

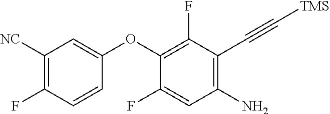 [SMILES: C[Si](C)(C)C#Cc1c(N)cc(F)c(Oc2ccc(F)c(C#N)c2)c1F]

To a stirred solution of 5-(4-amino-2,6-difluoro-3-iodophenoxy)-2-fluorobenzonitrile (1.0 g, 2.56 mmol) in DMF (15 mL) were added Pd(dppf)Cl₂ (182 mg, 0.26 mmol), CuI (48 mg, 0.26 mmol), triethylamine (0.54 mL, 3.84 mmol) and ethynyltrimethylsilane (0.47 mL, 3.33 mmol). The reaction mixture was stirred at 30° C. for three hours under nitrogen atmosphere, quenched with water (30 mL) and extracted with ethyl acetate (20 mL×3). The combined organic extracts were washed with brine (10 mL), dried over sodium sulfate, filtered and concentrated. The resulting residue was purified by silica gel column chromatography (20% ethyl acetate in petroleum ether) to afford the title compound as a yellow solid (900 mg, 97%). MS m/z: 359 [M−1]⁻.

### E. 5-((4,6-Difluoro-1H-indol-5-yl)oxy)-2-fluorobenzonitrile

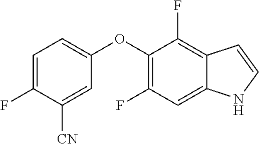 [SMILES: N#Cc1cc(Oc2c(F)cc3[nH]ccc3c2F)ccc1F]

A mixture of 5-(4-amino-2,6-difluoro-3-((trimethylsilyl)ethynyl)phenoxy)-2-fluorobenzonitrile (900 mg, 2.50 mmol) and CuI (950 mmol, 5.0 mmol) in DMF (5 mL) was heated at 100° C. in a glove box overnight. The insoluble material was removed by suction filtration and the filtrate was diluted with ethyl acetate (100 mL). The organic phase was washed with brine, dried over sodium sulfate, filtered and concentrated. The resulting residue was purified by silica gel column chromatography (20% ethyl acetate in petroleum ether) to afford the title compound as a yellow solid (440 mg, 59%). ¹H NMR (400 MHz, DMSO-d₆) δ 11.64 (s, 1H), 7.63-7.61 (m, 1H), 7.51-7.47 (m, 2H), 7.39-7.35 (m, 1H), 7.32 (d, J=10.4 Hz, 1H), 6.58-6.57 (m, 1H) ppm. MS m/z: 287 [M−1]⁻.

The following intermediate was prepared utilizing the procedures described for Intermediate 15.

| No. | Structure | Name | ¹H NMR | MS m/z |
|---|---|---|---|---|
| 15-1 | 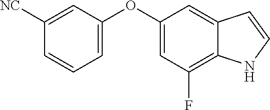 | 3-((7-fluoro- 1H-indol-5- yl)oxy) benzonitrile | (400 MHz, DMSO-d₆) δ 11.75 (br s, 1H), 7.56-7.52 (m, 2H), 7.49 (t, J = 2.8 Hz, 1H), 7.42-7.41 (m, 1H), 7.30-7.27 (m, 1H), 7.14 (d, J = 2.4 Hz, 1H), 6.85 (dd, J = 11.6, 2.0 Hz, 1H), 6.53 (dd, J = 4.8, 2.8 Hz, 1H) ppm. | 253 (M + H)⁺ |

### Intermediate 16

### 2-(3-((1H-Pyrrolo[3,2-b]pyridin-5-yl)oxy)phenyl)-1H-imidazole-5-carbaldehyde

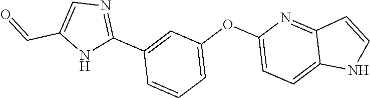 [SMILES: O=Cc1cnc(-c2cccc(Oc3ccc4[nH]ccc4n3)c2)[nH]1]

To a stirred solution of (2-(3-((1H-pyrrolo[3,2-b]pyridin-5-yl)oxy)phenyl)-1H-imidazol-5-yl)methanol (Intermediate 16A, 870 mg, 2.84 mmol) in dichloromethane (100 mL) was added manganese dioxide (4.9 g, 56.8 mmol). The reaction mixture was stirred at room temperature for sixteen hours, filtered through a pad of Celite and concentrated to afford the title compound (710 mg, 82%) as a yellow solid. The crude product was used without further purification. MS m/z: 305 [M+H]⁺.

### Intermediate 16A

### (2-(3-((1H-Pyrrolo[3,2-b]pyridin-5-yl)oxy)phenyl)-1H-imidazol-5-yl)methanol

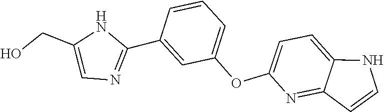 [SMILES: OCc1cnc(-c2cccc(Oc3ccc4[nH]ccc4n3)c2)[nH]1]

To a stirred solution of 3-((1H-pyrrolo[3,2-b]pyridin-5-yl)oxy)benzimidamide (Intermediate 8-5, 1.40 g, 5.6 mmol) in ammonium hydroxide solution (15 mL) were added 1,3-dihydroxypropan-2-one (1400 mg, 15.6 mmol) and ammonium chloride (1400 mg, 26.4 mmol) at room temperature. The reaction mixture was stirred at 80° C. for sixteen hours, cooled to room temperature and extracted with THF (25 mL×6). The combined organic extracts were washed with brine (30 mL), dried over sodium sulfate, filtered and concentrated to dryness. The residue was purified by silica gel column chromatography (dichloromethane/methanol/concentrated ammonium hydroxide, v/v, 20/1/0.1) to afford the title compound as a yellow solid (870 mg, 51%). MS m/z: 307 [M+H]⁺.

The following intermediate was prepared utilizing the procedures described for Intermediate 16.

| No. | Structure | Name | MS m/z |
|---|---|---|---|
| 16-1 | 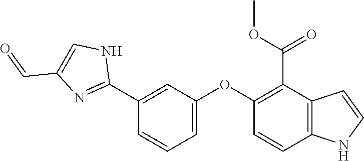 | methyl 5-(3-(4-formyl-1H-imidazol- 2-yl)phenoxy)-1H-indole-4- carboxylate | 362 [M + H]⁺ |

### PREPARATION OF EXAMPLES

### Example 1

### 5-((6-Fluoro-5-(4-fluoro-3-(4-(1-hydroxy-1-phenylethyl)-1H-imidazol-2-yl)phenoxy)-1H-indol-4-yl)methyl)thiazolidine-2,4-dione

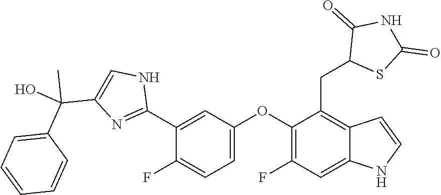 [SMILES: CC(O)(c1ccccc1)c1c[nH]c(-c2cc(Oc3c(F)cc4[nH]ccc4c3CC3SC(=O)NC3=O)ccc2F)n1]

### A. 1-(2-(2-Fluoro-5-((6-fluoro-4-vinyl-1H-indol-5-yl)oxy)phenyl)-1H-imidazol-4-yl)-1-phenylethan-1-ol

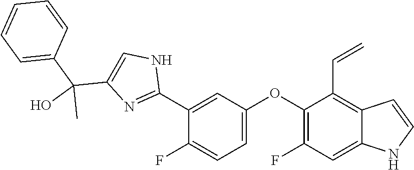 [SMILES: C=Cc1c(Oc2ccc(F)c(-c3nc(C(C)(O)c4ccccc4)c[nH]3)c2)c(F)cc2[nH]ccc12]

A mixture of 2-fluoro-5-((6-fluoro-4-vinyl-1H-indol-5-yl)oxy)benzimidamide (Intermediate 8-8, 2 g, 6.38 mmol) and 3-methyl-3-phenyloxirane-2-carbaldehyde (Intermediate 3, 1.04 g, 6.38 mmol) in DMF (20 mL) was heated at 75° C. overnight, quenched with water (100 mL) and extracted with ethyl acetate (30 mL×3). The combined organic extracts were washed with brine (10 mL), dried over magnesium sulfate and concentrated under reduced pressure. The resulting residue was purified by preparative TLC on SiO₂ (50% ethyl acetate in petroleum ether) to afford the title compound as a solid (2.68 g, 87%). MS m/z: 458 [M+H]⁺.

### B. 6-Fluoro-5-(4-fluoro-3-(4-(1-hydroxy-1-phenylethyl)-1H-imidazol-2-yl)phenoxy)-1H-indole-4-carbaldehyde

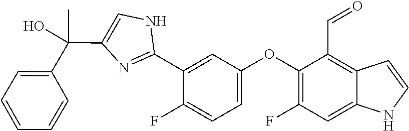 [SMILES: CC(O)(c1ccccc1)c1c[nH]c(-c2cc(Oc3c(F)cc4[nH]ccc4c3C=O)ccc2F)n1]

To a stirred solution of 1-(2-(2-fluoro-5-((6-fluoro-4-vinyl-1H-indol-5-yl)oxy)phenyl)-1H-imidazol-4-yl)-1-phenylethan-1-ol (2 g, 4.37 mmol) in a 1:1 mixture of THF and water (75 mL) was added NaIO₄ (2.8 g, 13.1 mmol). The reaction mixture was stirred at room temperature for four hours under a nitrogen atmosphere, quenched with aqueous sodium dithionite solution (20 mL×3) and extracted with ethyl acetate (20 mL×3). The combined organic layers were washed with brine (10 mL), dried (sodium sulfate), filtered and concentrated. The resulting crude product was purified by silica gel column chromatography (0-80% ethyl acetate in petroleum ether) to afford the title compound as solid (0.62 g, 31%). MS m/z: 460 [M+H]⁺.

### C. 5-((6-Fluoro-5-(4-fluoro-3-(4-(1-hydroxy-1-phenylethyl)-1H-imidazol-2-yl)phenoxy)-1H-indol-4-yl)methylene)thiazolidine-2,4-dione and 5-((6-fluoro-5-(4-fluoro-3-(4-(1-phenylvinyl)-1H-imidazol-2-yl)phenoxy)-1H-indol-4-yl)methylene)thiazolidine-2,4-dione

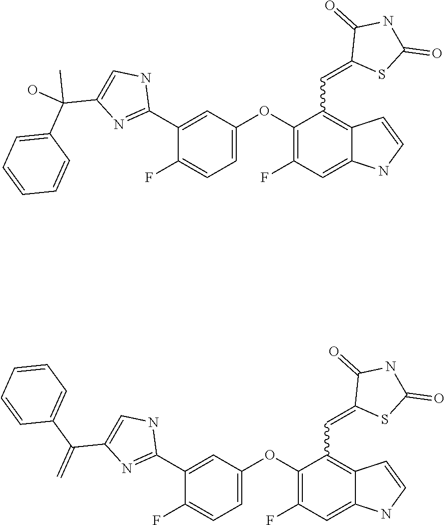

A reaction tube containing a mixture of tetrabutylammonium acetate (2.5 g), 6-fluoro-5-(4-fluoro-3-(4-(1-hydroxy-1-phenylethyl)-1H-imidazol-2-yl)phenoxy)-1H-indole-4-carbaldehyde (150 mg, 330 μmol) and thiazolidine-2,4-dione (450 mg, 4 mmol) was sealed and heated to 120° C. until the mixture melted and then maintained at 100° C. for three hours. The reaction mixture was cooled to room temperature, diluted with ethyl acetate (50 mL), washed with water and brine (20 mL×3), dried over sodium sulfate and concentrated. The resulting residue was purified by flash chromatography (0-60% ethyl acetate in petroleum ether) to afford the title compound as a solid (54 mg, 30%). LC-MS m/z: 559 [M+H]⁺, Retention time: 0.82 minutes. In addition to the desired product, 5-((6-fluoro-5-(4-fluoro-3-(4-(1-phenylvinyl)-1H-imidazol-2-yl)phenoxy)-1H-indol-4-yl)methylene)thiazolidine-2,4-dione was also obtained as a solid (30 mg, 17%). MS m/z: 541 [M+H]⁺.

### D. Example 1. 5-((6-Fluoro-5-(4-fluoro-3-(4-(1-hydroxy-1-phenylethyl)-1H-imidazol-2-yl)phenoxy)-1H-indol-4-yl)methyl)thiazolidine-2,4-dione

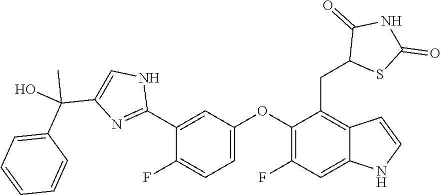 [SMILES: CC(O)(c1ccccc1)c1c[nH]c(-c2cc(Oc3c(F)cc4[nH]ccc4c3CC3SC(=O)NC3=O)ccc2F)n1]

To a stirred solution of 5-((6-fluoro-5-(4-fluoro-3-(4-(1-hydroxy-1-phenylethyl)-1H-imidazol-2-yl)phenoxy)-1H-indol-4-yl)methylene)thiazolidine-2,4-dione (90 mg, 161 μmol) in a 1:1 mixture of ethyl acetate and methanol (10 mL) was added Pd—C (10% on carbon, 45 mg). The reaction mixture was purged with hydrogen, stirred under hydrogen atmosphere overnight, filtered and rinsed with methanol (15 mL×3). The filtrate was concentrated and the resulting residue was purified by flash chromatography (0-60% ethyl acetate in petroleum ether) to afford the title compound as a solid (39.7 mg, 44%). MS m/z: 561 [M+H]⁺. ¹H NMR (400 MHz, DMSO-d₆) δ 12.08-12.00 (m 2H), 11.36 (s, 1H), 7.50-7.41 (m, 3H), 7.44 (t, J=2.8 Hz, 1H), 7.33 (d, J=10.2 Hz, 1H), 7.27-7.22 (m 3H), 7.18-7.12 (m, 1H), 695-6.88 (m, 1H), 6.81-6.71 (m, 1H), 6.63 (s, 1H), 4.88-4.84 (m, 1H), 3.59-3.52 (m, 1H), 3.41-3.37 (m, 1H), 1.73 (s, 3H) ppm.

### Example 2

### 5-((6-Fluoro-5-(4-fluoro-3-(4-(1-phenylethyl)-1H-imidazol-2-yl)phenoxy)-1H-indol-4-yl)methyl)thiazolidine-2,4-dione

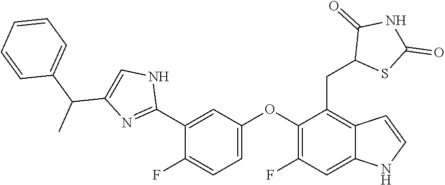 [SMILES: CC(c1ccccc1)c1c[nH]c(-c2cc(Oc3c(F)cc4[nH]ccc4c3CC3SC(=O)NC3=O)ccc2F)n1]

To a stirred solution of 5-((6-fluoro-5-(4-fluoro-3-(4-(1-phenylvinyl)-1H-imidazol-2-yl)phenoxy)-1H-indol-4-yl)methylene)thiazolidine-2,4-dione (Example 1 Step C, 50 mg, 93 μmol) in a 1:1 mixture of ethyl acetate and methanol (10 mL) was added Pd—C (10%, 25 mg).

The mixture was purged with hydrogen and stirred under hydrogen atmosphere overnight. The mixture was filtered, rinsed with methanol (5 mL×3) and the filtrate was concentrated. The resulting residue was purified by flash chromatography (0-60% ethyl acetate in petroleum ether) to afford the title compound as a solid (32.6 mg, 65%). MS m/z: 545 [M+H]⁺. ¹H NMR (400 MHz, CD₃OD) δ 7.44-7.41 (m, 1H), 7.34 (d, J=3.2 Hz, 1H), 7.30-7.22 (m, 5H), 7.20-7.14 (m, 2H), 6.89 (dt, J=9.2, 3.6 Hz, 1H), 6.86 (s, 1H), 6.64 (d, J=3.2 Hz, 1H), 4.86 (dd, J=10.4, 4.4 Hz, 1H), 4.15 (q, J=7.2 Hz, 1H), 3.76 (dd, J=13.6, 4.4 Hz, 1H), 3.40 (dd, J=13.6, 10.4 Hz, 1H), 1.61 (d, J=7.6 Hz, 3H) ppm.

### Example 3

### 5-((6-fluoro-5-(3-(5-(1-hydroxy-1-phenylethyl)-1H-imidazol-2-yl)phenoxy)-1H-indol-4-yl)methylene)thiazolidine-2,4-dione

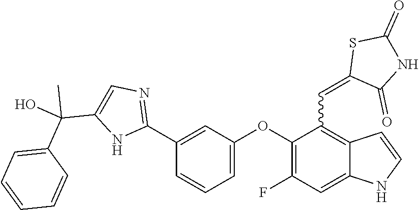 [SMILES: CC(O)(c1ccccc1)c1cnc(-c2cccc(Oc3c(F)cc4[nH]ccc4c3C=C3SC(=O)NC3=O)c2)[nH]1]

The title compound was prepared from 3-((6-fluoro-4-vinyl-1H-indol-5-yl)oxy)benzimidamide (Intermediate 8-14) and 3-methyl-3-phenyloxirane-2-carbaldehyde (Intermediate 3) utilizing the procedures described for Example 1, steps A to C. MS m/z: 541 [M+H]⁺, ¹H NMR (400 MHz, DMSO-d₆) δ 8.04 (s, 1H), 7.29-7.51 (m, 10H), 6.81-6.89 (m, 2H), 6.57-6.58 (m, 1H), 1.89 (s, 3H) ppm.

### Example 4

### 2-(6-Fluoro-5-(4-fluoro-3-(5-(2-phenylpropan-2-yl)-1H-imidazol-2-yl)phenoxy)-1H-indol-4-yl)acetic acid

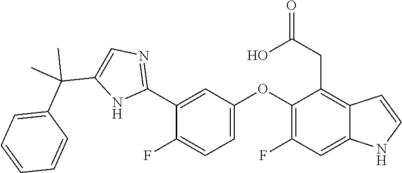 [SMILES: CC(C)(c1ccccc1)c1cnc(-c2cc(Oc3c(F)cc4[nH]ccc4c3CC(=O)O)ccc2F)[nH]1]

### A. (Z)-5-((4-(2-Ethoxyvinyl)-6-fluoro-1-tosyl-1H-indol-5-yl)oxy)-2-fluorobenzonitrile

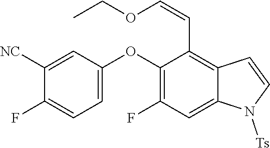 [SMILES: CCO/C=C\c1c(Oc2ccc(F)c(C#N)c2)c(F)cc2c1ccn2S(=O)(=O)c1ccc(C)cc1]

To a stirred mixture of 5-((4-bromo-6-fluoro-1-tosyl-1H-indol-5-yl)oxy)-2-fluorobenzonitrile (3.61 g, 7.2 mmol), Pd(PPh₃)₂Cl₂ (0.5 g; 0.72 mmol) and LiCl (0.9 g, 21.6 mmol) in DMF (72 mL) was added (Z)-tributyl(2-ethoxyvinyl)stannane (3.11 g, 8.6 mmol) under nitrogen atmosphere. The reaction mixture was stirred at 100° C. for three hours, cooled to room temperature, quenched with saturated potassium fluoride solution (75 mL) and extracted with ethyl acetate (60 mL×3). The combined organic extracts were washed with brine (30 mL×2), dried over sodium sulfate, filtered and concentrated. The resulting residue was purified by silica gel column chromatography to afford the title compound as yellow solid (2.4 g, 67%). ¹H NMR (400 MHz, CDCl₃) δ 7.82 (d, J=8.4 Hz, 2H), 7.76 (d, J=10.4 Hz, 1H), 7.60 (d, J=4.0 Hz, 1H), 7.31 (d, J=8.0 Hz, 2H), 7.14-7.11 (m, 2H), 7.01-6.99 (m, 1H), 6.75 (d, J=3.6 Hz, 1H), 6.27 (d, J=7.2 Hz, 1H), 5.29 (d, J=6.8 Hz, 1H), 3.90 (q, J=7.2 Hz, 2H), 2.14 (s, 3H), 1.22 (t, J=6.8 Hz, 3H) ppm

### B. (Z)-5-((4-(2-Ethoxyvinyl)-6-fluoro-1H-indol-5-yl)oxy)-2-fluorobenzonitrile

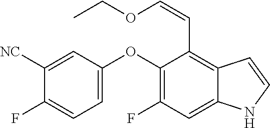 [SMILES: CCO/C=C\c1c(Oc2ccc(F)c(C#N)c2)c(F)cc2[nH]ccc12]

To a stirred solution of (Z)-5-((4-(2-ethoxyvinyl)-6-fluoro-1-tosyl-1H-indol-5-yl)oxy)-2-fluorobenzonitrile (1 g, 2 mmol) in methanol (15 mL) was added potassium carbonate (0.84 g, 6 mmol). The reaction mixture was stirred at 80° C. for 0.5 hours in a microwave reactor and then concentrated. The resulting residue was dissolved in ethyl acetate (150 mL), washed with water and brine, dried over sodium sulfate, filtered and concentrated. The resulting crude product was purified by silica gel column chromatography (petroleum ether/ethyl acetate, v/v, 10/1 to 3/1) to afford the title compound as a yellow solid (0.38 g, 56%). MS m/z: 341 [M+H]⁺.

### C. (Z)-5-((4-(2-Ethoxyvinyl)-6-fluoro-1H-indol-5-yl)oxy)-2-fluorobenzimidamide

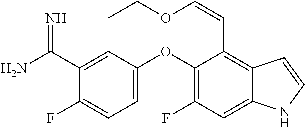 [SMILES: CCO/C=C\c1c(Oc2ccc(F)c(C(=N)N)c2)c(F)cc2[nH]ccc12]

To a stirred solution of (Z)-5-((4-(2-ethoxyvinyl)-6-fluoro-1H-indol-5-yl)oxy)-2-fluorobenzonitrile (0.38 g, 1.12 mmol) in THF (8 ml) was added a 1.0 M solution of lithium bis(trimethylsilyl)amide in THF (12 ml, 12 mmol). The reaction mixture was stirred at room temperature for 12 hours, quenched with water (5 mL) and extracted with THF (40 mL×3). The combined organic extracts were dried over sodium sulfate, filtered and evaporated to afford the title compound as yellow solid (0.39 g, 90%). The crude product was used without further purification. MS m/z: 358 [M+H]⁺.

### D. (Z)-4-(2-Ethoxyvinyl)-6-fluoro-5-(4-fluoro-3-(5-(2-phenylpropan-2-yl)-1H-imidazol-2-yl)phenoxy)-1H-indole

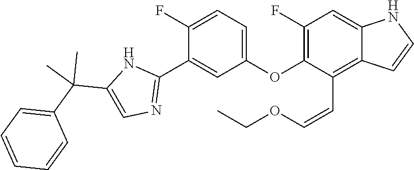 [SMILES: CCO/C=C\c1c(Oc2ccc(F)c(-c3ncc(C(C)(C)c4ccccc4)[nH]3)c2)c(F)cc2[nH]ccc12]

To a stirred solution of (Z)-5-((4-(2-ethoxyvinyl)-6-fluoro-1H-indol-5-yl)oxy)-2-fluorobenzimidamide (0.23 g, 0.64 mmol) and 1-bromo-3-methyl-3-phenylbutan-2-one (0.15 g, 0.64 mmol, Intermediate 4-2) in DMF (5 ml) was added potassium carbonate (0.27 g, 1.92 mmol). The reaction mixture was stirred at 75° C. for three hours, cooled to room temperature, diluted with water (30 mL) and extracted with ethyl acetate (30 ml×3). The combined organic extracts were washed with brine, dried over sodium sulfate, filtered and concentrated. The resulting residue was purified by flash chromatography (petroleum ether/ethyl acetate, v/v, 10/1 to 3/1) to afford the title compound as yellow solid (0.18 g, 58%). MS m/z: 500 [M+H]⁺

### E. 2-(6-Fluoro-5-(4-fluoro-3-(5-(2-phenylpropan-2-yl)-1H-imidazol-2-yl)phenoxy)-1H-indol-4-yl)acetaldehyde

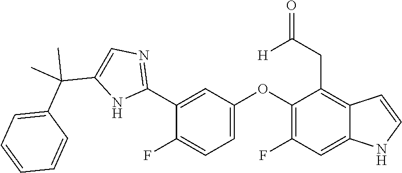 [SMILES: CC(C)(c1ccccc1)c1cnc(-c2cc(Oc3c(F)cc4[nH]ccc4c3CC=O)ccc2F)[nH]1]

Hydrochloric acid (1 M, 6 mL, 6 mmol) was added to a solution of (Z)-4-(2-ethoxyvinyl)-6-fluoro-5-(4-fluoro-3-(5-(2-phenylpropan-2-yl)-1H-imidazol-2-yl)phenoxy)-1H-indole (0.3 g, 0.6 mmol) in THF (6 mL) and stirred at 40° C. for one hour. The reaction mixture was cooled to room temperature, quenched with saturated sodium bicarbonate solution (50 mL) and extracted with dichloromethane (60 mL×3). The combined organic extracts were washed with brine, dried over sodium sulfate, filtered and evaporated. The resulting residue was purified by silica gel column chromatography (petroleum ether/ethyl acetate, v/v, 2/1) to afford the title compound as yellow solid (160 mg, 51%). MS m/z: 472 [M+H]⁺.

### F. Example 4. 2-(6-Fluoro-5-(4-fluoro-3-(5-(2-phenylpropan-2-yl)-1H-imidazol-2-yl)phenoxy)-1H-indol-4-yl)acetic acid

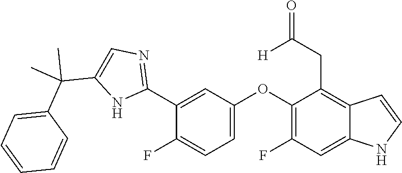 [SMILES: CC(C)(c1ccccc1)c1cnc(-c2cc(Oc3c(F)cc4[nH]ccc4c3CC=O)ccc2F)[nH]1]

To a mixture of 2-(6-fluoro-5-(4-fluoro-3-(5-(2-phenylpropan-2-yl)-1H-imidazol-2-yl)phenoxy)-1H-indol-4-yl)acetaldehyde (160 mg, 0.34 mmol), NaClO₂ (244 mg, 2.7 mmol) and NaH₂PO₄ (326 mg, 2.7 mmol) in a 3:3:1 mixture of THF, t-Butanol and water (14 mL) was added 2-methyl-2-butene (356 mg, 5.1 mmol). The mixture was stirred at room temperature overnight, quenched with 50 mL of water and extracted with ethyl acetate (60 mL×3). The combined organic extracts were dried over sodium sulfate and concentrated to dryness. The solid was collected and washed with ethyl acetate to afford the title compound as white solid (25 mg, 15%). MS m/z: 488 [M+H]⁺. ¹H NMR (400 MHz, CD₃OD) δ 7.39 (dd, J=5.6, 2.8 Hz, 1H), 7.30-7.28 (m, 5H), 7.21-7.14 (m, 3H), 7.06 (s, 1H), 6.98-6.95 (m, 1H), 6.53 (d, J=3.2 Hz, 1H), 3.85 (s, 2H), 1.71 (s, 6H) ppm.

### Example 5

### 1-(2-(3-((6-Fluoro-4-(methylsulfonyl)-1H-indol-5-yl)oxy)phenyl)-1H-imidazol-5-yl)-1-phenylethan-1-ol

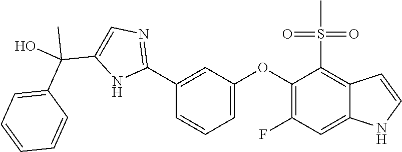 [SMILES: CC(O)(c1ccccc1)c1cnc(-c2cccc(Oc3c(F)cc4[nH]ccc4c3S(C)(=O)=O)c2)[nH]1]

### A. 1-(2-(3-((6-Fluoro-4-(methylthio)-1H-indol-5-yl)oxy)phenyl)-1H-imidazol-5-yl)-1-phenylethan-1-ol

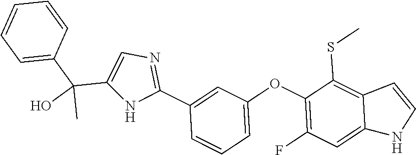 [SMILES: CSc1c(Oc2cccc(-c3ncc(C(C)(O)c4ccccc4)[nH]3)c2)c(F)cc2[nH]ccc12]

3-((6-Fluoro-4-(methylthio)-1H-indol-5-yl)oxy)benzimidamide (150 mg, 0.48 mmol, Intermediate 8-2) and 3-methyl-3-phenyloxirane-2-carbaldehyde (78 mg, 0.48 mmol, Intermediate 3) were dissolved in DMF (3 mL) in a glove box and the solution was then stirred at 75° C. for three hours. The reaction mixture was cooled to room temperature, quenched with water (20 mL) and extracted with ethyl acetate (20 mL×3). The combined organic extracts were washed with brine (20 mL), dried over sodium sulfate, filtered and concentrated. The resulting residue was purified by flash chromatography (0-100% ethyl acetate in petroleum ether) to afford the title compound as a white solid (87 mg, 47%). MS m/z: 460 [M+H]⁺. LCMS Retention time 1.76 minutes.

### B. Example 5. 1-(2-(3-((6-Fluoro-4-(methylsulfonyl)-1H-indol-5-yl)oxy)phenyl)-1H-imidazol-5-yl)-1-phenylethan-1-ol

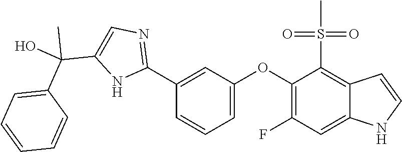 [SMILES: CC(O)(c1ccccc1)c1cnc(-c2cccc(Oc3c(F)cc4[nH]ccc4c3S(C)(=O)=O)c2)[nH]1]

To a stirred solution of 1-(2-(3-((6-fluoro-4-(methylthio)-1H-indol-5-yl)oxy)phenyl)-1H-imidazol-5-yl)-1-phenylethan-1-ol (65.3 mg, 0.14 mmol) in a mixture of dichloromethane (1.6 mL) and methanol (10 mL) was added a solution of ammonium molybdate tetrahydrate (200 mg) in hydrogen peroxide (30% in water, 1 mL). The reaction mixture was stirred at room temperature for eighty minutes, diluted with ethyl acetate (80 mL), washed with water (20 mL×3), aqueous sodium sulfite (20 mL) and brine (20 mL×2), dried and concentrated. The residue was purified by flash chromatography (0-100% ethyl acetate in petroleum ether) to afford the title compound as a white solid (35 mg, 51%). MS m/z: 492 [M+H]⁺. ¹H NMR (400 MHz, CD₃OD) δ 7.65 (d, J=10.4 Hz, 1H), 7.58 (d, J=8.0 Hz, 1H), 7.53 (d, J=3.2 Hz, 1H), 7.48-7.46 (m, 3H), 7.38 (t, J=8.0 Hz, 1H), 7.32 (t, J=7.6 Hz, 2H), 7.24 (d, J=7.2 Hz, 1H), 7.20 (d, J=3.2 Hz, 1H), 6.95 (dd, J=8.0, 2.4 Hz, 1H), 6.87 (br s, 1H), 3.33 (s, 3H), 1.89 (s, 3H) ppm.

The following example was prepared utilizing similar procedures as described in Example 5.

| Ex. No. | Structue | Name | ¹H NMR | MS m/z |
|---|---|---|---|---|
| 6 | 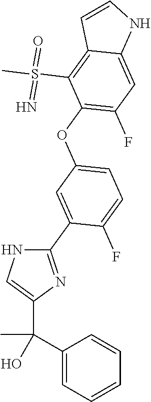 [SMILES: CC(O)(c1ccccc1)c1c[nH]c(-c2cc(Oc3c(F)cc4[nH]ccc4c3S(C)(=N)=O)ccc2F)n1] | (6-fluoro-5-(4-fluoro-3-(4- (1-hydroxy-1-phenylethyl)- 1H-imidazol-2-y1)phenoxy)- 1H-indol-4- y1)(imino)(methyl)-16- sulfanone | (400 MHz, CD₃OD) δ 7.59 (d, J = 10.8 Hz, 1H), 7.49 (d, J = 3.2 Hz, 1H), 7.44 (d, J = 8.0 Hz, 2H), 7.41-7.15 (m, 6H), 7.09-7.05 (m, 1H), 6.99-6.91 (m, 1H), 3.40 (s, 3H), 1.86 (s, 3H) ppm. | 509 [M + H]⁺ |

### Example 7

### Methyl 6-fluoro-5-(3-(5-(1-hydroxy-1-phenylethyl)-1H-imidazol-2-yl)phenoxy)-1H-indole-4-carboxylate

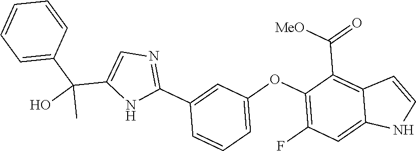 [SMILES: COC(=O)c1c(Oc2cccc(-c3ncc(C(C)(O)c4ccccc4)[nH]3)c2)c(F)cc2[nH]ccc12]

### A. Methyl 5-(3-cyanophenoxy)-6-fluoro-1-tosyl-1H-indole-4-carboxylate

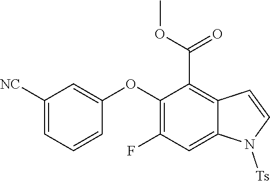 [SMILES: COC(=O)c1c(Oc2cccc(C#N)c2)c(F)cc2c1ccn2S(=O)(=O)c1ccc(C)cc1]

A mixture of 3-((4-bromo-6-fluoro-1-tosyl-1H-indol-5-yl)oxy)benzonitrile (3 g, 6.2 mmol), triethylamine (3.76 g, 37.2 mmol) and Pd(dppf)Cl₂ (2.52 g, 3.1 mmol) in methanol (30 mL) and DMF (50 mL) was heated at 70° C. under carbon monoxide atmosphere for three days. The reaction mixture was cooled to room temperature and filtered. The filtrate was concentrated and the residue was purified by silica gel column chromatography (0-100% dichloromethane in petroleum ether) to afford the title compound as a white solid (2 g, 69%). MS m/z: 465 [M+H]⁺.

### B. Methyl 5-(3-cyanophenoxy)-6-fluoro-1H-indole-4-carboxylate

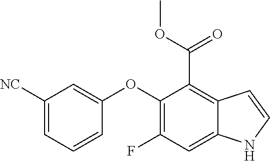 [SMILES: COC(=O)c1c(Oc2cccc(C#N)c2)c(F)cc2[nH]ccc12]

To a stirred solution of methyl 5-(3-cyanophenoxy)-6-fluoro-1-tosyl-1H-indole-4-carboxylate (500 mg, 1.08 mmol) in THF (3 mL) was added a 1.0 M solution of tetrabutylammonium fluoride in THF (10.8 mL, 10.8 mmol). The reaction mixture was heated at 70° C. for four hours and concentrated to dryness at 35° C. Water (20 mL) was added to the residue, the solid was collected by filtration, washed with water and purified by silica gel column chromatography (0-100% dichloromethane in petroleum ether) to afford the title compound as a white solid (260 mg, 84%). MS m/z: 311 [M+H]⁺.

### C. Methyl 5-(3-carbamimidoylphenoxy)-6-fluoro-1H-indole-4-carboxylate

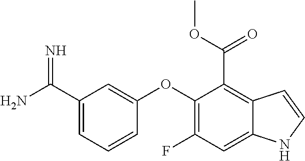 [SMILES: COC(=O)c1c(Oc2cccc(C(=N)N)c2)c(F)cc2[nH]ccc12]

To a stirred solution of methyl 5-(3-cyanophenoxy)-6-fluoro-1H-indole-4-carboxylate (680 mg, 2.19 mmol) in THF (15 mL) was added a 1.0 M solution of lithium bis(trimethylsilyl)amide in THF (8.8 mL, 8.77 mmol) at room temperature. The resulting mixture was stirred at room temperature for sixteen hours, quenched with methanol (10 mL) and concentrated. The resulting residue was purified by silica gel column chromatography (dichloromethane/methanol/concentrated ammonium hydroxide, v/v, 5/1/0.1) to afford the title compound as a yellow solid (490 mg, 68%). MS m/z: 328 [M+H]⁺.

### D. Example 7. Methyl 6-fluoro-5-(3-(5-(1-hydroxy-1-phenylethyl)-1H-imidazol-2-yl)phenoxy)-1H-indole-4-carboxylate

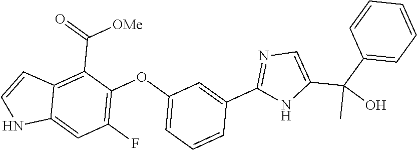 [SMILES: COC(=O)c1c(Oc2cccc(-c3ncc(C(C)(O)c4ccccc4)[nH]3)c2)c(F)cc2[nH]ccc12]

A mixture of methyl 5-(3-carbamimidoylphenoxy)-6-fluoro-1H-indole-4-carboxylate (490 mg, 1.5 mmol) and 3-methyl-3-phenyloxirane-2-carbaldehyde (Intermediate 3, 291 mg, 1.8 mmol) in dry DMF (10 mL) was stirred at 80° C. under nitrogen overnight and concentrated to dryness.

The resulting residue was purified by silica gel column chromatography (dichloromethane/methanol/concentrated ammonium hydroxide, v/v, 20/1/0.1) to afford the title compound as a yellow solid (250 mg, 35%). MS m/z: 472 [M+H]⁺. ¹H NMR (400 MHz, CD₃OD) δ 7.52-7.46 (m, 4H), 7.43 (d, J=3.2 Hz, 1H), 7.40 (s, 1H), 7.36-7.30 (m, 3H), 7.25-7.21 (m, 1H), 6.89-6.82 (m, 3H), 3.79 (s, 3H), 1.89 (s, 3H) ppm.

### Example 8

### 1-(2-(3-((6-Fluoro-4-(hydroxymethyl)-1H-indol-5-yl)oxy)phenyl)-1H-imidazol-5-yl)-1-phenylethan-1-ol

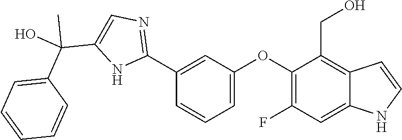 [SMILES: CC(O)(c1ccccc1)c1cnc(-c2cccc(Oc3c(F)cc4[nH]ccc4c3CO)c2)[nH]1]

To a stirred solution of methyl 6-fluoro-5-(3-(5-(1-hydroxy-1-phenylethyl)-1H-imidazol-2-yl)phenoxy)-1H-indole-4-carboxylate (Example 7, 80 mg, 0.17 mmol) in THF (10 mL) was added a 1.0 M solution of LiAlH₄ in THF (1.69 mL, 1.69 mmol) at 0° C. The mixture was stirred at room temperature for four hours, quenched with water and extracted with ethyl acetate. The combined organic extracts were washed with brine, dried over sodium sulfate and concentrated. The resulting residue was purified by preparative HPLC to afford the title compound as a white solid (33 mg, 44%). MS m/z: 444 [M+H]⁺. ¹H NMR (400 MHz, CD₃OD) δ 7.52-7.45 (m, 3H), 7.36-7.29 (m, 5H), 7.24-7.21 (m, 2H), 6.92 (dd, J=2.4, 8.0 Hz, 1H), 6.86 (s, 1H), 6.73 (d, J=3.2 Hz, 1H), 4.86 (s, 2H), 1.88 (s, 3H) ppm.

### Example 9

### 6-Fluoro-5-(3-(5-(1-hydroxy-1-phenylethyl)-1H-imidazol-2-yl)phenoxy)-1H-indole-4-carboxamide

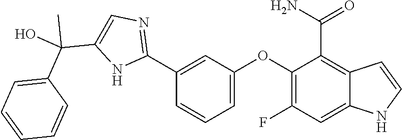 [SMILES: CC(O)(c1ccccc1)c1cnc(-c2cccc(Oc3c(F)cc4[nH]ccc4c3C(N)=O)c2)[nH]1]

### A. 6-Fluoro-5-(3-(5-(1-hydroxy-1-phenylethyl)-1H-imidazol-2-yl)phenoxy)-1H-indole-4-carboxylic acid

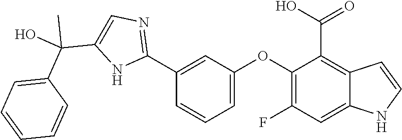 [SMILES: CC(O)(c1ccccc1)c1cnc(-c2cccc(Oc3c(F)cc4[nH]ccc4c3C(=O)O)c2)[nH]1]

To a stirred solution of methyl 6-fluoro-5-(3-(5-(1-hydroxy-1-phenylethyl)-1H-imidazol-2-yl)phenoxy)-1H-indole-4-carboxylate (Example 7, 150 mg, 0.32 mmol) in a mixture of THF (5 mL), methanol (5 mL) and water (2 mL) was added lithium hydroxide monohydrate (152 mg, 6.4 mmol). The resulting mixture was stirred at room temperature for two days and concentrated to remove THF. The residue was dissolved in 2 ml, of water and acidified to pH 3 with 3M hydrochloric acid. The precipitate was collected by filtration and purified by preparative HPLC to afford the title compound as a white solid (130 mg, 89%). MS m/z: 458 [M+H]⁺. ¹H NMR (400 MHz, CD₃OD) δ 7.54-7.51 (m, 5H), 7.45-7.44 (m, 3H), 7.38 (t, J=7.6 Hz, 1H), 7.30 (t, J=7.2 Hz, 1H), 7.16-7.14 (m, 1H), 6.88 (d, J=3.2 Hz, 1H), 1.95 (s, 3H) ppm.

### B. Example 9. 6-Fluoro-5-(3-(5-(1-hydroxy-1-phenylethyl)-1H-imidazol-2-yl)phenoxy)-1H-indole-4-carboxamide

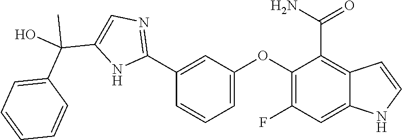 [SMILES: CC(O)(c1ccccc1)c1cnc(-c2cccc(Oc3c(F)cc4[nH]ccc4c3C(N)=O)c2)[nH]1]

A mixture of 6-fluoro-5-(3-(5-(1-hydroxy-1-phenylethyl)-1H-imidazol-2-yl)phenoxy)-1H-indole-4-carboxylic acid (80 mg, 0.18 mmol), ammonium chloride (185 mg, 3.5 mmol), HATU (133 mg, 0.35 mmol) and triethylamine (442 mg, 4.4 mmol) in DMF (5 mL) was stirred at room temperature for two hours. The mixture was poured into ice water (50 mL), the solid was collected and purified by preparative HPLC to afford the title compound as a white solid (31 mg, 39%). MS m/z: 457 [M+H]⁺. ¹H NMR (400 MHz, CD₃OD) δ 7.52 (d, J=8.0 Hz, 1H), 7.46 (d, J=7.6 Hz, 1H), 7.41-7.29 (m, 7H), 7.23 (t, J=7.2 Hz, 1H), 6.96 (d, J=8.4 Hz, 1H), 6.86 (s, 1H), 6.72 (d, J=2.8 Hz, 1H), 1.88 (s, 3H) ppm.

### Example 10

### 3-(6-Fluoro-5-(4-fluoro-3-(5-(2-phenylpropan-2-yl)-1H-imidazol-2-yl)phenoxy)-1H-indol-4-yl)propanoic acid

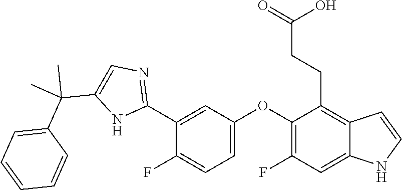 [SMILES: CC(C)(c1ccccc1)c1cnc(-c2cc(Oc3c(F)cc4[nH]ccc4c3CCC(=O)O)ccc2F)[nH]1]

### A. 4-Bromo-6-fluoro-5-(4-fluoro-3-(5-(2-phenylpropan-2-yl)-1H-imidazol-2-yl)phenoxy)-1H-indole

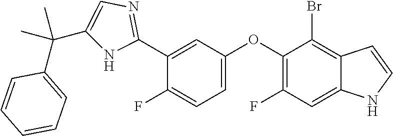 [SMILES: CC(C)(c1ccccc1)c1cnc(-c2cc(Oc3c(F)cc4[nH]ccc4c3Br)ccc2F)[nH]1]

A mixture of 5-((4-bromo-6-fluoro-1H-indol-5-yl)oxy)-2-fluorobenzimidamide (Intermediate 8-1, 3.07 g, 8.39 mmol), 1-chloro-3-methyl-3-phenylbutan-2-one (Intermediate 4, 1.5 g, 7.63 mmol) and sodium bicarbonate (1.28 g, 15.25 mmol) in acetonitrile (6 mL) was heated at 70° C. overnight. The solvent was evaporated and the residue was purified by column chromatography (30-50% ethyl acetate in heptane) to afford the title compound as a white solid (2.3 g, 59%). ¹H NMR (400 MHz, CD₃OD) δ 7.36 (d, J=3.3 Hz, 2H), 7.32-7.19 (m, 5H), 7.17-7.07 (m, 2H), 6.90 (s, 1H), 6.81 (s, 1H), 6.49 (dd, J=3.2, 0.9 Hz, 1H), 1.68 (s, 6H) ppm.

### B. Ethyl (E)-3-(6-fluoro-5-(4-fluoro-3-(5-(2-phenylpropan-2-yl)-1H-imidazol-2-yl)phenoxy)-1H-indol-4-yl)acrylate

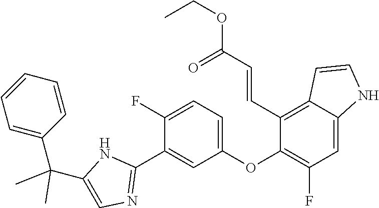 [SMILES: CCOC(=O)/C=C/c1c(Oc2ccc(F)c(-c3ncc(C(C)(C)c4ccccc4)[nH]3)c2)c(F)cc2[nH]ccc12]

A mixture of 4-bromo-6-fluoro-5-(4-fluoro-3-(5-(2-phenylpropan-2-yl)-1H-imidazol-2-yl)phenoxy)-1H-indole (300 mg, 590.13 μmol) and triethylamine (658 μl, 4.72 mmol) in DMF (1.5 mL) was degassed for ten minutes. To this mixture was added tri-o-tolylphosphine (36 mg, 118 μmol), palladium(II) acetate (6.6 mg, 30 μmol) and ethyl acrylate (75 μl, 708 μmol). The reaction vial was sealed and the mixture was heated at 130° C. for 30 minutes in a microwave reactor. The solvent was evaporated and the residue was purified by column chromatography (30-60% ethyl acetate in heptane) to afford the title compound as a yellow solid (110 mg, 35%). ¹H NMR (400 MHz, CD₃OD) δ 8.05 (d, J=16.3 Hz, 1H), 7.44 (d, J=3.2 Hz, 1H), 7.35 (d, J=10.4 Hz, 1H), 7.31-7.19 (m, 5H), 7.13 (dd, J=10.5, 9.0 Hz, 2H), 6.90 (s, 1H), 6.84 (s, 1H), 6.81-6.69 (m, 2H), 4.20 (q, J=7.1 Hz, 2H), 1.67 (s, 6H), 1.27 (t, J=7.1 Hz, 3H) ppm.

### C. Ethyl 3-(6-fluoro-5-(4-fluoro-3-(5-(2-phenylpropan-2-yl)-1H-imidazol-2-yl)phenoxy)-1H-indol-4-yl)propanoate

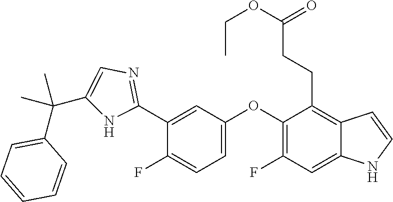 [SMILES: CCOC(=O)CCc1c(Oc2ccc(F)c(-c3ncc(C(C)(C)c4ccccc4)[nH]3)c2)c(F)cc2[nH]ccc12]

A reaction vessel containing a mixture of ethyl (E)-3-(6-fluoro-5-(4-fluoro-3-(5-(2-phenylpropan-2-yl)-1H-imidazol-2-yl)phenoxy)-1H-indol-4-yl)acrylate (100 mg, 190 μmol) and Pd—C (60 mg, 10% on carbon) in ethyl acetate (10 mL) was attached to a balloon filled with hydrogen and stirred for five hours at 50° C. The mixture was filtered through a pad of Celite and washed with ethyl acetate. The filtrate was concentrated and the resulting residue was chromatographed (20-60% ethyl acetate in heptane) to afford the tide compound as a white solid (57 mg, 57%). ¹H NMR (400 MHz, CD₃OD) δ 7.39 (s, 1H), 7.31-7.19 (m, 5H), 7.17-7.05 (m, 3H), 6.90 (s, 1H), 6.79 (d, J=8.7 Hz, 1H), 6.51 (dd, J=3.3, 0.9 Hz, 1H), 4.01 (q, J=7.1 Hz, 2H), 3.13 (dd, J=8.7, 7.1 Hz, 2H), 2.57 (dd, J=8.7, 7.2 Hz, 2H), 1.68 (s, 6H), 1.12 (t, J=7.1 Hz, 3H) ppm.

### D. Example 10. 3-(6-Fluoro-5-(4-fluoro-3-(5-(2-phenylpropan-2-yl)-1H-imidazol-2-yl)phenoxy)-1H-indol-4-yl)propanoic acid

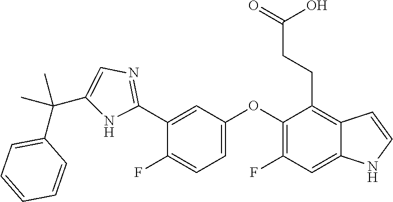 [SMILES: CC(C)(c1ccccc1)c1cnc(-c2cc(Oc3c(F)cc4[nH]ccc4c3CCC(=O)O)ccc2F)[nH]1]

A mixture of ethyl 3-(6-fluoro-5-(4-fluoro-3-(5-(2-phenylpropan-2-yl)-1H-imidazol-2-yl)phenoxy)-1H-indol-4-yl)propanoate (50 mg, 94 μmol) and lithium hydroxide (4.5 mg, 189 μmol) in THF (5 mL) and water (2 mL) was stirred overnight at room temperature. The solvent was evaporated, the residue was dissolved in 1 mL of water and acidified with 1N hydrochloric acid to pH ˜2. The white solid was collected by filtration and dried to afford the title compound (35 mg, 74%). MS m/z: 502 [M+H]⁺. ¹H NMR (400 MHz, CD₃OD) δ 7.42 (s, 1H), 7.38-7.18 (m, 8H), 7.15 (d, J=10.6 Hz, 1H), 7.06 (dd, J=8.5, 4.4 Hz, 1H), 6.56 (dd, J=3.3, 0.9 Hz, 1H), 3.17-3.07 (m, 2H), 2.60-2.51 (m, 2H), 1.75 (s, 6H) ppm.

### Example 11

### 2-(5-(3-(5-Benzyl-4H-1,2,4-triazol-3-yl)phenoxy)-1H-indol-4-yl)acetic acid

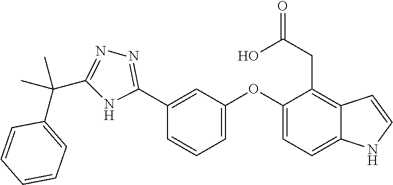 [SMILES: CC(C)(c1ccccc1)c1nnc(-c2cccc(Oc3ccc4[nH]ccc4c3CC(=O)O)c2)[nH]1]

### A. (Z)-3-((4-(2-Ethoxyvinyl)-1-tosyl-1H-indol-5-yl)oxy)benzonitrile

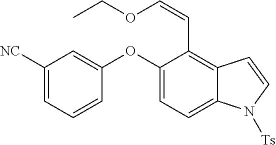 [SMILES: CCO/C=C\c1c(Oc2cccc(C#N)c2)ccc2c1ccn2S(=O)(=O)c1ccc(C)cc1]

(Z)-Tributyl(2-ethoxyvinyl)stannane (3.5 ml, 10.3 mmol) was added to a mixture of 3-((4-bromo-1-tosyl-1H-indol-5-yl)oxy)benzonitrile (Intermediate 5-2, 4.0 g, 8.56 mmol), Pd(PPh₃)Cl₂ (604 mg, 0.86 mmol) and LiCl (1.08 g, 25.8 mmol) in DMF (40 mL) under nitrogen atmosphere and the resulting mixture was stirred at 100° C. for three hours. The reaction mixture was cooled to room temperature, quenched with saturated potassium fluoride solution (20 mL) and extracted with ethyl acetate (20 mL×3). The combined organic extracts were washed with brine (20 mL×3), dried over sodium sulfate, filtered and concentrated. The resulting residue was purified by silica gel column chromatography (petroleum ether/ethyl acetate, v/v, 10/1) to afford the title compound as a yellow solid (3.4 g, 87%). MS m/z: 459 [M+H]⁺.

### B. 3-((4-(2-Oxoethyl)-1-tosyl-1H-indol-5-yl)oxy)benzonitrile

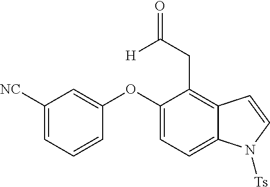 [SMILES: Cc1ccc(S(=O)(=O)n2ccc3c(CC=O)c(Oc4cccc(C#N)c4)ccc32)cc1]

To a stirred solution of (Z)-3-((4-(2-ethoxyvinyl)-1-tosyl-1H-indol-5-yl)oxy)benzonitrile (2 g, 4.37 mmol) in THF (20 mL) was added 1N hydrochloric acid (43.7 ml, 43.7 mmol). The reaction mixture was heated at 80° C. for two hours and concentrated to remove THF. The aqueous mixture was neutralized with saturated sodium bicarbonate solution and extracted with dichloromethane (20 mL×3). The combined organic phase was washed with brine (20 mL), dried over sodium sulfate, filtered and concentrated to afford the title compound as a yellow solid (1.81 g). The crude product was used in the next step without further purification. MS m/z: 431 [M+H]⁺.

### C. 2-(5-(3-Cyanophenoxy)-1-tosyl-1H-indol-4-yl)acetic acid

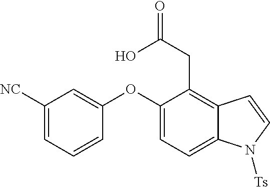 [SMILES: Cc1ccc(S(=O)(=O)n2ccc3c(CC(=O)O)c(Oc4cccc(C#N)c4)ccc32)cc1]

To a stirred solution of 3-((4-(2-oxoethyl)-1-tosyl-1H-indol-5-yl)oxy)benzonitrile (1.8 g, 4.18 mmol) in acetone (15 mL) was added Jones reagent (8 ml, 16 mmol, 2M CrO₃ in aqueous sulfuric acid). The reaction mixture was stirred at 10° C. for one hour, quenched with water (40 mL) and extracted with ethyl acetate (30 mL×3). The combined organic extracts were washed with brine (20 mL), dried over sodium sulfate, filtered and concentrated to afford the title compound as a yellow solid (2 g). The crude product was used without further purification. MS m/z: 447 [M+H]⁺.

### D. Example 11. 2-(5-(3-(5-Benzyl-4H-1,2,4-triazol-3-yl)phenoxy)-1H-indol-4-yl)acetic acid

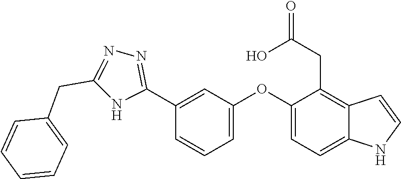 [SMILES: O=C(O)Cc1c(Oc2cccc(-c3nnc(Cc4ccccc4)[nH]3)c2)ccc2[nH]ccc12]

A mixture of 2-(5-(3-cyanophenoxy)-1-tosyl-1H-indol-4-yl)acetic acid (1.2 g, 2.69 mmol), 2-phenylacetohydrazide (2.02 g, 13.5 mmol) and potassium carbonate (1.86 g, 13.5 mmol) in n-Butanol (10 mL) was heated at 180° C. for three hours in a microwave reactor. The reaction mixture was diluted with ethyl acetate (100 mL), washed with water and brine, dried over sodium sulfate, filtered and concentrated. The resulting residue was purified by preparative HPLC to afford the tile compound as a yellow solid (120 mg, 10.5% from 3 steps). MS m/z: 425 [M+H]⁺. ¹H NMR (500 MHz, DMSO-d₆) δ 11.18 (s, 1H), 7.60 (d, J=7.5 Hz, 1H), 7.46 (s, 1H), 7.39-7.20 (m, 8H), 6.95 (dd, J=8.0, 2.0 Hz, 1H), 6.78 (d, J=8.5 Hz, 1H), 6.50 (s, 1H), 4.06 (s, 2H), 3.69 (s, 2H) ppm.

### Example 12

### 2-(5-(3-(5-Benzyl-4H-1,2,4-triazol-3-yl)phenoxy)-1H-indol-4-yl)-N-(cyclopropylsulfonyl)acetamide

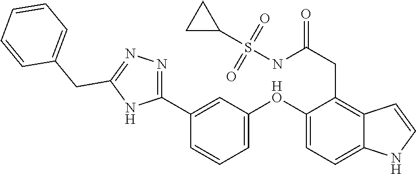

To a stirred solution of 2-(5-(3-(5-benzyl-4H-1,2,4-triazol-3-yl)phenoxy)-1H-indol-4-yl)acetic acid (80 mg, 0.19 mmol) in DMF (1 mL) were added DMAP (70 mg, 0.57 mmol), EDCI (109 mg, 0.57 mmol) and cyclopropanesulfonamide (92 mg, 0.76 mmol). The resulting mixture was stirred at 25° C. for one day, diluted with ethyl acetate (50 mL) and washed with brine. The organic layer was dried over sodium sulfate and concentrated. The resulting residue was purified by preparative HPLC to afford the title compound (12 mg, 12%). MS m/z: 528 [M+H]⁺. ¹H NMR (400 MHz, CD₃OD) δ 7.60 (d, J=8.0 Hz, 1H), 7.48 (s, 1H), 7.38 (t, J=8.0 Hz, 1H), 7.33-7.28 (m, 6H), 7.25-7.21 (m, 1H), 7.07 (dd, J=8.0, 1.2 Hz, 1H), 6.81 (d, J=8.4 Hz, 1H), 6.64 (d, J=2.8 Hz, 1H), 4.12 (s, 2H), 3.84 (s, 2H), 2.74-2.70 (m, 1H), 0.99-0.95 (m, 2H), 0.71-0.66 (m, 2H) ppm.

### Example 13

### 4-(2-(2-Fluoro-5-((6-fluoro-4-methyl-1H-indol-5-yl)oxy)phenyl)-1H-imidazol-5-yl)-4-phenylpentanoic acid

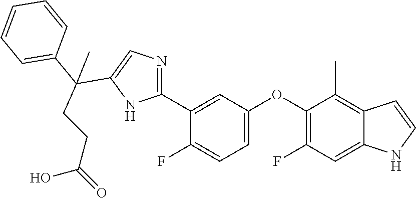 [SMILES: Cc1c(Oc2ccc(F)c(-c3ncc(C(C)(CCC(=O)O)c4ccccc4)[nH]3)c2)c(F)cc2[nH]ccc12]

### A. 2-Methyl-2-phenylhex-5-enoic acid

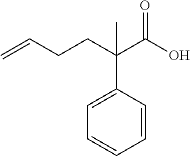

To a stirred solution of 2-phenylpropanoic acid (4.5 g, 30 mmol) in dry THF (45 mL) was added LDA (38 mL, 75 mmol) at −78° C. dropwise. The reaction mixture was warmed to room temperature, stirred for one hour and then cooled to 0° C. 4-Bromobut-1-ene (5.3 g, 39 mmol) was added to the reaction mixture and stirred at room temperature for 18 hours. Reaction was quenched with aqueous ammonium chloride solution and extracted with ethyl acetate (100 mL×2). The combined organic phases were dried, filtered and evaporated. The residue was purified by chromatography (petroleum ether/ethyl acetate, v/v, 10/1 to 6/1) to afford the title compound as white solid (4.3 g, 70%). MS m/z: 222 [M+NH₄]⁺. ¹H NMR (400 MHz, DMSO-d₆) δ 12.40 (br s, 1H), 7.30-7.36 (m, 4H), 7.21-7.25 (m, 1H), 5.76-5.83 (m, 1H), 4.90-5.00 (m, 2H), 1.85-1.99 (m, 4H), 1.46 (s, 3H) ppm.

### B. 1-Bromo-3-methyl-3-phenylhept-6-en-2-one

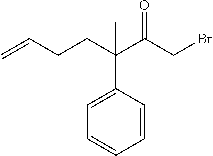 [SMILES: C=CCCC(C)(C(=O)CBr)c1ccccc1]

To a stirred solution of 2-methyl-2-phenylhex-5-enoic acid (1 g, 4.9 mmol) in dichloromethane (20 mL) was added DMF (2 drops) and oxalyl chloride (3.1 g, 24.5 mmol) at room temperature under nitrogen. The reaction mixture was stirred for one hour and evaporated under reduced pressure to remove the solvent and excess oxalyl chloride. The residue was taken in hexane and evaporated to remove any traces of oxalyl chloride. The crude 2-methyl-2-phenylhex-5-enoyl chloride was obtained as a yellow oil (1.3 g, 100%), which was used in the next step without further purification.

To a stirred solution of 2-methyl-2-phenylhex-5-enoyl chloride (1.3 g, 5.85 mmol) in a 1:1 mixture of acetonitrile and THF (20 mL) was added dropwise a 2M solution of TMSCHN₂ in hexane (5.9 mL, 11.7 mmol) at 0° C. After the addition was completed, the mixture was allowed to warm to room temperature, stirred for one hour and a solution of hydrobromic acid (4.2 g, 17.6 mmol, 32% in acetic acid) was added dropwise at 0° C. After stirring for one hour, the reaction mixture was diluted with ethyl acetate (100 mL), washed with brine, dried over sodium sulfate and concentrated. The resulting residue was purified by silica gel column chromatography (petroleum ether/ethyl acetate, v/v, 10/1 to 7/1) to afford the title compound as yellow oil (0.78 g, 57%). ¹H NMR (400 MHz, CDCl₃) δ 7.35-7.40 (m, 2H), 7.28-7.32 (m, 1H), 7.21-7.24 (m, 2H), 5.72-5.81 (m, 1H), 4.92-5.02 (m, 2H), 3.85 (m, 2H), 2.03-2.08 (m, 2H), 1.79-1.92 (m, 2H), 1.58 (s, 3H) ppm.

### C. 6-Fluoro-5-(4-fluoro-3-(5-(2-phenylhex-5-en-2-yl)-1H-imidazol-2-yl)phenoxy)-4-methyl-1H-indole

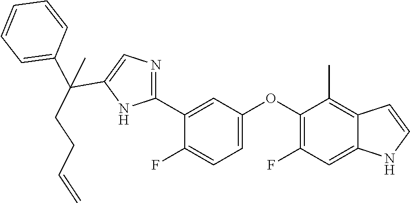 [SMILES: C=CCCC(C)(c1ccccc1)c1cnc(-c2cc(Oc3c(F)cc4[nH]ccc4c3C)ccc2F)[nH]1]

To a stirred solution of 1-bromo-3-methyl-3-phenylhept-6-en-2-one (650 mg, 2.31 mmol) and 2-fluoro-5-((6-fluoro-4-methyl-1H-indol-5-yl)oxy)benzimidamide (Intermediate 8-12, 0.7 g, 2.31 mmol) in DMF (10 mL) was added potassium carbonate (0.96 g, 6.94 mmol) under nitrogen. The reaction mixture was heated at 70° C. for four hours, cooled to room temperature, diluted with brine (10 mL) and extracted with ethyl acetate (100 mL×2). The combined organic phases were dried over sodium sulfate, filtered and concentrated. The resulting residue was purified by flash chromatography (dichloromethane/methanol, v/v, 25/1) to give a crude product, which was further purified by preparative HPLC to afford the title compound as a yellow solid (600 mg, 42%). MS m/z: 484 [M+H]⁺.

### D. 6-Fluoro-5-(4-fluoro-3-(5-(2-phenylhex-5-en-2-yl)-1-tosyl-1H-imidazol-2-yl)phenoxy)-4-methyl-1-tosyl-1H-indole

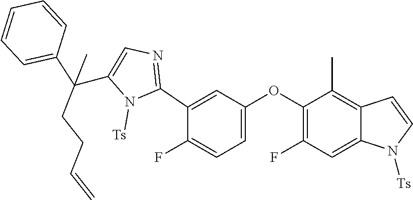 [SMILES: C=CCCC(C)(c1ccccc1)c1cnc(-c2cc(Oc3c(F)cc4c(ccn4S(=O)(=O)c4ccc(C)cc4)c3C)ccc2F)n1S(=O)(=O)c1ccc(C)cc1]

To a stirred solution of 6-fluoro-5-(4-fluoro-3-(5-(2-phenylhex-5-en-2-yl)-1H-imidazol-2-yl)phenoxy)-4-methyl-1H-indole (0.6 g, 1.24 mmol) in THF (20 mL) was added a 60% dispersion of sodium hydride in mineral oil (0.15 g, 3.73 mmol) at 0° C. The reaction mixture was stirred for one hour and p-toluene sulfonyl chloride (0.71 g, 3.73 mmol) was added. The mixture was stirred at room temperature for two hours, quenched with ice-water (10 mL) and extracted with ethyl acetate (100 mL×2). The combined organic extracts were washed with brine, dried over sodium sulfate, filtered and concentrated. The resulting residue was purified by flash chromatography (petroleum ether/ethyl acetate, v/v, 5/1 to 3/1) to afford the title compound as a white solid (0.7 g, 71%). MS m/z: 792 [M+H]⁺.

### E. 4-(2-(2-Fluoro-5-((6-fluoro-4-methyl-1-tosyl-1H-indol-5-yl)oxy)phenyl)-1-tosyl-1H-imidazol-5-yl)-4-phenylpentanal

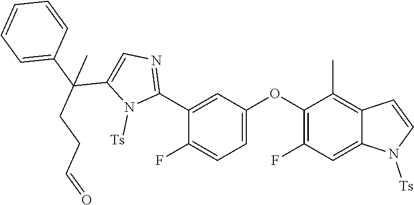 [SMILES: Cc1ccc(S(=O)(=O)n2c(C(C)(CCC=O)c3ccccc3)cnc2-c2cc(Oc3c(F)cc4c(ccn4S(=O)(=O)c4ccc(C)cc4)c3C)ccc2F)cc1]

To a stirred solution of 6-fluoro-5-(4-fluoro-3-(5-(2-phenylhex-5-en-2-yl)-1-tosyl-1H-imidazol-2-yl)phenoxy)-4-methyl-1-tosyl-1H-indole (600 mg, 0.76 mmol) and 2,6-lutidine (81 mg, 0.76 mmol) in THF (24 mL) was added OSO₄ (1 g/L in water, 10 drops), followed by dropwise addition of a solution of NaIO₄ (0.65 g, 3.03 mmol) in water (8 mL). The reaction mixture was stirred at room temperature overnight, acidified with 2N hydrochloric acid (10 mL) and extracted with ethyl acetate (100 mL×2). The combined organic extracts were washed with brine, dried over sodium sulfate, filtered and concentrated to afford the title compound as yellow oil (610 mg), which was used without further purification. MS m/z: 794 [M+H]⁺.

### F. 4-(2-(2-Fluoro-5-((6-fluoro-4-methyl-1-tosyl-1H-indol-5-yl)oxy)phenyl)-1H-imidazol-5-yl)-4-phenylpentanoic acid

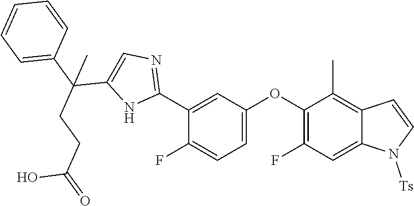 [SMILES: Cc1ccc(S(=O)(=O)n2ccc3c(C)c(Oc4ccc(F)c(-c5ncc(C(C)(CCC(=O)O)c6ccccc6)[nH]5)c4)c(F)cc32)cc1]

To a stirred solution of 4-(2-(2-fluoro-5-((6-fluoro-4-methyl-1-tosyl-1H-indol-5-yl)oxy)phenyl)-1-tosyl-1H-imidazol-5-yl)-4-phenylpentanal (610 mg, 0.77 mmol) in acetone (10 mL) was added John's reagent (2 mL, 2M in aqueous sulfuric acid) dropwise at 0° C. The mixture was stirred at 0° C. for one hour, diluted with ethyl acetate (200 mL), washed with water and brine, dried over sodium sulfate and concentrated to dryness. The residue was purified by chromatography (dichloromethane/methanol, v/v, 20/1 to 10/1) to afford the title compound as a yellow solid (350 mg, 70%). MS m/z: 656 [M+H]⁺.

### G. Example 13. 4-(2-(2-Fluoro-5-((6-fluoro-4-methyl-1H-indol-5-yl)oxy)phenyl)-1H-imidazol-5-yl)-4-phenylpentanoic acid

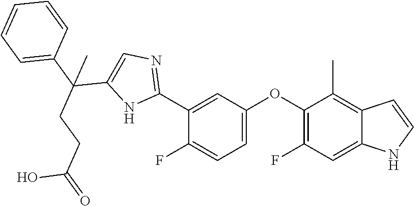 [SMILES: Cc1c(Oc2ccc(F)c(-c3ncc(C(C)(CCC(=O)O)c4ccccc4)[nH]3)c2)c(F)cc2[nH]ccc12]

A mixture of 4-(2-(2-fluoro-5-((6-fluoro-4-methyl-1-tosyl-1H-indol-5-yl)oxy)phenyl)-1H-imidazol-5-yl)-4-phenylpentanoic acid (0.35 g, 0.53 mmol) and potassium carbonate (0.37 g, 2.67 mmol) in methanol (8 mL) was heated at 80° C. for 60 minutes in a microwave reactor, diluted with water (10 mL) and extracted with ethyl acetate (25 mL×3). The combined organic extracts were washed with brine, dried over magnesium sulfate and evaporated to dryness. The resulting residue was purified by preparative HPLC to afford the title compound as pink solid (40 mg, 15%). MS m/z: 502 [M+H]⁺. ¹H NMR (400 MHz, CD₃OD) δ 7.40-7.38 (m, 1H), 7.29-7.26 (m, 5H), 7.20-7.17 (m, 1H), 7.15-7.08 (m, 2H), 7.00 (s, 1H), 6.81-6.77 (m, 1H), 6.51 (d, J=3.2 Hz, 1H), 2.48-2.44 (m, 2H), 2.38 (s, 3H), 2.18-2.13 (m, 2H), 1.68 (s, 3H) ppm.

### Example 14

### (2-(3-((6-Fluoro-4-(methylsulfonyl)-1H-indol-5-yl)oxy)phenyl)-1H-imidazol-5-yl)(phenyl)methanol

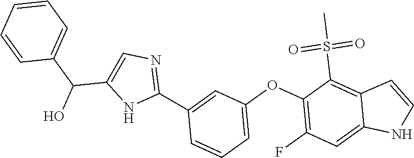 [SMILES: CS(=O)(=O)c1c(Oc2cccc(-c3ncc(C(O)c4ccccc4)[nH]3)c2)c(F)cc2[nH]ccc12]

### A. (2-(3-((6-Fluoro-4-(methylthio)-1H-indol-5-yl)oxy)phenyl)-1H-imidazol-5-yl)(phenyl)methanol

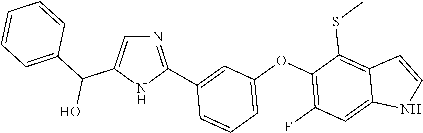 [SMILES: CSc1c(Oc2cccc(-c3ncc(C(O)c4ccccc4)[nH]3)c2)c(F)cc2[nH]ccc12]

3-((6-Fluoro-4-(methylthio)-1H-indol-5-yl)oxy)benzimidamide (390 mg, 1.24 mmol, Intermediate 8-2) and 3-phenyloxirane-2-carbaldehyde (183 mg, 1.24 mmol, Intermediate 3-1) were dissolved in DMF (6 mL) in a glove box. The reaction mixture was stirred at 75° C. overnight, cooled to room temperature, quenched with water (20 mL) and extracted with ethyl acetate (20 mL×3). The combined organic extracts were washed with brine (20 mL), dried over sodium sulfate, filtered and concentrated. The resulting residue was purified by flash chromatography (0-100% ethyl acetate in petroleum ether) to afford the tile compound as a white solid (155 mg, 40% from two steps). MS m/z: 446 [M+H]⁺.

### B. Example 14. (2-(3-((6-Fluoro-4-(methylsulfonyl)-1H-indol-5-yl)oxy)phenyl)-1H-imidazol-5-yl)(phenyl)methanol

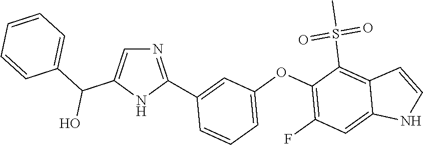 [SMILES: CS(=O)(=O)c1c(Oc2cccc(-c3ncc(C(O)c4ccccc4)[nH]3)c2)c(F)cc2[nH]ccc12]

To a stirred solution of (2-(3-((6-fluoro-4-(methylthio)-1H-indol-5-yl)oxy)phenyl)-1H-imidazol-5-yl)(phenyl)methanol (120 mg, 0.27 mmol) in methanol (10 mL) was added a solution of ammonium molybdate tetrahydrate (300 mg) in aqueous hydrogen peroxide (30% in water, 1.5 mL). The reaction mixture was stirred at room temperature for four hours, diluted with ethyl acetate (80 mL), washed with water (20 mL×3), aqueous sodium sulfite (20 mL) and brine (20 mL×2), dried over sodium sulfate and concentrated. The resulting residue was purified by flash chromatography (0-100% ethyl acetate in petroleum ether) to afford the title compound as a white solid (34 mg, 27%). MS m/z: 478 [M+H]⁺. ¹H NMR (400 MHz, CD₃OD) δ 7.66 (d, J=10.3 Hz, 1H), 7.58 (d, J=7.2 Hz, 1H), 7.53 (d, J=3.3 Hz, 1H), 7.49-7.25 (m, 7H), 7.21 (d, J=3.2 Hz, 1H), 6.98 (d, J=8.3 Hz, 1H), 6.79 (s, 1H), 5.79 (s, 1H), 3.33 (s, 3H) ppm.

### Example 15

### (2-(3-((6-Fluoro-4-(methylsulfonyl)-1H-indol-5-yl)oxy)phenyl)-1H-imidazol-5-yl)(phenyl)methanamine

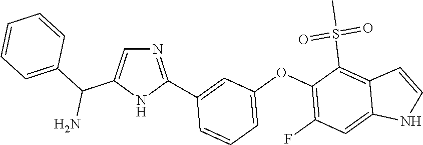 [SMILES: CS(=O)(=O)c1c(Oc2cccc(-c3ncc(C(N)c4ccccc4)[nH]3)c2)c(F)cc2[nH]ccc12]

### A. (2-(3-((6-Fluoro-4-(methylsulfonyl)-1H-indol-5-yl)oxy)phenyl)-1H-imidazol-5-yl)(phenyl)methanone

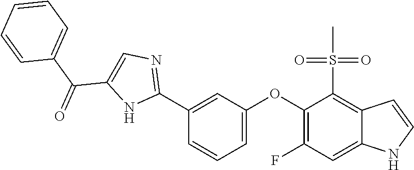 [SMILES: CS(=O)(=O)c1c(Oc2cccc(-c3ncc(C(=O)c4ccccc4)[nH]3)c2)c(F)cc2[nH]ccc12]

To a stirred solution of (2-(3-((6-fluoro-4-(methylsulfonyl)-1H-indol-5-yl)oxy)phenyl)-1H-imidazol-5-yl)(phenyl)methanol (24 mg, 0.05 mmol) in THF (5 ml) was added manganese dioxide (44 mg, 0.5 mmol). The mixture was stirred at room temperature overnight, filtered and rinsed with ethyl acetate (5 mL×3). The filtrate was concentrated to afford the title compound as a yellow solid (22 mg). MS m/z: 476 [M+H]⁺.

### B. Example 15. (2-(3-((6-Fluoro-4-(methylsulfonyl)-1H-indol-5-yl)oxy)phenyl)-1H-imidazol-5-yl)(phenyl)methanamine

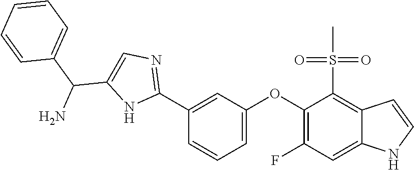 [SMILES: CS(=O)(=O)c1c(Oc2cccc(-c3ncc(C(N)c4ccccc4)[nH]3)c2)c(F)cc2[nH]ccc12]

To a microwave vial containing a solution of (2-(3-((6-fluoro-4-(methylsulfonyl)-1H-indol-5-yl)oxy)phenyl)-1H-imidazol-5-yl)(phenyl)methanone (22 mg, 0.05 mmol) in 7N ammonia in methanol (2 mL) was added titanium tetraisopropoxide (142 mg, 0.5 mmol). The vial was sealed, the reaction mixture was stirred at 50° C. overnight and then at 75° C. for another eight hours. The mixture was cooled in an ice bath, NaBH₄ (38 mg, 1 mmol) was added, stirred at 0° C. for fifteen minutes and then at room temperature for 0.5 hours. The reaction mixture was quenched with water (15 mL) and extracted with ethyl acetate (15 mL×3). The combined organic extracts were washed with brine (15 mL), dried over sodium sulfate, filtered and concentrated. The residue was purified by flash chromatography (0-10% methanol in dichloromethane containing 2% of 7N ammonia in methanol solution) to afford the title compound as a solid (10.6 mg, 44% two steps). MS m/z: 477 [M+H]⁺. ¹H NMR (400 MHz, CD₃OD) δ 7.64 (d, J=10.4 Hz, 1H), 7.56 (d, J=8.0 Hz, 1H), 7.52 (d, J=3.2 Hz, 1H), 7.45-7.41 (m, 3H), 7.39 (d, J=3.2 Hz, 1H), 7.36-7.24 (m, 3H), 7.19 (d, J=3.2 Hz, 1H), 6.97 (dd, J=8.0, 2.4 Hz, 1H), 6.85 (s, 1H), 5.21 (s, 1H), 3.32 (s, 3H) ppm.

### Example 16

### 1-(5-(3-((1H-Pyrrolo[3,2-b]pyridin-5-yl)oxy)phenyl)-4H-1,2,4-triazol-3-yl)-1-phenylethan-1-ol

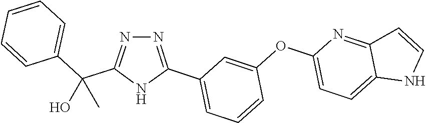 [SMILES: CC(O)(c1ccccc1)c1nnc(-c2cccc(Oc3ccc4[nH]ccc4n3)c2)[nH]1]

### A. Ethyl 3-((1H-pyrrolo[3,2-b]pyridin-5-yl)oxy)benzimidate

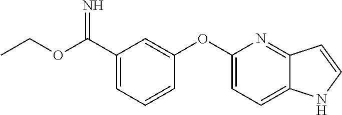 [SMILES: CCOC(=N)c1cccc(Oc2ccc3[nH]ccc3n2)c1]

Acetyl chloride (664 mg, 8.51 mmol) was added to a solution of 3-((1H-pyrrolo[3,2-b]pyridin-5-yl)oxy)benzonitrile (200 mg, 0.85 mmol) in ethanol (17 mL) at 0° C. The resulting mixture was stirred at 25° C. for 16 hours and concentrated in vacuo to afford the title compound as a yellow solid (230 mg). MS m/z: 282 [M+H]⁺.

### B. (5-(3-((1H-Pyrrolo[3,2-b]pyridin-5-yl)oxy)phenyl)-4H-1,2,4-triazol-3-yl)(phenyl) methanol

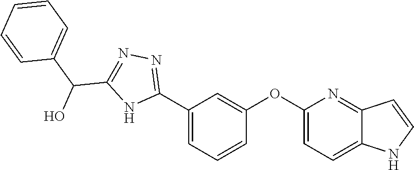 [SMILES: OC(c1ccccc1)c1nnc(-c2cccc(Oc3ccc4[nH]ccc4n3)c2)[nH]1]

A mixture of ethyl 3-((1H-pyrrolo[3,2-b]pyridin-5-yl)oxy)benzimidate (230 mg, 0.82 mmol), 2-hydroxy-2-phenylacetohydrazide (272 mg, 1.64 mmol) and triethylamine (661 mg, 6.55 mmol) in ethanol (20 mL) was stirred at 60° C. for 16 hours. The mixture was concentrated and the resulting residue was purified by silica gel column chromatography (dichloromethane/methanol, v/v, 30/1) to afford the title compound as a yellow solid (300 mg, 92% from two steps). MS m/z: 384 [M+H]⁺.

### C. (5-(3-((1H-Pyrrolo[3,2-b]pyridin-5-yl)oxy)phenyl)-4H-1,2,4-triazol-3-yl)(phenyl) methanone

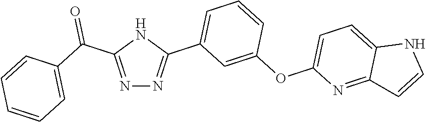 [SMILES: O=C(c1ccccc1)c1nnc(-c2cccc(Oc3ccc4[nH]ccc4n3)c2)[nH]1]

To a stirred solution of (5-(3-((1H-pyrrolo[3,2-b]pyridin-5-yl)oxy)phenyl)-4H-1,2,4-triazol-3-yl)(phenyl)methanol (300 mg, 0.78 mmol) in DMSO (6 mL) and THF (2 mL) was added manganese dioxide (681 mg, 7.83 mmol). The reaction mixture was stirred at 40° C. for sixteen hours, filtered through a pad of Celite and the filtrate was concentrated in vacuo. The resulting residue was purified by silica gel column chromatography (dichloromethane/methanol, v/v, 50/1) to afford the title compound as a yellow solid (292 mg, 96%). MS m/z: 382 [M+H]⁺.

### D. Example 16. 1-(5-(3-((1H-Pyrrolo[3,2-b]pyridin-5-yl)oxy)phenyl)-4H-1,2,4-triazol-3-yl)-1-phenylethan-1-ol

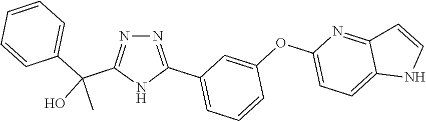 [SMILES: CC(O)(c1ccccc1)c1nnc(-c2cccc(Oc3ccc4[nH]ccc4n3)c2)[nH]1]

To a stirred solution of (5-(3-((1H-pyrrolo[3,2-b]pyridin-5-yl)oxy)phenyl)-4H-1,2,4-triazol-3-yl)(phenyl)methanone (150 mg, 0.39 mmol) in THF (10 mL) was added methylmagnesium bromide (1.31 mL, 1.31 mmol, 1N in THF) at 0° C. The resulting mixture was stirred at room temperature for two hours and quenched with saturated ammonium chloride solution (10 mL) and extracted with dichloromethane (25 mL×3). The combined organic extracts were washed with brine (10 mL), dried over sodium sulfate, filtered and concentrated in vacuo. The resulting residue was purified by preparative HPLC to afford the title compound as a white solid (13 mg, 13%). MS m/z: 398 [M+H]⁺. ¹H NMR (400 MHz, CD₃OD) δ 7.89 (d, J=8.4 Hz, 1H), 7.83 (d, J=7.6 Hz, 1H), 7.72 (s, 1H), 7.56-7.54 (m, 3H), 7.47 (t, J=8.0 Hz, 1H), 7.32 (t, J=7.6 Hz, 2H), 7.26-7.22 (m, 1H), 7.13 (dd, J=8.0, 1.6 Hz, 1H), 6.82 (d, J=8.4 Hz, 1H), 6.48 (d, J=3.2 Hz, 1H), 2.00 (s, 3H) ppm.

### Example 17

### (5-(3-((1H-Pyrrolo[3,2-b]pyridin-5-yl)oxy)phenyl)-4H-1,2,4-triazol-3-yl)(phenyl)methanamine

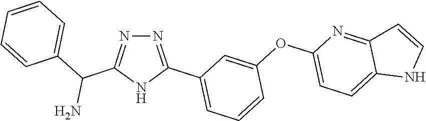 [SMILES: NC(c1ccccc1)c1nnc(-c2cccc(Oc3ccc4[nH]ccc4n3)c2)[nH]1]

A mixture of (5-(3-((1H-pyrrolo[3,2-b]pyridin-5-yl)oxy)phenyl)-4H-1,2,4-triazol-3-yl)(phenyl)methanone (Example 16 Step C, 142 mg, 0.37 mmol), titanium tetraisopropoxide (546 mg, 1.92 mmol) and NH₃ (7.0 N in MeOH, 0.45 mL, 3.2 mmol) in MeOH (5 mL) was stirred at 50° C. for 16 hours under nitrogen atmosphere. NaBH₄ (24 mg, 0.64 mmol) was added and then stirred at room temperature for one hour. The reaction mixture was filtered through a pad of Celite and concentrated in vacuo. The resulting residue was purified by preparative HPLC to afford the title compound as a white solid (18.7 mg, 13%). MS m/z: 383 [M+H]⁺. ¹H NMR (400 MHz, CD₃OD) δ 7.89 (d, J=8.8 Hz, 1H), 7.79 (d, J=8.0 Hz, 1H), 7.69 (t, J=2.4 Hz, 1H), 7.54 (d, J=3.2 Hz, 1H), 7.49 (t, J=8.0 Hz, 1H), 7.45-7.42 (m, 2H), 7.35 (t, J=7.2 Hz, 2H), 7.30-7.26 (m, 1H), 7.15 (dd, J=8.0, 2.0 Hz, 1H), 6.82 (d, J=8.4 Hz, 1H), 6.47 (d, J=2.8 Hz, 1H), 5.27 (s, 1H) ppm.

### Example 18

### N-((5-(3-(5-(Hydroxy(phenyl)methyl)-1H-imidazol-2-yl)phenoxy)-1H-indol-4-yl)methyl)acetamide

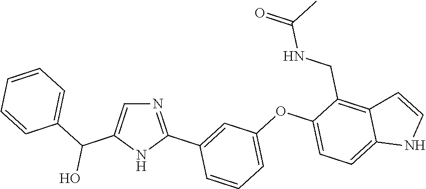 [SMILES: CC(=O)NCc1c(Oc2cccc(-c3ncc(C(O)c4ccccc4)[nH]3)c2)ccc2[nH]ccc12]

### A. (2-(3-((4-(Hydroxymethyl)-1H-indol-5-yl)oxy)phenyl)-1H-imidazol-5-yl)(phenyl)methanol

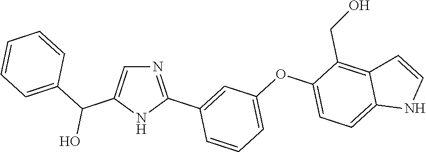 [SMILES: OCc1c(Oc2cccc(-c3ncc(C(O)c4ccccc4)[nH]3)c2)ccc2[nH]ccc12]

A mixture of 3-((4-(hydroxymethyl)-1H-indol-5-yl)oxy)benzimidamide (1 g, 3.79 mmol, Intermediate 8-3) and 3-phenyloxirane-2-carbaldehyde (Intermediate 3-1, 840 mg, 5.68 mmol) in DMF (10 mL) was stirred at 75° C. overnight. The mixture was cooled to room temperature, diluted with water (100 mL) and extracted with ethyl acetate (50 mL×4). The combined organic extracts were washed with brine (50 mL×2), dried over sodium sulfate, filtered and concentrated. The resulted residue was purified by silica gel column chromatography (dichloromethane/methanol, v/v, 10/1) to afford the title compound as a light yellow solid (320 mg, 21%). MS m/z: 412 [M+H]⁺.

### B. 5-(3-(5-Benzoyl-1H-imidazol-2-yl)phenoxy)-1H-indole-4-carbaldehyde

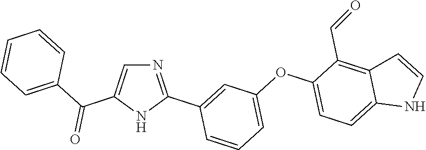 [SMILES: O=Cc1c(Oc2cccc(-c3ncc(C(=O)c4ccccc4)[nH]3)c2)ccc2[nH]ccc12]

To a stirred solution of (2-(3-((4-(hydroxymethyl)-1H-indol-5-yl)oxy)phenyl)-1H-imidazol-5-yl)(phenyl)methanol (320 mg, 0.78 mmol) in THF (10 mL) was added manganese dioxide (677 mg, 7.78 mmol) at room temperature. The resulting mixture was stirred at room temperature overnight, filtered through a pad of Celite and the filtrate was concentrated to afford the title compound as a yellow solid (310 mg). MS m/z: 408 [M+H]⁺.

### C. 5-(3-(5-Benzoyl-1H-imidazol-2-yl)phenoxy)-1H-indole-4-carbaldehyde oxime

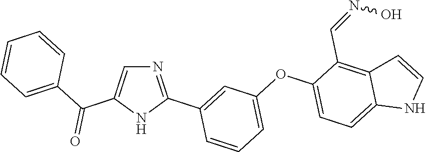 [SMILES: O=C(c1ccccc1)c1cnc(-c2cccc(Oc3ccc4[nH]ccc4c3C=NO)c2)[nH]1]

To a stirred solution of 5-(3-(5-benzoyl-1H-imidazol-2-yl)phenoxy)-1H-indole-4-carbaldehyde (310 mg, 0.76 mmol) in ethanol (5 mL) were added hydroxylamine hydrochloride (105 mg, 1.52 mmol) and NaOAc (187 mg, 2.29 mmol). The resulting mixture was stirred at room temperature for five hours and then concentrated in vacuo. The resulting residue was dissolved in ethyl acetate (50 mL), washed with water (10 mL×2) and brine (10 mL), dried over sodium sulfate and concentrated. The resulting residue was purified by silica gel column chromatography (dichloromethane/methanol, v/v, 10/1) to afford the title compound (273 mg, 82% from 2 steps). MS m/z: 423 [M+H]⁺.

### D. (2-(3-((4-(Aminomethyl)-1H-indol-5-yl)oxy)phenyl)-1H-imidazol-5-yl)(phenyl)methanone

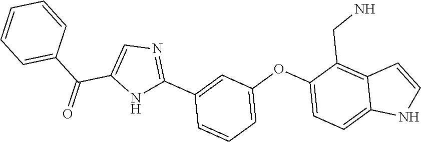 [SMILES: NCc1c(Oc2cccc(-c3ncc(C(=O)c4ccccc4)[nH]3)c2)ccc2[nH]ccc12]

To a stirred solution of 5-(3-(5-benzoyl-1H-imidazol-2-yl)phenoxy)-1H-indole-4-carbaldehyde oxime (270 mg, 0.64 mmol) in acetic acid (5 mL) and ethanol (5 mL) was added Raney Ni (100 mg). The mixture was stirred at room temperature under hydrogen atmosphere overnight and filtered through a pad of Celite. The filtrate was concentrated in vacuo to afford the title compound as a yellow solid (145 mg), which was used in the next step without further purification. MS m/z: 409 [M+H]⁺.

### E. N-((5-(3-(5-Benzoyl-1H-imidazol-2-yl)phenoxy)-1H-indol-4-yl)methyl)acetamide

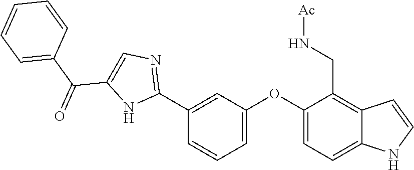 [SMILES: CC(=O)NCc1c(Oc2cccc(-c3ncc(C(=O)c4ccccc4)[nH]3)c2)ccc2[nH]ccc12]

To a stirred solution of (2-(3-((4-(aminomethyl)-1H-indol-5-yl)oxy)phenyl)-1H-imidazol-5-yl)(phenyl)methanone (145 mg, 0.36 mmol) in THF (5 mL) were added AcOH (26 mg, 0.42 mmol), HATU (203 mg, 0.53 mmol) and diisopropylethylamine (137 mg, 1.06 mmol). The resulting mixture was stirred at room temperature overnight, quenched with water (10 mL) and extracted with ethyl acetate (10 mL×3). The combined organic extracts were dried over sodium sulfate, filtered and concentrated in vacuo to afford the title compound as a yellow solid (120 mg), which was used in the next step without further purification. MS m/z: 451 [M+H]⁺.

### F. Example 18. N-((5-(3-(5-(Hydroxy(phenyl)methyl)-1H-imidazol-2-yl)phenoxy)-1H-indol-4-yl)methyl)acetamide

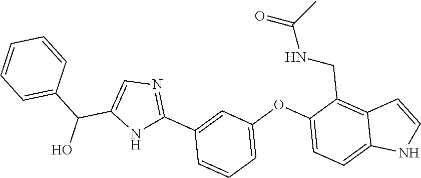 [SMILES: CC(=O)NCc1c(Oc2cccc(-c3ncc(C(O)c4ccccc4)[nH]3)c2)ccc2[nH]ccc12]

To a stirred solution of N-((5-(3-(5-benzoyl-1H-imidazol-2-yl)phenoxy)-1H-indol-4-yl)methyl)acetamide (120 mg, 0.27 mmol) in methanol (5 mL) was added NaBH₄ (30 mg, 0.8 mmol). The resulting mixture was stirred at 25° C. for three hours, quenched with saturated aqueous ammonium chloride solution (20 mL) and extracted with ethyl acetate (20 mL×3). The combined organic phase was washed with brine (20 mL), dried over sodium sulfate, filtered and concentrated in vacuo. The resulting residue was purified by preparative HPLC to afford the title compound as a white solid (32 mg, 11% from 3 steps). MS m/z: 453 [M+H]⁺. ¹H NMR (400 MHz, CD₃OD) δ 7.47-7.27 (m, 10H), 6.96 (dd, J=8.0, 2.0 Hz, 1H), 6.85 (d, J=8.8 Hz, 1H), 6.74 (s, 1H), 6.60 (d, J=3.2 Hz, 1H), 5.77 (s, 1H), 4.64 (s, 2H), 1.72 (s, 3H) ppm.

### Example 19

### 5-(3-(5-Benzyl-4H-1,2,4-triazol-3-yl)phenoxy)-1H-indole-4-carboxylic acid

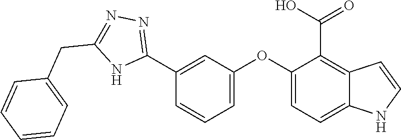 [SMILES: O=C(O)c1c(Oc2cccc(-c3nnc(Cc4ccccc4)[nH]3)c2)ccc2[nH]ccc12]

A mixture of methyl 5-(3-cyanophenoxy)-1H-indole-4-carboxylate (Intermediate 1-3, 500 mg, 1.71 mmol), 2-phenylacetohydrazide (385 mg, 2.57 mmol) and potassium carbonate (945 mg, 6.85 mmol) in n-Butanol (10 mL) was irradiated at 160° C. for two hours in a Biotage microwave synthesizer. The reaction mixture was cooled to room temperature, quenched with water (20 mL) and extracted with ethyl acetate (20 mL×3). The combined organic extracts were washed with brine (30 mL), dried over sodium sulfate, filtered and concentrated in vacuo. The resulting residue was purified by preparative HPLC to afford the title compound as a white solid (100 mg, 14%). MS m/z: 411 [M+H]⁺. ¹H NMR (500 MHz, DMSO-d₆) δ 11.35 (s, 1H), 7.59 (d, J=7.5 Hz, 1H), 7.51 (d, J=8.5 Hz, 1H), 7.46 (s, 1H), 7.37-7.33 (m, 2H), 7.31-7.26 (m, 4H), 7.21 (t, J=6.5 Hz, 1H), 6.91 (dd, J=8.0, 2.0 Hz, 1H), 6.81 (d, J=8.5 Hz, 1H), 6.69 (s, 1H), 4.06 (s, 2H) ppm.

### Example 20

### (5-(3-(5-Benzyl-4H-1,2,4-triazol-3-yl)phenoxy)-1H-indol-4-yl)methanol

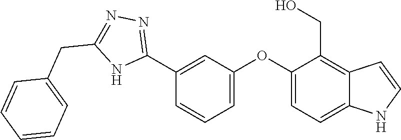 [SMILES: OCc1c(Oc2cccc(-c3nnc(Cc4ccccc4)[nH]3)c2)ccc2[nH]ccc12]

To a stirred solution of 5-(3-(5-benzyl-4H-1,2,4-triazol-3-yl)phenoxy)-1H-indole-4-carboxylic acid (500 mg, 1.2 mmol, Example 19) in THF (5 mL) was added LiAlH₄ (12 mL, 1N in THF, 12 mmol) at 0° C. The reaction mixture was stirred at room temperature overnight, carefully quenched with solid Na₂SO₄·10H₂O filtered through Celite and rinsed with THF (100 mL). The filtrate was concentrated and the resulting crude product was purified by reverse phase combi-flash to afford the title compound as a light yellow solid (285 mg, 60%). MS m/z: 397 [M+H]⁺. ¹H NMR (400 MHz, CD₃OD) δ 7.60 (d, J=7.8 Hz, 1H), 7.46 (d, J=1.8 Hz, 1H), 7.40-7.17 (m, 8H), 6.98 (dd, J=8.2, 1.8 Hz, 1H), 6.80 (d, J=8.7 Hz, 1H), 6.74-6.65 (m, 1H), 4.87 (s, 2H), 4.10 (s, 2H) ppm.

### Example 21

### N-((5-(3-(5-Benzyl-4H-1,2,4-triazol-3-yl)henoxy)-1H-indol-4-yl)methyl)propan-2-amine 2,2,2-trifluoroacetate

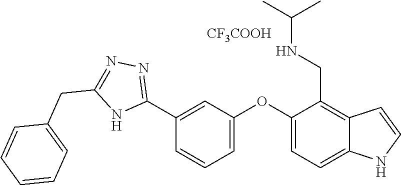 [SMILES: CC(C)NCc1c(Oc2cccc(-c3nnc(Cc4ccccc4)[nH]3)c2)ccc2[nH]ccc12 ; "CF3COOH"]

### A. 5-(3-(5-Benzyl-4H-1,2,4-triazol-3-yl)phenoxy)-1H-indole-4-carbaldehyde

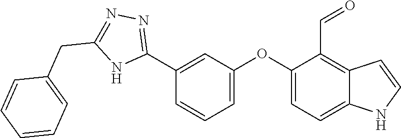 [SMILES: O=Cc1c(Oc2cccc(-c3nnc(Cc4ccccc4)[nH]3)c2)ccc2[nH]ccc12]

To a stirred solution of (5-(3-(5-benzyl-4H-1,2,4-triazol-3-yl)phenoxy)-1H-indol-4-yl)methanol (Example 20, 100 mg, 0.25 mmol) in a mixture of dichloromethane (3 mL) and THF (5 mL) was added manganese dioxide (218 mg, 2.5 mmol) at room temperature. The resulting mixture was stirred at room temperature, filtered through a pad of Celite and rinsed with THF (50 mL). The filtrate was concentrated and the resulting crude product was purified by preparative TLC (petroleum ether/ethyl acetate, v/v, 2/3) to afford the title compound as light yellow oil (55 mg, 56%). MS m/z: 395 [M+H]⁺.

### B. Example 21. N-((5-(3-(5-Benzyl-4H-1,2,4-triazol-3-yl)phenoxy)-1H-indol-4-yl)methyl)propan-2-amine 2,2,2-trifluoroacetate

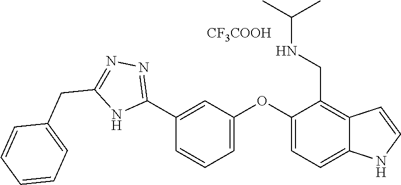 [SMILES: CC(C)NCc1c(Oc2cccc(-c3nnc(Cc4ccccc4)[nH]3)c2)ccc2[nH]ccc12 ; "CF3COOH"]

A mixture of 5-(3-(5-benzyl-4H-1,2,4-triazol-3-yl)phenoxy)-1H-indole-4-carbaldehyde (68 mg, 0.17 mmol), propan-2-amine (21 mg, 0.35 mmol) and acetic acid (2 drops) in THF (10 mL) was stirred at room temperature for three hours. Sodium cyanoborohydride (33 mg, 0.52 mmol) was added and the reaction mixture was stirred at room temperature overnight. The mixture was diluted with ethyl acetate (80 mL), washed with brine (50 mL), dried over sodium sulfate, filtered and concentrated. The resulting residue was purified by preparative HPLC to afford the title compound as a white solid (50 mg, 66%). MS/z: 438 [M+H]⁺. ¹H NMR (500 MHz, DMSO-d₆) δ 11.49 (s, 1H), 8.69 (s, 2H), 7.72 (d, J=7.5 Hz, 1H), 7.57 (t, J=3.0 Hz, 1H), 7.54 (d, J=9.5 Hz, 1H), 7.51 (s, 1H), 7.46 (t, J=8.5 Hz, 1H), 7.32-7.26 (m, 4H), 7.24-7.21 (m, 1H), 7.05 (dd, J=9.5, 2.5 Hz, 1H), 6.86 (d, J=9.0 Hz, 1H), 6.76 (s, 1H), 4.32 (t, J=5.5 Hz, 2H), 4.08 (s, 2H), 3.44-3.39 (m, 1H), 1.29 (d, J=7.0 Hz, 6H) ppm.

### Example 22

### 6-Fluoro-5-(4-fluoro-3-(5-(1-hydroxy-1-phenylethyl)-1H-imidazol-2-yl)phenoxy)-N-methyl-1H-indole-4-sulfonamide

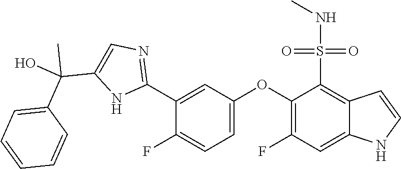 [SMILES: CNS(=O)(=O)c1c(Oc2ccc(F)c(-c3ncc(C(C)(O)c4ccccc4)[nH]3)c2)c(F)cc2[nH]ccc12]

### A. 5-(3-Cyano-4-fluorophenoxy)-6-fluoro-N-methyl-1-tosyl-1H-indole-4-sulfonamide

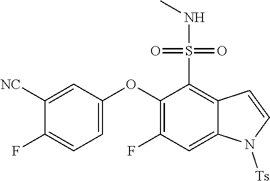 [SMILES: CNS(=O)(=O)c1c(Oc2ccc(F)c(C#N)c2)c(F)cc2c1ccn2S(=O)(=O)c1ccc(C)cc1]

To a stirred solution of 2-fluoro-5-((6-fluoro-4-((4-methoxybenzyl)thio)-1-tosyl-1H-indol-5-yl)oxy)benzonitrile (1 g, 1.7 mmol, Intermediate 6A) in a mixture of THF (20 mL), acetic acid (60 μL) and water (120 μL) at 0° C. was added 1,3-dichloro-5,5-dimethyl-imidazolidine-2,4-dione (536 mg, 2.7 mmol). The reaction mixture was stirred at 0° C. for one hour, poured into a solution of methylamine in THF (17 mL, 2M in THF) and stirred at room temperature for 30 minutes. The mixture was extracted with ethyl acetate (20 mL×2) and the ethyl acetate layer was washed with water and brine, dried over sodium sulfate, filtered and concentrated. The residue was purified by flash chromatography (0-40% ethyl acetate in petroleum ether) to afford the title compound as a yellow solid (590 mg, 66%). MS m/z: 518 [M+H]⁺.

### B. 5-(3-Cyano-4-fluorophenoxy)-6-fluoro-N-methyl-1H-indole-4-sulfonamide

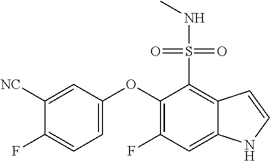 [SMILES: CNS(=O)(=O)c1c(Oc2ccc(F)c(C#N)c2)c(F)cc2[nH]ccc12]

To a stirred solution of 5-(3-cyano-4-fluorophenoxy)-6-fluoro-N-methyl-1-tosyl-1H-indole-4-sulfonamide (490 mg, 0.95 mmol) in THF (5 mL) was added a 1M solution of tetrabutylammonium fluoride in THF (5 mL). The reaction mixture was stirred at 75° C. for two hours, diluted with water and extracted with ethyl acetate. The combined organic extracts were washed with water and brine, dried over sodium sulfate, filtered and concentrated. The resulting residue was purified by flash chromatography over silica gel (0-30% ethyl acetate in petroleum ether) to afford the title compound as a white solid (170 mg, 49%). MS m/z: 364 [M+H]⁺.

### C. 2-Fluoro-5-((6-fluoro-4-(N-methylsulfamoyl)-1H-indol-5-yl)oxy)benzimidamide

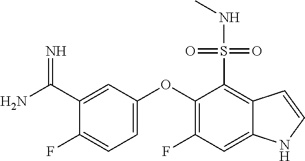 [SMILES: CNS(=O)(=O)c1c(Oc2ccc(F)c(C(=N)N)c2)c(F)cc2[nH]ccc12]

To a stirred solution of 5-(3-cyano-4-fluorophenoxy)-6-fluoro-N-methyl-1H-indole-4-sulfonamide (170 mg, 0.47 mmol) in anhydrous THF (5 mL) was added lithium bis(trimethylsilyl)amide (5 mL, 1M in THF) at 25° C. under nitrogen. The reaction mixture was stirred at room temperature overnight, quenched with water and extracted with dichloromethane (20 mL×3). The combined organic extracts were dried and concentrated to afford the title compound as a yellow solid (70 mg, 39%). MS m/z: 381 [M+H]⁺.

### D. Example 22. 6-Fluoro-5-(4-fluoro-3-(5-(1-hydroxy-1-phenylethyl)-1H-imidazol-2-yl)phenoxy)-N-methyl-1H-indole-4-sulfonamide

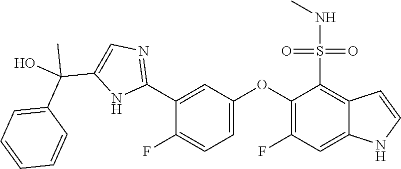 [SMILES: CNS(=O)(=O)c1c(Oc2ccc(F)c(-c3ncc(C(C)(O)c4ccccc4)[nH]3)c2)c(F)cc2[nH]ccc12]

To a stirred solution of 2-fluoro-5-((6-fluoro-4-(N-methylsulfamoyl)-1H-indol-5-yl)oxy)benzimidamide (70 mg, 0.18 mmol) in DMF (2 mL) was added 3-methyl-3-phenyloxirane-2-carbaldehyde (Intermediate 3, 45 mg, 0.28 mmol). The reaction mixture was stirred at 75° C. overnight, cooled to room temperature, quenched with water and extracted with ethyl acetate. The combined organic extracts were washed with water, brine, dried over sodium sulfate, filtered and concentrated. The resulting residue was purified by preparative HPLC to afford the title compound as a white solid (11.8 mg, 12%). MS m/z: 525 [M+H]⁺. ¹H NMR (400 MHz, CD₃OD): δ 7.58-7.46 (m, 5H), 7.31-7.12 (m, 5H), 6.98-6.92 (m, 2H), 2.59 (s, 3H), 1.89 (s, 3H) ppm.

### Example 23

### (5-(3-(1-Benzyl-1H-1,2,4-triazol-3-yl)phenoxy)-1H-indol-4-yl)methanol

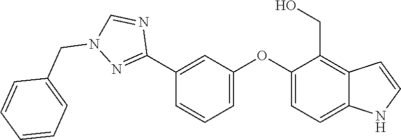 [SMILES: OCc1c(Oc2cccc(-c3ncn(Cc4ccccc4)n3)c2)ccc2[nH]ccc12]

### A. Methyl 5-(3-(imino(methoxy)methyl)phenoxy)-1-tosyl-1H-indole-4-carboxylate

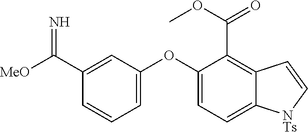 [SMILES: COC(=N)c1cccc(Oc2ccc3c(ccn3S(=O)(=O)c3ccc(C)cc3)c2C(=O)OC)c1]

To a stirred solution of methyl 5-(3-cyanophenoxy)-1-tosyl-1H-indole-4-carboxylate (Intermediate 5-3, 1 g, 2.24 mmol) in methanol (12 ml, 296.61 mmol) was added acetyl chloride (7 ml, 98 mmol) at 0° C. and the reaction mixture was stirred overnight at room temperature. The solvent was evaporated and the resulting residue was triturated with ethyl acetate to afford the title compound as white solid (0.95 g, 82%). MS m/z: 479 [M+H]⁺.

### B. Methyl 5-(3-(hydrazineyl(imino)methyl)phenoxy)-1-tosyl-1H-indole-4-carboxylate

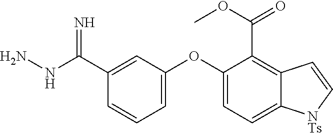 [SMILES: COC(=O)c1c(Oc2cccc(C(=N)NN)c2)ccc2c1ccn2S(=O)(=O)c1ccc(C)cc1]

To a mixture of methyl 5-(3-(imino(methoxy)methyl)phenoxy)-1-tosyl-1H-indole-4-carboxylate hydrochloride (0.5 g, 971 μmol) in methanol (5 ml) was added hydrazine hydrate (152 μl, 4.85 mmol) at −78° C. The reaction mixture was slowly warmed to room temperature and stirred for five hours. The solvent was evaporated and the residue was used in the next step without further purification. MS m/z: 479 [M+H]⁺.

### C. Methyl 5-(3-(1H-1,2,4-triazol-3-yl)phenoxy)-1-tosyl-1H-indole-4-carboxylate

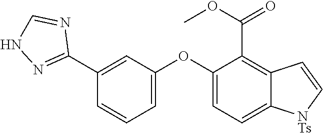 [SMILES: COC(=O)c1c(Oc2cccc(-c3nc[nH]n3)c2)ccc2c1ccn2S(=O)(=O)c1ccc(C)cc1]

A mixture of methyl 5-(3-(hydrazinyl(imino)methyl)phenoxy)-1-tosyl-1H-indole-4-carboxylate (0.45 g, 940 μmol) in formic acid (5 ml, 130 mmol) was heated at 135° C. for one hour in a microwave reactor. The solvent was evaporated and the residue was purified by column chromatography (0-100% ethyl acetate in heptane) to afford the title compound as a white solid (0.25 g, 54%).

### D. Methyl 5-(3-(1-benzyl-1H-1,2,4-triazol-3-yl)phenoxy)-1-tosyl-1H-indole-4-carboxylate

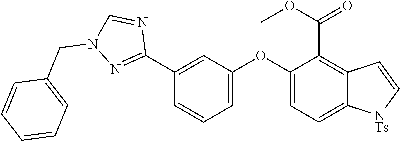 [SMILES: COC(=O)c1c(Oc2cccc(-c3ncn(Cc4ccccc4)n3)c2)ccc2c1ccn2S(=O)(=O)c1ccc(C)cc1]

A mixture of methyl 5-(3-(4H-1,2,4-triazol-3-yl)phenoxy)-1-tosyl-1H-indole-4-carboxylate (120 mg, 246 μmol), benzyl bromide (35 μl, 294.77 μmol) and DBU (74 μl, 491 μmol) in THF (1 ml) was stirred overnight at room temperature. The solvent was evaporated and the residue was chromatographed on a silica gel column (0-100% ethyl acetate in heptane) to afford the title compound (120 mg, 84%).

### E. Methyl 5-(3-(1-benzyl-1H-1,2,4-triazol-3-yl)phenoxy)-1H-indole-4-carboxylate

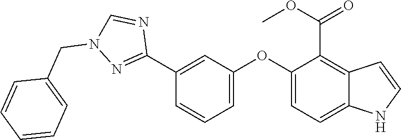 [SMILES: COC(=O)c1c(Oc2cccc(-c3ncn(Cc4ccccc4)n3)c2)ccc2[nH]ccc12]

A mixture of methyl 5-(3-(1-benzyl-1H-1,2,4-triazol-3-yl)phenoxy)-1-tosyl-1H-indole-4-carboxylate (120 mg, 207 μmol) and potassium carbonate (57 mg, 415 μmol) in methanol (5 ml) was heated at 130° C. for fifteen minutes. The solvent was removed and the residue was chromatographed on silica gel column (0-100% ethyl acetate in heptane) to afford the title compound as a white solid (80 mg, 91%). MS m/z: 425 [M+H]⁺.

### F. Example 23. (5-(3-(1-Benzyl-1H-1,2,4-triazol-3-yl)phenoxy)-1H-indol-4-yl)methanol

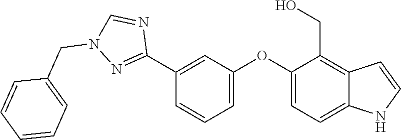 [SMILES: OCc1c(Oc2cccc(-c3ncn(Cc4ccccc4)n3)c2)ccc2[nH]ccc12]

To a mixture of methyl 5-(3-(1-benzyl-1H-1,2,4-triazol-3-yl)phenoxy)-1H-indole-4-carboxylate (25 mg, 59 μmol) in methylene chloride (2 mL) was added DIBAL-H (236 μl, 236 μmol, 1M in hexane) at −78° C. The reaction mixture was stirred for one hour at this temperature, quenched with saturated sodium potassium tartrate (10 mL), stirred overnight and extracted with dichloromethane. The combined organic extracts were washed with brine, dried over sodium sulfate, filtered and concentrated. The resulting residue was chromatographed over silica gel (0-100% ethyl acetate in heptane) to afford the title compound as a white solid (13 mg, 56%). LC-MS: Retention time: 1.11 min. MS m/z: 397 [M+H]⁺.

### Example 24

### N1-((5-(3-((1H-Pyrrolo[3,2-b]pyridin-5-yl)oxy)phenyl)-4H-1,2,4-triazol-3-yl)(phenyl)methyl)-N2,N2-dimethylethane-1,2-diamine

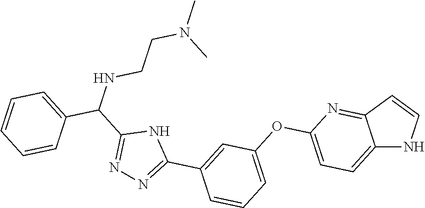 [SMILES: CN(C)CCNC(c1ccccc1)c1nnc(-c2cccc(Oc3ccc4[nH]ccc4n3)c2)[nH]1]

### A. (5-(3-((1H-Pyrrolo[3,2-b]pyridin-5-yl)oxy)phenyl)-4H-1,2,4-triazol-3-yl)(phenyl) methanol

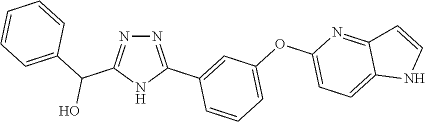 [SMILES: OC(c1ccccc1)c1nnc(-c2cccc(Oc3ccc4[nH]ccc4n3)c2)[nH]1]

A mixture of ethyl 3-((1H-pyrrolo[3,2-b]pyridin-5-yl)oxy)benzimidate (2.2 g, 7.8 mmol, product of Step A, Example 16), 2-hydroxy-2-phenylacetohydrazide (2.6 g, 15.6 mmol) and triethylamine (8.7 mL, 62.4 mmol) in ethanol (30 mL) was heated at 60° C. overnight. The reaction mixture was concentrated and the resulting residue was purified by silica gel column chromatography (dichloromethane/methanol, v/v, 50/1) to afford the title compound as a yellow solid (1.8 g, 60%). MS m/z: 384 [M+H]⁺.

### B. (5-(3-((1H-Pyrrolo[3,2-b]pyridin-5-yl)oxy)phenyl)-4H-1,2,4-triazol-3-yl)(phenyl)methanone

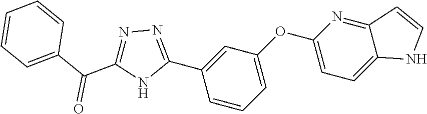 [SMILES: O=C(c1ccccc1)c1nnc(-c2cccc(Oc3ccc4[nH]ccc4n3)c2)[nH]1]

A mixture of (5-(3-((1H-pyrrolo[3,2-b]pyridin-5-yl)oxy)phenyl)-4H-1,2,4-triazol-3-yl)(phenyl)methanol (1.8 g, 4.7 mmol) and 2-iodoxybenzoic acid (5.3 g, 18.8 mmol) in DMSO (20 mL) was stirred at 30° C. for three hours. The reaction mixture was quenched with water (20 mL) and extracted with ethyl acetate (20 mL×5). The combined organic extracts were washed with brine (10 mL×2), dried over sodium sulfate, filtered and concentrated. The resulting residue was purified by silica gel column chromatography (petroleum ether/ethyl acetate, v/v, 10/1) to afford the title compound as a yellow solid (800 mg, 44%). MS m/z: 382 [M+H]⁺.

### C. Example 24. N¹-((5-(3-((1H-Pyrrolo[3,2-b]pyridin-5-yl)oxy)phenyl)-4H-1,2,4-triazol-3-yl)(phenyl)methyl)-N²,N²-dimethylethane-1,2-diamine

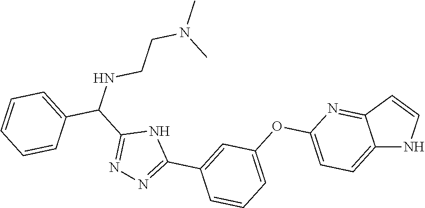 [SMILES: CN(C)CCNC(c1ccccc1)c1nnc(-c2cccc(Oc3ccc4[nH]ccc4n3)c2)[nH]1]

A mixture of (5-(3-((1H-pyrrolo[3,2-b]pyridin-5-yl)oxy)phenyl)-4H-1,2,4-triazol-3-yl)(phenyl)methanone (250 mg, 0.66 mmol), N¹,N¹-dimethylethane-1,2-diamine (581 mg, 6.6 mmol) and titanium tetraisopropoxide (1.9 g, 6.6 mmol) in methanol (5 ml) was stirred in a sealed tube at 60° C. overnight. NaBH₄ (100 mg, 2.6 mmol) was added at 0° C., the reaction mixture was stirred at room temperature for one hour and then filtered through a pad of Celite. The filtrate was concentrated and the resulting residue was purified by preparative HPLC to afford the title compound as a yellow solid (80 mg, 27%). MS m/z: 454 [M+H]⁺. ¹H NMR (400 MHz, CD₃OD) δ 8.60 (br s, 1H), 7.91 (d, J=8.4 Hz, 1H), 7.78 (d, J=8.0 Hz, 1H), 7.68 (s, 1H), 7.56-7.47 (m, 4H), 7.40-7.30 (m, 3H), 7.18 (dd, J=8.0, 2.0 Hz, 1H), 6.84 (d, J=8.4 Hz, 1H), 6.47 (d, J=3.2 Hz, 1H), 5.11 (s, 1H), 3.18-3.07 (m, 2H), 2.88 (t, J=8.0 Hz, 2H), 2.79 (s, 6H) ppm.

### Example 25

### 1-(2-(5-((4-(2,2-Difluoroethyl)-6-fluoro-1H-indol-5-yl)oxy)-2-fluorophenyl)-1H-imidazol-4-yl)-1-phenylethan-1-ol

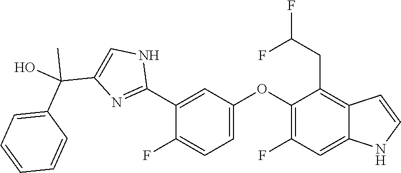 [SMILES: CC(O)(c1ccccc1)c1c[nH]c(-c2cc(Oc3c(F)cc4[nH]ccc4c3CC(F)F)ccc2F)n1]

### A. 5-((4-(2,2-Difluoroethyl)-6-fluoro-1H-indol-5-yl)oxy)-2-fluorobenzonitrile

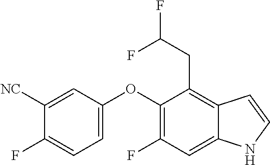 [SMILES: N#Cc1cc(Oc2c(F)cc3[nH]ccc3c2CC(F)F)ccc1F]

To a stirred solution of 5-((4-(2,2-difluoroethyl)-6-fluoro-1-tosyl-1H-indol-5-yl)oxy)-2-fluorobenzonitrile (340 mg, 0.7 mmol) in methanol (5 mL) was added potassium carbonate (388 mg, 2.1 mmol). The reaction mixture was stirred at 80° C. for 45 minutes in a microwave reactor, diluted with water and extracted with dichloromethane (30 mL×2). The combined organic extracts were washed with brine, dried over magnesium sulfate and evaporated in vacuo. The resulting residue was purified by flash column chromatography to afford the title compound as a white solid (150 mg, 64%). MS m/z: 335 [M+H]⁺.

### B. 5-((4-(2,2-Difluoroethyl)-6-fluoro-1H-indol-5-yl)oxy)-2-fluorobenzimidamide

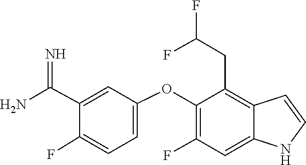 [SMILES: N=C(N)c1cc(Oc2c(F)cc3[nH]ccc3c2CC(F)F)ccc1F]

To a stirred solution of 5-((4-(2,2-difluoroethyl)-6-fluoro-1H-indol-5-yl)oxy)-2-fluorobenzonitrile (150 mg, 0.45 mmol) in THF (4 mL) was added lithium bis(trimethylsilyl)amide (1.0 M in THF, 3.6 mL, 3.6 mmol) at room temperature. The resulting mixture was stirred at room temperature for sixteen hours, quenched with methanol (10 mL) and concentrated. The resulting residue was purified by silica gel column chromatography (dichloromethane/methanol/concentrated ammonium hydroxide, v/v, 5/1/0.1) to afford the title compound as a yellow solid (120 mg, 76%). MS m/z: 352 [M+H]⁺.

### C. Example 25. 1-(2-(5-((4-(2,2-Difluoroethyl)-6-fluoro-1H-indol-5-yl)oxy)-2-fluorophenyl)-1H-imidazol-4-yl)-1-phenylethan-1-ol

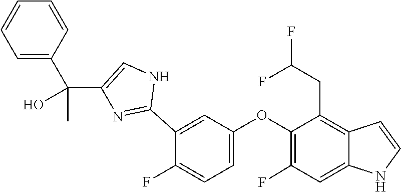 [SMILES: CC(O)(c1ccccc1)c1c[nH]c(-c2cc(Oc3c(F)cc4[nH]ccc4c3CC(F)F)ccc2F)n1]

A mixture of 5-((4-(2,2-difluoroethyl)-6-fluoro-1H-indol-5-yl)oxy)-2-fluorobenzimidamide (120 mg, 0.34 mmol) and 3-methyl-3-phenyloxirane-2-carbaldehyde (Intermediate 3, 72 mg, 0.44 mmol) in dry DMF (6 mL) was heated overnight at 80° C. under nitrogen atmosphere. The reaction mixture was concentrated to dryness and the resulting residue was purified by preparative HPLC to afford the title compound as a yellow solid (46 mg, 27%). LC-MS: Retention time: 0.95 min. MS m/z: 496 [M+H]⁺.

### Example 26

### (5-(3-(5-Benzyl-4H-1,2,4-triazol-3-yl)phenoxy)-1H-pyrrolo[3,2-b]pyridin-3-yl)methanol

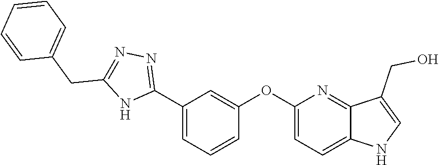 [SMILES: OCc1c[nH]c2ccc(Oc3cccc(-c4nnc(Cc5ccccc5)[nH]4)c3)nc12]

### A. 5-(3-(5-Benzyl-4H-1,2,4-triazol-3-yl)phenoxy)-1H-pyrrolo[3,2-b]pyridine

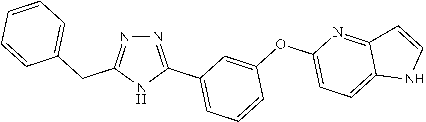 [SMILES: c1ccc(Cc2nnc(-c3cccc(Oc4ccc5[nH]ccc5n4)c3)[nH]2)cc1]

A mixture of 2-phenylacetonitrile (87 mg, 745 μmol), 3-((1H-pyrrolo[3,2-b]pyridin-5-yl)oxy)benzohydrazide (50 mg, 186 μmol) and potassium carbonate (52 mg, 373 μmol) in 2 mL of n-Butanol was heated to 165° C. for 80 minutes. The solvent was evaporated and the residue was chromatographed over silica gel column (0-100% ethyl acetate in heptane) twice to afford the title compound as a white solid (28 mg, 41%). MS m/z: 368 [M+H]⁺. ¹H NMR (400 MHz, CDCl₃) δ7.88 (dd, J=8.6, 0.8 Hz, 1H), 7.78 (d, J=8.5 Hz, 1H), 7.67 (s, 1H), 7.55-7.43 (m, 2H), 7.34-7.19 (m, 5H), 7.13 (dd, J=7.7, 2.0 Hz, 1H), 6.81 (d, J=8.6 Hz, 1H), 6.46 (dd, J=3.2, 0.8 Hz, 1H), 4.13 (s, 2H) ppm.

### B. 5-(3-(5-Benzyl-4H-1,2,4-triazol-3-yl)phenoxy)-1H-pyrrolo[3,2-b]pyridine-3-carbaldehyde

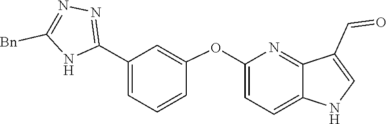 [SMILES: O=Cc1c[nH]c2ccc(Oc3cccc(-c4nnc(Cc5ccccc5)[nH]4)c3)nc12]

To a stirred solution of 5-(3-(5-benzyl-4H-1,2,4-triazol-3-yl)phenoxy)-1H-pyrrolo[3,2-b]pyridine (480 mg, 1.31 mmol) in a mixture of acetic acid (5 mL) and water (10 ml) was added hexamethylenetetramine (275 mg, 1.96 mmol). The resulting mixture was refluxed for two hours, cooled to room temperature, diluted with water (20 mL) and extracted with THF (30 mL×3). The combined organic layers were dried over sodium sulfate, filtered and concentrated to afford the title compound as a yellow solid (60.5 mg, 11%), which was used without further purification. MS m/z: 396 [M+H]⁺.

### C. Example 26. (5-(3-(5-Benzyl-4H-1,2,4-triazol-3-yl)phenoxy)-1H-pyrrolo[3,2-b]pyridin-3-yl)methanol

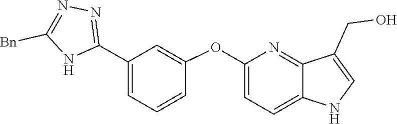 [SMILES: OCc1c[nH]c2ccc(Oc3cccc(-c4nnc(Cc5ccccc5)[nH]4)c3)nc12]

To a stirred solution of 5-(3-(5-benzyl-4H-1,2,4-triazol-3-yl)phenoxy)-1H-pyrrolo[3,2-b]pyridine-3-carbaldehyde (60.5 mg, 0.15 mmol) in methanol (15 mL) was added NaBH₄ (141 mg, 3.72 mmol). The reaction mixture was stirred at 65° C. for two hours and then concentrated. The resulting residue was diluted with water (50 ml) and extracted with THF (30 mL×3). The combined organic extracts were dried over sodium sulfate, filtered and concentrated. The crude product was purified by preparative HPLC to afford the title compound as a white solid (15 mg, 24%). MS m/z: 398 [M+H]⁺. ¹H NMR (400 MHz, CD₃OD) δ 8.42 (d, J=0.8 Hz, 1H), 7.75 (d, J=7.2 Hz, 1H), 7.61 (s, 1H), 7.59 (s, 1H), 7.47 (t, J=7.6 Hz, 1H), 7.34-7.24 (m, 6H), 7.10 (d, J=7.6 Hz, 1H), 4.77 (s, 2H), 4.61 (s, 1H), 4.15 (s, 2H) ppm.

### Example 27

### 5-(3-(5-(Hydroxy(phenyl)methyl)-1H-imidazol-2-yl)phenoxy)-1H-indazole-4-carboxylic acid

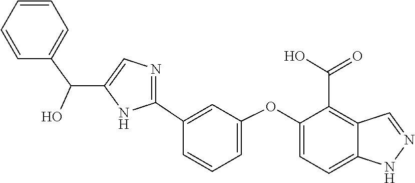 [SMILES: O=C(O)c1c(Oc2cccc(-c3ncc(C(O)c4ccccc4)[nH]3)c2)ccc2[nH]ncc12]

### A. Methyl 6-(3-cyanophenoxy)-2-methyl-3-nitrobenzoate

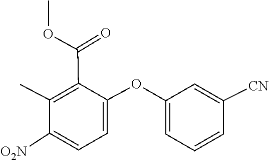 [SMILES: COC(=O)c1c(Oc2cccc(C#N)c2)ccc([N+](=O)[O-])c1C]

A mixture of methyl 6-fluoro-2-methyl-3-nitrobenzoate (9.7 g, 45.5 mmol), 3-hydroxybenzonitrile (5.5 g, 45.5 mmol) and potassium carbonate (7.5 g, 54.6 mmol) in acetonitrile (200 mL) was stirred at 80° C. overnight. The solvent evaporated, the residue was dissolved in ethyl acetate (300 mL) and washed with water and brine. The organic layer was dried over sodium sulfate and concentrated under vacuum to afford the title compound as a yellow solid (14 g, 100%). The crude product was used in next step without further purification. MS m/z: 313 [M+H]⁺. ¹H NMR (400 MHz, DMSO-d₆) δ 8.11 (d, J=9.2 Hz, 1H), 7.69-7.74 (m, 2H), 7.65 (t, J=8.0 Hz, 1H), 7.44-7.47 (m, 1H), 7.06 (d, J=9.2 Hz, 1H), 3.86 (s, 3H), 2.44 (s, 3H) ppm.

### B. Methyl 3-amino-6-(3-cyanophenoxy)-2-methylbenzoate

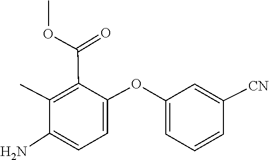 [SMILES: COC(=O)c1c(Oc2cccc(C#N)c2)ccc(N)c1C]

A mixture of methyl 6-(3-cyanophenoxy)-2-methyl-3-nitrobenzoate (6.5 g, 20.8 mmol), zinc powder (10.8 g, 166.4 mmol) and acetic acid (10 g, 166.4 mmol) in methanol (250 mL) was stirred at room temperature overnight. Solid was filtered off and washed with THF and methanol twice. The filtrate was concentrated under vacuum and the residue was purified by silica gel chromatography (petroleum ether/ethyl acetate, v/v, 4/1) to afford the title compound as yellow oil (4.6 g, 79%). MS m/z: 283 [M+H]⁺. ¹H NMR (400 MHz, DMSO-d₆) δ 7.46-7.52 (m, 2H), 7.23-7.26 (m, 1H), 7.14-7.16 (m, 1H), 6.76 (s, 2H), 3.64 (s, 3H), 1.98 (s, 3H) ppm.

### C. Methyl 5-(3-cyanophenoxy)-1H-indazole-4-carboxylate

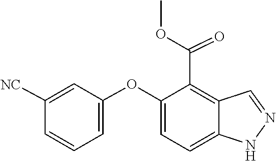 [SMILES: COC(=O)c1c(Oc2cccc(C#N)c2)ccc2[nH]ncc12]

To a stirred solution of methyl 3-amino-6-(3-cyanophenoxy)-2-methylbenzoate (3.0 g, 10.6 mmol) in acetic acid (100 mL) was added sodium nitrite (741 mg, 10.7 mmol) in water (5 mL) and the reaction mixture was stirred at 40° C. overnight. Sodium hydroxide (2M) solution was added to adjust the pH to 7-8 and extracted with ethyl acetate (50 mL×3). The combined organic extracts were washed with brine, dried over sodium sulfate and concentrated. The resulting residue was purified by silica gel chromatography (petroleum ether/ethyl acetate, v/v, 3/1 to 1/1) to afford the title compound as a yellow solid (2.4 g, 77%). MS m/z: 294 [M+H]⁺. ¹H NMR (400 MHz, DMSO-d₆) δ 13.52 (s, 1H), 8.32 (s, 1H), 7.89 (dd, J=8.8, 0.8 Hz, 1H), 7.50-7.53 (m, 2H), 7.33-7.34 (m, 1H), 7.21-7.39 (m, 2H), 3.80 (s, 3H) ppm.

### D. Methyl 5-(3-carbamimidoylphenoxy)-1H-indazole-4-carboxylate

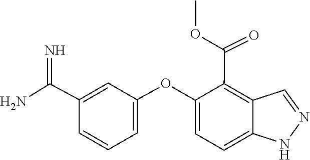 [SMILES: COC(=O)c1c(Oc2cccc(C(=N)N)c2)ccc2[nH]ncc12]

To a stirred solution of methyl 5-(3-cyanophenoxy)-1H-indazole-4-carboxylate (4 g, 13.7 mmol) in THF (100 mL) was added a 1M solution of lithium bis(trimethylsilyl)amide in THF (68 mL, 68 mmol). The mixture was stirred at room temperature under nitrogen atmosphere overnight, slowly quenched with aqueous ammonium chloride at 0° C. and extracted with THF (100 mL×3). The organic phase was washed with brine, dried over sodium sulfate and concentrated under vacuum to afford the title compound as a yellow solid (6.8 g, 100%). MS m/z: 311 [M+H]⁺.

### E. Methyl 5-(3-(5-(hydroxymethyl)-1H-imidazol-2-yl)phenoxy)-1H-indazole-4-carboxylate

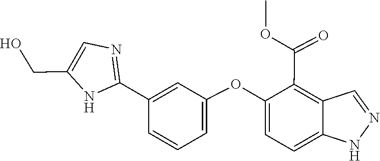 [SMILES: COC(=O)c1c(Oc2cccc(-c3ncc(CO)[nH]3)c2)ccc2[nH]ncc12]

A mixture of methyl 5-(3-carbamimidoylphenoxy)-1H-indazole-4-carboxylate (2 g, 6.5 mmol), 1,3-dihydroxyacetone (1.2 g, 13 mmol), ammonium chloride (1.7 g, 32 mmol) and ammonia in methanol (7M, 30 mL) was stirred in a sealed tube at 100° C. overnight and then concentrated. The resulting residue was purified by silica gel column chromatography (dichloromethane/methanol/concentrated ammonium hydroxide, v/v, 15/1/0.1) to afford the title compound as a yellow solid (1.15 g, 35%). MS m/z: 365 [M+H]⁺.

### F. Methyl 5-(3-(5-formyl-1H-imidazol-2-yl)phenoxy)-1H-indazole-4-carboxylate

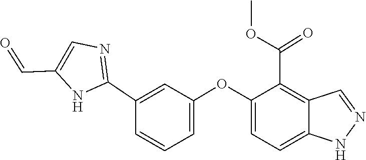 [SMILES: COC(=O)c1c(Oc2cccc(-c3ncc(C=O)[nH]3)c2)ccc2[nH]ncc12]

A mixture of methyl 5-(3-(5-(hydroxymethyl)-1H-imidazol-2-yl)phenoxy)-1H-indazole-4-carboxylate (400 mg, 1.1 mmoL) and manganese dioxide (957 mg, 11 mmol) in THF (50 mL) was stirred at room temperature overnight. Solid was filtered off and washed with methanol and THF. The filtrates were combined and concentrated to afford the title compound as a yellow oil (331 mg, 76%). MS m/z: 363 [M+H]⁺.

### G. Methyl 5-(3-(5-(hydroxy(phenyl)methyl)-1H-imidazol-2-yl)phenoxy)-1H-indazole-4-carboxylate

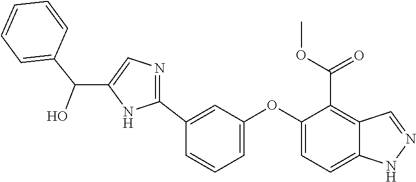 [SMILES: COC(=O)c1c(Oc2cccc(-c3ncc(C(O)c4ccccc4)[nH]3)c2)ccc2[nH]ncc12]

To a stirred solution of methyl 5-(3-(5-formyl-1H-imidazol-2-yl)phenoxy)-1H-indazole-4-carboxylate (330 mg, 0.91 mmol) in THF (150 mL) was added phenylmagnesium bromide (1M in THF, 7.28 mL, 7.28 mmol) at 0° C. The reaction mixture was stirred overnight at room temperature under nitrogen, quenched with methanol (50 mL) and concentrated. The resulting residue was purified by preparative HPLC to afford the title compound as a white solid (10 mg, 6%). MS m/z: 441 [M+H]⁺. ¹H NMR (400 MHz, CD₃OD) δ 8.37 (s, 1H), 7.81 (d, J=9.2 Hz, 1H), 7.56 (d, J=7.6 Hz, 1H), 7.26-7.46 (m, 7H), 7.22 (d, J=8.8 Hz, 1H), 6.93 (dd, J=2.4, 8.8 Hz, 1H), 6.77 (s, 1H), 5.80 (s, 1H), 3.87 (s, 3H) ppm.

### H. Example 27. 5-(3-(5-(Hydroxy(phenyl)methyl)-1H-imidazol-2-yl)phenoxy)-1H-indazole-4-carboxylic acid

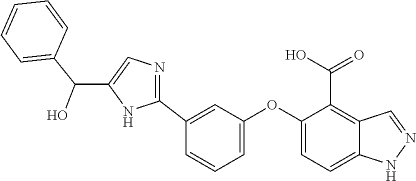 [SMILES: O=C(O)c1c(Oc2cccc(-c3ncc(C(O)c4ccccc4)[nH]3)c2)ccc2[nH]ncc12]

A mixture of methyl 5-(3-(5-(hydroxy(phenyl)methyl)-1H-imidazol-2-yl)phenoxy)-1H-indazole-4-carboxylate (30 mg, 0.07 mmol) and potassium hydroxide (78 mg, 1.4 mmol) in methanol (5 mL) and water (2 mL) was stirred at 60° C. overnight. The reaction mixture was concentrated under vacuum and purified by preparative HPLC to afford the title compound as a white solid (4 mg, 14%). MS m/z: 427 [M+H]⁺. ¹H NMR (400 MHz, CD₃OD) δ 8.31 (s, 1H), 7.68 (d, J=8.8 Hz, 1H), 7.53 (d, J=7.6 Hz, 1H), 7.29-7.46 (m, 7H), 7.17 (d, J=8.8 Hz, 1H), 7.04 (dd, J=8.0, 2.0 Hz, 1H), 6.82 (s, 1H), 5.81 (s, 1H) ppm.

### Example 28

### 5-(3-(5-(Hydroxy(phenyl)methyl)-1H-imidazol-2-yl)phenoxy)-N-(2-(methylamino)-2-oxoethyl)-1H-indazole-4-carboxamide

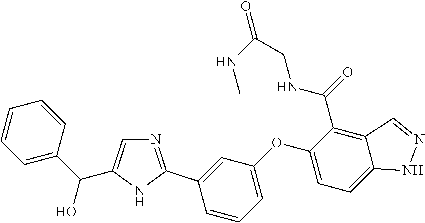 [SMILES: CNC(=O)CNC(=O)c1c(Oc2cccc(-c3ncc(C(O)c4ccccc4)[nH]3)c2)ccc2[nH]ncc12]

A mixture of 5-(3-(5-(hydroxy(phenyl)methyl)-1H-imidazol-2-yl)phenoxy)-1H-indazole-4-carboxylic acid (30 mg, 0.07 mmol), 2-amino-N-methylacetamide hydrochloride (17 mg, 0.14 mmol), triethylamine (14 mg, 0.14 mmol) and HATU (53 mg, 0.14 mmol) in DMF (5 mL) was stirred at room temperature for two hours. The mixture was diluted with water (20 mL) and extracted with ethyl acetate (20 mL×3). The combined organic extracts were washed with brine, dried over sodium sulfate and concentrated under vacuum. The residue was purified by preparative HPLC to afford the title compound as a white solid (3 mg, 9%). MS m/z: 497 [M+H]⁺. ¹H NMR (400 MHz, CD₃OD) δ 8.51 (s, 1H), 7.73 (d, J=9.2 Hz, 1H), 7.63 (d, J=8.4 Hz, 1H), 7.44-7.52 (m, 4H), 7.25-7.37 (m, 3H), 7.19 (d, J=9.2 Hz, 1H), 7.08 (dd, J=8.0, 1.6 Hz, 1H), 6.82 (s, 1H), 5.80 (s, 1H), 4.04 (s, 2H), 2.69 (s, 3H) ppm.

### Example 29

### 5-((5-(5-Benzyl-4H-1,2,4-triazol-3-yl)pyridin-3-yl)oxy)-1H-indole

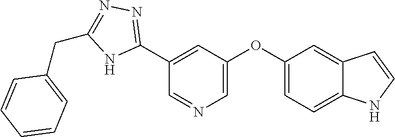 [SMILES: c1ccc(Cc2nnc(-c3cncc(Oc4ccc5[nH]ccc5c4)c3)[nH]2)cc1]

### A. Methyl 5-((1-tosyl-1H-indol-5-yl)oxy)nicotinate

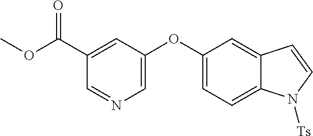 [SMILES: COC(=O)c1cncc(Oc2ccc3c(ccn3S(=O)(=O)c3ccc(C)cc3)c2)c1]

A mixture of 5-bromo-1-tosyl-1H-indole (4 g, 11.5 mmol), methyl 5-hydroxynicotinate (3.5 g, 23 mmol), CuI (1.1 g, 5.7 mmol), Cs₂CO₃ (7.5 g, 23 mmol) and 2-amino-N,N-dimethylacetamide hydrochloride (862 mg, 5.7 mmol) in a mixture of 1,4-dioxane (10 mL) and DMF (2.5 mL) was heated at 160° C. for 0.5 hour in a Biotage microwave synthesizer. The mixture was diluted with water (30 mL) and extracted with ethyl acetate (50 mL×4). The combined organic extracts were washed with water (50 mL×2) and brine (50 mL), dried over sodium sulfate and concentrated in vacuo. The resulting crude product was purified by silica gel column chromatography (petroleum ether/ethyl acetate, v/v, 10/1-3/1) to afford the title compound as a light yellow solid (340 mg, 7%). MS m/z: 423 [M+H]⁺.

### B. 5-((1-Tosyl-1H-indol-5-yl)oxy)nicotinohydrazide

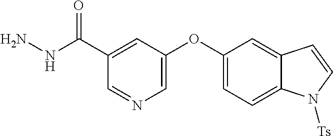 [SMILES: Cc1ccc(S(=O)(=O)n2ccc3cc(Oc4cncc(C(=O)NN)c4)ccc32)cc1]

To a stirred solution of methyl 5-((1-tosyl-1H-indol-5-yl)oxy)nicotinate (340 mg, 0.81 mmol) in methanol (10 mL) was added hydrazine hydrate (4 mL). The resulting mixture was stirred at 90° C. for five hours and concentrated in vacuo. Water (20 mL) was added to the residue and the mixture was extracted with ethyl acetate (30 mL×3). The combined organic extracts were washed with brine (30 mL), dried over sodium sulfate, filtered and concentrated in vacuo to afford the title compound as a light white solid (320 mg, 94%), which was used in the next step without further purification. MS m/z: 423 [M+H]⁺.

### C. Example 29. 5-((5-(5-Benzyl-4H-1,2,4-triazol-3-yl)pyridin-3-yl)oxy)-1H-indole

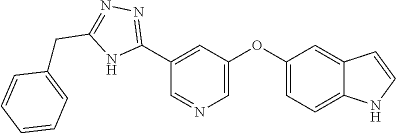 [SMILES: c1ccc(Cc2nnc(-c3cncc(Oc4ccc5[nH]ccc5c4)c3)[nH]2)cc1]

A mixture of 5-((1-tosyl-1H-indol-5-yl)oxy)nicotinohydrazide (310 mg, 0.73 mmol), 2-phenylacetonitrile (129 mg, 1.1 mmol) and potassium carbonate (507 mg, 3.67 mmol) in n-Butanol (15 mL) was stirred at 135° C. for eight hours under nitrogen atmosphere. The reaction mixture was concentrated, dissolved in ethyl acetate (50 mL) and washed with water (50 mL) and brine (50 mL). The organic layer was dried over sodium sulfate, filtered and concentrated in vacuo. The resulting crude product was purified by preparative HPLC to afford the title compound as a white solid (60 mg, 22%). MS m/z: 368 [M+H]⁺. ¹H NMR (500 MHz, DMSO-d₆) δ 11.26 (s, 1H), 8.83 (d, J=2.0 Hz, 1H), 8.41 (d, J=3.0 Hz, 1H), 7.61 (s, 1H), 7.48 (d, J=9.0 Hz, 1H), 7.44 (t, J=2.5 Hz, 1H), 7.35 (d, J=2.5 Hz, 1H), 7.30-7.22 (m, 5H), 6.93 (dd, J=8.5, 2.0 Hz, 1H), 6.45 (t, J=2.0 Hz, 1H), 4.09 (s, 2H) ppm.

### Example 30

### 2-(5-(3-(5-Benzyl-4H-1,2,4-triazol-3-yl)phenoxy)-1H-indol-3-yl)acetic acid

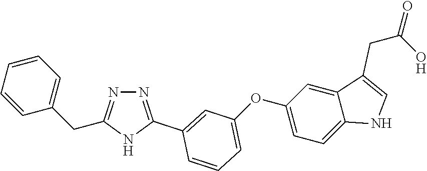 [SMILES: O=C(O)Cc1c[nH]c2ccc(Oc3cccc(-c4nnc(Cc5ccccc5)[nH]4)c3)cc12]

### A. 3-((1H-Indol-5-yl)oxy)benzohydrazide

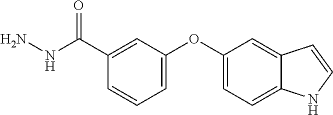 [SMILES: NNC(=O)c1cccc(Oc2ccc3[nH]ccc3c2)c1]

To a stirred solution of methyl 3-((1H-indol-5-yl)oxy)benzoate (Intermediate 1-5, 20.0 g, 0.12 mol) in ethanol (100 mL) was added hydrazine hydrate (75%, 100 g, 2.34 mol). The reaction mixture was refluxed for four hours and then concentrated in vacuo. The resulting residue was diluted with water (200 mL) and the pH was adjusted to 5-6 with 1N hydrochloric acid solution. The precipitate was filtered and washed with water (100 mL×2) to afford the title compound as a light brown solid (13.6 g, 74%), which was used without further purification. MS m/z: 268 [M+H]⁺.

### B. 5-(3-(5-Benzyl-4H-1,2,4-triazol-3-yl)phenoxy)-1H-indole

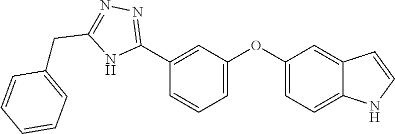 [SMILES: c1ccc(Cc2nnc(-c3cccc(Oc4ccc5[nH]ccc5c4)c3)[nH]2)cc1]

To a stirred solution of 3-((1H-indol-5-yl)oxy)benzohydrazide (13.6 g, 89.5 mmol) and 2-phenylacetonitrile (20.9 g, 179 mmol) in n-butanol (150 mL) was added potassium carbonate (37 g, 268 mmol). The resulting mixture was refluxed overnight and quenched with 1N sodium hydroxide solution (200 mL). The aqueous layer was washed with dichloromethane (200 mL×2) to remove any basic/neutral impurities and then the pH of the solution was adjusted to 9-10 with 1N hydrochloric acid solution. The precipitate was collected by filtration and washed with dichloromethane (100 mL×2) to afford the title compound as a solid (14.4 g, 64%), which was used without further purification.

### C. 2-(5-(3-(5-Benzyl-4H-1,2,4-triazol-3-yl)phenoxy)-1H-indol-3-yl)-2-oxoacetic acid

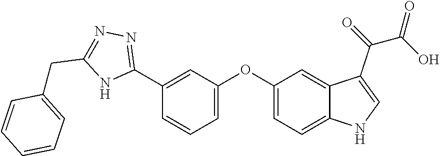 [SMILES: O=C(O)C(=O)c1c[nH]c2ccc(Oc3cccc(-c4nnc(Cc5ccccc5)[nH]4)c3)cc12]

To a stirred solution of 5-(3-(5-benzyl-4H-1,2,4-triazol-3-yl)phenoxy)-1H-indole (2.9 g, 9.89 mmol) in diethyl ether (15 mL) was added a solution of oxalyl chloride (422.7 mg, 286 mL, 3.3 mmol) in diethyl ether (5 mL) dropwise under nitrogen atmosphere at 0° C. The resulting mixture was stirred at 0° C. for three hours and then quenched with water (30 mL). The precipitate was filtered and washed with water (30 mL×2) to afford the title compound as a yellow solid (2.43 g, 71%), which was used without further purification. MS m/z: 439 [M+H]⁺.

### D. Example 30. 2-(5-(3-(5-Benzyl-4H-1,2,4-triazol-3-yl)phenoxy)-1H-indol-3-yl)acetic acid

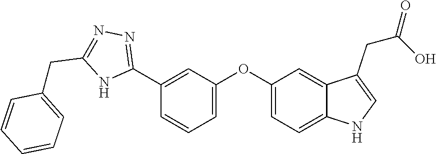 [SMILES: O=C(O)Cc1c[nH]c2ccc(Oc3cccc(-c4nnc(Cc5ccccc5)[nH]4)c3)cc12]

To a stirred solution of 2-(5-(3-(5-benzyl-4H-1,2,4-triazol-3-yl)phenoxy)-1H-indol-3-yl)-2-oxoacetic acid (400 mg, 9.89 mmol) in 2-methoxyethanol (5 mL) was added hydrazine hydrate (122 mg, 2.44 mmol). The temperature of the mixture was increased to 60° C. and sodium methoxide (283.2 mg, 5.24 mmol) was added in small portions The mixture was heated at 150° C. for three hours, cooled to room temperature and diluted with water (10 mL). The pH of the mixture was adjusted to 5-6 with 1N hydrochloric acid and extracted with ethyl acetate (20 mL×3). The combined organic layers were washed with brine, dried over sodium sulfate, filtered and concentrated in vacuo. The resulting residue was purified by preparative HPLC to afford the title compound as a white solid (80 mg, 21%). MS m/z: 425 [M+H]⁺. ¹H NMR (500 MHz, DMSO-d₆): δ 10.99 (s, 1H), 7.64 (d, J=7.5 Hz, 1H), 7.46 (s, 1H), 7.42-7.38 (m, 2H), 7.32-7.24 (m, 6H), 7.23-7.21 (m, 1H), 6.99 (dd, J=8.5, 2.0 Hz, 1H), 6.85 (dd, J=8.5, 2.0 Hz, 1H), 4.06 (s, 2H), 3.53 (s, 2H) ppm.

### Example 31

### 5-(3-(5-(Phenylsulfinyl)-1H-imidazol-2-yl)phenoxy)-1H-indole

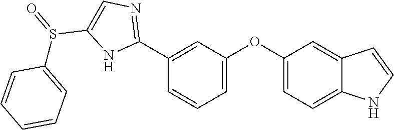 [SMILES: O=S(c1ccccc1)c1cnc(-c2cccc(Oc3ccc4[nH]ccc4c3)c2)[nH]1]

### A. 1-((Benzyloxy)methyl)-2-(3-methoxyphenyl)-1H-imidazole

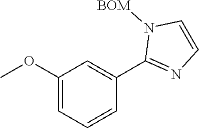

To a stirred solution of 2-(3-methoxyphenyl)-1H-imidazole (800 mg, 4.6 mmol) in DMF (10 mL) was added sodium hydride (60% in mineral oil, 276 mg, 6.9 mmol) at 0° C. and the resulting mixture was stirred for one hour. Benzyloxymethyl chloride (0.95 mL, 6.9 mmol) was added at 0° C. and then stirred at room temperature overnight. The reaction mixture was quenched with water (20 mL) and extracted with ethyl acetate (30 mL×3). The combined organic extracts were washed with water (30 mL) and brine (30 mL), dried over sodium sulfate, filtered and concentrated in vacuo. The resulting residue was purified by silica gel column chromatography (petroleum ether/ethyl acetate, v/v, 3/1-1/1) to afford the title compound as a yellow oil (1.15 g, 85%). MS m/z: 295 [M+H]⁺.

### B. 1-((Benzyloxy)methyl)-2-(3-methoxyphenyl)-5-(phenylthio)-1H-imidazole

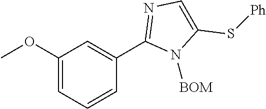

To a stirred solution of 1-((benzyloxy)methyl)-2-(3-methoxyphenyl)-1H-imidazole (370 mg, 1.26 mmol) in anhydrous THF (4 mL) was added n-BuLi (0.8 mL, 2.0 mmol, 2.5 M in hexane) over thirty minutes at −78° C. and the reaction mixture was stirred at −78° C. for one hour. A solution of diphenyl disulfide (412 mg, 1.89 mmol) in anhydrous THF (4 mL) was added over thirty minutes at −78° C. The mixture was warmed to room temperature, stirred overnight, quenched with water (20 mL) and extracted with ethyl acetate (30 mL×3). The combined organic extracts were washed with brine, dried over sodium sulfate, filtered and concentrated. The resulting crude product was purified by silica gel column chromatography (petroleum ether/ethyl acetate, v/v, 10/1-5/1) to afford the title compound as a yellow oil (350 mg, 70%). MS m/z: 403 [M+H]⁺.

### C. 3-(5-(Phenylthio)-1H-imidazol-2-yl)phenol

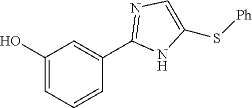 [SMILES: Oc1cccc(-c2ncc(Sc3ccccc3)[nH]2)c1]

To a stirred solution of 1-((benzyloxy)methyl)-2-(3-methoxyphenyl)-5-(phenylthio)-1H-imidazole (350 mg, 0.87 mmol) in dichloromethane (5 mL) was added a solution of boron tribromide (3N in dichloromethane, 0.87 mL, 2.61 mmol) dropwise at 0° C. over thirty minutes. The resulting mixture was warmed to room temperature, stirred for 18 hours and then quenched with methanol at −78° C. The mixture was concentrated in vacuo. The resulting residue was dissolved in ethyl acetate (50 mL), washed with water (20 mL) and brine (20 mL), dried over sodium sulfate, filtered and concentrated. The resulting crude product was purified by silica gel column chromatography (dichloromethane/methanol, v/v, 30/1) to afford the title compound as a yellow solid (170 mg, 73%). MS m/z: 269 [M+H]⁺.

### D. 3-(5-(Phenylthio)-1H-imidazol-2-yl)phenyl trifluoromethanesulfonate

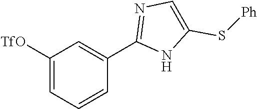 [SMILES: O=S(=O)(Oc1cccc(-c2ncc(Sc3ccccc3)[nH]2)c1)C(F)(F)F]

To a stirred solution of 3-(5-(phenylthio)-1H-imidazol-2-yl)phenol (100 mg 0.37 mmol) and triethylamine (75 mg, 0.74 mmol) in THF (4 mL) was added N,N-bis[[(trifluoromethyl)sulfonyl]oxy]-benzenamine (288 mg, 0.74 mmol) at 0° C. The reaction mixture was stirred at room temperature overnight and then concentrated in vacuo. The resulting residue was purified by preparative TLC (petroleum ether/ethyl acetate, v/v, 3/1) to afford the title compound as a white solid (110 mg, 74%). MS m/z: 401 [M+H]⁺.

### E. 5-(3-(5-(Phenylthio)-1H-imidazol-2-yl)phenoxy)-1H-indole

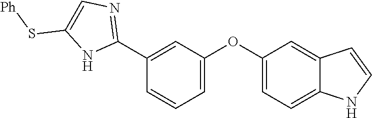 [SMILES: c1ccc(Sc2cnc(-c3cccc(Oc4ccc5[nH]ccc5c4)c3)[nH]2)cc1]

A mixture of 3-(5-(phenylthio)-1H-imidazol-2-yl)phenyl trifluoromethanesulfonate (200 mg, 0.5 mmol), tert-butyl 5-hydroxy-1H-indole-1-carboxylate (234 mg, 1 mmol), 2-di-t-butylphosphino-2′,4′,6′-tri-i-propyl-1,1′-biphenyl (36 mg, 0.04 mmol), Pd₂(dba)₃ (18 mg, 0.02 mmol) and K₃PO₄ (266 mg, 1 mmol) in toluene (5 mL) was heated at 150° C. for two hours in a in a Biotage microwave synthesizer. The reaction mixture was cooled to room temperature, quenched with water (20 mL) and extracted with ethyl acetate (20 mL×3). The combined organic extracts were washed with water (20 mL) and brine (20 mL), dried over sodium sulfate, filtered and concentrated. The resulting crude product was purified by silica gel column chromatography (petroleum ether/ethyl acetate, v/v, 10/1-5/1) to afford the title compound as a yellow oil (40 mg, 20%). MS m/z: 384 [M+H]⁺. ¹H NMR (500 MHz, DMSO-d₆) δ 11.40 (s, 1H), 9.47 (s, 1H), 7.79 (s, 1H), 7.59 (s, 1H), 7.49-7.46 (m, 2H), 7.35-7.32 (m, 4H), 7.20-7.18 (m, 1H), 7.02-6.98 (m, 2H), 6.88 (s, 1H), 6.67 (d, J=7.5 Hz, 2H), 6.50 (s, 1H) ppm. ¹³C NMR (100 MHz, DMSO-d₆) δ 162.2, 152.8, 142.9, 140.5, 136.3, 135.1, 134.7, 134.3, 134.2, 133.3, 132.8, 132.6, 132.0, 130.8, 124.5, 123.9, 122.7, 120.7, 120.3, 117.3, 107.0 ppm.

### F. Example 31. 5-(3-(5-(Phenylsulfinyl)-1H-imidazol-2-yl)phenoxy)-1H-indole

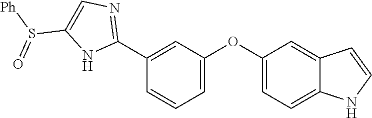 [SMILES: O=S(c1ccccc1)c1cnc(-c2cccc(Oc3ccc4[nH]ccc4c3)c2)[nH]1]

To a stirred solution of 5-(3-(5-(phenylthio)-1H-imidazol-2-yl)phenoxy)-1H-indole (100 mg, 0.26 mmol) in dichloromethane (2 mL) was added m-CPBA (53 mg, 0.26 mmol) at −78° C. The reaction mixture was stirred at −78° C. for one hour, quenched with saturated sodium sulfite solution (10 mL) and extracted with dichloromethane (20 mL×3). The combined organic extracts were washed with saturated sodium carbonate solution (20 mL×2) and brine (20 mL), dried over sodium sulfate, filtered and concentrated in vacuo. The resulting residue was purified by preparative HPLC to afford the title compound as white solid (20 mg, 19%). MS m/z: 400 [M+H]⁺. ¹H NMR (500 MHz, DMSO-d₆): δ 11.19 (s, 1H), 7.67 (dd, J=8.0, 2.0 Hz, 2H), 7.64 (s, 1H), 7.59-7.51 (m, 4H), 7.48-7.38 (m, 4H), 7.24 (d, J=2.0 Hz, 1H), 6.94 (dd, J=8.0, 2.0 Hz, 1H), 6.85 (dd, J=8.0, 2.0 Hz, 1H), 6.43-6.40 (m, 1H) ppm.

### Example 32

### 5-(3-(5-(Phenylsulfonyl)-1H-imidazol-2-yl)phenoxy)-1H-indole

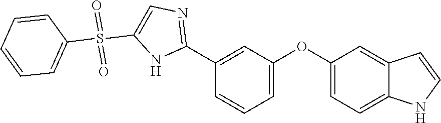 [SMILES: O=S(=O)(c1ccccc1)c1cnc(-c2cccc(Oc3ccc4[nH]ccc4c3)c2)[nH]1]

To a stirred solution of 5-(3-(5-(phenylthio)-1H-imidazol-2-yl)phenoxy)-1H-indole (Example 31 Step E, 80 mg, 0.21 mmol) in dichloromethane (2 mL) was added m-CPBA (42 mg, 0.21 mmol) at −78° C. The reaction mixture was stirred at −78° C. for one hour, another portion of m-CPBA (42 mg, 0.21 mmol) was added and stirred at 0° C. for another four hours. The reaction was quenched with saturated sodium sulfite solution (10 mL) and extracted with dichloromethane (20 mL×3). The combined organic extracts were washed with saturated sodium carbonate solution (20 mL×2) and brine (20 mL), dried over sodium sulfate, filtered and concentrated. The resulting residue was purified by preparative HPLC to afford the title compound as a white solid (2 mg, 2.3%). MS m/z: 416 [M+H]⁺. ¹H NMR (500 MHz, DMSO-d₆) δ 11.17 (s, 1H), 7.87 (m, 3H), 7.61-7.53 (m, 5H), 7.47 (s, 1H), 7.41 (d, J=9.0 Hz, 1H), 7.39 (t, J=2.5 Hz, 1H), 7.33 (t, J=7.5 Hz, 1H), 7.21 (d, J=2.0 Hz, 1H), 6.87-6.83 (m, 2H), 6.40 (s, 1H) ppm.

### Example 33

### (5-(3-((1H-Benzo[d]imidazol-5-yl)oxy)phenyl)-4H-1,2,4-triazol-3-yl)(1-methyl-1H-pyrazol-4-yl)methanol

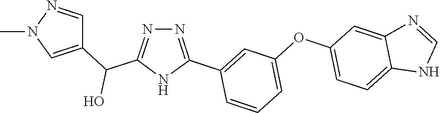 [SMILES: Cn1cc(C(O)c2nnc(-c3cccc(Oc4ccc5[nH]cnc5c4)c3)[nH]2)cn1]

### A. 2-Hydroxy-2-(1-methyl-1H-pyrazol-4-yl)acetohydrazide

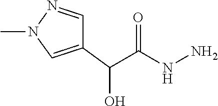

To a stirred solution of methyl 2-hydroxy-2-(1-methyl-1H-pyrazol-4-yl)acetate (850 mg, 5 mmol) in methanol (20 mL) was added hydrazine hydrate (85%, 5 mL). The reaction mixture was heated overnight and concentrated to afford the title compound as a white solid (860 mg, 100%), which was used without further purification. MS m/z: 171 [M+H]⁺.

### B. 3-(3-Amino-4-nitrophenoxy)benzonitrile

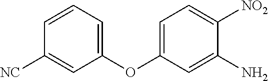 [SMILES: N#Cc1cccc(Oc2ccc([N+](=O)[O-])c(N)c2)c1]

To a suspension of 5-fluoro-2-nitrobenzenamine (10 g, 64.1 mmol) and potassium carbonate (17.7 g 128.2 mmol) in DMF (100 mL) was added 3-hydroxybenzonitrile (8.40 g 70.5 mmol). The resulting mixture was stirred at 100° C. for three hours and then quenched with water (200 mL). The precipitate was filtered, washed with water (100×2) and dried to afford the title compound as a yellow solid (15 g, 94%). The solid was used without further purification. MS m/z: 256 [M+H]⁺.

### C. 3-(3,4-Diaminophenoxy)benzonitrile

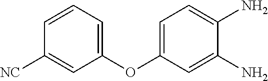 [SMILES: N#Cc1cccc(Oc2ccc(N)c(N)c2)c1]

To a solution of 3-(3-amino-4-nitrophenoxy)benzonitrile (10 g, 39.2 mmol) in ethyl acetate (200 mL) was added Pd/C (1 g, 10% Pd on carbon). The reaction mixture was stirred overnight at room temperature under hydrogen atmosphere, filtered through a pad of Celite and washed with ethyl acetate (20 mL×3). The filtrate was concentrated to afford the title compound as yellow oil (9.05 g). The product was used in the next step without further purification. MS m/z: 226 [M+H]⁺.

### D. 3-((1H-Benzo[d]imidazol-5-yl)oxy)benzonitrile

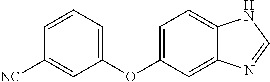 [SMILES: N#Cc1cccc(Oc2ccc3[nH]cnc3c2)c1]

To a solution of 3-(3,4-diaminophenoxy)benzonitrile (9.05 g, 40.2 mmol) in ethanol (100 mL) was added p-toluene sulfonic acid (1.38 g, 8.04 mmol) and triethyl orthoformate (20.1 mL, 120.6 mmol). The resulting mixture was refluxed for three hours and then concentrated. The resulting residue was purified by silica gel column chromatography (dichloromethane/methanol, v/v, 40/1-20/1) to afford the tile compound as a yellow solid (8.36 g, 88% from 2 steps). MS m/z: 236 [M+H]⁺.

### E. Ethyl 3-((1H-benzo[d]imidazol-5-yl)oxy)benzimidate

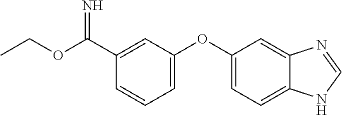 [SMILES: CCOC(=N)c1cccc(Oc2ccc3[nH]cnc3c2)c1]

To a stirred solution of 3-((1H-benzo[d]imidazol-5-yl)oxy)benzonitrile (235 mg, 1 mmol) in ethanol (5 mL) was added acetyl chloride (3 mL) dropwise at 0° C. under nitrogen. The reaction mixture was stirred at room temperature overnight and concentrated to afford the title compound as a yellow solid (350 mg, purity 45%). The product was used in the next step without further purification. MS m/z: 282 [M+H]⁺.

### F. Example 33. (5-(3-((1H-Benzo[d]imidazol-5-yl)oxy)phenyl)-4H-1,2,4-triazol-3-yl)(1-methyl-1H-pyrazol-4-yl)methanol

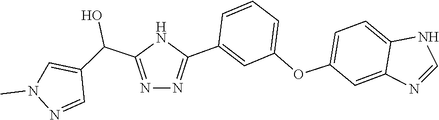 [SMILES: Cn1cc(C(O)c2nnc(-c3cccc(Oc4ccc5[nH]cnc5c4)c3)[nH]2)cn1]

Triethylamine (404 mg, 4.0 mmol) was added to a solution of ethyl 3-((1H-benzo[d]imidazol-5-yl)oxy)benzimidate (350 mg, crude) in anhydrous ethanol (10 mL) at room temperature under nitrogen atmosphere. After stirring for ten minutes, 2-hydroxy-2-(1-methyl-1H-pyrazol-4-yl)acetohydrazide (170 mg, 1 mmol) was added and the resulting mixture was heated at 60° C. for 16 hours. The solvent was removed under reduced pressure and the resulting crude product was purified by preparative HPLC to afford the title compound as a white solid (45 mg, 12%). MS m/z: 388 [M+H]⁺. ¹H NMR (400 MHz, CD₃OD) δ 8.19 (s, 1H), 7.74 (d, J=7.6 Hz, 1H), 7.65-7.62 (m, 2H), 7.60 (s, 1H), 7.51 (s, 1H), 7.44 (t, J=8.0 Hz, 1H), 7.28 (d, J=2.0 Hz, 1H), 7.09-7.05 (m, 2H), 5.94 (s, 1H), 3.85 (s, 3H) ppm. ¹³C NMR (100 MHz, CD₃OD) δ 161.9, 159.2, 159.0, 152.6, 141.9, 137.3, 131.2, 129.9, 129.5, 122.6, 120.3, 118.8, 115.6, 115.2, 61.7, 37.4 ppm.

### Example 34

### (3-(5-Benzyl-4H-1,2,4-triazol-3-yl)phenyl)(1H-indol-5-yl)methanone

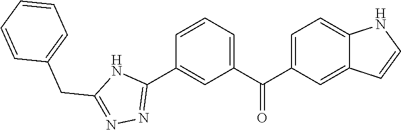 [SMILES: O=C(c1cccc(-c2nnc(Cc3ccccc3)[nH]2)c1)c1ccc2[nH]ccc2c1]

### A. (3-Bromophenyl)(1-tosylindolin-5-yl)methanone

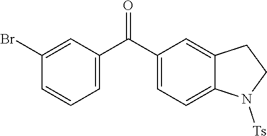 [SMILES: Cc1ccc(S(=O)(=O)N2CCc3cc(C(=O)c4cccc(Br)c4)ccc32)cc1]

To a suspension of aluminum chloride (9.7 g, 73.3 mmol) in dichloromethane (150 mL) was added 3-bromobenzoyl chloride (12.8 g, 58.6 mmol) at 0° C. and stirred at room temperature for one hour. A solution of 1-tosylindoline (10 g, 36.6 mmol) in dichloromethane (30 mL) was added dropwise and stirred overnight at room temperature. The reaction mixture was poured onto crushed ice (200 g) and dichloromethane (100 mL) and extracted with dichloromethane (100 mL×2) The combined organic extracts were washed with brine and dried over sodium sulfate. The solvent was evaporated and the residue was purified by silica gel column chromatography (petroleum ether/ethyl acetate, v/v, 10/1) to afford the title compound as a white solid (11.6 g, 70%). MS m/z: 456, 458 [M+H]⁺.

### B. Methyl 3-(1-tosylindoline-5-carbonyl)benzoate

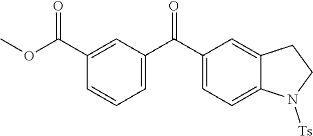 [SMILES: COC(=O)c1cccc(C(=O)c2ccc3c(c2)CCN3S(=O)(=O)c2ccc(C)cc2)c1]

A mixture of (3-bromophenyl)(1-tosylindolin-5-yl)methanone (4 g, 8.8 mmol), triethylamine (2.7 g, 26.4 mmol) and Pd(dppf)Cl₂ (1.8 g, 2.19 mmol) in methanol (100 mL) was heated at 60° C. under carbon monoxide atmosphere overnight. The reaction mixture was cooled to room temperature and filtered. The filtrate was concentrated and the residue was purified by silica gel column chromatography (petroleum ether/ethyl acetate, v/v, 5/1) to afford the title compound as a white solid (2.75 g, 72%). MS m/z: 436 [M+H]⁺.

### C. 3-(1-Tosylindoline-5-carbonyl)benzoic acid

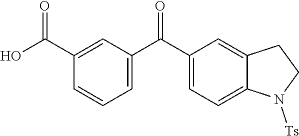 [SMILES: Cc1ccc(S(=O)(=O)N2CCc3cc(C(=O)c4cccc(C(=O)O)c4)ccc32)cc1]

Methyl 3-(1-tosylindoline-5-carbonyl)benzoate (6.3 g, 14.5 mmol) was dissolved in THF and water (1:1, 50 mL) and lithium hydroxide monohydrate (1.82 g, 43.4 mmol) was added. The reaction mixture was stirred overnight at room temperature and concentrated to remove THF. The pH of the aqueous residue was adjusted to ˜3 with 3M hydrochloric acid and the solid was collected by filtration to afford the title compound (5.7 g, 93%). The product was used without further purification. MS m/z: 422 [M+H]⁺.

### D. 3-(1-Tosylindoline-5-carbonyl)benzohydrazide

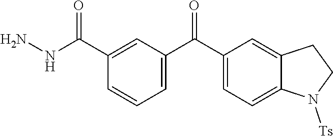 [SMILES: Cc1ccc(S(=O)(=O)N2CCc3cc(C(=O)c4cccc(C(=O)NN)c4)ccc32)cc1]

To a stirred solution of 3-(1-tosylindoline-5-carbonyl)benzoic acid (5.7 g, 13.1 nmol) and DMF (2 drops) in dichloromethane (60 mL) was added oxalyl chloride (6.6 g, 52.4 mmol) at 0° C. The reaction mixture was stirred for one hour at room temperature and concentrated to dryness. The residue was dissolved in dichloromethane (30 mL) and hydrazine hydrate (6.55 mL, 131 mmol) was added dropwise at 0° C. The mixture was stirred at 0° C. for 15 minutes, quenched with water (50 ml) and extracted with THF (50 mL×3). The combined organic extracts were dried, filtered and concentrated to afford the title compound as a white solid (5.1 g, 87%). The product was used without further purification. MS m/z: 436 [M+H]⁺.

### E. (3-(5-Benzyl-4H-1,2,4-triazol-3-yl)phenyl)(1-tosylindolin-5-yl)methanone

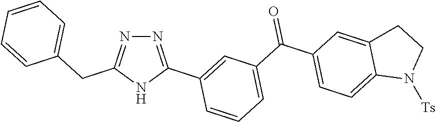 [SMILES: Cc1ccc(S(=O)(=O)N2CCc3cc(C(=O)c4cccc(-c5nnc(Cc6ccccc6)[nH]5)c4)ccc32)cc1]

To a stirred solution of phenylacetonitrile (3 g, 25.6 mmol) in ethyl alcohol (14.2 g, 308 nmol) was added acetyl chloride (16 g, 205 mmol) dropwise. The reaction mixture was stirred for five hours at 0° C. and concentrated to dryness to afford ethyl 2-phenylacetimidate hydrochloride (5.1 g). The product was used without further purification. MS m/z: 164 [M+H]⁺. A mixture of ethyl 2-phenylacetimidate hydrochloride (4.7 g, 23.4 mmol), 3-(1-tosylindoline-5-carbonyl)benzohydrazide (5.1 g, 11.7 mmol) and triethylamine (11.8 g, 117 mmol) in ethanol (100 mL) was stirred at 70° C. overnight and concentrated to dryness. The resulting residue was purified by silica gel column chromatography (petroleum ether/ethyl acetate, v/v, 5/1) to afford the title compound as a white solid (4.6 g, 73%). MS m/z: 535 [M+H]⁺.

### F. (3-(5-Benzyl-4H-1,2,4-triazol-3-yl)phenyl)(1-tosyl-1H-indol-5-yl)methanone

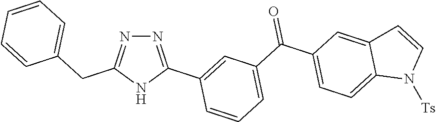 [SMILES: Cc1ccc(S(=O)(=O)n2ccc3cc(C(=O)c4cccc(-c5nnc(Cc6ccccc6)[nH]5)c4)ccc32)cc1]

To a stirred solution of (3-(5-Benzyl-4H-1,2,4-triazol-3-yl)phenyl)(1-tosylindolin-5-yl)methanone (4.6 g, 8.61 mmol) in acetic acid (20 ml) was added manganese(III) acetate (6.93 g, 25.8 mmol). The reaction mixture was stirred at 70° C. overnight and concentrated to dryness. The residue was purified by silica gel column chromatography (petroleum ether/ethyl acetate, v/v, 2:1) to afford the title compound as a white solid (2.5 g, 54%). MS m/z: 533 [M+H]⁺.

### G. Example 34. (3-(5-Benzyl-4H-1,2,4-triazol-3-yl)phenyl)(1H-indol-5-yl)methanone

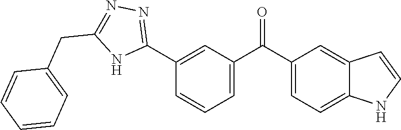 [SMILES: O=C(c1cccc(-c2nnc(Cc3ccccc3)[nH]2)c1)c1ccc2[nH]ccc2c1]

A mixture of (3-(5-benzyl-4H-1,2,4-triazol-3-yl)phenyl)(1-tosyl-1H-indol-5-yl)methanone (2.4 g, 4.5 mmol) and sodium hydroxide (720 mg, 18 mmol) in methanol (30 mL) was heated at 60° C. for three hours and concentrated to dryness. The resulting residue was triturated with water (30 mL) to form a solid, which was collected by filtration and purified by silica gel column chromatography (petroleum ether/ethyl acetate, v/v, 2/1) to afford the title compound as a white solid (1.45 g, 85%). MS m/z: 379 [M+H]⁺. ¹H NMR (400 MHz, DMSO-d₆) δ 13.99 (br s, 1H), 11.56 (br s, 1H), 8.29 (s, 1H), 8.22 (d, J=7.6 Hz, 1H), 8.02 (s, 1H), 7.75 (d, J=7.2 Hz, 1H), 7.67-7.60 (m, 2H), 7.55 (d, J=8.8 Hz, 1H), 7.50 (t, J=2.8 Hz, 1H), 7.32-7.31 (m, 4H), 7.25-7.22 (m, 1H), 6.62 (s, 1H), 4.12 (s, 2H) ppm.

### Example 35A

### Enantiomer 1 of (3-(5-Benzyl-4H-1,2,4-triazol-3-yl)phenyl)(1H-indol-5-yl)methanol and

### Example 35B

### Enantiomer 2 of (3-(5-Benzyl-4H-1,2,4-triazol-3-yl)phenyl)(1H-indol-5-yl)methanol

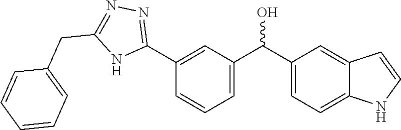 [SMILES: OC(c1cccc(-c2nnc(Cc3ccccc3)[nH]2)c1)c1ccc2[nH]ccc2c1]

To a stirred solution of (3-(5-benzyl-4H-1,2,4-triazol-3-yl)phenyl)(1-tosyl-1H-indol-5-yl)methanone (Example 34 Step F, 300 mg, 0.79 mmol) in methanol (20 mL) was added NaBH₄ (180 mg, 4.76 mmol) at 0° C. The resulting mixture was stirred at room temperature for 16 hours and concentrated to dryness under 35° C. The resulting residue was triturated with water (15 mL), the solid was collected by filtration and purified by chiral preparative HPLC to afford Peak 1, enantiomer 1 of (3-(5-benzyl-4H-1,2,4-triazol-3-yl)phenyl)(1H-indol-5-yl)methanol (71 mg, 23%) and Peak 2, enantiomer 2 of (3-(5-benzyl-4H-1,2,4-triazol-3-yl)phenyl)(1H-indol-5-yl)methanol (82 mg, 27%) as white solids. MS m/z: 381 [M+H]⁺.

Example 35A: Peak 1, Enantiomer 1 of (3-(5-benzyl-4H-1,2,4-triazol-3-yl)phenyl)(1H-indol-5-yl)methanol: MS m/z: 381 [M+H]⁺. Retention time 0.99 minutes.

Example 35B: Peak 2, Enantiomer 2 of (3-(5-benzyl-4H-1,2,4-triazol-3-yl)phenyl)(1H-indol-5-yl)methanol: MS m/z: 381 [M+H]⁺. Retention time 0.99 minutes.

### Example 36

### 3-(3-(5-Benzyl-4H-1,2,4-triazol-3-yl)phenyl)-3-(1H-indol-5-yl)propanenitrile

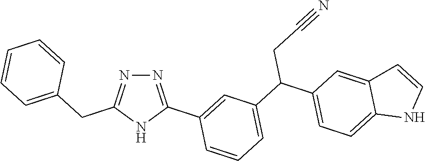 [SMILES: N#CCC(c1cccc(-c2nnc(Cc3ccccc3)[nH]2)c1)c1ccc2[nH]ccc2c1]

### A. 3-(3-(5-Benzyl-4H-1,2,4-triazol-3-yl)phenyl)-3-hydroxy-3-(1H-indol-5-yl)propanenitrile

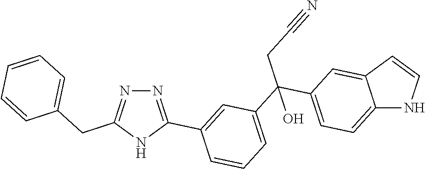 [SMILES: N#CCC(O)(c1cccc(-c2nnc(Cc3ccccc3)[nH]2)c1)c1ccc2[nH]ccc2c1]

Acetonitrile (1.52 g, 37 mmol) was added over a period of one minute to n-butyllithium (2.5M in THF, 8.8 mL, 22.2 mmol) in dry THF (60 mL) at −78° C. and the reaction mixture was stirred at −78° C. under nitrogen for 30 minutes. A solution of (3-(5-benzyl-4H-1,2,4-triazol-3-yl)phenyl)(1-tosyl-1H-indol-5-yl)methanone (Example 34 Step F, 0.7 g, 1.85 mmol) in dry THF (10 mL) was added to the reaction mixture over a period of five minutes. The solution was stirred at −78° C. for 30 minutes, then for another 30 minutes at room temperature, quenched with saturated ammonium chloride (60 mL) solution and extracted with ethyl acetate (50 mL×3). The combined organic extracts were washed with brine (2×25 mL), dried over sodium sulfate and evaporated under reduced pressure. The resulting solid was purified by silica gel column chromatography (dichloromethane/methanol, v/v, 20/1) to afford the title compound as a white solid (700 mg, 84%). MS m/z: 420 [M+H]⁺.

### B. 3-(3-(5-Benzyl-4H-1,2,4-triazol-3-yl)phenyl)-3-(1H-indol-5-yl)acrylonitrile

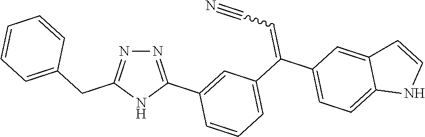 [SMILES: N#CC=C(c1cccc(-c2nnc(Cc3ccccc3)[nH]2)c1)c1ccc2[nH]ccc2c1]

A mixture of 3-(3-(5-benzyl-4H-1,2,4-triazol-3-yl)phenyl)-3-hydroxy-3-(1H-indol-5-yl)propanenitrile (700 mg, 1.67 mmol) and p-toluene sulfonic acid (20 mg) in dioxane (10 mL) was stirred at 110° C. for two hours and concentrated to dryness. The resulting residue was purified by silica gel column chromatography (dichloromethane/methanol, v/v, 20/1) to afford the title compound as a white solid (570 mg, 85%). MS m/z: 402 [M+H]⁺.

### C. Example 36. 3-(3-(5-Benzyl-4H-1,2,4-triazol-3-yl)phenyl)-3-(1H-indol-5-yl)propanenitrile

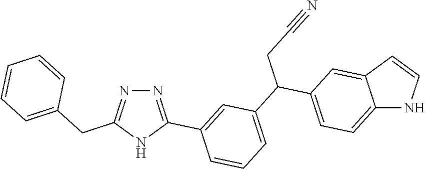 [SMILES: N#CCC(c1cccc(-c2nnc(Cc3ccccc3)[nH]2)c1)c1ccc2[nH]ccc2c1]

A mixture of 3-(3-(5-benzyl-4H-1,2,4-triazol-3-yl)phenyl)-3-(1H-indol-5-yl)acrylonitrile (570 mg, 1.42 mmol) and Pd/C (10%, 100 mg) in ethanol (10 mL) was stirred at 50° C. for 16 hours. The mixture was filtered through a pad of Celite and the filtrate was evaporated under reduced pressure. The residue was purified by column chromatography (dichloromethane/methanol, v/v, 20/1) to afford the title compound as a white solid (400 mg, 75%). MS m/z: 404 [M+H]⁺. ¹H NMR (400 MHz, DMSO-d₆) δ 8.02 (s, 1H), 7.86 (d, J=5.6 Hz, 1H), 7.56 (s, 1H), 7.46-7.44 (m, 2H), 7.35-7.22 (m, 7H), 7.06 (dd, J=8.4, 1.6 Hz, 1H), 6.43 (d, J=2.8 Hz, 1H), 4.57 (t, J=8.0 Hz, 1H), 4.17 (s, 2H), 3.29 (s, 2H) ppm.

### Example 37

### 3-(3-(5-Benzyl-4H-1,2,4-triazol-3-yl)phenyl)-3˜(1H-indol-5-yl)propanoic acid

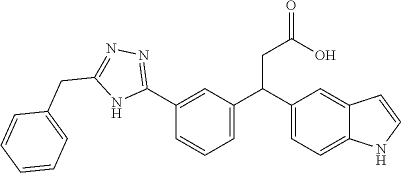 [SMILES: O=C(O)CC(c1cccc(-c2nnc(Cc3ccccc3)[nH]2)c1)c1ccc2[nH]ccc2c1]

A mixture of 3-(3-(5-benzyl-4H-1,2,4-triazol-3-yl)phenyl)-3-(1H-indol-5-yl)propanenitrile (Example 36, 170 mg, 0.42 mmol) and sodium hydroxide (250 mg, 6.33 mmol) in water (10 mL) and ethanol (5 mL) was heated at 120° C. overnight in a sealed tube and concentrated to remove ethanol. The residue was dissolved in 2 mL of water and the pH was adjusted to 3 with concentrated HCl. The solid was collected and dried to afford the title compound as a white solid (150 mg, 84%). MS m/z: 423 [M+H]⁺. ¹H NMR (400 MHz, DMSO-d₆) δ 10.99 (s, 1H), 7.91 (s, 1H), 7.76 (d, J=7.2 Hz, 1H), 7.47 (s, 1H), 7.42-7.21 (m, 9H), 7.01 (dd, J=8.4, 1.2 Hz, 1H), 6.35 (s, 1H), 4.53 (t, J=8.0 Hz, 1H), 4.10 (s, 2H), 3.08-3.05 (m, 2H) ppm.

### Example 38

### 3-(3-(5-Benzyl-4H-1,2,4-triazol-3-yl)phenyl)-3-(1H-indol-5-yl)propanamide

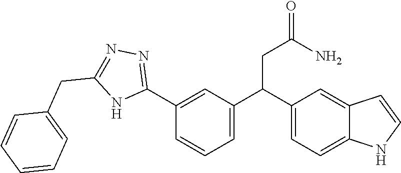 [SMILES: NC(=O)CC(c1cccc(-c2nnc(Cc3ccccc3)[nH]2)c1)c1ccc2[nH]ccc2c1]

To a stirred solution of 3-(3-(5-benzyl-4H-1,2,4-triazol-3-yl)phenyl)-3-(1H-indol-5-yl)propanenitrile (Example 36, 70 mg, 0.17 mmol) in DMSO (1 mL) were added potassium carbonate (48 mg, 0.35 mmol) and hydrogen peroxide (30 wt %, 60 mg, 1.7 mmol). The mixture was stirred overnight and then diluted with water (10 mL). The crystalline solid was collected by filtration, washed with water and dried under vacuum to afford the title compound as a white solid (36 mg, 49%). MS m/z: 422 [M+H]⁺. LC-MS retention time: 0.91 minutes.

### Example 39

### 1-(2-(3-((1H-Indol-5-yl)thio)phenyl)-1H-imidazol-5-yl)-1-phenylethan-1-ol

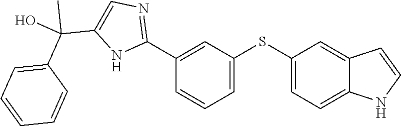 [SMILES: CC(O)(c1ccccc1)c1cnc(-c2cccc(Sc3ccc4[nH]ccc4c3)c2)[nH]1]

### A. 3-Mercaptobenzonitrile

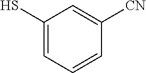

To a degassed mixture of 3-bromobenzonitrile (5.4 g, 30 mmol), CuSO₄·5 H₂O (375 mg, 1.5 mmol), Cs₂CO₃ (48.75 g, 150 mmol) in DMSO (60 mL) was added 1,2-ethanedithiol (5.64 g, 60 mmol). The reaction mixture was stirred at 100° C. overnight, cooled to ambient temperature, acidified with aqueous hydrochloric acid (5%) and extracted with ethyl acetate. The combined organic extracts were washed with water and brine, dried over MgSO₄ and concentrated. The resulting residue was purified by silica gel column chromatography (petroleum ether/ethyl acetate, v/v, 8/1 to 2/1) to afford the title compound as a yellow oil (1.3 g, 32%). ¹H NMR (400 MHz, CDCl₃) δ 7.54 (s, 1H), 7.48 (d, J=8.0 Hz, 1H), 7.44 (d, J=8.0 Hz, 1H), 7.34 (t, J=8.0 Hz, 1H) ppm.

### B. 3-((3-Methyl-4-nitrophenyl)thio)benzonitrile

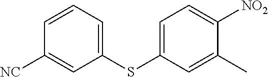 [SMILES: Cc1cc(Sc2cccc(C#N)c2)ccc1[N+](=O)[O-]]

A mixture of 3-mercaptobenzonitrile (1.3 g, 9.63 mmol), 4-fluoro-2-methyl-1-nitrobenzene (1.8 g, 11.56 mmol) and potassium carbonate (2 g, 14.44 mmol) in DMF (20 ml) was stirred at 60° C. overnight and then quenched with ice water (20 ml). The solid was collected by filtration, dried and triturated with a mixture of petroleum ether and ethyl acetate (v/v, 50 ml/3 ml) to afford the title compound as a yellow solid (2.2 g, 84%). ¹H NMR (400 MHz, CDCl₃) δ 7.95 (d, J=8.4 Hz, 1H), 7.72 (s, 1H), 7.66 (d, J=7.6 Hz, 2H), 7.52 (t, J=7.6 Hz, 1H), 7.18 (s, 1H), 7.13 (d, J=8.4 Hz, 1H), 2.60 (s, 3H) ppm.

### C. 3-((1H-Indol-5-yl)thio)benzonitrile

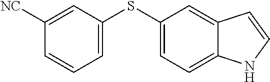 [SMILES: N#Cc1cccc(Sc2ccc3[nH]ccc3c2)c1]

A mixture of DMF-DMA (5.8 g, 48.9 mmol) and 3-((3-methyl-4-nitrophenyl)thio)benzonitrile (2.2 g, 8.15 mmol) in DMF (15 ml) was stirred at 80° C. for three hours under nitrogen. The mixture was poured into water (60 ml) and extracted with ethyl acetate (50 ml×3). The combined organic extracts were washed with brine, dried over sodium sulfate and concentrated. The resulting residue was dissolved in acetic acid (20 ml) and toluene (12 ml) and iron powder (9.1 g, 163 mmol) was added. The reaction mixture was stirred at 80° C. overnight, cooled to room temperature, poured into water (100 ml) and extracted with ethyl acetate (70 ml×3). The combined organic extracts were washed with brine, dried over sodium sulfate, filtered and concentrated. The resulting residue was purified by silica gel column chromatography (petroleum ether/dichloromethane, v/v, 1/1) to afford the title compound as a yellow solid (1.2 g, 59%). MS m/z: 251 [M+H]⁺.

### D. 3-((1H-Indol-5-yl)thio)benzimidamide

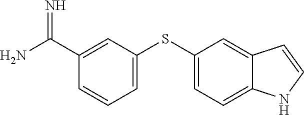 [SMILES: N=C(N)c1cccc(Sc2ccc3[nH]ccc3c2)c1]

To a stirred solution of 3-((1H-indol-5-yl)thio)benzonitrile (310 mg, 1.24 mmol) in THF (5 mL) was added lithium bis(trimethylsilyl)amide (1N in THF, 12.4 mL, 12.4 mmol) at room temperature. The resulting mixture was stirred at room temperature for 16 hours, quenched with methanol (10 mL) and concentrated. The resulting residue was purified by silica gel column chromatography (dichloromethane/methanol/concentrated ammonium hydroxide, v/v, 5/1/0.1) to afford the title compound as a yellow solid (260 mg, 79%). MS m/z: 268 [M+H]⁺.

### E. Example 39 1-(2-(3-((1H-Indol-5-yl)thio)phenyl)-1H-imidazol-5-yl)-1-phenylethan-1-ol

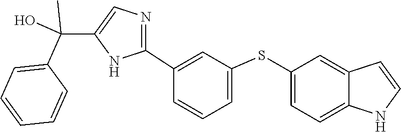 [SMILES: CC(O)(c1ccccc1)c1cnc(-c2cccc(Sc3ccc4[nH]ccc4c3)c2)[nH]1]

A mixture of 3-((1H-indol-5-yl)thio)benzimidamide (260 mg, 1.11 mmol) and 3-methyl-3-phenyloxirane-2-carbaldehyde (Intermediate 3, 160 mg, 1.11 mmol) in dry DMF (5 mL) was stirred at 80° C. under nitrogen overnight and concentrated to dryness. The resulting residue was purified by silica gel column chromatography (dichloromethane/methanol/concentrated ammonium hydroxide, v/v, 20/1/0.1) to afford the title compound as a white solid (210 mg, 52%). MS m/z: 412 [M+H]⁺. ¹H NMR (400 MHz, CD₃OD) δ 7.79 (s, 1H), 7.75 (s, 1H), 7.57 (d, J=7.6 Hz, 1H), 7.48 (d, J=7.6 Hz, 2H), 7.44 (d, J=8.4 Hz, 1H), 7.34-7.31 (m, 3H), 7.26-7.23 (m, 3H), 7.00 (d, J=8.0 Hz, 1H), 6.88 (s, 1H), 6.48 (d, J=2.8 Hz, 1H), 1.90 (s, 3H) ppm.

### Example 40

### 1-(2-(3-((1H-Indol-5-yl)sulfinyl)phenyl)-1H-imidazol-5-yl)-1-phenylethan-1-ol

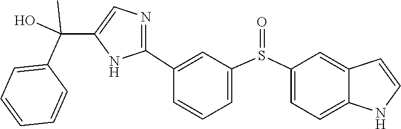 [SMILES: CC(O)(c1ccccc1)c1cnc(-c2cccc(S(=O)c3ccc4[nH]ccc4c3)c2)[nH]1]

To a stirred solution of 1-(2-(3-((1H-indol-5-yl)thio)phenyl)-1H-imidazol-5-yl)-1-phenylethan-1-ol (Example 39, 80 mg, 0.19 mmol) in acetic acid (5 mL) was added H₂O₂ (30% in water, 1 mL) at 0° C. The mixture was stirred at room temperature for 30 minutes, poured into water (20 ml) and extracted with ethyl acetate (20 mL×3). The combined organic extracts were washed with brine, dried over sodium sulfate, filtered and concentrated to dryness. The resulting residue was purified by preparative HPLC to afford the title compound as white solid (40 mg, 48%). MS m/z: 428 [M+H]⁺. ¹H NMR (400 MHz, CD₃OD) δ 8.17 (s, 1H), 8.07 (s, 1H), 7.97 (d, J=7.6 Hz, 1H), 7.64-7.57 (m, 2H), 7.54-7.49 (m, 3H), 7.40 (d, J=3.2 Hz, 1H), 7.38-7.31 (m, 3H), 7.25-7.22 (m, 1H), 6.96 (br s, 1H), 6.61 (d, J=2.8 Hz, 1H), 1.92 (s, 3H) ppm.

### Example 41

### 1-(2-(3-((1H-Indol-5-yl)sulfonyl)phenyl)-1H-imidazol-5-yl)-1-phenylethan-1-ol

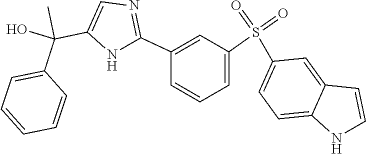 [SMILES: CC(O)(c1ccccc1)c1cnc(-c2cccc(S(=O)(=O)c3ccc4[nH]ccc4c3)c2)[nH]1]

To a stirred solution of 1-(2-(3-((1H-indol-5-yl)thio)phenyl)-1H-imidazol-5-yl)-1-phenylethan-1-ol (Example 39, 100 mg, 0.24 mmol) in methanol (10 mL) was added a solution of ammonium molybdate (200 mg, 0.16 mmol) in hydrogen peroxide (1 mL, 30% in water).

The mixture was stirred at room temperature for ten minutes, quenched with saturated sodium sulfite and extracted with ethyl acetate (30 mL×3). The combined organic extracts were washed with brine, dried over sodium sulfate, filtered and concentrated. The resulting residue was purified by preparative HPLC to afford the title compound as an off-white solid (50 mg, 44%). MS m/z: 444 [M+H]⁺. ¹H NMR (400 MHz, CD₃OD) δ 8.52 (s, 1H), 8.33 (d, J=1.2 Hz, 1H), 8.06 (d, J=8.0 Hz, 1H), 7.93 (d, J=8.8 Hz, 1H), 7.71 (dd, J=8.8, 1.6 Hz, 1H), 7.61 (t, J=8.0 Hz, 1H), 7.50-7.55 (m, 3H), 7.43 (d, J=2.8 Hz, 1H), 7.32-7.36 (m, 2H), 7.25 (t, J=7.2 Hz, 1H), 6.98 (br s, 1H), 6.67 (d, J=2.8 Hz, 1H), 1.93 (s, 3H) ppm.

### Example 42

### 6-(3-(5-Benzyl-4H-1,2,4-triazol-3-yl)phenoxy)-3,4-dihydropyrrolo[4,3,2-de]isoquinolin-5(1H)-one

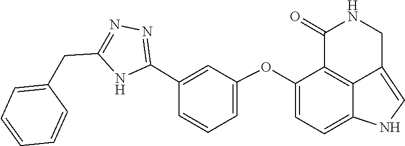 [SMILES: O=C1NCc2c[nH]c3ccc(Oc4cccc(-c5nnc(Cc6ccccc6)[nH]5)c4)c1c23]

### A. Methyl 3-formyl-5-(3-(methoxycarbonyl)phenoxy)-1H-indole-4-carboxylate

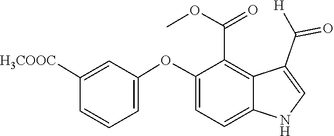

Phosphorus(V) oxychloride (451 mg, 2.95 mmol) was added dropwise to DMF (3 mL) at 0° C. and stirred for 20 minutes. To this mixture was added a solution of methyl 5-(3-(methoxycarbonyl)phenoxy)-1H-indole-4-carboxylate (Intermediate 1-7, 800 mg, 2.46 mmol) in DMF (3 mL) dropwise. The reaction mixture was stirred at room temperature overnight, poured into ice water, neutralized with 20% aqueous sodium hydroxide and extracted with ethyl acetate (60 mL×2). The combined organic extracts were washed with water (50 mL) and brine (50 mL), dried over sodium sulfate and concentrated to afford the title compound as a light yellow solid (810 mg, 93%). The product was used without further purification. MS m/z: 354 [M+H]⁺.

### B. Methyl 3-((hydroxyimino)methyl)-5-(3-(methoxycarbonyl)phenoxy)-1H-indole-4-carboxylate

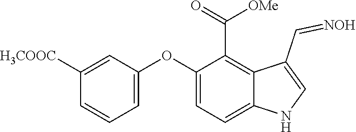

A mixture of methyl 3-formyl-5-(3-(methoxycarbonyl)phenoxy)-1H-indole-4-carboxylate (800 mg, 2.27 mmol), hydroxylamine hydrochloride (174 mg, 2.50 mmol) and sodium acetate (280 mg, 3.41 mmol) in ethanol (10 mL) was stirred at room temperature for one hour. The reaction mixture was diluted with ethyl acetate (50 mL), washed with water (40 mL), dried over sodium sulfate and concentrated to afford the title compound as a white solid (700 mg, 84%). The product was used without further purification. MS m/z: 367 [M−H]⁻.

### C. 3-((5-Oxo-1,3,4,5-tetrahydropyrrolo[4,3,2-de]isoquinolin-6-yl)oxy)benzoic acid

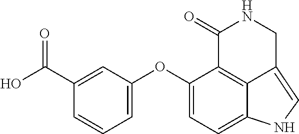 [SMILES: O=C(O)c1cccc(Oc2ccc3[nH]cc4c3c2C(=O)NC4)c1]

A solution of methyl 3-((hydroxyimino)methyl)-5-(3-(methoxycarbonyl)phenoxy)-1H-indole-4-carboxylate (620 mg, 1.68 mmol), Pd/C (10%, 62 mg) and hydrogen chloride (0.84 mL, 3.36 mmol, 4M in dioxane) in ethanol (5 mL) was stirred overnight at room temperature under hydrogen atmosphere. The resulting mixture was filtered through a pad of Celite and washed with ethanol (5 mL×2). Sodium methoxide (454 mg, 8.40 mmol) was added to the filtrate and the reaction mixture was stirred at 60° C. for three hours. The reaction mixture was cooled to room temperature and concentrated to dryness in vacuo. The residue was dissolved in water (20 mL), pH was adjusted to 5-6 with 1M hydrochloric acid and extracted with ethyl acetate (30 mL×3). The combined organic phase was washed with brine (50 mL), dried over sodium sulfate, filtered and concentrated to dryness in vacuo. The resulting residue was purified by reverse phase chromatography to afford the title compound as a white solid (350 mg, 68%). MS m/z: 309 [M+H]⁺.

### D. 3-((5-Oxo-1,3,4,5-tetrahydropyrrolo[4,3,2-de]isoquinolin-6-yl)oxy)benzohydrazide

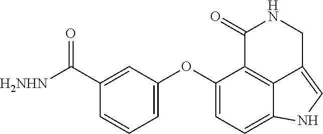

A mixture of 3-((5-oxo-1,3,4,5-tetrahydropyrrolo[4,3,2-de]isoquinolin-6-yl)oxy)benzoic acid (400 mg, 1.3 mmol), methanol (125 mg, 3.9 mmol), HATU (988 mg, 2.6 mmol) and diisopropylethylamine (671 mg, 5.2 mmol) in DMF (5 mL) was stirred at room temperature overnight. The reaction mixture was quenched with water (50 mL) and extracted with ethyl acetate (50 mL×3). The combined organic extracts were washed with water (50 mL) and brine (50 mL), dried over sodium sulfate and concentrated to dryness in vacuo. The resulting residue was dissolved in methanol (5 mL) and hydrazine hydrate (125 mg, 3.9 mmol) was added. The resulting mixture was stirred at 60° C. for three hours and then concentrated in vacuo to afford the title compound as a white solid (300 mg, 72% from 2 steps). The product was used without further purification. MS m/z: 323 [M+H]⁺.

### E. Example 42. 6-(3-(5-Benzyl-4H-1,2,4-triazol-3-yl)phenoxy)-3,4-dihydropyrrolo[4,3,2-de]isoquinolin-5(1H)-one

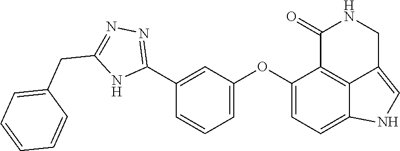 [SMILES: O=C1NCc2c[nH]c3ccc(Oc4cccc(-c5nnc(Cc6ccccc6)[nH]5)c4)c1c23]

A mixture of 3-((5-oxo-1,3,4,5-tetrahydropyrrolo[4,3,2-de]isoquinolin-6-yl)oxy)benzohydrazide (150 mg, 0.47 mmol), 2-phenylacetonitrile (83 mg, 0.71 mmol) and potassium carbonate (130 mg, 0.94 mmol) in “Butanol (6 mL) was stirred at 130° C. overnight under nitrogen atmosphere. The mixture was concentrated and diluted with ethyl acetate (50 mL). The organic layer was washed with water (50 mL) and brine (50 mL), dried over sodium sulfate, filtered and concentrated. The residue was purified by preparative HPLC to afford the title compound as a white solid (60 mg, 31%). MS m/z: 422 [M+H]⁺. ¹H NMR (500 MHz, CD₃OD) δ 7.63 (d, J=7.5 Hz, 1H), 7.51 (d, J=9.0 Hz, 1H), 7.49 (t, J=2.0 Hz, 1H), 7.39 (t, J=8.0 Hz, 1H), 7.33-7.27 (m, 4H), 7.25-7.22 (m, 1H), 7.19 (s, 1H), 7.03 (dd, J=8.0, 2.0 Hz, 1H), 6.85 (d, J=8.5 Hz, 1H), 4.92 (s, 2H), 4.12 (s, 2H) ppm.

### Example 43

### 6-(3-(5-Benzyl-4H-1,2,4-triazol-3-yl)phenoxy)-1,3,4,5-tetrahydropyrrolo[4,3,2-de]isoquinoline

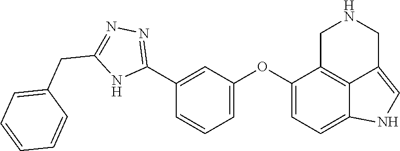 [SMILES: c1ccc(Cc2nnc(-c3cccc(Oc4ccc5[nH]cc6c5c4CNC6)c3)[nH]2)cc1]

A mixture of 6-(3-(5-benzyl-4H-1,2,4-triazol-3-yl)phenoxy)-3,4-dihydropyrrolo[4,3,2-de]isoquinolin-5(1H)-one (Example 42, 140 mg, 0.33 mmol) and BH₃-THF (3.3 mL, 3.3 mmol, 1M in THF) in THF (3 mL) was stirred at 70° C. overnight under nitrogen atmosphere.

To this mixture was added methanol (5 mL) followed by 1N hydrochloric acid (10 mL, 10 mmol). The resulting mixture was refluxed for one hour, cooled to room temperature and water (20 mL) was added. The mixture was basified to pH to 7-8 with saturated sodium bicarbonate solution and extracted with ethyl acetate (20 mL×3). The combined organic extracts were washed with brine (30 mL), dried over sodium sulfate and concentrated. The resulting residue was purified by preparative HPLC to afford the title compound as a white solid (15 mg, 11%). MS m/z: 408 [M+H]⁺. ¹H NMR (500 MHz, CD₃OD) δ 7.65 (d, J=7.5 Hz, 1H), 7.45 (t, J=1.5 Hz, 1H), 7.42 (t, J=8.0 Hz, 1H), 7.34-7.27 (m, 5H), 7.26-7.24 (m, 1H), 7.12 (s, 1H), 7.04 (dd, J=8.0, 2.0 Hz, 1H), 6.88 (d, J=9.0 Hz, 1H), 4.27 (s, 2H), 4.14 (s, 2H), 4.11 (s, 2H) ppm.

### Example 44

### 4(1H-Indol-5-yl)oxy)-6-(5-benzyl-4H-1,2,4-triazol-3-yl)pyridin-3-amine

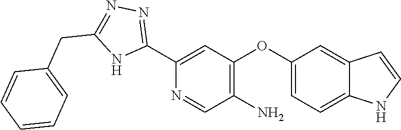 [SMILES: Nc1cnc(-c2nnc(Cc3ccccc3)[nH]2)cc1Oc1ccc2[nH]ccc2c1]

### A. 5-((2-Chloro-5-nitropyridin-4-yl)oxy)-1-tosyl-1H-indole

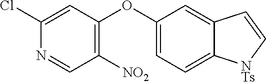 [SMILES: Cc1ccc(S(=O)(=O)n2ccc3cc(Oc4cc(Cl)ncc4[N+](=O)[O-])ccc32)cc1]

To a solution of 1-tosyl-1H-indol-5-ol (299 mg, 1.04 mmol) in DMF (5 mL) was added sodium hydride (60% in mineral oil, 50 mg, 1.2 mmol) at 0° C. The mixture was stirred at 0° C. for 30 minutes and 2,4-dichloro-5-nitropyridine (200 mg, 1.04 mmol) was added at 0° C. The reaction mixture was warmed to room temperature, stirred for two hours and diluted with ethyl acetate. The mixture was washed with water and brine, dried over sodium sulfate, filtered and evaporated in vacuo. The resulting residue was purified by silica gel column chromatography (petroleum ether/ethyl acetate, v/v, 3/1) to afford the title compound as a yellow solid (198 mg, 43%). MS m/z: 444 [M+H]⁺. ¹H NMR (500 MHz, CDCl₃) δ 8.94 (s, 1H), 8.09 (d, J=9.2 Hz, 1H), 7.80 (d, J=8.4 Hz, 2H), 7.70 (d, J=4.0 Hz, 1H), 7.32 (d, J=2.0 Hz, 1H), 7.29 (d, J=8.0 Hz, 1H), 7.09 (dd, J=8.8, 2.4 Hz, 1H), 6.70-6.68 (m, 2H), 2.38 (s, 3H).

### B. 5-Nitro-4-((1-tosyl-1H-indol-5-yl)oxy)picolinonitrile

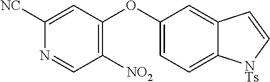 [SMILES: Cc1ccc(S(=O)(=O)n2ccc3cc(Oc4cc(C#N)ncc4[N+](=O)[O-])ccc32)cc1]

To a stirred solution of 5-((2-chloro-5-nitropyridin-4-yl)oxy)-1-tosyl-1H-indole (198 mg, 0.44 mmol) in dry DMF (10 mL) were added Zn(CN)₂ (52 mg, 0.44 mmol), DPPF (25 mg, 0.044 mmol) and Pd₂dba₃ (21 mg, 0.022 mmol). The reaction mixture was stirred overnight at 110° C. under nitrogen, diluted with ethyl acetate and washed with water and brine. The organic phase was dried over sodium sulfate and evaporated in vacuo. The resulting residue was purified by silica column chromatography (petroleum ether/ethyl acetate, v/v, 6/1) to afford the title compound as a yellow solid (97 mg, 51%). MS m/z: 435 [M+H]⁺.

### C. 5-Amino-4-((1-tosyl-1H-indol-5-yl)oxy)picolinonitrile

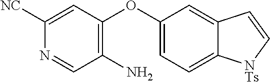 [SMILES: Cc1ccc(S(=O)(=O)n2ccc3cc(Oc4cc(C#N)ncc4N)ccc32)cc1]

Iron powder (45 mg, 0.8 mmol) and ammonium chloride (100 mg, 2 mmol) were added to a solution of 5-nitro-4-((1-tosyl-1H-indol-5-yl)oxy)picolinonitrile (70 mg, 0.16 mmol) in a 2:1 mixture of ethanol and water (9 mL) and refluxed for 30 minutes. The hot reaction mixture was filtered, the filtrate was diluted with ethyl acetate and washed with water and brine. The organic phase was dried over sodium sulfate and evaporated to afford the title compound (75 mg, 100%), which was used without further purification in the next step. MS m/z: 405 [M+H]⁺.

### D. Example 44. 4-((1H-Indol-5-yl)oxy)-6-(5-benzyl-4H-1,2,4-triazol-3-yl)pyridin-3-amine

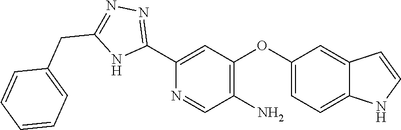 [SMILES: Nc1cnc(-c2nnc(Cc3ccccc3)[nH]2)cc1Oc1ccc2[nH]ccc2c1]

A microwave vial containing a mixture of 5-amino-4-((1-tosyl-1H-indol-5-yl)oxy)picolinonitrile (200 mg, 0.48 mmol), 2-phenylacetohydrazide (297 mg, 1.98 mmol) and potassium carbonate (410 mg, 2.97 mmol) in “Butanol (20 mL) was heated at 160° C. for four hours. The reaction mixture was filtered and the filtrate was evaporated in vacuo. The crude product was purified by preparative HPLC to afford the title compound as a white solid (25 mg, 14%). MS m/z: 383 [M+H]⁺. ¹H NMR (400 MHz, CD₃OD) δ 8.00 (s, 1H), 7.36 (d, J=8.4 Hz, 1H), 7.24 (d, J=2.0 Hz, 1H), 7.21 (d, J=3.2 Hz, 1H), 7.13-7.05 (m, 6H), 6.81 (dd, J=8.8, 2.0 Hz, 1H), 6.36 (d, J=3.2 Hz, 1H), 3.88 (s, 2H).

### Example 45

### 9-(3-(5-Benzyl-4H-1,2,4-triazol-3-yl)phenoxy)-2,3,4,6-tetrahydro-1H-azepino[5,4,3-cd]indol-1-one

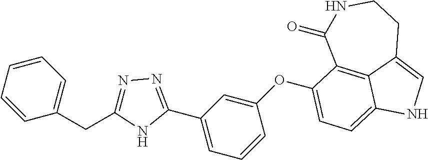 [SMILES: O=C1NCCc2c[nH]c3ccc(Oc4cccc(-c5nnc(Cc6ccccc6)[nH]5)c4)c1c23]

### A. Methyl 5-(3-(methoxycarbonyl)phenoxy)-3-(2-nitroethyl)-1H-indole-4-carboxylate

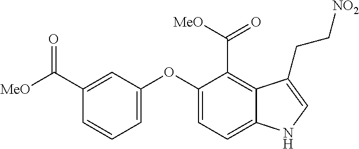 [SMILES: COC(=O)c1cccc(Oc2ccc3[nH]cc(CC[N+](=O)[O-])c3c2C(=O)OC)c1]

A mixture of methyl 5-(3-(methoxycarbonyl)phenoxy)-1H-indole-4-carboxylate (Intermediate 1-7, 700 mg, 2.15 mmol), 2-nitroethyl acetate (430 mg, 3.23 mmol) and 4-tert-butylcatechol (36 mg, 0.215 mmol) in xylene (8 mL) was refluxed for eight hours under nitrogen atmosphere. The mixture was concentrated and the residue was purified by silica gel column chromatography (petroleum ether/ethyl acetate, v/v, 1/1) to afford the title compound as a brown solid (650 mg, 76%). MS m/z: 399 [M+H]⁺.

### B. Methyl 3-((1-oxo-2,3,4,6-tetrahydro-1H-azepino[5,4,3-cd]indol-9-yl)oxy)benzoate

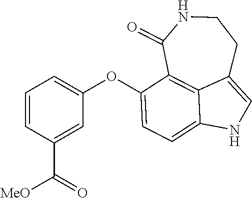 [SMILES: COC(=O)c1cccc(Oc2ccc3[nH]cc4c3c2C(=O)NCC4)c1]

A solution of methyl 5-(3-(methoxycarbonyl)phenoxy)-3-(2-nitroethyl)-1H-indole-4-carboxylate (1.45 g, 3.64 mmol) and Pd/C (10%, 145 mg) in methanol (20 mL) was stirred at 30° C. under hydrogen atmosphere (50 psi) for 24 hours. The resulting mixture was filtered through a pad of Celite and washed with ethanol (5 mL×2). The filtrate was heated at reflux for four days with stirring and then concentrated in vacuo. The resulting residue was purified by reverse phase chromatography to afford the title compound as a white solid (200 mg, 16% from 2 steps). MS m/z: 337 [M+H]⁺.

### C. 3-((1-Oxo-2,3,4,6-tetrahydro-1H-azepino[5,4,3-cd]indol-9-yl)oxy)benzohydrazide

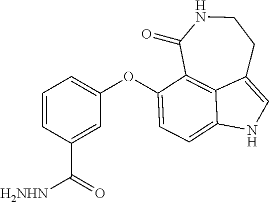

To a solution of methyl 3-((1-oxo-2,3,4,6-tetrahydro-1H-azepino[5,4,3-cd]indol-9-yl)oxy)benzoate (200 mg, 0.6 mmol) in methanol (8 mL) was added hydrazine hydrate (58 mg, 1.8 mmol). The resulting mixture was stirred at reflux for three hours and concentrated in vacuo to afford the title compound as a white solid (180 mg, 90%). The product was used in the next step without further purification. MS m/z: 337 [M+H]⁺.

### D. Example 45. 9-(3-(5-Benzyl-4H-1,2,4-triazol-3-yl)phenoxy)-2,3,4,6-tetrahydro-1H-azepino[5,4,3-cd]indol-1-one

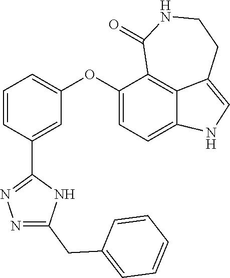 [SMILES: O=C1NCCc2c[nH]c3ccc(Oc4cccc(-c5nnc(Cc6ccccc6)[nH]5)c4)c1c23]

A mixture of 3-((1-oxo-2,3,4,6-tetrahydro-1H-azepino[5,4,3-cd]indol-9-yl)oxy)benzohydrazide (90 mg, 0.27 mmol), 2-phenylacetonitrile (48 mg, 0.41 mmol) and potassium carbonate (75 mg, 0.54 mmol) in n-Butanol (5 mL) was stirred at 130° C. overnight under nitrogen atmosphere. The mixture was concentrated in vacuo and then diluted with ethyl acetate (50 mL). The mixture was washed with water (50 mL) and brine (50 mL), dried over sodium sulfate, filtered and concentrated. The resulting residue was purified by preparative HPLC to afford the title compound as a white solid (45 mg, 39%). MS m/z: 436 [M+H]⁺. ¹H NMR (500 MHz, DMSO-d₆) δ 11.21 (s, 1H), 7.85 (t, J=7.0 Hz, 1H), 7.56 (d, J=7.5 Hz, 1H), 7.52 (d, J=8.5 Hz, 1H), 7.38-7.23 (m, 8H), 7.22 (t, J=7.0 Hz, 1H), 6.92-6.89 (m, 1H), 6.86 (d, J=8.5 Hz, 1H), 4.05 (s, 2H), 3.35 (s, 2H), 2.86 (s, 2H) ppm.

### Example 46

### 9-(3-(5-Benzyl-4H-1,2,4-triazol-3-yl)phenoxy)-2,3,4,6-tetrahydro-1H-azepino[5,4,3-cd]indole

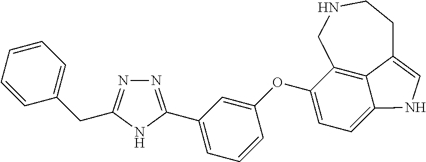 [SMILES: c1ccc(Cc2nnc(-c3cccc(Oc4ccc5[nH]cc6c5c4CNCC6)c3)[nH]2)cc1]

A mixture of 9-(3-(5-benzyl-4H-1,2,4-triazol-3-yl)phenoxy)-2,3,4,6-tetrahydro-1H-azepino[5,4,3-cd]indol-1-one (135 mg, 0.31 mmol) and BH₃-THF (1M in THF, 3.1 mL, 3.1 mmol) in THF (3 mL) was stirred at 70° C. overnight under nitrogen atmosphere. The reaction was quenched with methanol (5 mL) and 1N hydrochloric acid (10 mL, 10 mmol) was added. The resulting mixture was refluxed for one hour, cooled to room temperature and water (20 mL) was added. Saturated sodium bicarbonate was added to adjust the pH to 7-8 and the aqueous phase was extracted with ethyl acetate (20 mL×3). The combined organic extracts were washed with brine (30 mL), dried over sodium sulfate and concentrated. The resulting residue was purified by preparative HPLC to afford the title compound as a white solid (4.0 mg, 3%). MS m/z: 422 [M+H]⁺. ¹H NMR (500 MHz, CD₃OD) δ 7.62 (d, J=8.0 Hz, 1H), 7.44 (t, J=2.0 Hz, 1H), 7.39 (t, J=8.0 Hz, 1H), 7.33-7.24 (m, 6H), 7.16 (s, 1H), 6.97 (dd, J=8.0, 2.0 Hz, 1H), 6.82 (d, J=8.5 Hz, 1H), 4.28 (s, 2H), 4.14 (s, 2H), 3.23 (t, J=5.0 Hz, 2H), 3.15 (t, J=5.0 Hz, 2H) ppm.

### Example 47

### 2-(3-((1H-Indol-5-yl)oxy)phenyl)-4-benzyloxazole

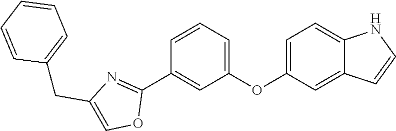 [SMILES: c1ccc(Cc2coc(-c3cccc(Oc4ccc5[nH]ccc5c4)c3)n2)cc1]

### A. Ethyl 3-((1H-indol-5-yl)oxy)benzoate

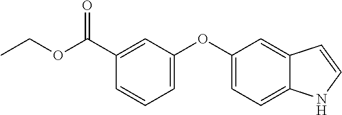 [SMILES: CCOC(=O)c1cccc(Oc2ccc3[nH]ccc3c2)c1]

A mixture of 5-bromo-1H-indole (4.39 g, 22.5 mmol), ethyl 3-hydroxybenzoate (7.47 g, 45 mmol), CuI (428 mg, 2.25 mmol), Cs₂CO₃ (14.7 g, 45 mmol) and 2-amino-N,N-dimethylacetamide hydrochloride (313 mg, 2.25 mmol) in a mixture of 1,4-dioxane (10 mL) and DMF (2.5 mL) was heated at 160° C. for 0.5 hour in a Biotage microwave synthesizer. The reaction mixture was cooled to room temperature, quenched with water (30 mL) and extracted with ethyl acetate (50 mL×4). The combined organic phases were washed with water (50 mL×2) and brine (50 mL), dried over sodium sulfate, filtered and concentrated in vacuo. The resulting crude product was purified by silica gel column chromatography (petroleum ether/ethyl acetate, v/v, 10/1) to afford the title compound as a yellow solid (1 g, 17%). MS m/z: 282 [M+H]⁺.

### B. Ethyl 3-((1-tosyl-1H-indol-5-yl)oxy)benzoate

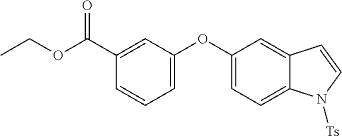 [SMILES: CCOC(=O)c1cccc(Oc2ccc3c(ccn3S(=O)(=O)c3ccc(C)cc3)c2)c1]

To a solution of ethyl 3-((1H-indol-5-yl)oxy)benzoate (540 mg, 2.13 mmol) in DMF (5 mL) was added sodium hydride (60% in mineral oil, 103 mg, 2.56 mmol) at 0° C. The resulting mixture was stirred at room temperature for 0.5 hours and then p-toluene sulfonyl chloride (487 mg, 2.56 mmol) was added at 0° C. The reaction mixture was stirred at room temperature for two hours, quenched with saturated sodium carbonate solution (15 mL) and extracted with ethyl acetate (30 mL×3). The combined organic layers were washed with brine, dried over sodium sulfate, filtered and concentrated in vacuo. The resulting crude product was purified by reverse phase chromatography to afford the tile compound as light red oil (380 mg, 49%). MS m/z: 436 [M+H]⁺.

### C. 3-((1-Tosyl-1H-indol-5-yl)oxy)benzoic acid

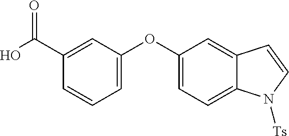 [SMILES: Cc1ccc(S(=O)(=O)n2ccc3cc(Oc4cccc(C(=O)O)c4)ccc32)cc1]

To a solution of ethyl 3-((1-tosyl-1H-indol-5-yl)oxy)benzoate (370 mg, 3.51 mmol) in THF (5 mL) was added a solution of lithium hydroxide (102 mg, 4.25 mmol) in water (1 mL). The reaction mixture was stirred at 40° C. for two hours, concentrated to remove THF and diluted with water (20 mL). The mixture was acidified to pH to 5-6 with 1N hydrochloric acid and extracted with ethyl acetate (30 mL×3). The combined organic layers were washed with brine, dried over sodium sulfate, filtered and concentrated to afford the title compound as colorless oil (280 mg, 89%). The product was used directly in the next step. MS m/z: 408 [M+H]⁺.

### D. 2-Oxo-3-phenylpropyl 3-((1-tosyl-1H-indol-5-yl)oxy)benzoate

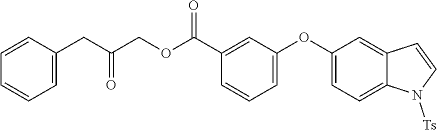 [SMILES: Cc1ccc(S(=O)(=O)n2ccc3cc(Oc4cccc(C(=O)OCC(=O)Cc5ccccc5)c4)ccc32)cc1]

A mixture of 3-((1-tosyl-1H-indol-5-yl)oxy)benzoic acid (270 mg, 0.66 mmol), 1-bromo-3-phenylpropan-2-one (169 mg, 0.8 mmol) and triethylamine (134 mg, 1.33 mmol) in acetonitrile (5 mL) was stirred at room temperature overnight. The resulting mixture was concentrated in vacuo to give a residue, which was diluted with water (20 mL) and extracted with ethyl acetate (30 mL×3). The combined organic extracts were washed with brine, dried over sodium sulfate, filtered and concentrated in vacuo. The resulting crude product was purified by silica gel column chromatography (petroleum ether/ethyl acetate, v/v, 10/1) to afford the title compound as a solid (245 mg, 74%). MS m/z: 540 [M+H]⁺.

### E. 4-Benzyl-2-(3-((1-tosyl-1H-indol-5-yl)oxy)phenyl)oxazole

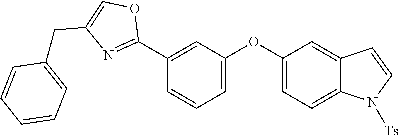 [SMILES: Cc1ccc(S(=O)(=O)n2ccc3cc(Oc4cccc(-c5nc(Cc6ccccc6)co5)c4)ccc32)cc1]

Ammonium acetate (129 mg, 1.67 mmol) was added to a solution of 2-oxo-3-phenylpropyl 3-((1-tosyl-1H-indol-5-yl)oxy)benzoate (180 mg, 0.33 mmol) in acetic acid (2 mL), the mixture was refluxed for three hours and then concentrated in vacuo. The residue was dissolved in ethyl acetate (30 mL), washed with water (30 mL) and brine (30 mL), dried over sodium sulfate and concentrated in vacuo. The resulting residue was purified by silica gel column chromatography (petroleum ether/ethyl acetate, v/v, 5/1) to afford the title compound as a solid (86 mg, 50%). MS m/z: 521 [M+H]⁺.

### F. Example 47. 2-(3-((1H-Indol-5-yl)oxy)phenyl)-4-benzyloxazole

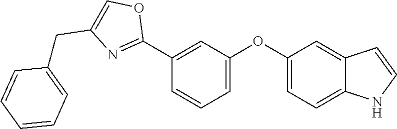 [SMILES: c1ccc(Cc2coc(-c3cccc(Oc4ccc5[nH]ccc5c4)c3)n2)cc1]

A mixture of 4-benzyl-2-(3-((1-tosyl-1H-indol-5-yl)oxy)phenyl)oxazole (86 mg, 0.17 mmol) and potassium carbonate (507 mg, 0.33 mmol) in methanol (1 mL) was irradiated at 130° C. for 0.5 hour in a Biotage microwave synthesizer. The reaction mixture was concentrated in vacuo, diluted with water (10 mL) and extracted with ethyl acetate (20 mL×3). The combined organic layer was washed with brine, dried over sodium sulfate, filtered and concentrated in vacuo. The residue was purified by preparative HPLC to afford the title compound as brown oil (20 mg, 33%). MS m/z: 367 [M+H]⁺. ¹H NMR (500 MHz, CDCl₃) δ 8.36 (br s, 1H), 7.75-7.72 (m, 1H), 7.67-7.65 (m, 1H), 7.40-7.31 (m, 7H), 7.28-7.24 (m, 3H), 7.09-7.06 (m, 1H), 6.98 (dd, J=8.5, 2.0 Hz, 1H), 6.55-6.53 (m, 1H), 3.95 (s, 2H) ppm. ¹³C NMR (100 MHz, CDCl₃) δ 161.4, 159.7, 149.8, 141.9, 138.3, 135.1, 132.9, 129.9, 128.93, 128.87, 128.6, 128.5, 126.5, 125.5, 120.1, 119.4, 115.6, 115.2, 112.0, 111.3, 102.8, 33.0 ppm.

### Example 48

### (5-(3-(4-Benzylthiazol-2-yl)phenoxy)-1H-indol-4-yl)methanol

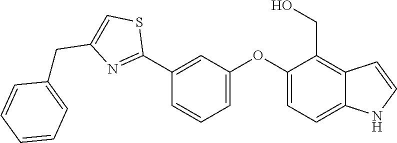 [SMILES: OCc1c(Oc2cccc(-c3nc(Cc4ccccc4)cs3)c2)ccc2[nH]ccc12]

### A. 3-((4-(Methoxycarbonyl)-1H-indol-5-yl)oxy)benzimidothioic acid

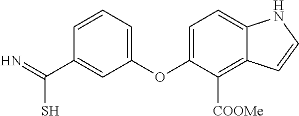 [SMILES: COC(=O)c1c(Oc2cccc(C(=N)S)c2)ccc2[nH]ccc12]

To a slurry of sodium hydrogensulfide (287.68 mg, 5.13 mmol) and magnesium chloride hexahydrate (347.76 mg, 1.71 mmol) in DMF (10 mL) was added methyl 5-(3-cyanophenoxy)-1H-indole-4-carboxylate (Intermediate 1-3, 0.5 g, 1.71 mmol) in one portion and the mixture was stirred at room temperature for 90 minutes. The resulting green slurry was poured into water (20 mL), the precipitate was collected by filtration and dried. The crude product was used without further purification.

### B. Methyl 5-(3-(4-(3-iodobenzyl)thiazol-2-yl)phenoxy)-1H-indole-4-carboxylate

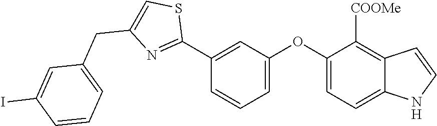 [SMILES: COC(=O)c1c(Oc2cccc(-c3nc(Cc4cccc(I)c4)cs3)c2)ccc2[nH]ccc12]

A mixture of 1-chloro-3-(3-iodophenyl)propan-2-one (451.2 mg, 1.53 mmol) and 3-((4-(methoxycarbonyl)-1H-indol-5-yl)oxy)benzimidothioic acid (0.5 g, 1.53 mmol) in ethanol (5 mL) was stirred at room temperature overnight and then heated at 100° C. for 15 minutes. The solvent was evaporated and the residue was chromatographed (0-100% ethyl acetate in heptane) to afford the title compound as a white solid (320 mg, 37%). MS m/z: 567 [M+H]⁺.

### C. (5-(3-(4-(3-iodobenzyl)thiazol-2-yl)phenoxy)-1H-indol-4-yl)methanol

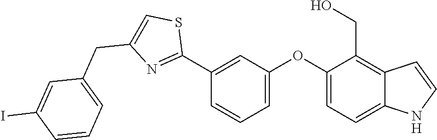 [SMILES: OCc1c(Oc2cccc(-c3nc(Cc4cccc(I)c4)cs3)c2)ccc2[nH]ccc12]

DIBAL-H (3.18 ml, 3.18 mmol, 1M in hexanes) was added to a solution of methyl 5-(3-(4-(3-iodobenzyl)thiazol-2-yl)phenoxy)-1H-indole-4-carboxylate (300 mg, 529.65 μmol) in dichloromethane (4 mL) and the mixture was stirred at −78° C. for one hour. The reaction was quenched with saturated sodium potassium tartrate (15 mL), stirred overnight at room temperature and extracted with ethyl acetate. The combined organic extracts were washed with water and brine, dried and purified by column chromatography (0-100% ethyl acetate in heptane) to afford the title compound as a white solid (260 mg, 91%). MS m/z: 539 [M+H]⁺. ¹H NMR (400 MHz, CD₃OD) δ 7.65 (d, J=1.9 Hz, 1H), 7.58-7.47 (m, 2H), 7.46-7.29 (m, 4H), 7.26 (d, J=8.9 Hz, 1H), 7.10-6.92 (m, 3H), 6.82 (d, J=8.6 Hz, 1H), 6.71 (d, J=2.2 Hz, 1H), 4.87 (s, 2H), 4.04 (s, 2H) ppm.

### D. Example 48. (5-(3-(4-Benzylthiazol-2-yl)phenoxy)-1H-indol-4-yl)methanol

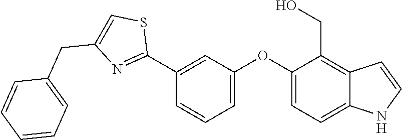 [SMILES: OCc1c(Oc2cccc(-c3nc(Cc4ccccc4)cs3)c2)ccc2[nH]ccc12]

A mixture of (5-(3-(4-(3-iodobenzyl)thiazol-2-yl)phenoxy)-1H-indol-4-yl)methanol (60 mg, 111 μmol), sodium bicarbonate (37 mg, 446 μmol) and Pd—C (24 mg, 10% on carbon, 50% wet) in ethyl acetate (8 mL) was stirred under hydrogen for two days. The mixture was filtered through a pad of Celite, the solvent was evaporated and the residue was chromatographed (0-100% ethyl acetate in heptane) to afford the title compound as a white solid (37 mg, 81%). MS m/z: 413 [M+H]⁺. ¹H NMR (400 MHz, CDCl₃) δ 8.32 (s, 1H), 7.62-7.56 (m, 2H), 7.38-7.28 (m, 7H), 6.93-6.90 (m, 2H), 6.78-6.72 (m, 2H), 4.98 (s, 2H), 4.17 (s, 2H) ppm.

### Example 49

### 2-((1H-Indol-5-yl)oxy)-4-(5-benzyl-1H-1,2,4-triazol-3-yl)aniline

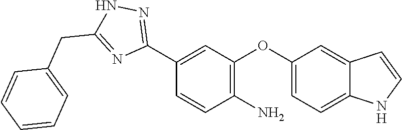 [SMILES: Nc1ccc(-c2n[nH]c(Cc3ccccc3)n2)cc1Oc1ccc2[nH]ccc2c1]

### A. Methyl 3-((1H-indol-5-yl)oxy)-4-nitrobenzoate

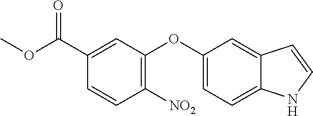 [SMILES: COC(=O)c1ccc([N+](=O)[O-])c(Oc2ccc3[nH]ccc3c2)c1]

Potassium carbonate (5.19 g, 37.55 mmol) was added to a solution of 1H-indol-5-ol (2.5 g, 18.78 mmol) and methyl 3-fluoro-4-nitrobenzoate (3.74 g, 18.78 mmol) in DMF (10 mL) and heated at 70° C. overnight. The reaction mixture was diluted with ethyl acetate (150 mL), washed with water and brine and dried over sodium sulfate. After removal of solvent, the reddish residue was used in the next step without further purification. MS m/z: 313 [M+H]⁺.

### B. 3-((1H-Indol-5-yl)oxy)-4-nitrobenzohydrazide

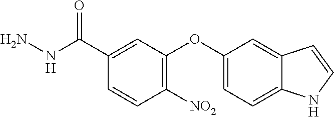 [SMILES: NNC(=O)c1ccc([N+](=O)[O-])c(Oc2ccc3[nH]ccc3c2)c1]

A solution of methyl 3-((1H-indol-5-yl)oxy)-4-nitrobenzoate (4.5 g, 14.41 mmol) and hydrazine hydrate (2.26 ml, 72.05 mmol) was stirred at room temperature overnight. The solvent was evaporated and the residue was chromatographed (0-10% methanol in dichloromethane containing 0.5% ammonium hydroxide) to give the title compound as a yellow solid (4 g, 88%). MS m/z: 313 [M+H]⁺.

### C. 5-(5-(5-Benzyl-1H-1,2,4-triazol-3-yl)-2-nitrophenoxy)-1H-indole

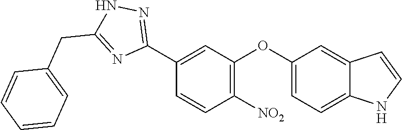 [SMILES: O=[N+]([O-])c1ccc(-c2n[nH]c(Cc3ccccc3)n2)cc1Oc1ccc2[nH]ccc2c1]

This reaction was carried out in two 20 mL microwave reaction vials.

A mixture of 2-phenylacetonitrile (4.5 g, 38 mmol), 3-((1H-indol-5-yl)oxy)-4-nitrobenzohydrazide (3 g, 9.6 mmol) and potassium carbonate (2.66 g, 19 mmol) in n-Butanol (15 mL×2) was heated at 150° C. for one hour in a microwave reactor. The solvent was evaporated and the residue was chromatographed (0-100% ethyl acetate in heptane) to afford the title compound as a yellow solid (300 mg. 8%). MS m/z: 412 [M+H]⁺.

### D. Example 49. 2-((1H-Indol-5-yl)oxy)-4-(5-benzyl-1H-1,2,4-triazol-3-yl)aniline

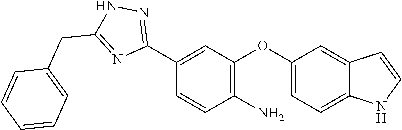 [SMILES: Nc1ccc(-c2n[nH]c(Cc3ccccc3)n2)cc1Oc1ccc2[nH]ccc2c1]

A mixture of 5-(5-(5-benzyl-1H-1,2,4-triazol-3-yl)-2-nitrophenoxy)-1H-indole (400 mg, 972 μmol) and Pd—C (350 mg, 10% on carbon, 50% wet) in 10 mL of ethyl acetate was stirred under hydrogen overnight. The mixture was filtered through a pad of Celite and concentrated. The resulting residue was purified by silica gel column chromatography (0-100% ethyl acetate in heptane) twice to afford the title compound (0.17 g, 45%). MS m/z: 382 [M+H]⁺. ¹H NMR (400 MHz, CD₃OD) δ 7.47 (dd, J=8.2, 1.5 Hz, 1H), 7.39-7.13 (m, 9H), 6.93-6.84 (m, 2H), 6.38 (dd, J=3.1, 0.8 Hz, 1H), 4.00 (s, 2H) ppm.

### Example 50

### N-((5-(3-(5-Benzyl-4H-1,2,4-triazol-3-yl)phenoxy)-1H-indol-4-yl)methyl)acetamide

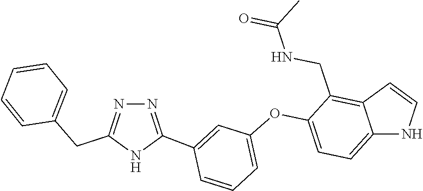 [SMILES: CC(=O)NCc1c(Oc2cccc(-c3nnc(Cc4ccccc4)[nH]3)c2)ccc2[nH]ccc12]

### A. (5-(3-(5-Benzyl-4H-1,2,4-triazol-3-yl)phenoxy)-1H-indol-4-yl)methanamine

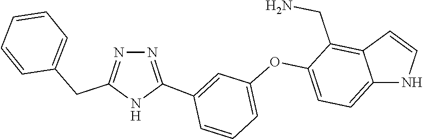 [SMILES: NCc1c(Oc2cccc(-c3nnc(Cc4ccccc4)[nH]3)c2)ccc2[nH]ccc12]

To a stirred solution of 5-(3-(5-benzyl-4H-1,2,4-triazol-3-yl)phenoxy)-1H-indole-4-carbaldehyde (Example 21 Step A, 150 mg, 0.38 mmol) in ethanol (15 mL) was added hydroxylamine hydrochloride (53 mg, 0.76 mmol) and NaOAc (63 mg, 0.76 mmol). The reaction mixture was refluxed for 0.5 hours and concentrated in vacuo. The resulting residue was dissolved in THF (15 mL) 10% Pd/C (100 mg) and concentrated hydrochloric acid (2 drops) were added and stirred at room temperature under hydrogen atmosphere overnight. The reaction mixture was filtered through a pad of Celite and the filtrate was concentrated to afford the title compound as a yellow solid (128 mg, 85%). The crude product was used without further purification. MS m/z: 394 [M−1]⁻.

### B. Example 50. N-((5-(3-(5-Benzyl-4H-1,2,4-triazol-3-yl)phenoxy)-1H-indol-4-yl)methyl)acetamide

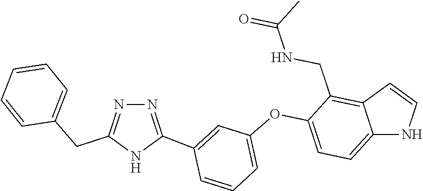 [SMILES: CC(=O)NCc1c(Oc2cccc(-c3nnc(Cc4ccccc4)[nH]3)c2)ccc2[nH]ccc12]

To a stirred solution of (5-(3-(5-benzyl-4H-1,2,4-triazol-3-yl)phenoxy)-1H-indol-4-yl)methanamine (128 mg, 0.32 mmol) in dichloromethane (10 mL) at −78° C. were added acetyl chloride (13 mg, 0.16 mmol) and triethylamine (97 mg, 0.96 mmol). The reaction mixture was stirred at −78° C. for 0.5 hours, quenched with water (10 mL) and extracted with dichloromethane (20 mL×3). The combined organic extracts were concentrated and purified by preparative HPLC to afford the title compound as a white solid (27 mg, 19%). MS m/z: 438 [M+H]⁺. ¹H NMR (500 MHz, DMSO-d₆) δ 13.92 (br s, 1H), 11.26 (s, 1H), 8.05 (t, J=5.0 Hz, 1H), 7.62 (d, J=7.0 Hz, 1H), 7.43-7.37 (m, 4H), 7.33-7.26 (m, 4H), 7.24-7.20 (m, 1H), 6.94 (dd, J=8.0, 2.0 Hz, 1H), 6.79 (d, J=8.5 Hz, 1H), 6.56-6.53 (m, 1H), 4.46 (d, J=5.0 Hz, 2H), 4.07 (s, 2H), 1.72 (s, 3H) ppm.

### Example 51

### 2-(5-(3-(5-Benzyl-4H-1,2,4-triazol-3-yl)phenoxy)-1H-indol-4-yl)acetamide

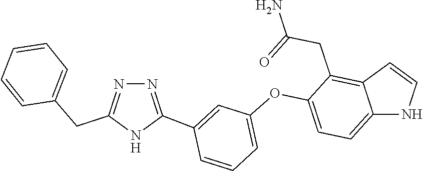 [SMILES: NC(=O)Cc1c(Oc2cccc(-c3nnc(Cc4ccccc4)[nH]3)c2)ccc2[nH]ccc12]

A mixture of 2-(5-(3-(5-benzyl-4H-1,2,4-triazol-3-yl)phenoxy)-1H-indol-4-yl)acetic acid (Example 11, 42 mg, 0.10 mmol), ammonium chloride (22 mg, 0.40 mmol), HATU (76 mg, 0.20 mmol) and diisopropylethylamine (52 mg, 0.40 mmol) in DMF (10 mL) was stirred at room temperature overnight. The reaction mixture was diluted with ethyl acetate (80 mL), washed with water (30 mL×3) and brine (30 mL), dried over sodium sulfate and concentrated. The resulting residue was purified by preparative HPLC to afford the title compound as a white solid (8.2 mg, 20%). MS m/z: 424 [M+H]⁺. ¹H NMR (500 MHz, CD₃OD) δ 7.64 (br d, J=5.0 Hz, 1H), 7.51 (br s, 1H), 7.41-7.39 (m, 2H), 7.35 (d, J=3.0 Hz, 1H), 7.34-7.27 (m, 4H), 7.24 (t, J=7.0 Hz, 1H), 7.05-7.02 (m, 1H), 6.88 (d, J=8.5 Hz, 1H), 6.58 (d, J=2.0 Hz, 1H), 4.13 (s, 2H), 3.82 (s, 2H) ppm.

### Example 52

### (2-(3-((1H-Pyrrolo[3,2-b]pyridin-5-yl)oxy)phenyl)-1H-imidazol-5-yl)(phenyl)methanol

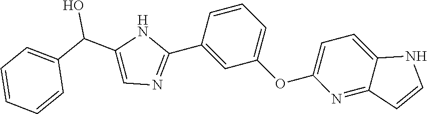 [SMILES: OC(c1ccccc1)c1cnc(-c2cccc(Oc3ccc4[nH]ccc4n3)c2)[nH]1]

To a stirred solution of 2-(3-((1H-pyrrolo[3,2-b]pyridin-5-yl)oxy)phenyl)-1H-imidazole-5-carbaldehyde (Intermediate 16, 300 mg, 1 mmol) in THF (100 mL) was added phenylmagnesium bromide (1.0 N in THF, 8 mL, 8 mmol) at room temperature. The reaction mixture was stirred at room temperature for six hours, quenched with methanol (10 mL) and concentrated to dryness under 35° C. The resulting residue was purified by silica gel column chromatography (dichloromethane/methanol/concentrated ammonium hydroxide, v/v, 20/1/0.01) to afford the title compound as a white solid (260 mg, 69%). MS m/z: 383 [M+H]⁺. ¹H NMR (400 MHz, CD₃OD) δ 7.88 (d, J=8.8 Hz, 1H), 7.66 (d, J=7.6 Hz, 1H), 7.60 (t, J=2.0 Hz, 1H), 7.54 (d, J=2.8 Hz, 1H), 7.47-7.43 (m, 3H), 7.35 (t, J=7.2 Hz, 2H), 7.30-7.25 (m, 1H), 7.06 (d, J=8.0 Hz, 1H), 6.83-6.78 (m, 2H), 6.47 (d, J=2.8 Hz, 1H), 5.48 (s, 1H) ppm.

### Example 53

### 1-(2-(3-((1H-Pyrrolo[3,2-b]pyridin-5-yl)oxy)phenyl)-1H-imidazol-5-yl)-1-phenylethan-1-ol

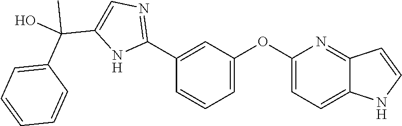 [SMILES: CC(O)(c1ccccc1)c1cnc(-c2cccc(Oc3ccc4[nH]ccc4n3)c2)[nH]1]

### A. (2-(3-((1H-Pyrrolo[3,2-b]pyridin-5-yl)oxy)phenyl)-1H-imidazol-5-yl)(phenyl)methanone

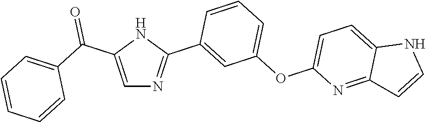 [SMILES: O=C(c1ccccc1)c1cnc(-c2cccc(Oc3ccc4[nH]ccc4n3)c2)[nH]1]

To a stirred solution of (2-(3-((1H-pyrrolo[3,2-b]pyridin-5-yl)oxy)phenyl)-1H-imidazol-5-yl)(phenyl)methanol (150 mg, 0.39 mmol) in THF (20 mL) was added manganese dioxide (150 mg, 3.9 mmol). The reaction mixture was stirred at room temperature for four hours, filtered through Celite and concentrated in vacuo. The resulting residue was purified by silica gel column chromatography (dichloromethane/methanol, v/v, 50/1) to afford the title compound as a yellow solid (135 mg, 90%). MS m/z: 381[M+H]⁺. ¹H NMR (400 MHz, DMSO-d₆) δ 13.42 (br s, 1H), 11.37 (s, 1H), 8.24-7.08 (m, 6H), 7.64-7.49 (m, 5H), 7.16 (d, J=7.6 Hz, 1H), 6.86 (d, J=8.8 Hz, 1H), 6.39 (s, 1H) ppm.

### B. Example 53. 1-(2-(3-((1H-Pyrrolo[3,2-b]pyridin-5-yl)oxy)phenyl)-1H-imidazol-5-yl)-1-phenylethan-1-ol

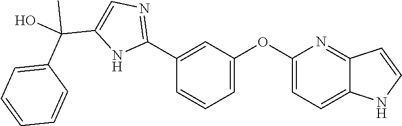 [SMILES: CC(O)(c1ccccc1)c1cnc(-c2cccc(Oc3ccc4[nH]ccc4n3)c2)[nH]1]

To a stirred solution of (2-(3-((1H-pyrrolo[3,2-b]pyridin-5-yl)oxy)phenyl)-1H-imidazol-5-yl)(phenyl)methanone (135 mg, 0.36 mmol) in THF (10 mL) was added methylmagnesium bromide (3.0 N in THF, 0.71 mL, 2.13 mmol) at room temperature. The reaction mixture was stirred at room temperature for one hour, quenched with methanol (3 mL) and concentrated to dryness under 35° C. The resulting residue was purified by silica gel column chromatography (dichloromethane/methanol/concentrated ammonium hydroxide, v/v, 20/1/0.01) to give 90 mg of impure product. The impure product was further purified by preparative HPLC to afford the title compound as a white solid (37 mg, 26%). MS m/z: 397 [M+H]⁺. ¹H NMR (400 MHz, CD₃OD) δ 7.87 (d, J=8.8 Hz, 1H), 7.66 (d, J=8.0 Hz, 1H), 7.60 (t, J=2.0 Hz, 1H), 7.53 (d, J=3.2 Hz, 1H), 7.49-7.47 (m, 2H), 7.44 (t, J=8.0 Hz, 1H), 7.32 (t, J=8.0 Hz, 2H), 7.25-7.23 (m, 1H), 7.05 (dd, J=8.0, 1.6 Hz, 1H), 6.90 (s, 1H), 6.80 (d, J=8.8 Hz, 1H), 6.47 (d, J=3.2 Hz, 1H), 1.90 (s, 3H) ppm.

### Example 54

### N1-((2-(3-((1H-pyrrolo[3,2-b]pyridin-5-yl)oxy)phenyl)-1H-imidazol-5-yl)(phenyl)methyl)-N2,N2-dimethylethane-1,2-diamine

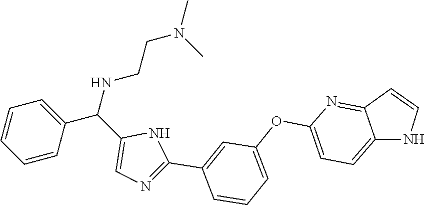 [SMILES: CN(C)CCNC(c1ccccc1)c1cnc(-c2cccc(Oc3ccc4[nH]ccc4n3)c2)[nH]1]

A mixture of (2-(3-((1H-pyrrolo[3,2-b]pyridin-5-yl)oxy)phenyl)-1H-imidazol-5-yl)(phenyl)methanone (Example 53 Step A, 140 mg, 0.37 mmol), N¹,N¹-dimethylethane-1,2-diamine (325 mg, 3.7 mmol) and titanium tetraisopropoxide (628 mg, 2.2 mmol) in methanol (30 mL) was stirred at 35° C. under nitrogen overnight. The reaction mixture was cooled, NaBH₄ (56 mg, 1.47 mmol) was added and stirred at room temperature for two hours. The solid filtered, the filtrate concentrated and purified by silica gel column chromatography (dichloromethane/methanol/ammonium hydroxide, v/v, 15/1/0.1) to afford the title compound as a yellow solid (81 mg, 48%). MS m/z: 453 [M+H]⁺. ¹H NMR (400 MHz, CD₃OD) δ 7.89 (d, J=8.4 Hz, 1H), 7.65 (d, J=7.6 Hz, 1H), 7.58 (s, 1H), 7.55 (d, J=3.2 Hz, 1H), 7.78-7.45 (m, 3H), 7.37 (t, J=7.6 Hz, 2H), 7.29-7.26 (m, 1H), 7.07 (dd, J=7.6, 1.6 Hz, 1H), 6.84-6.81 (m, 2H), 6. 47 (d, J=2.8 Hz, 1H), 4.88 (s, 1H), 2.75-2.64 (m, 2H), 2.56-2.53 (m, 2H), 2.55 (s, 6H) ppm.

### Example 55

### (2-(3-((4-(Methylsulfinyl)-1H-indol-5-yl)oxy)phenyl)-1H-imidazol-5-yl)(phenyl)methanol

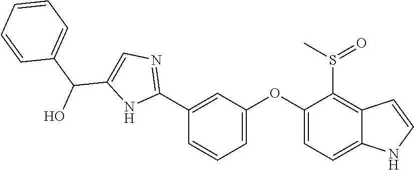 [SMILES: CS(=O)c1c(Oc2cccc(-c3ncc(C(O)c4ccccc4)[nH]3)c2)ccc2[nH]ccc12]

### A. (2-(3-((4-(Methylthio)-1H-indol-5-yl)oxy)phenyl)-1H-imidazol-5-yl)(phenyl)methanol

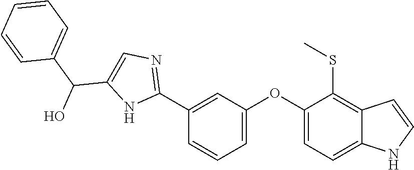 [SMILES: CSc1c(Oc2cccc(-c3ncc(C(O)c4ccccc4)[nH]3)c2)ccc2[nH]ccc12]

To a stirred solution of 2-(3-((4-(methylthio)-1H-indol-5-yl)oxy)phenyl)-1H-imidazole-5-carbaldehyde (Intermediate 14, 65 mg, 0.20 mmol) in THF (10 mL) was added phenylmagnesium bromide (1.0 N in THF, 2.0 mL, 2.0 mmol) dropwise at 0° C. The reaction mixture was stirred at room temperature for five hours, quenched with saturated ammonium chloride (5 mL) solution and extracted with dichloromethane (20 mL×2). The combined organic extracts were washed with brine (10 mL), dried over sodium sulfate, filtered and concentrated. The resulting residue was purified by silica gel column chromatography (dichloromethane/methanol, v/v, 30/1 to 20/1) to afford the title compound as a white solid (50 mg, 88%). MS-ESI m/z: 428 [M+H]⁺.

### B. Example 55. (2-(3-((4-(Methylsulfinyl)-1H-indol-5-yl)oxy)phenyl)-1H-imidazol-5-yl)(phenyl)methanol

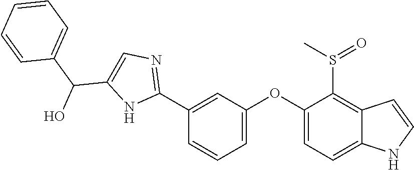 [SMILES: CS(=O)c1c(Oc2cccc(-c3ncc(C(O)c4ccccc4)[nH]3)c2)ccc2[nH]ccc12]

To a stirred solution of (2-(3-((4-(methylthio)-1H-indol-5-yl)oxy)phenyl)-1H-imidazol-5-yl)(phenyl)methanol (62 mg, 0.14 mmol) in methanol (4.0 mL) was added H₂O₂ (30% in water, 80 mg, 0.70 mmol). The reaction mixture was stirred at 50° C. for 20 hours and concentrated under reduced pressure. The resulting residue was purified by preparative HPLC to afford the title compound as a white solid (25 mg, 39%). MS-ESI m/z: 444 [M+H]⁺. ¹H NMR (400 MHz, CD₃OD) δ 7.63 (d, J=8.4 Hz, 1H), 7.59 (d, J=8.0 Hz, 1H), 7.50-7.28 (m, 8H), 7.18 (d, J=3.2 Hz, 1H), 6.96 (dd, J=8.4, 2.4 1H), 6.89 (d, J=9.2 Hz, 1H), 6.77 (br s, 1H), 5.80 (s, 1H), 3.02 (s, 3H) ppm.

### Example 56

### 2-(5-(3-(5-Benzyl-4H-1,2,4-triazol-3-yl)phenoxy)-1H-indol-4-yl)-N-(methylsulfonyl)acetamide

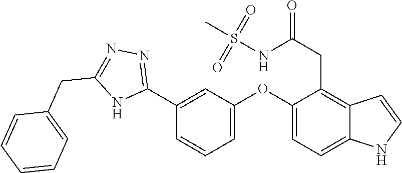 [SMILES: CS(=O)(=O)NC(=O)Cc1c(Oc2cccc(-c3nnc(Cc4ccccc4)[nH]3)c2)ccc2[nH]ccc12]

A mixture of 2-(5-(3-(5-benzyl-4H-1,2,4-triazol-3-yl)phenoxy)-1H-indol-4-yl)acetic acid (148 mg, 0.35 mmol), methanesulfonamide (67 mg, 0.69 mmol, Example 11), EDCI (136 mg, 0.69 mmol) and 4-dimethylaminopyridine (128 mg, 1.04 mmol) in dichloromethane (20 mL) was stirred at room temperature overnight. The reaction mixture was diluted with dichloromethane (50 mL), washed with water (20 mL×2) and brine (20 mL×1), dried over sodium sulfate and concentrated. The resulting crude product was purified by preparative TLC to afford the title compound as a white solid (16 mg, 9%). MS m/z: 502 [M+H]⁺. ¹H NMR (400 MHz, CD₃OD) δ 7.63 (br s, 1H), 7.53 (br s, 1H), 7.39 (d, J=8.8 Hz, 2H), 7.34-7.27 (m, 5H), 7.26-7.23 (m, 1H), 7.03-6.96 (m, 1H), 6.86 (d, J=8.4 Hz, 1H), 6.55 (s, 1H), 4.14 (s, 2H), 3.92 (s, 2H), 2.95 (s, 3H) ppm.

### Example 57

### 5-(3-(5-Benzyl-1-methyl-1H-1,2,4-triazol-3-yl)phenoxy)-1H-indole-4-carboxamide

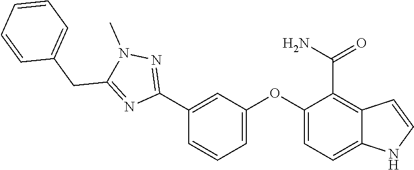 [SMILES: Cn1nc(-c2cccc(Oc3ccc4[nH]ccc4c3C(N)=O)c2)nc1Cc1ccccc1]

### A. Methyl 5-(3-(5-benzyl-1H-1,2,4-triazol-3-yl)phenoxy)-1-tosyl-1H-indole-4-carboxylate

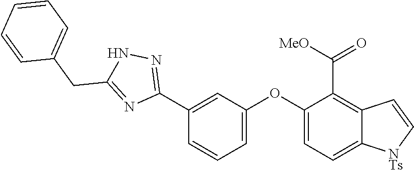 [SMILES: COC(=O)c1c(Oc2cccc(-c3n[nH]c(Cc4ccccc4)n3)c2)ccc2c1ccn2S(=O)(=O)c1ccc(C)cc1]

To a mixture of methyl 5-(3-cyanophenoxy)-1-tosyl-1H-indole-4-carboxylate (Intermediate 5-3, 1 g, 2.24 mmol) in methanol (12 ml) was added acetyl chloride (7 ml, 98 mmol) at 0° C. The mixture was stirred overnight and the solvent was evaporated. The resulting residue was triturated with ethyl acetate and the white solid was collected by filtration to afford methyl 5-(3-(imino(methoxy)methyl)phenoxy)-1-tosyl-1H-indole-4-carboxylate hydrochloride (0.95 g, 82%), which was used without further purification. MS m/z: 479 [M+H]⁺.

A mixture of methyl 5-(3-(imino(methoxy)methyl)phenoxy)-1-tosyl-1H-indole-4-carboxylate hydrochloride (0.5 g, 971 μmol), 2-phenylacetohydrazide (175 mg, 1.17 mmol) and triethylamine (541 μl, 3.88 mmol) in ethanol (5 ml, 86 mmol) was heated to 120° C. for one hour. The solvent was evaporated and the residue was chromatographed over silica gel (0-100% ethyl acetate in heptane) to afford the title compound as a white solid (0.4 g, 71%). MS m/z: 579 [M+H]⁺.

### B. Methyl 5-(3-(5-benzyl-1-methyl-1H-1,2,4-triazol-3-yl)phenoxy)-1-tosyl-1H-indole-4-carboxylate

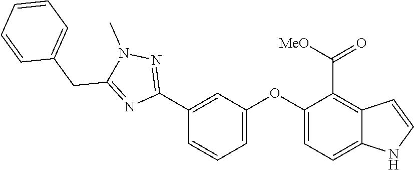 [SMILES: COC(=O)c1c(Oc2cccc(-c3nc(Cc4ccccc4)n(C)n3)c2)ccc2[nH]ccc12]

A mixture of methyl 5-(3-(5-benzyl-4H-1,2,4-triazol-3-yl)phenoxy)-1-tosyl-1H-indole-4-carboxylate (120 mg, 207 μmol), potassium carbonate (57 mg, 415 μmol) and iodomethane (16 μl, 249 μmol) in DMF (5 ml) was stirred at room temperature overnight. The solvent was evaporated and the residue was chromatographed over silica gel (0-100% ethyl acetate in heptane) to afford the title compound as a white solid (100 mg, 81%). MS m/z: 439 [M+H]⁺.

### C. Example 57. 5-(3-(5-Benzyl-1-methyl-1H-1,2,4-triazol-3-yl)phenoxy)-1H-indole-4-carboxamide

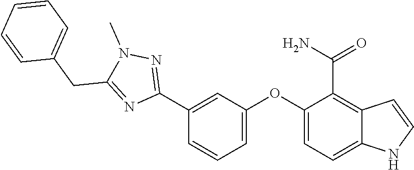 [SMILES: Cn1nc(-c2cccc(Oc3ccc4[nH]ccc4c3C(N)=O)c2)nc1Cc1ccccc1]

A mixture of methyl 5-(3-(5-benzyl-1-methyl-1H-1,2,4-triazol-3-yl)phenoxy)-1-tosyl-1H-indole-4-carboxylate (100 mg, 168 μmol) and a 7M solution of ammonia in methanol (19 ml, 135 mmol) was heated at 50° C. for four days. The solvent was evaporated and the residue was chromatographed over silica gel (0-100% ethyl acetate in heptane) to afford the title compound as a white solid (9 mg, 13%). MS m/z: 424 [M+H]⁺. ¹H NMR (400 MHz, CD₃OD) δ 7.68 (d, J=7.3 Hz, 1H), 7.58-7.49 (m, 2H), 7.41-7.17 (m, 7H), 7.00 (dd, J=8.2, 2.5 Hz, 1H), 6.91-6.80 (m, 2H), 4.22 (s, 2H), 3.74 (s, 3H) ppm.

### Example 58

### 3-(5-(3-(5-Benzyl-4H-1,2,4-triazol-3-yl)phenoxy)-1H-indol-4-yl)propanoic acid

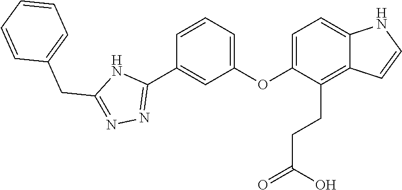 [SMILES: O=C(O)CCc1c(Oc2cccc(-c3nnc(Cc4ccccc4)[nH]3)c2)ccc2[nH]ccc12]

### A. Ethyl (E)-3-(5-(3-cyanophenoxy)-1-tosyl-1H-indol-4-yl)acrylate

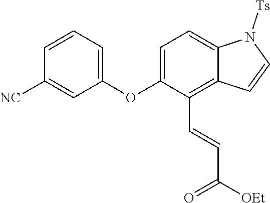 [SMILES: CCOC(=O)/C=C/c1c(Oc2cccc(C#N)c2)ccc2c1ccn2S(=O)(=O)c1ccc(C)cc1]

A sealed tube containing a mixture of 3-((4-bromo-1-tosyl-1H-indol-5-yl)oxy)benzonitrile (800 mg, 1.72 mmol, Intermediate 5-2), ethyl acrylate (515 mg, 5.15 mmol), Pd(OAc)₂ (77 mg, 0.34 mmol), P(o-tolyl)₃ (209 mg, 0.69 mmol) and diisopropylethylamine (443 mg, 3.43 mmol) in DMF (20 mL) was stirred at 100° C. under nitrogen atmosphere overnight and the reaction mixture was concentrated in vacuo. The residue was dissolved with ethyl acetate (150 mL), washed with water (30 mL×3) and brine (30 mL×2), dried over sodium sulfate, filtered and concentrated. The resulting crude product was purified by silica gel column (petroleum ether/ethyl acetate, v/v, 20/1-5/1) to afford the title compound as a light yellow solid (693 mg, 83%). MS m/z: 487 [M+H]⁺.

### B. (E)-3-(5-(3-(5-benzyl-4H-1,2,4-triazol-3-yl)phenoxy)-1H-indol-4-yl)acrylic acid

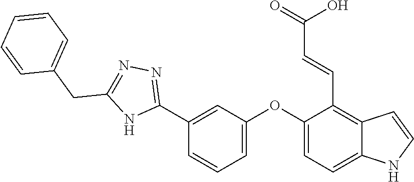 [SMILES: O=C(O)/C=C/c1c(Oc2cccc(-c3nnc(Cc4ccccc4)[nH]3)c2)ccc2[nH]ccc12]

A mixture of ethyl (E)-3-(5-(3-cyanophenoxy)-1-tosyl-1H-indol-4-yl)acrylate (500 mg, 1.03 mmol), 2-phenylacetohydrazide (618 mg, 4.12 mmol) and potassium carbonate (710 mg, 5.14 mmol) in n-Butanol (15 mL) was irradiated at 180° C. for six hours in a Biotage microwave synthesizer under nitrogen atmosphere. The reaction mixture was concentrated and the residue was dissolved with ethyl acetate (100 mL). The mixture was washed with water (30 mL×3) and brine (30 mL×1), dried over sodium sulfate and concentrated. The resulting crude product was purified by preparative TLC (dichloromethane/methanol, v/v, 8/1) to afford the title compound as a light yellow solid (103 mg, 23%). MS m/z: 437 [M+H]⁺.

### C. Example 58. 3-(5-(3-(5-Benzyl-4H-1,2,4-triazol-3-yl)phenoxy)-1H-indol-4-yl)propanoic acid

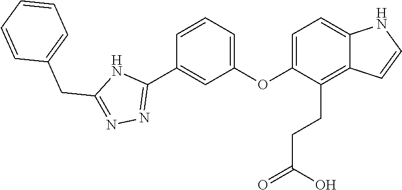 [SMILES: O=C(O)CCc1c(Oc2cccc(-c3nnc(Cc4ccccc4)[nH]3)c2)ccc2[nH]ccc12]

A mixture of (E)-3-(5-(3-(5-benzyl-4H-1,2,4-triazol-3-yl)phenoxy)-1H-indol-4-yl)acrylic acid (250 mg, 0.58 mmol) and 10% Pd/C (120 mg) in ethyl acetate (15 mL) and THF (5 mL) was stirred at room temperature under hydrogen atmosphere overnight. The reaction mixture was filtered through a pad of Celite and the filtrate was concentrated to afford the title compound as light yellow oil (180 mg, 93%). MS m/z: 439 [M+H]⁺. LC-MS retention time: 1.11 minutes.

### Example 59

### 3-(5-(3-(5-Benzyl-4H-1,2,4-triazol-3-yl)phenoxy)-1H-indol-4-yl)-N-(methylsulfonyl)propanamide

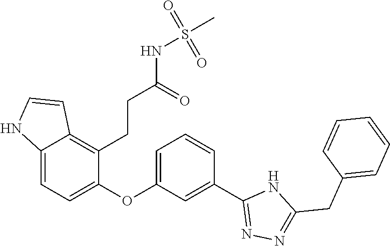 [SMILES: CS(=O)(=O)NC(=O)CCc1c(Oc2cccc(-c3nnc(Cc4ccccc4)[nH]3)c2)ccc2[nH]ccc12]

A mixture of 3-(5-(3-(5-benzyl-4H-1,2,4-triazol-3-yl)phenoxy)-1H-indol-4-yl)propanoic acid (Example 58, 130 mg, 0.30 mmol), methanesulfonamide (142 mg, 1.49 mmol), 1-hydroxybenzotriazole hydrate (61 mg, 0.45 mmol), HATU (171 mg, 0.45 mmol) and triethylamine (91 mg, 0.89 mmol) in DMF (15 mL) was stirred at room temperature overnight, diluted with water (10 mL) and extracted with ethyl acetate (30 mL×3). The combined organic extracts were washed with brine (10 mL×2), dried over sodium sulfate, filtered and concentrated. The crude product was purified by preparative HPLC to afford the title compound as a white solid (14 mg, 9%). MS m/z: 516 [M+H]⁺. ¹H NMR (400 MHz, CD₃OD) δ 7.58 (d, J=7.6 Hz, 1H), 7.48 (s, 1H), 7.37-7.35 (m, 1H), 7.33-7.25 (m, 6H), 7.23-7.21 (m, 1H), 6.94 (dd, J=8.0, 2.0 Hz, 1H), 6.77 (d, J=8.4 Hz, 1H), 6.64 (d, J=2.8 Hz, 1H), 4.11 (s, 2H), 3.12 (t, J=8.0 Hz, 2H), 2.93 (s, 3H), 2.52 (t, J=8.4 Hz, 2H) ppm.

### Example 60

### 2-(5-(3-(5-Benzyl-4H-1,2,4-triazol-3-yl)phenoxy)-1H-indol-4-yl)-N-((trifluoromethyl)sulfonyl)acetamide

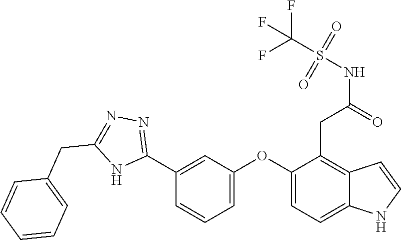 [SMILES: O=C(Cc1c(Oc2cccc(-c3nnc(Cc4ccccc4)[nH]3)c2)ccc2[nH]ccc12)NS(=O)(=O)C(F)(F)F]

To a stirred solution of 2-(5-(3-(5-benzyl-4H-1,2,4-triazol-3-yl)phenoxy)-1H-indol-4-yl)acetic acid (Example 11, 20 mg, 0.28 mmol) in DMF (2 mL) were added DMAP (102 mg, 0.84 mmol), EDCI (160 mg, 0.84 mmol) and CF₃SO₂NH₂ (167 mg, 1.12 mmol). The reaction mixture was stirred at 25° C. for three hours, quenched with water (10 mL) and extracted with ethyl acetate (30 mL×3). The combined organic extracts were washed with brine, dried over sodium sulfate, filtered and concentrated. The resulting residue was purified by preparative HPLC to afford the title compound as a white solid (28.9 mg, 22%). MS m/z: 556 [M+H]⁺. ¹H NMR (400 MHz, CD₃OD) δ 7.60 (d, J=7.6 Hz, 1H), 7.47 (s, 1H), 7.38 (t, J=8.0 Hz, 1H), 7.34-7.29 (m, 6H), 7.25-7.23 (m, 1H), 7.04 (d, J=6.8 Hz, 1H), 6.81 (d, J=8.8 Hz, 1H), 6.54 (d, J=2.8 Hz, 1H), 4.13 (s, 2H), 3.90 (s, 2H) ppm.

### Example 61

### 1-(2-(3-((6-Fluoro-4-((2-methoxyethyl)sulfonyl)-1H-indol-5-yl)oxy)phenyl)-1H-imidazol-5-yl)-1-phenylethan-1-ol

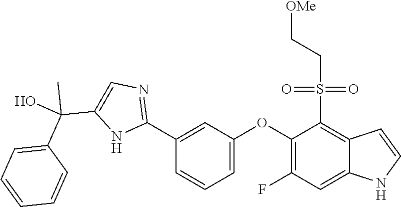 [SMILES: COCCS(=O)(=O)c1c(Oc2cccc(-c3ncc(C(C)(O)c4ccccc4)[nH]3)c2)c(F)cc2[nH]ccc12]

### A. 1-(2-(3-((6-Fluoro-4-((2-methoxyethyl)thio)-1H-indol-5-yl)oxy)phenyl)-1H-imidazol-5-yl)-1-phenylethan-1-ol

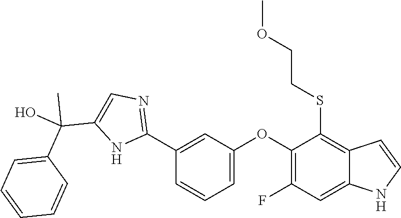 [SMILES: COCCSc1c(Oc2cccc(-c3ncc(C(C)(O)c4ccccc4)[nH]3)c2)c(F)cc2[nH]ccc12]

In glove box, 3-((6-fluoro-4-((2-methoxyethyl)thio)-1H-indol-5-yl)oxy)benzimidamide (Intermediate 8, 280 mg, 0.78 mmol) and 3-methyl-3-phenyloxirane-2-carbaldehyde (Intermediate 3, 126 mg, 0.78 mmol) was dissolved in DMF (6 mL). The mixture was stirred at 75° C. overnight, quenched with water (20 mL) and extracted with ethyl acetate (20 mL×3). The combined organic extracts were washed with brine (20 mL), dried over sodium sulfate, filtered and concentrated. The resulting residue was purified by flash chromatography (0-100% ethyl acetate in petroleum ether) to afford the title compound as a white solid (150 mg, 43%). MS m/z: 504 [M+H]⁺. There should be only one aromatic singlet. ¹H NMR (400 MHz, CD₃OD) δ 7.52 (d, J=7.6 Hz, 1H), 7.49 (s, 1H), 7.47 (s, 1H), 7.42 (s, 1H), 7.37-7.28 (m, 5H), 7.23 (t, J=7.0 Hz, 1H), 6.87 (br s, 1H), 6.82 (dd, J=8.0, 2.0 Hz, 1H), 6.68 (d, J=3.2 Hz, 1H), 3.38 (t, J=6.8 Hz, 2H), 3.18 (s, 3H), 3.05 (t, J=6.8 Hz, 2H), 1.89 (s, 3H) ppm.

### B. Example 61. 1-(2-(3-((6-Fluoro-4-((2-methoxyethyl)sulfonyl)-1H-indol-5-yl)oxy)phenyl)-1H-imidazol-5-yl)-1-phenylethan-1-ol

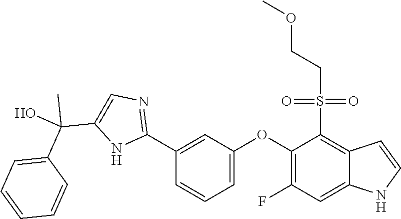 [SMILES: COCCS(=O)(=O)c1c(Oc2cccc(-c3ncc(C(C)(O)c4ccccc4)[nH]3)c2)c(F)cc2[nH]ccc12]

To a stirred solution of 1-(2-(3-((6-fluoro-4-((2-methoxyethyl)thio)-1H-indol-5-yl)oxy)phenyl)-1H-imidazol-5-yl)-1-phenylethan-1-ol (90 mg, 0.18 mmol) in methanol (9 mL) was added ammonium molybdate tetrahydrate (300 mg) in 1.5 mL of hydrogen peroxide (30% in water). The reaction mixture was stirred at room temperature for five hours, diluted with ethyl acetate (80 mL) and washed with water (20 mL×3), aqueous sodium sulfite (20 mL) and brine (20 mL×2). The organic phase was dried over sodium sulfate and concentrated. The resulting residue was purified by flash chromatography (0-100% ethyl acetate in petroleum ether) to afford the title compound as a yellow solid (16.4 mg, 18%). MS m/z: 536 [M+H]⁺. ¹H NMR (400 MHz, CD₃OD) δ 7.65 (d, J=10.4 Hz, 1H), 7.58 (d, J=7.6 Hz, 1H), 7.52 (d, J=3.2 Hz, 1H), 7.48-7.47 (m, 3H), 7.38 (t, J=8.0 Hz, 1H), 7.32 (t, J=7.2 Hz, 2H), 7.24 (d, J=6.8 Hz, 1H), 7.20 (d, J=2.8 Hz, 1H), 6.94 (dd, J=8.4, 2.4 Hz, 1H), 6.88 (br s, 1H), 3.78-3.70 (m, 4H), 3.10 (s, 3H), 1.89 (s, 3H) ppm.

### Example 62

### 1-(2-(3-((6-Fluoro-4-((2-methoxyethyl)sulfinyl)-1H-indol-5-yl)oxy)phenyl)-1H-imidazol-5-yl)-1-phenylethan-1-ol

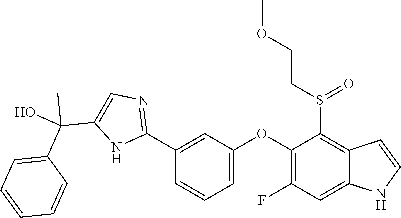 [SMILES: COCCS(=O)c1c(Oc2cccc(-c3ncc(C(C)(O)c4ccccc4)[nH]3)c2)c(F)cc2[nH]ccc12]

To a stirred solution of 1-(2-(3-((6-fluoro-4-((2-methoxyethyl)thio)-1H-indol-5-yl)oxy)phenyl)-1H-imidazol-5-yl)-1-phenylethan-1-ol (40 mg, 0.08 mmol) in methanol (3 mL) was added ammonium molybdate tetrahydrate (60 mg) in hydrogen peroxide (0.3 mL, 30% in water). The reaction mixture was stirred at room temperature 40 minutes, diluted with ethyl acetate (80 mL), washed with water (20 mL×3), aqueous sodium sulfite (20 mL) and brine, dried over sodium sulfate and concentrated. The resulting residue was purified by flash chromatography (0-100% ethyl acetate in petroleum ether) to afford the title compound as a white solid (20.5 mg, 50%). MS m/z: 520 [M+H]⁺. ¹H NMR (400 MHz, CD₃OD) δ 7.59 (d, J=8.4 Hz, 1H), 7.53 (d, J=10.0 Hz, 1H), 7.50-7.45 (m, 4H), 7.40 (t, J=8.0 Hz, 1H), 7.32 (t, J=8.0 Hz, 2H), 7.25-7.21 (m, 1H), 7.13 (d, J=2.8 Hz, 1H), 6.95-6.92 (m, 1H), 6.91-6.86 (m, 1H), 3.88-3.83 (m, 1H), 3.68-3.63 (m, 1H), 3.59-3.54 (m, 1H), 3.30-3.28 (m, 1H), 3.26 (s, 3H), 1.89 (s, 3H) ppm.

### Example 63

### 1-(2-(3-((4-(methylsulfonyl)-1H-indol-5-yl)oxy)phenyl)-1H-imidazol-5-yl)-1-phenylethan-1-ol (racemate) and

### Example 63A

### Enantiomer 1 of 1-(2-(3-((4-(methylsulfonyl)-1H-indol-5-yl)oxy)phenyl)-1H-imidazol-5-yl)-1-phenylethan-1-ol and

### Example 63B

### Enantiomer 2 of 1-(2-(3-((4-(methylsulfonyl)-1H-indol-5-yl)oxy)phenyl)-1H-imidazol-5-yl)-1-phenylethan-1-ol

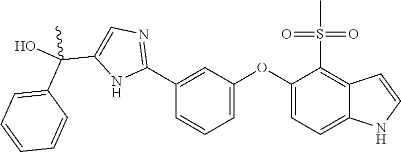 [SMILES: CC(O)(c1ccccc1)c1cnc(-c2cccc(Oc3ccc4[nH]ccc4c3S(C)(=O)=O)c2)[nH]1]

### A. 1-(2-(3-((4-(Methylthio)-1H-indol-5-yl)oxy)phenyl)-1H-imidazol-5-yl)-1-phenylethan-1-ol

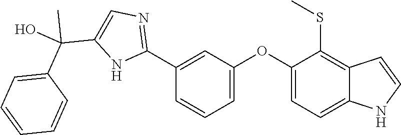 [SMILES: CSc1c(Oc2cccc(-c3ncc(C(C)(O)c4ccccc4)[nH]3)c2)ccc2[nH]ccc12]

In glove box, 3-((4-(methylthio)-1H-indol-5-yl)oxy)benzimidamide (750 mg, 2 mmol) and 3-methyl-3-phenyloxirane-2-carbaldehyde (Intermediate 3, 360 mg, 2.2 mmol) was dissolved in DMF (10 mL). The reaction mixture was stirred at 75° C. overnight, quenched with water (20 mL) and extracted with ethyl acetate (20 mL×3). The organic extracts were washed with brine (20 mL), dried over sodium sulfate, filtered and concentrated. The resulting residue was purified by flash chromatography (0-100% ethyl acetate in petroleum ether) to afford the title compound as a yellow solid (510 mg). MS m/z: 442 [M+H]⁺.

### B. Example 63. 1-(2-(3-((4-(Methylsulfonyl)-1H-indol-5-yl)oxy)phenyl)-1H-imidazol-5-yl)-1-phenylethan-1-ol

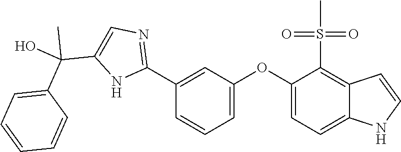 [SMILES: CC(O)(c1ccccc1)c1cnc(-c2cccc(Oc3ccc4[nH]ccc4c3S(C)(=O)=O)c2)[nH]1]

To a stirred solution of 1-(2-(3-((4-(methylthio)-1H-indol-5-yl)oxy)phenyl)-1H-imidazol-5-yl)-1-phenylethan-1-ol (510 mg, 1.16 mmol) in methanol (40 mL) was added ammonium molybdate tetrahydrate (1.2 g) in 6 mL of hydrogen peroxide (6 mL, 30% in water). The reaction mixture was stirred at room temperature for 3.5 hours, diluted with water (400 mL) and extracted with ethyl acetate (100 mL×3). The combined organic extracts were washed with aqueous sodium sulfite (100 mL) and brine (50 mL×2), dried over sodium sulfate and concentrated. The resulting residue was purified by flash chromatography (0-100% ethyl acetate in petroleum ether) to afford the title compound as a yellow solid (260 mg, 47%). %). MS m/z: 474 [M+H]⁺. ¹H NMR (400 MHz, CD₃OD) δ 7.71 (d, J=7.2 Hz, 1H), 7.63 (d, J=6.4 Hz, 1H), 7.56 (t, J=2.0 Hz, 1H), 7.51 (d, J=2.8 Hz, 1H), 7.48 (d, J=6.8 Hz, 2H), 7.43 (t, J=6.8 Hz, 1H), 7.32 (t, J=6.4 Hz, 2H), 7.25-7.20 (m, 2H), 7.05 (dd, J=6.8, 1.6 Hz, 1H), 6.92-6.90 (m, 2H), 3.35 (s, 3H), 1.90 (s, 3H) ppm.

### C. Example 63A, enantiomer 1, and Example 63B, Enantiomer 2 of 1-(2-(3-((4-(Methylsulfonyl)-1H-indol-5-yl)oxy)phenyl)-1H-imidazol-5-yl)-1-phenylethan-1-ol

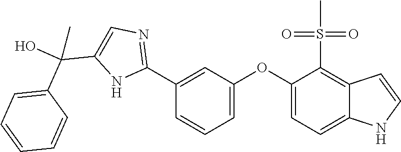 [SMILES: CC(O)(c1ccccc1)c1cnc(-c2cccc(Oc3ccc4[nH]ccc4c3S(C)(=O)=O)c2)[nH]1]

Racemic 1-(2-(3-((4-(Methylsulfonyl)-1H-indol-5-yl)oxy)phenyl)-1H-imidazol-5-yl)-1-phenylethan-1-ol (312 mg) was separated by Chiral SFC (Instrument: SFC-80 (Thar, Waters); Chiral Column: OJ 20*250 mm, 5 μm; Column temperature: 35° C.; Mobile phase: carbon dioxide/methanol {Modifier: 0.2% Ammonia (7M methonal)}=70/30; Flow rate: 80 g/min; Back pressure: 100 bar) to give the title compounds

Example 63A. Peak 1, Enantiomer 1 of 1-(2-(3-((4-(Methylsulfonyl)-1H-indol-5-yl)oxy)phenyl)-1H-imidazol-5-yl)-1-phenylethan-1-ol obtained as white solid (104.7 mg). MS m/z: 474 [M+H]⁺. ¹H NMR (400 MHz, CD₃OD) δ 7.71 (d, J=8.8 Hz, 1H), 7.63 (d, J=8.0 Hz, 1H), 7.56 (t, J=2.0 Hz, 1H), 7.51 (d, J=3.2 Hz, 1H), 7.49 (s, 1H), 7.47 (s, 1H), 7.43 (t, J=8.0 Hz, 1H), 7.32 (t, J=7.6 Hz, 2H), 7.24 (d, J=7.2 Hz, 1H), 7.21 (d, J=3.2 Hz, 1H), 7.05 (dd, J=8.0, 2.0 Hz, 1H), 6.92 (s, 1H), 6.91 (d, J=8.0 Hz, 1H), 3.35 (s, 3H), 1.90 (s, 3H) ppm.

Example 63B. Peak 2, Enantiomer 2 of 1-(2-(3-((4-(methylsulfonyl)-1H-indol-5-yl)oxy)phenyl)-1H-imidazol-5-yl)-1-phenylethan-1-ol obtained as white solid (95.8 mg). MS m/z: 474 [M+H]⁺. ¹H NMR (400 MHz, CD₃OD) δ 7.71 (d, J=8.4 Hz, 1H), 7.63 (d, J=7.6 Hz, 1H), 7.56 (t, J=1.6 Hz, 1H), 7.51 (d, J=3.2 Hz, 1H), 7.49 (s, 1H), 7.47 (s, 1H), 7.43 (t, J=8.0 Hz, 1H), 7.32 (t, J=7.6 Hz, 2H), 7.24 (d, J=7.2 Hz, 1H), 7.21 (d, J=3.2 Hz, 1H), 7.04 (dd, J=8.0, 2.0 Hz, 1H), 6.91 (d, J=8.4 Hz, 1H), 6.91 (s, 1 h), 3.35 (s, 3H), 1.90 (s, 3H) ppm.

### Example 64

### 1-(2-(3-((6-Fluoro-4-((2-(methylamino)ethyl)sulfonyl)-1H-indol-5-yl)oxy)phenyl)-1H-imidazol-5-yl)-1-phenylethan-1-ol

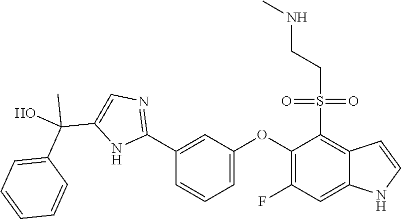 [SMILES: CNCCS(=O)(=O)c1c(Oc2cccc(-c3ncc(C(C)(O)c4ccccc4)[nH]3)c2)c(F)cc2[nH]ccc12]

### A. Benzyl (2-((6-fluoro-5-(3-(5-(1-hydroxy-1-phenylethyl)-1H-imidazol-2-yl)phenoxy)-1H-indol-4-yl)thio)ethyl)(methyl)carbamate

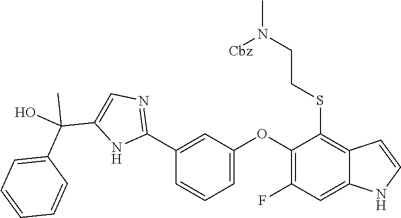 [SMILES: CN(CCSc1c(Oc2cccc(-c3ncc(C(C)(O)c4ccccc4)[nH]3)c2)c(F)cc2[nH]ccc12)C(=O)OCc1ccccc1]

Benzyl (2-((5-(3-carbamimidoylphenoxy)-6-fluoro-1H-indol-4-yl)thio)ethyl)(methyl)carbamate (Intermediate 8-6, 600 mg, 1.22 mmol) and 3-methyl-3-phenyloxirane-2-carbaldehyde (Intermediate 3, 247 mg, 1.22 mmol) were dissolved in DMF (10 mL) in a glove box. The reaction mixture was stirred at 75° C. overnight, quenched with water (50 mL) and extracted with ethyl acetate (30 mL×3). The combined organic extracts were washed with brine (40 mL), dried over sodium sulfate and concentrated. The resulting residue was purified by flash chromatography (0-100% ethyl acetate in petroleum ether) to afford the title compound as a yellow solid (295 mg, 38%). MS m/z: 637 [M+H]⁺.

### B. Benzyl (2-((6-fluoro-5-(3-(5-(1-hydroxy-1-phenylethyl)-1H-imidazol-2-yl)phenoxy)-1H-indol-4-yl)sulfonyl)ethyl)(methyl)carbamate

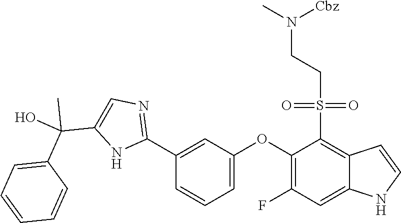 [SMILES: CN(CCS(=O)(=O)c1c(Oc2cccc(-c3ncc(C(C)(O)c4ccccc4)[nH]3)c2)c(F)cc2[nH]ccc12)C(=O)OCc1ccccc1]

To a stirred solution of benzyl (2-((6-fluoro-5-(3-(5-(1-hydroxy-1-phenylethyl)-1H-imidazol-2-yl)phenoxy)-1H-indol-4-yl)thio)ethyl)(methyl)carbamate (150 mg, 0.24 mmol) in methanol (10 mL) was added ammonium molybdate (300 mg) in hydrogen peroxide (1.5 mL, 30% in water). The reaction mixture was stirred at room temperature for 3.5 hours, diluted with ethyl acetate (30 mL), washed with water (10 mL×3), aqueous sodium sulfite (10 mL) and brine (10 mL×2), dried over sodium sulfate and concentrated. The resulting residue was purified by flash chromatography (0-100% ethyl acetate in petroleum ether) to afford the title compound as a yellow solid (57 mg, 36%). MS m/z: 669 [M+H]⁺.

### C. Example 64. 1-(2-(3-((6-Fluoro-4-((2-(methylamino)ethyl)sulfonyl)-1H-indol-5-yl)oxy)phenyl)-1H-imidazol-5-yl)-1-phenylethan-1-ol

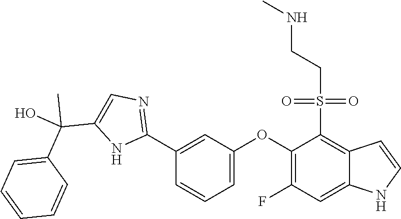 [SMILES: CNCCS(=O)(=O)c1c(Oc2cccc(-c3ncc(C(C)(O)c4ccccc4)[nH]3)c2)c(F)cc2[nH]ccc12]

To a stirred solution of benzyl (2-((6-fluoro-5-(3-(5-(1-hydroxy-1-phenylethyl)-1H-imidazol-2-yl)phenoxy)-1H-indol-4-yl)sulfonyl)ethyl)(methyl)carbamate (57 mg) in ethyl acetate (5 mL) was added 10% Pd(OH)₂ on carbon (6 mg), purged with hydrogen and stirred under hydrogen atmosphere overnight. The reaction mixture was filtered and rinsed with methanol (5 mL×2) to remove catalyst and the filtrate was concentrated. The resulting residue was purified by flash chromatography (0-10% methanol in dichloromethane) to afford the title compound as a yellow solid (12.5 mg, 27%). MS m/z: 535 [M+H]⁺. ¹H NMR (400 MHz, CD₃OD) δ 7.70 (d, J=10.4 Hz, 1H), 7.59-7.56 (m, 2H), 7.48-7.46 (m, 3H), 7.39 (t, J=8.0 Hz, 1H), 7.32 (t, J=7.6 Hz, 2H), 7.25-7.19 (m, 2H), 6.95 (dd, J=8.4, 2.4 Hz, 1H), 6.89 (s, 1H), 3.72 (t, J=7.2 Hz, 2H), 3.07 (t, J=7.2 Hz, 2H), 2.42 (s, 3H), 1.89 (s, 3H) ppm.

### Example 65

### 1-(6-Fluoro-5-(3-(4-(1-hydroxy-1-phenylethyl)-1H-imidazol-2-yl)phenoxy)-1H-indol-4-yl)ethan-1-one

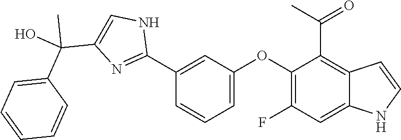 [SMILES: CC(=O)c1c(Oc2cccc(-c3nc(C(C)(O)c4ccccc4)c[nH]3)c2)c(F)cc2[nH]ccc12]

### A. 1-(2-(3-((4-(1-Ethoxyvinyl)-6-fluoro-1H-indol-5-yl)oxy)phenyl)-1H-imidazol-4-yl)-1-phenylethan-1-ol

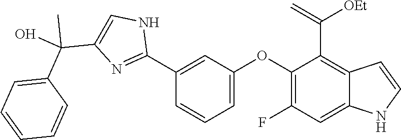 [SMILES: C=C(OCC)c1c(Oc2cccc(-c3nc(C(C)(O)c4ccccc4)c[nH]3)c2)c(F)cc2[nH]ccc12]

A solution of 3-((4-(1-ethoxyvinyl)-6-fluoro-1H-indol-5-yl)oxy)benzimidamide (Intermediate 8-7, 300 mg, 0.885 mmol), 3-methyl-3-phenyloxirane-2-carbaldehyde (Intermediate 3, 220 mg, 1.33 mmol) in DMF (10 mL) was stirred at 75° C. overnight and then concentrated. The resulting residue was purified by silica gel column chromatography (0-3.5% methanol in dichloromethane with 0.5% ammonium hydroxide) to afford the title compound as a brown solid (193 mg, 45% from two steps). MS m/z: 484 [M+H]⁺.

### B. Example 65. 1-(6-Fluoro-5-(3-(4-(1-hydroxy-1-phenylethyl)-1H-imidazol-2-yl)phenoxy)-1H-indol-4-yl)ethan-1-one

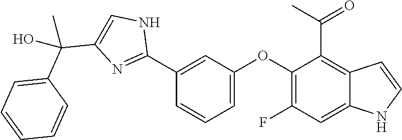 [SMILES: CC(=O)c1c(Oc2cccc(-c3nc(C(C)(O)c4ccccc4)c[nH]3)c2)c(F)cc2[nH]ccc12]

To a stirred solution of 1-(6-fluoro-5-(3-(4-(1-hydroxy-1-phenylethyl)-1H-imidazol-2-yl)phenoxy)-1H-indol-4-yl)ethan-1-one (193 mg, 0.4 mmol) in 1,4-dioxane (10 mL) was added 2M hydrochloric acid (2 mL, 4 mmol). The solution was stirred at room temperature for two hours and quenched by dropwise addition of sodium bicarbonate solution at 0° C. After water was added, the crude product was filtered and further purified by silica gel column chromatography (0-2.5% methanol in dichloromethane with 0.5% ammonium hydroxide) to afford the title compound as a light yellow solid (180 mg, 99%). MS m/z: 456 [M+H]⁺. LC-MS retention time: 0.83 minutes.

### Example 66

### 1-(2-(3-((6-Fluoro-4-(1-hydroxyethyl)-1H-indol-5-yl)oxy)phenyl)-1H-imidazol-4-yl)-1-phenylethan-1-ol

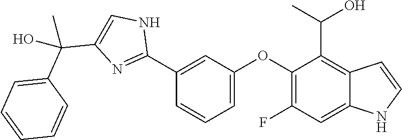 [SMILES: CC(O)c1c(Oc2cccc(-c3nc(C(C)(O)c4ccccc4)c[nH]3)c2)c(F)cc2[nH]ccc12]

A mixture of 1-(6-fluoro-5-(3-(4-(1-hydroxy-1-phenylethyl)-1H-imidazol-2-yl)phenoxy)-1H-indol-4-yl)ethan-1-one (Example 65, 80 mg, 0.176 mmol) and NaBH₄ (10 mg, 0.264 mmol) in methanol (2 mL) was stirred at room temperature for five minutes. The reaction was quenched with acetone, concentrated in vacuo and the residue was purified by column chromatography over silica gel (0-2.5% methanol in dichloromethane with 0.5% ammonium hydroxide) to afford the title compound as a white solid (50 mg, 62%). MS m/z: 458 [M+H]⁺. LC-MS retention time: 0.78 minutes.

### Example 67

### 5-(3-(3-(2,2,2-Trifluoro-1-hydroxy-1-phenylethyl)-1H-1,2,4-triazol-5-yl)phenoxy)-1H-indole-4-carboxylic acid

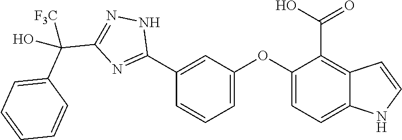 [SMILES: O=C(O)c1c(Oc2cccc(-c3nc(C(O)(c4ccccc4)C(F)(F)F)n[nH]3)c2)ccc2[nH]ccc12]

### A. Methyl 5-(3-(ethoxy(imino)methyl)phenoxy)-1H-indole-4-carboxylate

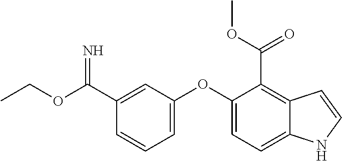 [SMILES: CCOC(=N)c1cccc(Oc2ccc3[nH]ccc3c2C(=O)OC)c1]

To a suspension of methyl 5-(3-cyanophenoxy)-1H-indole-4-carboxylate (Intermediate 1-3, 5.0 g, 17.1 mmol) in ethanol (50 mL) was added acetyl chloride (50 mL) at 0° C. The mixture was stirred at room temperature overnight and concentrated to afford the title compound as a pink solid (5.79 g, 100%). The product was used without further purification. MS m/z: 339 [M+H]⁺.

### B. Methyl 5-(3-(5-(hydroxy(phenyl)methyl)-4H-1,2,4-triazol-3-yl)phenoxy)-1H-indole-4-carboxylate

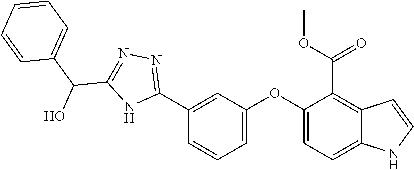 [SMILES: COC(=O)c1c(Oc2cccc(-c3nnc(C(O)c4ccccc4)[nH]3)c2)ccc2[nH]ccc12]

A mixture of methyl 5-(3-(ethoxy(imino)methyl)phenoxy)-1H-indole-4-carboxylate (13.9 g, 41.0 mmol), 2-hydroxy-2-phenylacetohydrazide (12.0 g, 74.0 mmol) and triethylamine (57 mL, 410 mmol) in ethanol (150 mL) was stirred at 80° C. overnight and then concentrated. The resulting residue was purified by silica gel column chromatography (dichloromethane/methanol, v/v, 50/1) to afford the title compound as yellow oil (12.8 g, 71%). MS m/z: 441 [M+H]⁺.

### C. Methyl 5-(3-(5-benzoyl-4H-1,2,4-triazol-3-yl)phenoxy)-1H-indole-4-carboxylate

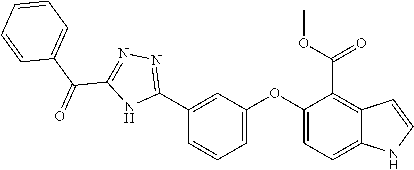 [SMILES: COC(=O)c1c(Oc2cccc(-c3nnc(C(=O)c4ccccc4)[nH]3)c2)ccc2[nH]ccc12]

A mixture of methyl 5-(3-(5-(hydroxy(phenyl)methyl)-4H-1,2,4-triazol-3-yl)phenoxy)-1H-indole-4-carboxylate (10.0 g, 22.7 mmol) and manganese dioxide (19.7 g, 227 mmol) in THF (150 mL) was stirred at room temperature for 48 hours, filtered and washed with THF and methanol. The combined filtrate was concentrated and the resulting residue was purified by silica gel column chromatography (petroleum ether/dichloromethane/ethyl acetate, v/v, 2/2/1) to afford the title compound as a yellow solid (7.2 g, 69%). MS mz: 439 [M+H]⁺.

### D. Methyl 5-(3-(3-benzoyl-1-((2-(trimethylsilyl)ethoxy)methyl)-1H-1,2,4-triazol-5-yl)phenoxy)-1-((2-(trimethylsilyl)ethoxy)methyl)-1H-indole-4-carboxylate

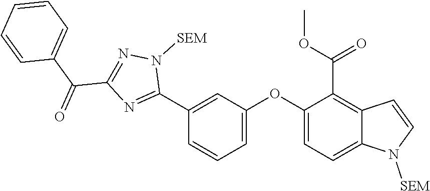 [SMILES: COC(=O)c1c(Oc2cccc(-c3nc(C(=O)c4ccccc4)nn3COCC[Si](C)(C)C)c2)ccc2c1ccn2COCC[Si](C)(C)C]

To a stirred solution of methyl 5-(3-(5-benzoyl-4H-1,2,4-triazol-3-yl)phenoxy)-1H-indole-4-carboxylate (5.0 g, 11.4 mmol) in THF (50 mL) was added sodium hydride (60% in mineral oil, 1.37 g, 34.2 mmol) at 0° C. and the mixture was stirred for one hour. SEMCl (1.7 g, 102.6 mmol) was added at 0° C. and the reaction mixture was stirred for another hour. The reaction was quenched with saturated ammonium chloride solution and extracted with ethyl acetate (50 mL×3). The combined organic extracts were washed with brine, dried over sodium sulfate and concentrated. The resulting residue was purified by silica gel column chromatography (petroleum ether/dichloromethane/ethyl acetate, v/v, 8/2/1) to afford the title compound as yellow oil (3.75 g, 47%). MS m/z: 699 [M+H]⁺. ¹H NMR (400 MHz, CD₃OD) δ 8.37 (d, J=7.2 Hz, 1H), 7.67-7.59 (m, 4H), 7.51 (t, J=8.0 Hz, 2H), 7.44 (t, J=8.0 Hz, 1H), 7.32 (d, J=2.8 Hz, 1H), 7.09 (dd, J=8.0 Hz, 1.6 Hz, 1H), 7.02 (d, J=8.8 Hz, 1H), 6.96 (d, J=3.2 Hz, 1H), 5.59 (s, 2H), 5.51 (s, 2H), 3.85 (s, 3H), 3.79 (t, J=8.0 Hz, 2H), 3.49 (t, J=8.0 Hz, 2H), 0.84-0.93 (m, 4H), 0.01 (s, 9H), −0.08 (s, 9H) ppm.

### E. Methyl 5-(3-(3-(2,2,2-trifluoro-1-hydroxy-1-phenylethyl)-1-((2-(trimethylsilyl)ethoxy)methyl)-1H-1,2,4-triazol-5-yl)phenoxy)-1-((2-(trimethylsilyl)ethoxy)methyl)-1H-indole-4-carboxylate

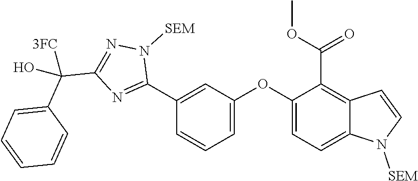

A mixture of methyl 5-(3-(3-benzoyl-1-((2-(trimethylsilyl)ethoxy)methyl)-1H-1,2,4-triazol-5-yl)phenoxy)-1-((2-(trimethylsilyl)ethoxy)methyl)-1H-indole-4-carboxylate (700 mg, 1.0 mmol), trimethyl(trifluoromethyl)silane (426 mg, 3.0 mmol) and potassium carbonate (83 mg, 0.6 mmol) in DMF (30 mL) was stirred at room temperature under nitrogen atmosphere overnight, quenched with water and extracted with ethyl acetate (50 mL×3). The combined organic extracts were washed with brine, dried over sodium sulfate and concentrated. The resulting residue was purified by silica gel column chromatography (petroleum ether/ethyl acetate, v/v, 8/1) to afford the title compound as a yellow solid (510 mg, 66%). MS m/z: 769 [M+H]⁺.

### F. Methyl 5-(3-(3-(2,2,2-trifluoro-1-hydroxy-1-phenylethyl)-1H-1,2,4-triazol-5-yl)phenoxy)-1H-indole-4-carboxylate

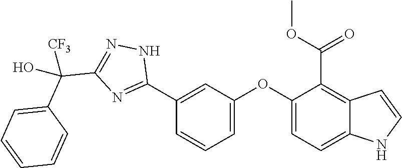 [SMILES: COC(=O)c1c(Oc2cccc(-c3nc(C(O)(c4ccccc4)C(F)(F)F)n[nH]3)c2)ccc2[nH]ccc12]

A mixture of methyl 5-(3-(3-(2,2,2-trifluoro-1-hydroxy-1-phenylethyl)-1-((2-(trimethylsilyl)ethoxy)methyl)-1H-1,2,4-triazol-5-yl)phenoxy)-1-((2-(trimethylsilyl)ethoxy)methyl)-1H-indole-4-carboxylate (510 mg, 0.66 mmol), tetrabutylammonium fluoride (1M in THF, 6.6 mL, 6.6 mmol) in THF (20 mL) was stirred at 80° C. overnight and concentrated to remove THF. Water (5 mL) was added and acidified with 1N hydrochloric acid to pH ˜2. The precipitate was collected by filtration and purified by silica column chromatography (dichloromethane/methanol, v/v, 5/1) afford the title compound as a yellow solid (200 mg, 59%). MS-ESI m/z: 509 [M+H]⁺.

### G. Example 67. 5-(3-(3-(2,2,2-Trifluoro-1-hydroxy-1-phenylethyl)-1H-1,2,4-triazol-5-yl)phenoxy)-1H-indole-4-carboxylic acid

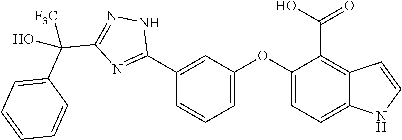 [SMILES: O=C(O)c1c(Oc2cccc(-c3nc(C(O)(c4ccccc4)C(F)(F)F)n[nH]3)c2)ccc2[nH]ccc12]

To a stirred solution of methyl 5-(3-(3-(2,2,2-trifluoro-1-hydroxy-1-phenylethyl)-1H-1,2,4-triazol-5-yl)phenoxy)-1H-indole-4-carboxylate (185 mg, 0.36 mmol) in THF (15 mL) and water (5 mL) was added lithium hydroxide (38 mg, 1.0 mmol). The mixture was stirred at room temperature overnight, acidified with 1M hydrochloric acid to pH ˜4, diluted with water and extracted with ethyl acetate (50 mL×3). The combined organic extracts were washed with brine, dried over sodium sulfate and concentrated. The resulting residue was purified by preparative HPLC to afford the title compound as a white solid (80 mg, 44%). MS-ESI m/z: 495 [M+H]⁺. ¹H NMR (400 MHz, DMSO-d₆) δ 11.32 (br s, 1H), 7.90 (br s, 1H), 7.63-7.61 (m, 3H), 7.53-7.45 (m, 3H), 7.41-7.36 (m, 4H), 6.89 (dd, J=8.0 Hz, 2.0 Hz, 1H), 6.82 (d, J=8.4 Hz, 1H), 6.68 (s, 1H) ppm.

### Example 68

### 5-(3-(3-(2,2,2-Trifluoro-1-hydroxy-1-phenylethyl)-1H-1,2,4-triazol-5-yl)phenoxy)-1H-indole-4-carboxamide

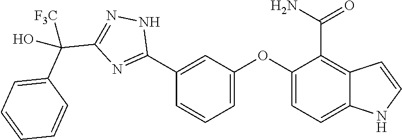 [SMILES: NC(=O)c1c(Oc2cccc(-c3nc(C(O)(c4ccccc4)C(F)(F)F)n[nH]3)c2)ccc2[nH]ccc12]

A mixture of 5-(3-(3-(2,2,2-trifluoro-1-hydroxy-1-phenylethyl)-1H-1,2,4-triazol-5-yl)phenoxy)-1H-indole-4-carboxylic acid (50 mg, 0.13 mmol), HATU (247 mg, 0.65 mmol), ammonium chloride (35 mg, 0.65 mmol) and triethylamine (66 mg, 0.65 mmol) in DMF (15 mL) was stirred at room temperature for three hours, diluted with water and extracted with ethyl acetate (30 mL×3). The combined organic extracts were washed with brine, dried over sodium sulfate and concentrated. The resulting residue was purified by preparative HPLC to afford the title compound as a white solid (31 mg, 62%). MS m/z: 494 [M+H]⁺. ¹H NMR (400 MHz, DMSO-d₆) δ 14.48 (s, 1H), 11.31 (s, 1H), 7.33-7.71 (m, 13H), 6.96 (d, J=7.2 Hz, 1H), 6.82 (d, J=8.8 Hz, 1H), 6.62 (s, 1H) ppm.

### Example 69

### 1-(2-(3-((7-Fluoro-1H-indol-5-yl)oxy)phenyl)-1H-imidazol-5-yl)-1-phenylethan-1-ol

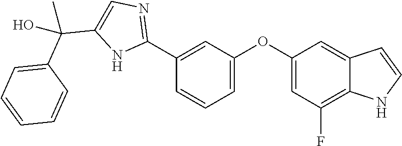 [SMILES: CC(O)(c1ccccc1)c1cnc(-c2cccc(Oc3cc(F)c4[nH]ccc4c3)c2)[nH]1]

A mixture of 3-((7-fluoro-1H-indol-5-yl)oxy)benzimidamide (Intermediate 8-10, 110 mg, 0.41 mmol) and 3-methyl-3-phenyloxirane-2-carbaldehyde (Intermediate 3, 73 mg, 0.45 mmol) in DMF (2 mL) was heated at 70° C. in a glove box for 18 hours, cooled to room temperature and extracted with ethyl acetate (25 mL×3). The combined organic phases were dried over sodium sulfate, concentrated and the resulting residue was purified by silica gel column chromatography (dichloromethane/methanol, v/v, 30/1) to afford the title compound as a white solid (64 mg, 38%). MS m/z: 414 [M+H]⁺. Retention time: 0.87 minutes.

### Example 70

### 5-((6-fluoro-5-(4-fluoro-3-(4-(1-hydroxy-1-phenylethyl)-1H-imidazol-2-yl)phenoxy)-1H-indol-4-yl)methyl)-3-methylthiazolidine-2,4-dione

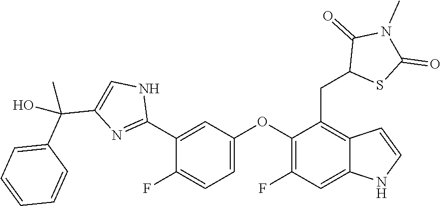 [SMILES: CN1C(=O)SC(Cc2c(Oc3ccc(F)c(-c4nc(C(C)(O)c5ccccc5)c[nH]4)c3)c(F)cc3[nH]ccc23)C1=O]

To a reaction tube were added tetrabutylammonium acetate (3 g), 6-fluoro-5-(4-fluoro-3-(4-(1-hydroxy-1-phenylethyl)-1H-imidazol-2-yl)phenoxy)-1H-indole-4-carbaldehyde (Example 1 Step B, 200 mg, 436 ummol) and 3-methylthiazolidine-2,4-dione (124 mg, 1 mmol). The reaction mixture was heated to 120° C. until melted and then stirred at 100° for three hours. The mixture was cooled to room temperature, diluted water (20 mL) and extracted with ethyl acetate (50 mL×2). The combined organic extracts were washed with brine (20 mL×3), dried over sodium sulfate, filtered and concentrated. The resulting residue was purified by flash chromatography (0-100% ethyl acetate in petroleum ether) to give 5-((6-fluoro-5-(4-fluoro-3-(4-(1-hydroxy-1-phenylethyl)-1H-imidazol-2-yl)phenoxy)-1H-indol-4-yl)methyl)-3-methylthiazolidine-2,4-dione (55 mg, 16%) as a solid.

Example 70. 5-((6-Fluoro-5-(4-fluoro-3-(4-(1-hydroxy-1-phenylethyl)-1H-imidazol-2-yl)phenoxy)-1H-indol-4-yl)methyl)-3-methylthiazolidine-2,4-dione (55 mg): MS m/z: 575 [M+H]⁺. ¹H NMR (500 MHz, CD₃OD) δ 7.51-7.44 (m, 3H), 7.35 (d, J=3.0 Hz, 1H), 7.33-7.28 (m, 2H), 7.25-7.21 (m, 2H), 7.18 (dd, J=10.5, 9.0 Hz, 1H), 6.96-6.89 (m, 2H), 6.62 (d, J=3.5 Hz, 1H), 4.84 (dd, J=10.0, 4.5 Hz, 1H), 3.79 (dd, J=14.0, 4.5 Hz, 1H), 3.40 (dd, J=14.0, 10.5 Hz, 1H), 3.04 (s, 3H), 1.88 (s, 3H) ppm.

### Example 71

### 3-(6-fluoro-5-(4-fluoro-3-(5-(1-hydroxy-1-phenylethyl)-1H-imidazol-2-yl)phenoxy)-1H-indol-4-yl)propanoic acid

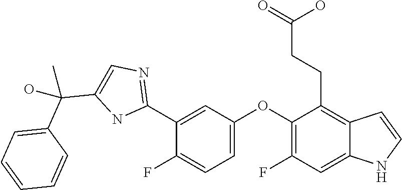 [SMILES: CC(O)(c1ccccc1)c1cnc(-c2cc(Oc3c(F)cc4[nH]ccc4c3CCC(=O)O)ccc2F)[nH]1]

### A. Ethyl (E)-3-(6-fluoro-5-(4-fluoro-3-(5-(1-hydroxy-1-phenylethyl)-1H-imidazol-2-yl)phenoxy)-1H-indol-4-yl)acrylate

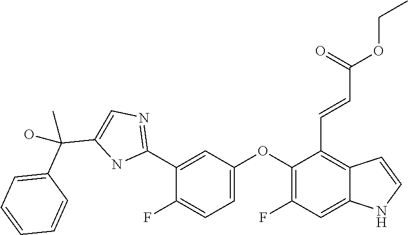 [SMILES: CCOC(=O)/C=C/c1c(Oc2ccc(F)c(-c3ncc(C(C)(O)c4ccccc4)[nH]3)c2)c(F)cc2[nH]ccc12]

To a stirred solution of 6-fluoro-5-(4-fluoro-3-(4-(1-hydroxy-1-phenylethyl)-1H-imidazol-2-yl)phenoxy)-1H-indole-4-carbaldehyde (Example 1 Step B, 0.11 g, 0.24 mmol) in toluene (10 mL) was added ethyl (triphenylphosphanylidene)acetate (0.13 g; 0.36 mmol) and the mixture was stirred at 110° C. overnight. The solvent was removed under vacuum and the residue was purified by column chromatography (0-45% ethyl acetate in petroleum ether) to afford the title compound as a solid (0.11 g, 87%). MS m/z: 530 [M+H]⁺.

### B. Ethyl 3-(6-fluoro-5-(4-fluoro-3-(5-(1-hydroxy-1-phenylethyl)-1H-imidazol-2-yl)phenoxy)-1H-indol-4-yl)propanoate

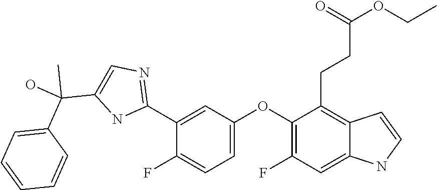

To a stirred solution of ethyl (E)-3-(6-fluoro-5-(4-fluoro-3-(5-(1-hydroxy-1-phenylethyl)-1H-imidazol-2-yl)phenoxy)-1H-indol-4-yl)acrylate (106 mg, 0.2 mmol) in methanol (10 mL) was added Pd/C (22 mg, 10% on carbon). The solution was degassed with H₂, stirred overnight at room temperature under hydrogen atmosphere, filtered through a pad of Celite and rinsed with methanol. The filtrate was concentrated and purified by flash chromatography (0-45% ethyl acetate in petroleum ether) to afford the title compound as a solid (93 mg, 88%). MS m/z: 532 [M+H]⁺.

### C. Example 71. 3-(6-Fluoro-5-(4-fluoro-3-(5-(1-hydroxy-1-phenylethyl)-1H-imidazol-2-yl)phenoxy)-1H-indol-4-yl)propanoic acid

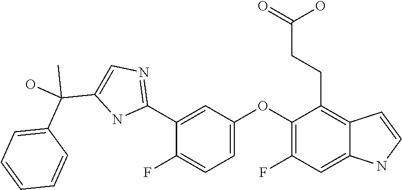

To a stirred solution of ethyl 3-(6-fluoro-5-(4-fluoro-3-(5-(1-hydroxy-1-phenylethyl)-1H-imidazol-2-yl)phenoxy)-1H-indol-4-yl)propanoate (85 mg, 0.16 mmol) in methanol (1.6 mL) and THF (4.5 mL) was added lithium hydroxide (1M in water, 1.6 mL). The reaction mixture was stirred at room temperature for two hours, acidified with 1M hydrochloric acid to pH ˜4 and diluted with ethyl acetate (70 mL). The organic phase was washed with water (15 mL×2) and brine (15 mL), dried over sodium sulfate, concentrated and lyophilized to afford the title compound as a white solid (46 mg, 57%). MS m/z: 504 [M+H]⁺. ¹H NMR (400 MHz, CD₃OD) δ 7.46-7.41 (m, 3H), 7.31-7.28 (m, 3H), 7.23-7.20 (m, 1H), 7.17-7.12 (m, 2H), 6.94 (s, 1H), 6.87 (dt, J=9.2, 3.6 Hz, 1H), 6.55 (dd, J=3.2, 0.8 Hz, 1H), 3.13 (t, J=7.6 Hz, 2H), 2.56 (t, J=7.6 Hz, 2H), 1.87 (s, 3H) ppm.

### Example 72

### 2-((2-(2-Fluoro-5-((6-fluoro-4-(methylsulfonyl)-1H-indol-5-yl)oxy)phenyl)-1H-imidazol-4-yl)(phenyl)methoxy)ethan-1-ol

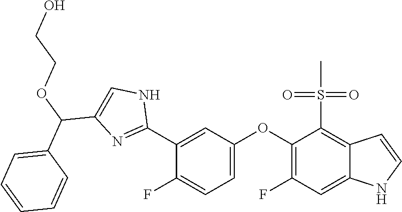 [SMILES: CS(=O)(=O)c1c(Oc2ccc(F)c(-c3nc(C(OCCO)c4ccccc4)c[nH]3)c2)c(F)cc2[nH]ccc12]

### A. (2-(2-Fluoro-5-((6-fluoro-4-(methylthio)-1H-indol-5-yl)oxy)phenyl)-1H-imidazol-5-yl)(phenyl)methanol

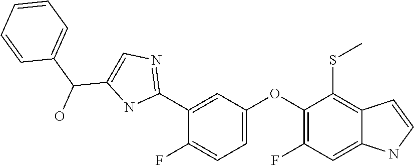 [SMILES: CSc1c(Oc2ccc(F)c(-c3ncc(C(O)c4ccccc4)[nH]3)c2)c(F)cc2[nH]ccc12]

To a stirred solution of 2-fluoro-5-((6-fluoro-4-(methylthio)-1H-indol-5-yl)oxy)benzimidamide (Intermediate 8-11, 600 mg, 1.8 mmol) in DMF (15 mL) was added 3-phenyloxirane-2-carbaldehyde (Intermediate 3-1, 266 mg, 1.8 mmol). The reaction mixture was stirred at 75° C. overnight, quenched with water and extracted with ethyl acetate. The combined organic extracts were washed with brine, dried over sodium sulfate, filtered and concentrated. The resulting residue was purified by flash chromatography (0-30% ethyl acetate in petroleum ether) to afford the title compound as yellow oil (470 mg, 56%). MS m/z: 464 [M+H]⁺.

### B. (2-(2-Fluoro-5-((6-fluoro-4-(methylsulfonyl)-1H-indol-5-yl)oxy)phenyl)-1H-imidazol-5-yl)(phenyl)methanol

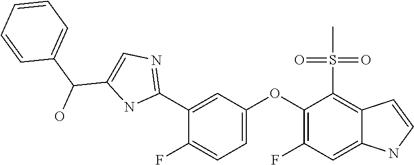 [SMILES: CS(=O)(=O)c1c(Oc2ccc(F)c(-c3ncc(C(O)c4ccccc4)[nH]3)c2)c(F)cc2[nH]ccc12]

To a stirred solution of (2-(2-fluoro-5-((6-fluoro-4-(methylthio)-1H-indol-5-yl)oxy)phenyl)-1H-imidazol-5-yl)(phenyl)methanol (470 mg, 1 mmol) in methanol (50 mL) was added a mixture of 1 g ammonium molybdate tetrahydrate in 5 mL of H₂O₂ (30% in water). The reaction mixture was stirred at room temperature for one hour, diluted with water and extracted with ethyl acetate. The combined organic extracts were washed with aqueous sodium sulfite solution and brine, dried over sodium sulfate, filtered and concentrated. The resulting residue was purified by flash chromatography (0-70% ethyl acetate in petroleum ether) to afford the title compound as a brown solid (220 mg, 40%). MS m/z: 496 [M+H]⁺.

### C. Example 72. 2-((2-(2-Fluoro-5-((6-fluoro-4-(methylsulfonyl)-1H-indol-5-yl)oxy)phenyl)-1H-imidazol-4-yl)(phenyl)methoxy)ethan-1-ol

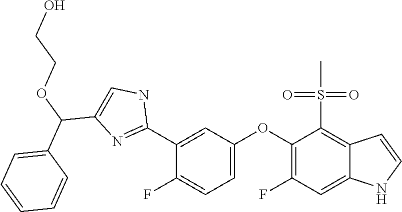 [SMILES: CS(=O)(=O)c1c(Oc2ccc(F)c(-c3nc(C(OCCO)c4ccccc4)c[nH]3)c2)c(F)cc2[nH]ccc12]

To a stirred solution of (2-(2-fluoro-5-((6-fluoro-4-(methylsulfonyl)-1H-indol-5-yl)oxy)phenyl)-1H-imidazol-5-yl)(phenyl)methanol (154 mg, 0.33 mmol) in 10 mL of ethylene glycol was added pyridinium p-toluenesulfonate (83 mg, 0.33 mmol) and the reaction mixture was stirred at room temperature over weekend.

The reaction was diluted with water and extracted with ethyl acetate. The combined organic extracts were washed with water and brine, dried over sodium sulfate, filtered and concentrated. The resulting residue was purified by silica gel column chromatography (0-80% ethyl acetate in petroleum ether) to afford the title compound as a white solid (90 mg, 45%). MS m/z: 540 [M+H]⁺. ¹H NMR (400 MHz, CD₃OD) δ 7.67 (d, J=10.4 Hz, 1H), 7.60-7.53 (m, 1H), 7.53 (d, J=3.2 Hz, 1H), 7.47-7.45 (m, 2H), 7.38 (t, J=7.2 Hz, 2H), 7.35-7.29 (m, 1H), 7.24-7.19 (m, 2H), 6.99 (dt, J=9.2, 3.6 Hz, 1H), 6.86-6.60 (m, 1H), 5.49 (s, 1H), 3.71 (t, J=4.4 Hz, 2H), 3.55 (m, 2H), 3.34 (s, 3H) ppm.

### Example 73

### 2-((2-(2-Fluoro-5-((6-fluoro-4-(methylsulfonyl)-1H-indol-5-yl)oxy)phenyl)-1H-imidazol-4-yl)(phenyl)methoxy)acetic acid

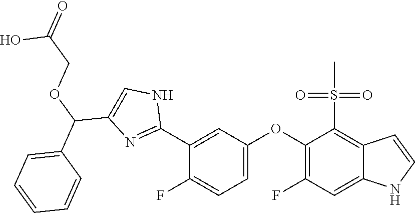 [SMILES: CS(=O)(=O)c1c(Oc2ccc(F)c(-c3nc(C(OCC(=O)O)c4ccccc4)c[nH]3)c2)c(F)cc2[nH]ccc12]

### A. Ethyl 2-((2-(2-fluoro-5-((6-fluoro-4-(methylsulfonyl)-1H-indol-5-yl)oxy)phenyl)-1H-imidazol-4-yl)(phenyl)methoxy)acetate

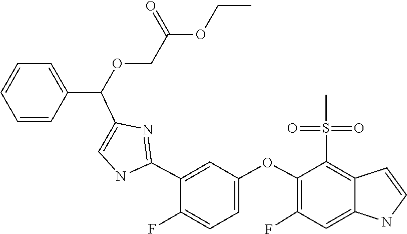

To a stirred solution of (2-(2-fluoro-5-((6-fluoro-4-(methylsulfonyl)-1H-indol-5-yl)oxy)phenyl)-1H-imidazol-5-yl)(phenyl)methanol (50 mg, 0.108 mmol) in ethyl 2-hydroxyacetate (1 mL) was added PPTS (27 mg, 0.108 mmol). The reaction mixture was stirred at 50° C. overnight, diluted with water and extracted with ethyl acetate. The combined organic extracts were washed with water and brine, dried over sodium sulfate and concentrated. The resulting residue was purified by flash chromatography (0-70% ethyl acetate in petroleum ether) to afford the title compound as yellow solid (19 mg, 30%). MS m/z: 582 [M+H]⁺.

### B. Example 73. 2-((2-(2-Fluoro-5-((6-fluoro-4-(methylsulfonyl)-1H-indol-5-yl)oxy)phenyl)-1H-imidazol-4-yl)(phenyl)methoxy)acetic acid

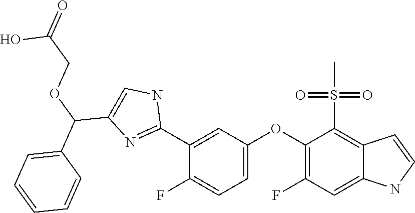

To a stirred solution of ethyl 2-((2-(2-fluoro-5-((6-fluoro-4-(methylsulfonyl)-1H-indol-5-yl)oxy)phenyl)-1H-imidazol-4-yl)(phenyl)methoxy)acetate (50 mg) in THF (1.5 mL) and methanol (0.5 mL) was added lithium hydroxide (1M in water, 0.86 mL). The mixture was stirred at room temperature for two hours, acidified with 1N hydrochloric acid to pH ˜4 and extracted with ethyl acetate (60 mL×2). The combined organic extracts were washed with brine (15 mL×3), dried over sodium sulfate, filtered and concentrated. The resulting residue was purified by flash chromatography (0-100% ethyl acetate in petroleum ether) to afford the title compound as a white solid (7 mg, 30%). MS m/z: 554 [M+H]⁺. ¹H NMR (400 MHz, CD₃OD) δ 7.67 (d, J=10.4 Hz, 1H), 7.56 (dd, J=6.0, 3.2 Hz, 1H), 7.54 (d, J=3.2 Hz, 1H), 7.49-7.47 (m, 2H), 7.42-7.39 (m, 2H), 7.37-7.36 (m, 1H), 7.26 (dd, J=10.4, 9.2 Hz, 1H), 7.20 (d, J=3.2 Hz, 1H), 7.04 (dt, J=9.2, 3.6 Hz, 1H), 6.83 (s, 1H), 5.63 (s, 1H), 4.06 (s, 2H), 3.34 (s, 3H) ppm.

### Example 74

### 2-(6-Fluoro-5-(4-fluoro-3-(5-(1-hydroxy-1-phenylethyl)-1H-imidazol-2-yl)phenoxy)-4-methyl-1H-indol-3-yl)acetic acid

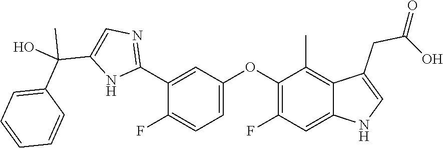 [SMILES: Cc1c(Oc2ccc(F)c(-c3ncc(C(C)(O)c4ccccc4)[nH]3)c2)c(F)cc2[nH]cc(CC(=O)O)c12]

### A. (2-(2-Fluoro-5-((6-fluoro-4-methyl-1H-indol-5-yl)oxy)phenyl)-1H-imidazol-5-yl)(phenyl)methanol

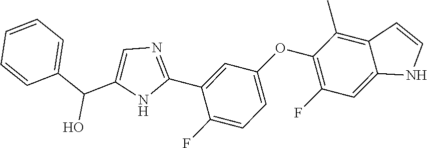 [SMILES: Cc1c(Oc2ccc(F)c(-c3ncc(C(O)c4ccccc4)[nH]3)c2)c(F)cc2[nH]ccc12]

A mixture of 2-fluoro-5-((6-fluoro-4-methyl-1H-indol-5-yl)oxy)benzimidamide (Intermediate 8-12, 1.5 g, 4.98 mmol) and 3-phenyloxirane-2-carbaldehyde (Intermediate 3-1, 811 mg, 5.48 mmol) in DMF (10 ml) was stirred at 75° C. overnight and cooled to room temperature. The mixture was diluted with ethyl acetate (100 mL), washed with water and brine, dried over sodium sulfate, filtered and concentrated. The resulting residue was purified by silica gel column chromatography (petroleum ether/ethyl acetate, v/v, 2/1-1/1) to afford the title compound as a light yellow solid (1.4 g, 34%). MS m/z: 432 [M+H]⁺.

### B. (2-(2-Fluoro-5-((6-fluoro-4-methyl-1H-indol-5-yl)oxy)phenyl)-1H-imidazol-5-yl)(phenyl)methanone

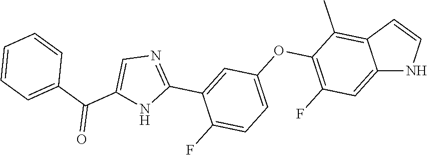 [SMILES: Cc1c(Oc2ccc(F)c(-c3ncc(C(=O)c4ccccc4)[nH]3)c2)c(F)cc2[nH]ccc12]

To a stirred solution of (2-(2-fluoro-5-((6-fluoro-4-methyl-1H-indol-5-yl)oxy)phenyl)-1H-imidazol-5-yl)(phenyl)methanol (1.40 g, 1.35 mmol) in THF (20 mL) was added manganese dioxide (1.18 g, 13.5 mmol) at room temperature. The resulting mixture was stirred at room temperature overnight and filtered through a pad of Celite. The filtrate was concentrated and the resulting residue was purified by silica gel column chromatography (petroleum ether/ethyl acetate, v/v, 3/1-2/1) to afford the title compound as a light yellow solid (900 mg, 83%). MS m/z: 430 [M+H]⁺.

### C. 5-(3-(5-Benzoyl-1H-imidazol-2-yl)-4-fluorophenoxy)-6-fluoro-4-methyl-1H-indole-3-carbaldehyde

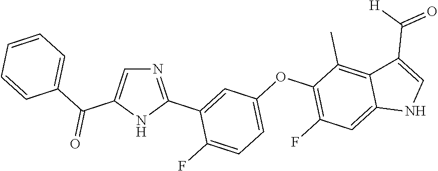 [SMILES: Cc1c(Oc2ccc(F)c(-c3ncc(C(=O)c4ccccc4)[nH]3)c2)c(F)cc2[nH]cc(C=O)c12]

To a stirred solution of DMF (3 mL) was added phosphorus(V) oxychloride (510 mg, 3.36 mmol) dropwise at 0° C. The reaction mixture was stirred at 0° C. for 20 minutes and then a solution of (2-(2-fluoro-5-((6-fluoro-4-methyl-1H-indol-5-yl)oxy)phenyl)-1H-imidazol-5-yl)(phenyl)methanone (1.2 g, 2.80 mmol) in DMF (3 mL) was added dropwise. The resulting mixture was stirred at room temperature overnight, poured into ice water and neutralized with 20% aqueous sodium hydroxide. The mixture was diluted with ethyl acetate (60 mL), washed with water (50 mL) and brine (50 mL), dried over sodium sulfate and concentrated. The resulting residue was purified by silica gel column chromatography (petroleum ether/ethyl acetate, v/v, 3/1-2/1) to afford the title compound as a light yellow solid (1.08 g, 85%). MS m/z: 458 [M+H]⁺.

### D. (Z)-(2-(2-Fluoro-5-((6-fluoro-3-(2-methoxyvinyl)-4-methyl-1H-indol-5-yl)oxy)phenyl)-1H-imidazol-5-yl)(phenyl)methanone

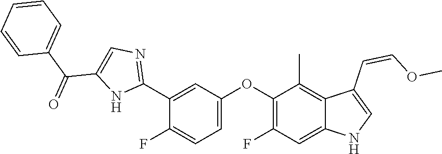 [SMILES: CO/C=C\c1c[nH]c2cc(F)c(Oc3ccc(F)c(-c4ncc(C(=O)c5ccccc5)[nH]4)c3)c(C)c12]

To a stirred solution of triphenyl(methoxymethyl)phosphonium chloride (1.80 g, 5.25 mmol) in THF (10 mL) was added n-butyllithium (2.3 mL, 2.5 N in hexane, 5.75 mmol) dropwise at 0° C. The reaction mixture was stirred at 0° C. for 30 minutes and a solution of 5-(3-(5-benzoyl-1H-imidazol-2-yl)-4-fluorophenoxy)-6-fluoro-4-methyl-1H-indole-3-carbaldehyde (1.2 g, 2.63 mmol) in THF (5 mL) was added dropwise. The reaction mixture was refluxed overnight, cooled to room temperature, quenched with saturated ammonium chloride (30 mL) and extracted with ethyl acetate (50 mL×3). The combined organic extracts were washed with water (50 mL) and brine (30 mL), dried over sodium sulfate, filtered and concentrated. The resulting residue was purified by silica gel column chromatography (petroleum ether/ethyl acetate, v/v, 3/1-2/1) to afford the title compound as a light yellow solid (220 mg, 26%). MS m/z: 486 [M+H]⁺.

### E. 2-(5-(3-(5-Benzoyl-1H-imidazol-2-yl)-4-fluorophenoxy)-6-fluoro-4-methyl-1H-indol-3-yl)acetaldehyde

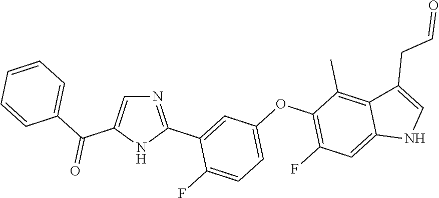 [SMILES: Cc1c(Oc2ccc(F)c(-c3ncc(C(=O)c4ccccc4)[nH]3)c2)c(F)cc2[nH]cc(CC=O)c12]

To a stirred solution of (Z)-(2-(2-fluoro-5-((6-fluoro-3-(2-methoxyvinyl)-4-methyl-1H-indol-5-yl)oxy)phenyl)-1H-imidazol-5-yl)(phenyl)methanone (220 mg, 0.45 mmol) in THF (5 mL) was added 1N hydrochloric acid (4.5 mL, 4.5 mmol). The resulting mixture was stirred at 60° C. for one hour, quenched with saturated sodium bicarbonate solution (30 mL) and extracted with ethyl acetate (30 mL×2). The combined organic extracts were washed with water (50 mL) and brine (50 mL), dried over sodium sulfate, filtered and concentrated to afford the title compound as a yellow solid (180 mg, 84%). The product was used without further purification. MS m/z: 472 [M+H]⁺.

### F. 2-(5-(3-(5-Benzoyl-1H-imidazol-2-yl)-4-fluorophenoxy)-6-fluoro-4-methyl-1H-indol-3-yl)acetic acid

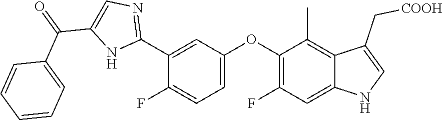 [SMILES: Cc1c(Oc2ccc(F)c(-c3ncc(C(=O)c4ccccc4)[nH]3)c2)c(F)cc2[nH]cc(CC(=O)O)c12]

To a stirred solution of 2-(5-(3-(5-benzoyl-1H-imidazol-2-yl)-4-fluorophenoxy)-6-fluoro-4-methyl-1H-indol-3-yl)acetaldehyde (180 mg, 0.38 mmol) in t-Butanol (5 mL) were added 2-methyl-2-butene (1.34 g, 19.1 mmol), KH₂PO₄ (363 mg, 2.68 mmol) and NaClO₂ (310 mg, 3.44 mmol) and stirred at room temperature overnight. The mixture was concentrated in vacuo to give the residue, which was purified by reverse phase chromatography to afford the title compound as a white solid (113 mg, 61%). MS m/z: 488 (M+H⁺).

### G. Example 74. 2-(6-Fluoro-5-(4-fluoro-3-(5-(1-hydroxy-1-phenylethyl)-1H-imidazol-2-yl)phenoxy)-4-methyl-1H-indol-3-yl)acetic acid

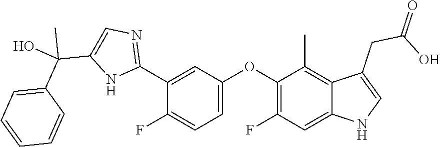 [SMILES: Cc1c(Oc2ccc(F)c(-c3ncc(C(C)(O)c4ccccc4)[nH]3)c2)c(F)cc2[nH]cc(CC(=O)O)c12]

To a stirred solution of 2-(5-(3-(5-benzoyl-1H-imidazol-2-yl)-4-fluorophenoxy)-6-fluoro-4-methyl-1H-indol-3-yl)acetic acid (113 mg, 0.23 mmol) in THF (10 mL) was added a 3.0 N solution of methylmagnesium bromide in THF (0.8 mL, 2.4 mmol) at 0° C. The resulting mixture was stirred at room temperature for one hour, quenched with saturated ammonium chloride solution (10 mL) and extracted with ethyl acetate (35 mL×3). The combined organic extracts were washed with brine (10 mL), dried over sodium sulfate, filtered and concentrated. The resulting residue was purified by preparative HPLC to afford the title compound as a white solid (16.8 mg, 16%). MS m/z: 504 [M+H]⁺. ¹H NMR (400 MHz, CD₃OD) δ 7.47 (d, J=7.6 Hz, 2H), 7.43-7.39 (m, 1H), 7.32 (t, J=7.6 Hz, 2H), 7.27-7.21 (m, 1H), 7.19-7.11 (m, 2H), 7.06 (d, J=10.4 Hz, 1H), 6.96 (s, 1H), 6.89-6.81 (m, 1H), 3.87 (s, 2H), 2.50 (s, 3H), 1.89 (s, 3H) ppm.

### Example 75

### 2-(6-Fluoro-5-(4-fluoro-3-(5-(2-phenylpropan-2-yl)-1H-imidazol-2-yl)phenoxy)-1H-indol-4-yl)ethane-1-sulfonamide

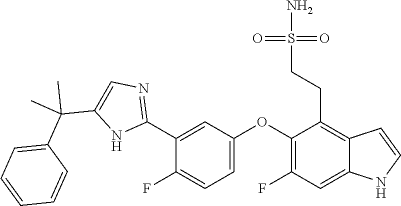 [SMILES: CC(C)(c1ccccc1)c1cnc(-c2cc(Oc3c(F)cc4[nH]ccc4c3CCS(N)(=O)=O)ccc2F)[nH]1]

### A. (E)-2-(6-fluoro-5-(4-fluoro-3-(5-(2-phenylpropan-2-yl)-1H-imidazol-2-yl)phenoxy)-1H-indol-4-yl)ethene-1-sulfonamide

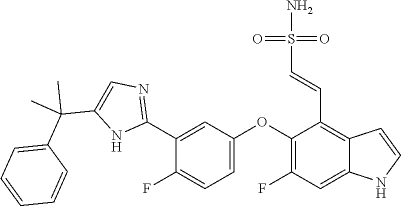 [SMILES: CC(C)(c1ccccc1)c1cnc(-c2cc(Oc3c(F)cc4[nH]ccc4c3/C=C/S(N)(=O)=O)ccc2F)[nH]1]

A solution of 4-bromo-6-fluoro-5-(4-fluoro-3-(5-(2-phenylpropan-2-yl)-1H-imidazol-2-yl)phenoxy)-1H-indole (Example 10 Step A, 300 mg, 590 μmol), triethylamine (823 μl, 5.90 mmol) in 1.2 mL of DMF was degassed for 10 minutes. Tri-o-tolylphosphine (36 mg, 118 μmol), palladium (II) acetate (7 mg, 30 μmol) and ethenesulfonamide (76 mg, 708 μmol) were added And the reaction mixture was heated at 145° C. for 30 minutes in a microwave reactor. The solvent was evaporated and the residue was chromatographed over silica gel (0-100% ethyl acetate in heptane) to afford the title compound as a yellow solid (0.2 g, 63%).

### B. Example 75. 2-(6-Fluoro-5-(4-fluoro-3-(5-(2-phenylpropan-2-yl)-1H-imidazol-2-yl)phenoxy)-1H-indol-4-yl)ethane-1-sulfonamide

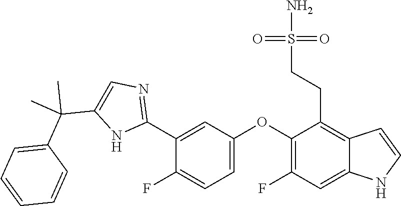 [SMILES: CC(C)(c1ccccc1)c1cnc(-c2cc(Oc3c(F)cc4[nH]ccc4c3CCS(N)(=O)=O)ccc2F)[nH]1]

A mixture of (E)-2-(6-fluoro-5-(4-fluoro-3-(5-(2-phenylpropan-2-yl)-1H-imidazol-2-yl)phenoxy)-1H-indol-4-yl)ethene-1-sulfonamide (0.20 g, 374 μmol) and Pd—C (159 mg, 75 μmol) in 5 mL of ethyl acetate was stirred under hydrogen balloon for five hours, filtered through a pad of Celite and washed with ethyl acetate. The filtrate was evaporated and the residue was chromatographed over 0-50% ethyl acetate in heptane to afford the title compound as a white solid (85 mg, 42%). MS m/z: 537 [M+H]⁺. ¹H NMR (400 MHz, CD₃OD) δ 7.46-7.05 (m, 9H), 6.91 (s, 1H), 6.84 (d, J=8.7 Hz, 1H), 6.57 (dd, J=3.3, 0.9 Hz, 1H), 3.39-3.22 (m, 4H), 1.67 (s, 6H) ppm.

### Example 76

### 2-(2-(5-((4,6-Difluoro-1H-indol-5-yl)oxy)-2-fluorophenyl)-1H-imidazol-4-yl)-2-phenylpropanoic acid

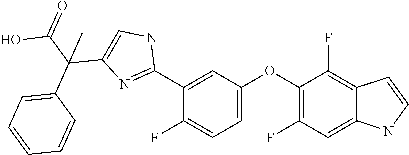

### A. 2-(2-(5-((4,6-Difluoro-1H-indol-5-yl)oxy)-2-fluorophenyl)-1H-imidazol-4-yl)-2-phenylpropyl acetate

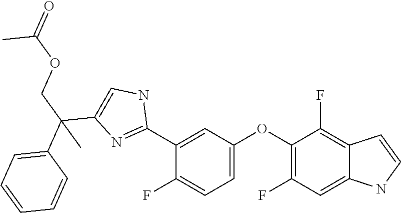

To a solution of 5-((4,6-difluoro-1H-indol-5-yl)oxy)-2-fluorobenzimidamide (Intermediate 8-9, 0.46 g, 1.5 mmol) and 4-bromo-2-methyl-3-oxo-2-phenylbutyl acetate (0.48 g, 1.6 mmol) in acetonitrile (6 mL) in a glove box was added sodium bicarbonate (0.25 g, 3.0 mmol). The mixture was stirred at 75° C. overnight, quenched with water and extracted with ethyl acetate. The combined extracts were washed with brine, dried over sodium sulfate, filtered and concentrated. The residue was purified by flash chromatography (0-50% ethyl acetate in petroleum ether) to afford the title compound as a solid (0.46 g, 60%). MS m/z: 506 [M+H]⁺.

### B. 2-(2-(5-((4,6-Difluoro-1H-indol-5-yl)oxy)-2-fluorophenyl)-1H-imidazol-4-yl)-2-phenylpropan-1-ol

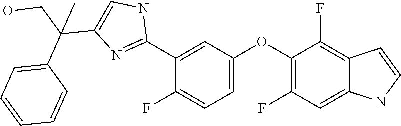

To a stirred solution of 2-(2-(5-((4,6-difluoro-1H-indol-5-yl)oxy)-2-fluorophenyl)-1H-imidazol-4-yl)-2-phenylpropyl acetate (0.44 g, 0.87 mmol) in methanol (3.5 mL) and THF (10 mL) was added a 1M solution of lithium hydroxide in water (3.5 mL). The reaction mixture was stirred at room temperature for two hours, acidified with 1M hydrochloric acid to pH ˜ 4 and diluted with ethyl acetate (90 mL). The mixture was washed with water (25 mL×2) and brine (15 mL), dried over sodium sulfate, filtered and concentrated to afford the title compound as a white solid (0.398 g, 99%). The product was used in the next step without further purification. MS m/z: 464 [M+H]⁺.

### C. 2-(2-(5-((4,6-Difluoro-1H-indol-5-yl)oxy)-2-fluorophenyl)-1H-imidazol-4-yl)-2-phenylpropanal

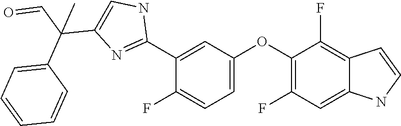

To a stirred solution of 2-(2-(5-((4,6-difluoro-1H-indol-5-yl)oxy)-2-fluorophenyl)-1H-imidazol-4-yl)-2-phenylpropan-1-ol (0.394 g, 0.85 mmol) in dichloromethane (20 mL) was added Dess-Martin periodinane (0.394 g, 0.94 mmol) in portions at 0° C. The reaction mixture was stirred at room temperature for two hours, diluted with dichloromethane and washed with saturated sodium sulfite and sodium bicarbonate solutions. The organic phase was dried over sodium sulfate, filtered and concentrated. The resulting residue was purified by silica gel column (0-60% ethyl acetate in petroleum ether) to afford the title compound as a white solid (0.23 g, 58%). MS m/z: 462 [M+H]⁺.

### D. Example 76. 2-(2-(5-((4,6-Difluoro-1H-indol-5-yl)oxy)-2-fluorophenyl)-1H-imidazol-4-yl)-2-phenylpropanoic acid

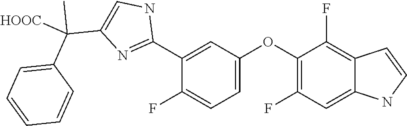

To a stirred solution of 2-(2-(5-((4,6-difluoro-1H-indol-5-yl)oxy)-2-fluorophenyl)-1H-imidazol-4-yl)-2-phenylpropanal (0.21 g, 0.45 mmol) and 2-methyl-2-butene (0.53 mL, 6.3 mmol) in t-butanol (15 mL) was added dropwise a solution of sodium chlorite (0.16 g, 1.8 mmol) and sodium phosphate dihydrogen monohydrate (0.27 g, 2.25 mmol) in water (3 mL) and the reaction mixture was stirred at room temperature for 1.5 hours. The mixture was diluted with dichloromethane (70 mL) and washed with saturated sodium sulfite and saturated sodium bicarbonate solutions. The organic phase was dried over sodium sulfate, filtered and concentrated. The resulting residue was purified with preparative HPLC to afford the title compound as a white solid (74 mg, 35%). MS m/z: 478 [M+H]⁺. ¹H NMR (400 MHz, CD₃OD) δ 7.51 (dd, J=6.0, 3.2 Hz, 1H), 7.30-7.29 (m, 5H), 7.26-7.19 (m, 2H), 7.13 (d, J=10.0 Hz, 1H), 6.99 (dt, J=9.2, 3.6 Hz, 1H), 6.91 (s, 1H), 6.54 (d, J=2.8 Hz, 1H), 1.90 (s, 3H) ppm.

### Example 77

### 2-(2-(2-Fluoro-5-((6-fluoro-4-methyl-1H-indol-5-yl)oxy)phenyl)-1H-imidazol-4-yl)-2-phenylpropan-1-ol

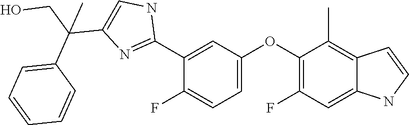

The title compound was prepared from 2-fluoro-5-((6-fluoro-4-methyl-1H-indol-5-yl)oxy)benzimidamide (Intermediate 8-12) and 4-bromo-2-methyl-3-oxo-2-phenylbutyl acetate (Intermediate 4-1) as described for Example 76, steps A to B. MS m/z: 460 [M+H]⁺. Retention time: 0.94 min. ¹H NMR (400 MHz, CD₃OD) δ 7.43-7.41 (m, 1H), 7.27-7.25 (m, 5H), 7.19-7.16 (m, 1H), 7.14-7.07 (m, 2H), 6.93 (s, 1H), 6.81-6.78 (m, 1H), 6.50 (d, J=3.2 Hz, 1H), 4.14 (d, J=10.8 Hz, 1H), 3.91 (d, J=10.8 Hz, 1H), 2.37 (s, 3H), 1.66 (s, 3H) ppm.

### Example 78

### 2-(5-((4,6-Difluoro-1H-indol-5-yl)oxy)-2-fluorophenyl)-5-(2-fluorobenzyl)-1H-imidazole-4-carboxylic acid

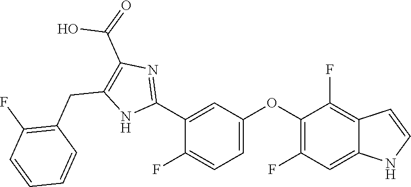 [SMILES: O=C(O)c1nc(-c2cc(Oc3c(F)cc4[nH]ccc4c3F)ccc2F)[nH]c1Cc1ccccc1F]

### A. Methyl 4-(3-bromo-2-fluorophenyl)-3-oxobutanoate

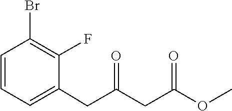 [SMILES: COC(=O)CC(=O)Cc1cccc(Br)c1F]

To a stirred solution of potassium; 3-ethoxy-3-oxo-propanoate (7.67 g, 45.1 mmol) in acetonitrile (100 mL) were added MgCl₂ (5.11 g, 53.6 mmol) and triethylamine (6.95 g, 68.7 mmol) and the mixture was stirred at room temperature for two hours (solution A). To a solution of 2-(3-bromo-2-fluoro-phenyl)acetic acid (5 g, 21.5 mmol) in acetonitrile (50 mL) was added carbonyldiimidazole (3.83 g, 23.6 mmol) at room temperature and the mixture was stirred for 1.5 hours. To the reaction mixture was added the solution A prepared above at room temperature, stirred for 30 minutes and then heated at 80° C. for two hours. The reaction mixture was cooled to room temperature and acidified with 2N hydrochloric acid solution (80 mL). The mixture was diluted with ethyl acetate (300 mL), washed with saturated sodium hydrogencarbonate solution and, brine, dried over magnesium sulfate and concentrated. The crude product was purified by column chromatography on silica gel (petroleum ether/ethyl acetate, v/v, 1/0 to 6/1) to afford the title compound as a liquid (6 g, 97%). MS m/z: 289/291 [M+H]⁺.

### B. Methyl 4-(3-bromo-2-fluorophenyl)-2-(hydroxyimino)-3-oxobutanoate

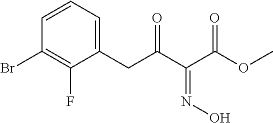 [SMILES: COC(=O)/C(=N\O)C(=O)Cc1cccc(Br)c1F]

A solution of sodium nitrite (1.2 g, 17.4 mmol) in water (25 mL) was added to a solution of methyl 4-(3-bromo-2-fluoro-phenyl)-3-oxo-butanoate (5 g, 16.6 mmol) in acetic acid (50 mL) at 0° C. The mixture was stirred at 0° C. for three hours, diluted with ethyl acetate (300 mL), washed with saturated sodium bicarbonate (100 mL×5) and brine (200 mL), dried over sodium sulfate, filtered and concentrated. The resulting residue was purified by flash chromatography (0-30% ethyl acetate in petroleum ether) to afford the title compound as a yellow oil (3.9 g, 70%). MS m/z: 340/342 [M+Na]⁺.

### C. [5-[(4,6-Difluoro-1H-indol-5-yl)oxy]-2-fluoro-phenyl]methanamine

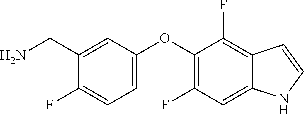 [SMILES: NCc1cc(Oc2c(F)cc3[nH]ccc3c2F)ccc1F]

To a stirred solution of 5-((4,6-difluoro-1H-indol-5-yl)oxy)-2-fluoro-benzonitrile (Intermediate 15, 5 g, 17.3 mmol) in THF (100 mL) were added ammonium hydroxide (28% in water, 10 mL) and Raney Ni (5 g). The reaction mixture was stirred overnight at room temperature under hydrogen atmosphere and then filtered. The filtrate was concentrated and the resulting residue was purified by flash chromatography (0-10% methanol in dichloromethane) to afford the title compound as a yellow solid (4.13 g, 78%). MS m/z: 293 [M+H]⁺.

### D. Methyl 5-(3-Bromo-2-fluorobenzyl)-2-(5-((4,6-difluoro-1H-indol-5-yl)oxy)-2-fluorophenyl)-1H-imidazole-4-carboxylate

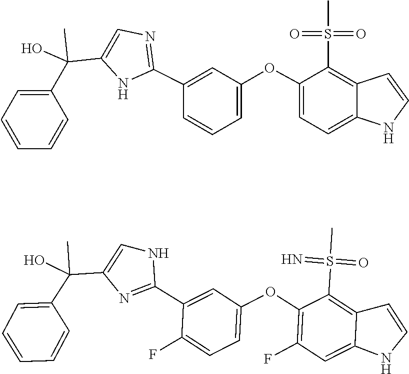 [SMILES: CC(O)(c1ccccc1)c1cnc(-c2cccc(Oc3ccc4[nH]ccc4c3S(C)(=O)=O)c2)[nH]1 ; CC(O)(c1ccccc1)c1c[nH]c(-c2cc(Oc3c(F)cc4[nH]ccc4c3S(C)(=N)=O)ccc2F)n1]

A solution of methyl 4-(3-bromo-2-fluoro-phenyl)-2-hydroxyimino-3-oxo-butanoate (3.2 g, 9.56 mmol) and [5-[(4,6-difluoro-1H-indol-5-yl)oxy]-2-fluoro-phenyl]methanamine (Example 78 Step C, 2.91 g, 9.56 mmol) in acetonitrile (120 mL) was heated to reflux overnight. The reaction mixture was concentrated and the residue was purified by flash chromatography (0-30% ethyl acetate in petroleum ether) to afford the title compound as a yellow oil (2 g, 34%). MS m/z: 574, 576 [M+H]⁺.

### E. 5-(3-Bromo-2-fluorobenzyl)-2-(5-((4,6-difluoro-1H-indol-5-yl)oxy)-2-fluorophenyl)-1H-imidazole-4-carboxylic acid

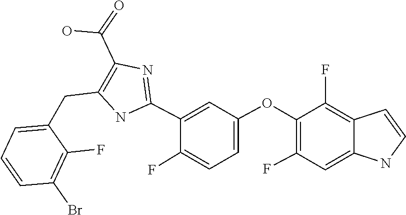

To a stirred solution of methyl 5-[(3-bromo-2-fluoro-phenyl)methyl]-2-[5-[(4,6-difluoro-1H-indol-5-yl)oxy]-2-fluoro-phenyl]-1H-imidazole-4-carboxylate (1.7 g, 2.78 mmol) in THF (87 mL) and methanol (29 mL) was added a solution of lithium hydroxide (0.66 g, 27.8 mmol) in water (29 mL). The reaction mixture was stirred at 50° C. for four days, cooled to room temperature, diluted with water (40 mL), acidified with 1N hydrochloric acid to pH 4-5 and extracted with ethyl acetate (50 mL×4). The combined organic extracts were washed with brine, dried over sodium sulfate, filtered and concentrated to afford the title compound as a yellow solid (1.47 g, 79%). The product was used in the next step without further purification. MS m/z: 560 [M+H]⁺.

### F. Example 78. 2-(5-((4,6-Difluoro-1H-indol-5-yl)oxy)-2-fluorophenyl)-5-(2-fluorobenzyl)-1H-imidazole-4-carboxylic acid

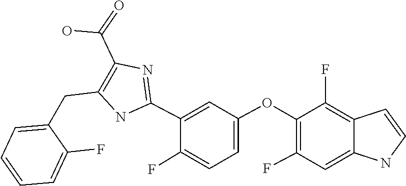

To a stirred solution of 5-[(3-bromo-2-fluoro-phenyl)methyl]-2-[5-[(4,6-difluoro-1H-indol-5-yl)oxy]-2-fluoro-phenyl]-1H-imidazole-4-carboxylic acid (0.3 g, 0.535 mmol) in ethyl acetate (20 mL) was added Pd/C (1 g, 5% on carbon, wet). The reaction mixture was stirred at 45° C. under hydrogen atmosphere for three hours, filtered and rinsed with ethyl acetate (20 mL×3).

The filtrate was concentrated and the residue was purified by preparative HPLC to afford the title compound as a white powder (53.5 mg, 21%). MS m/z: 482 [M+H]⁺. ¹H NMR (400 MHz, CD₃OD) δ 7.54 (d, J=3.1 Hz, 1H), 7.31 (d, J=3.2 Hz, 1H), 7.26-6.99 (m, 7H), 6.56 (d, J=3.2 Hz, 1H), 4.40 (s, 2H) ppm.

### Example 79

### 2-(5-Benzyl-2-(2-fluoro-5-((6-fluoro-4-(methylsulfonyl)-1H-indol-5-yl)oxy)phenyl)-1H-imidazol-4-yl)propan-2-ol

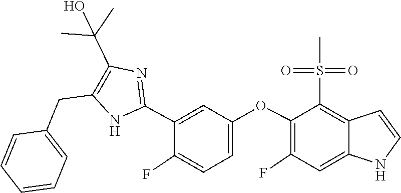 [SMILES: CC(C)(O)c1nc(-c2cc(Oc3c(F)cc4[nH]ccc4c3S(C)(=O)=O)ccc2F)[nH]c1Cc1ccccc1]

### A. (2-Fluoro-5-((6-fluoro-4-(methylthio)-1H-indol-5-yl)oxy)phenyl)methanamine

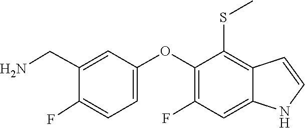 [SMILES: CSc1c(Oc2ccc(F)c(CN)c2)c(F)cc2[nH]ccc12]

To a stirred solution of 2-fluoro-5-[(6-fluoro-4-methylsulfanyl-1H-indol-5-yl)oxy]benzonitrile (Intermediate 6, 1 g, 3.16) in THF (20 mL) were added ammonium hydroxide (2 mL) and Raney Ni (1 g). The reaction mixture was stirred at room temperature under hydrogen atmosphere overnight, filtered and concentrated. The resulting residue was purified by flash chromatography (0-10% methanol in dichloromethane) to afford the title compound as a yellow solid (1.06 g, 80%). MS m/z: 321 [M+H]⁺.

### B. Methyl 2-(hydroxyimino)-3-oxo-4-phenylbutanoate

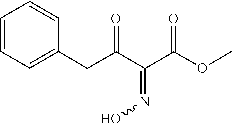 [SMILES: COC(=O)C(=NO)C(=O)Cc1ccccc1]

To a solution of methyl 3-oxo-4-phenyl-butanoate (5 g, 26 mmol) in acetic acid (50 mL) was added a solution of sodium nitrite (1.88 g, 27.3 mmol) in water (25 mL) at 0° C. The mixture was stirred at 0° C. for three hours and diluted with ethyl acetate (300 mL). The organic phase was washed with saturated sodium bicarbonate solution (100 mL×5) and brine (200 mL), dried over sodium sulfate, filtered and concentrated. The resulting residue was purified by flash chromatography (0-30% ethyl acetate in petroleum ether) to afford the title compound as a yellow oil (4 g, 64%). MS m/z: 222 [M+H]⁺.

### C. Methyl 5-benzyl-2-(2-fluoro-5-((6-fluoro-4-(methylthio)-1H-indol-5-yl)oxy)phenyl)-1H-imidazole-4-carboxylate

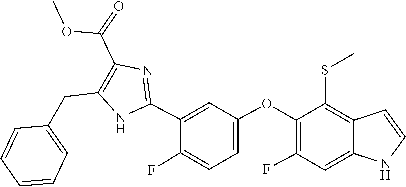

A solution of methyl 2-hydroxyimino-3-oxo-4-phenyl-butanoate (0.732 g, 3.04 mmol) and [2-fluoro-5-[(6-fluoro-4-methylsulfanyl-1H-indol-5-yl)oxy]phenyl]methanamine (1.28 g, 3.04 mmol) in acetonitrile (20 mL) was heated at reflux overnight and concentrated. The resulting residue was purified by flash chromatography (0-40% ethyl acetate in petroleum ether) to afford the title compound as a yellow oil (0.88 g, 43%). MS m/z: 506 [M+H]⁺.

### D. 2-(5-Benzyl-2-(2-fluoro-5-((6-fluoro-4-(methylthio)-1H-indol-5-yl)oxy)phenyl)-1H-imidazol-4-yl)propan-2-ol

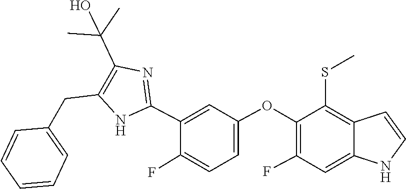 [SMILES: CSc1c(Oc2ccc(F)c(-c3nc(C(C)(C)O)c(Cc4ccccc4)[nH]3)c2)c(F)cc2[nH]ccc12]

A 3M solution of methylmagnesium bromide in ether (5 mL, 15 mmol) was added to a solution of methyl 5-benzyl-2-[2-fluoro-5-[(6-fluoro-4-methylsulfanyl-1H-indol-5-yl)oxy]phenyl]-1H-imidazole-4-carboxylate (0.2 g, 0.297 mmol) in THF (5 mL) at room temperature. The reaction mixture was stirred at room temperature overnight, quenched with saturated ammonium chloride solution (15 mL) and extracted with ethyl acetate (2×30 mL). The combined organic extracts were dried over sodium sulfate, filtered and concentrated. The resulting residue was purified by flash chromatography to afford the title compound as a yellow oil (0.2 g, 96%). MS m/z: 506 [M+H]⁺.

### E. Example 79. 2-(5-Benzyl-2-(2-fluoro-5-((6-fluoro-4-(methylsulfonyl)-1H-indol-5-yl)oxy)phenyl)-1H-imidazol-4-yl)propan-2-ol

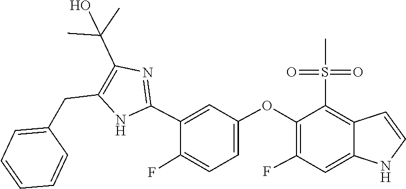 [SMILES: CC(C)(O)c1nc(-c2cc(Oc3c(F)cc4[nH]ccc4c3S(C)(=O)=O)ccc2F)[nH]c1Cc1ccccc1]

To a solution of 2-(5-benzyl-2-(2-fluoro-5-((6-fluoro-4-(methylthio)-1H-indol-5-yl)oxy)phenyl)-1H-imidazol-4-yl)propan-2-ol (150 mg, 0.214 mmol) in methanol (15 mL) was added a solution of ammonium molybdate tetrahydrate (0.3 g, 1.53 mmol) in hydrogen peroxide (1.5 mL, 30% in water) at room temperature. The reaction was stirred at room temperature for two hours, diluted with ethyl acetate (100 mL), washed with water (100 mL×3), saturated sodium sulfite solution (50 mL) and brine (100 mL), dried over sodium sulfate, filtered and concentrated. The resulting residue was purified by silica gel column (0-50% methanol in dichloromethane) to afford the title compound as a pale yellow solid (35.7 mg, 30%). MS m/z: 538 [M+H]⁺. ¹H NMR (400 MHz, CD₃OD) δ 7.66 (d, J=10.4 Hz, 1H), 7.52 (d, J=3.2 Hz, 1H), 7.52-7.49 (m, 1H), 7.29-7.23 (m, 2H), 7.21-7.11 (m, 5H), 6.96-6.90 (m, 1H), 4.32-4.06 (m, 2H), 3.34 (s, 3H), 1.52 (s, 6H) ppm.

### Example 80

### (5-Benzyl-2-(5-((4,6-difluoro-1H-indol-5-yl)oxy)-2-fluorophenyl)-1H-imidazol-4-yl)methanamine

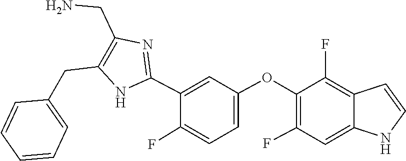 [SMILES: NCc1nc(-c2cc(Oc3c(F)cc4[nH]ccc4c3F)ccc2F)[nH]c1Cc1ccccc1]

### A. N-Hydroxy-2-oxo-3-phenylpropanimidoyl cyanide

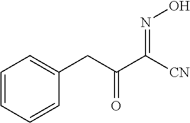 [SMILES: N#C/C(=N\O)C(=O)Cc1ccccc1]

To a stirred solution of 3-oxo-4-phenyl-butanenitrile (2.1 g, 13.2 mmol) in acetic acid (21 mL) was added a solution of sodium nitrite (956 mg, 13.9 mmol) in water (10.5 mL) at 0° C. The mixture was stirred at 0° C. for one hour, water (50 mL) was added and extracted with ethyl acetate (30 mL×3). The combined organic extracts were washed with saturated sodium bicarbonate solution (30 mL×2) and brine, dried over sodium sulfate and concentrated. The resulting crude product was purified by flash chromatography (0-50% ethyl acetate in petroleum ether) to afford the title compound (1.6 g, 44%). ¹H NMR (400 MHz, CDCl₃): δ 7.35-7.27 (m, 3H), 7.23-7.19 (m, 2H), 4.12 (s, 2H) ppm.

### B. 5-Benzyl-2-[5-[(4,6-difluoro-1H-indol-5-yl)oxy]-2-fluoro-phenyl]-1H-imidazole-4-carbonitrile

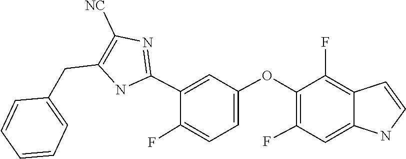 [SMILES: N#Cc1nc(-c2cc(Oc3c(F)cc4[nH]ccc4c3F)ccc2F)[nH]c1Cc1ccccc1]

A solution of N-hydroxy-2-oxo-3-phenylpropanimidoyl cyanide (193 mg, 1.03 mmol) and [5-[(4,6-difluoro-1H-indol-5-yl)oxy]-2-fluoro-phenyl]methanamine (Example 78 RA15371979 Step C, 200 mg, 0.68 mmol) in acetonitrile (5 mL) was refluxed overnight. Water (30 mL) was added and the mixture was extracted with ethyl acetate (20 mL×3). The combined organic extracts were washed with brine, dried over sodium sulfate, filtered and concentrated. The resulting crude product was purified by flash chromatography (0-50% ethyl acetate in petroleum ether) to afford the title compound (110 mg, 30%). MS m/z: 445 [M+H]⁺.

### C. Example 80. (5-Benzyl-2-(5-((4,6-difluoro-1H-indol-5-yl)oxy)-2-fluorophenyl)-1H-imidazol-4-yl)methanamine

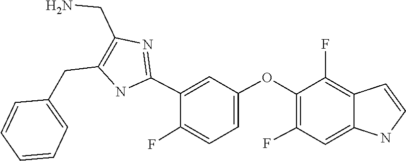 [SMILES: NCc1nc(-c2cc(Oc3c(F)cc4[nH]ccc4c3F)ccc2F)[nH]c1Cc1ccccc1]

To a solution of 5-benzyl-2-[5-[(4,6-difluoro-1H-indol-5-yl)oxy]-2-fluoro-phenyl]-1H-imidazole-4-carbonitrile (110 mg, 0.25 mmol) in THF (10 mL) and concentrate ammonium hydroxide (28% in water, 1 mL) was added Raney Nickel (200 mg). The reaction mixture was stirred at room temperature under hydrogen atmosphere for three hours, filtered and concentrated. The resulting crude product was purified by flash chromatography (0-15% methanol in dichloromethane) to afford the title compound (39.6 mg, 34%). MS m/z: 449 [M+H]⁺. ¹H NMR (400 MHz, CD₃OD) δ 7.37 (dd, J=6.0, 3.2 Hz, 1H), 7.19 (d, J=3.2 Hz, 1H), 7.17 (s, 1H), 7.15 (s, 1H), 7.11-7.08 (m, 3H), 7.07-7.05 (m, 1H), 7.03 (d, J=10.0 Hz, 1H), 6.87 (dt, J=8.0, 3.6 Hz, 1H), 6.44 (dd, J=3.2, 0.8 Hz, 1H), 3.93 (s, 2H), 3.61 (s, 2H) ppm.

### Example 81

### 5-(3-(4-(Hydroxy(phenyl)methyl)-1H-imidazol-2-yl)phenoxy)-1H-indole-4-carboxylic acid

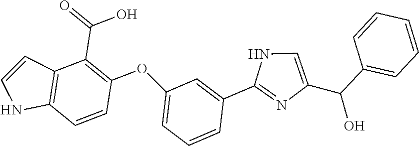 [SMILES: O=C(O)c1c(Oc2cccc(-c3nc(C(O)c4ccccc4)c[nH]3)c2)ccc2[nH]ccc12]

### A. Methyl 5-(3-(4-(hydroxy(phenyl)methyl)-1H-imidazol-2-yl)phenoxy)-1H-indole-4-carboxylate

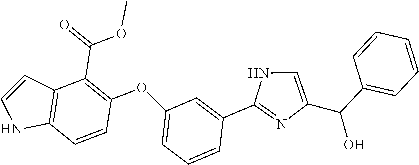 [SMILES: COC(=O)c1c(Oc2cccc(-c3nc(C(O)c4ccccc4)c[nH]3)c2)ccc2[nH]ccc12]

To a stirred solution of methyl 5-(3-(4-formyl-1H-imidazol-2-yl)phenoxy)-1H-indole-4-carboxylate (Intermediate 16-1, 540 mg, 1.49 mmol) in THF (20 mL) was added a 1M solution of phenyl magnesium bromide in THF (8.97 mL, 8.97 mmol) at 0° C. After 10 minutes, the ice bath was removed and the reaction mixture was allowed to stir at room temperature for 2.5 hours. The reaction was quenched with water, acidified with 1N hydrochloric acid and extracted with ethyl acetate. The combined organic extracts were dried over MgSO₄, filtered and concentrated to give a yellow oil, which was purified on silica gel column (50-70% ethyl acetate in heptane) to afford the title compound as a pale yellow solid (451 mg, 69%). MS m/z: 440 [M+H]⁺.

### B. Example 81. 5-(3-(4-(Hydroxy(phenyl)methyl)-1H-imidazol-2-yl)phenoxy)-1H-indole-4-carboxylic acid

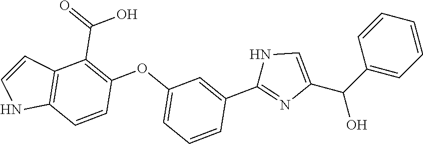 [SMILES: O=C(O)c1c(Oc2cccc(-c3nc(C(O)c4ccccc4)c[nH]3)c2)ccc2[nH]ccc12]

To a 5 mL microwave vial containing a solution of methyl 5-(3-(4-(hydroxy(phenyl)methyl)-1H-imidazol-2-yl)phenoxy)-1H-indole-4-carboxylate (414 mg, 0.94 mmol) in THF (5 mL) was added potassium trimethylsilanolate (604 mg, 4.7 mmol). The reaction mixture was heated at 85° C. for three hours, cooled to room temperature, quenched with water and extracted with ethyl acetate to remove neutral impurity. The aqueous phase was acidified with 1N hydrochloric acid and extracted with ethyl acetate (50 mL×2). The combined organic extracts were dried over MgSO₄, filtered and concentrated to afford the title compound as a yellowish solid (220 mg, 55%). The product was used without further purification. MS m/z: 426 [M+H]⁺. ¹H NMR (500 MHz, DMSO-d₆) δ 14.61 (br s, 1H), 12.701 (br s, 1H), 11.49 (s, 1H), 7.66-7.62 (m, 2H), 7.59 (s, 1H), 7.54 (t, J=2.5 Hz, 1H), 7.50 (t, J=8.5 Hz, 1H), 7.47 (d, J=7.0 Hz, 2H), 7.41-7.31 (m, 4H), 6.98 (dd, J=8.0, 2.0 Hz, 1H), 6.88 (d, J=9.0 Hz, 1H), 6.77 (t, J=2.0 Hz, 1H), 6.42 (br s, 1H), 5.85 (s, 1H) ppm.

### Example 82

### 5-(3-(4-(Hydroxy(phenyl)methyl)-1H-imidazol-2-yl)phenoxy)-N-(2-methoxyethyl)-1H-indole-4-carboxamide

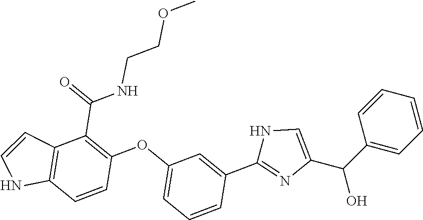 [SMILES: COCCNC(=O)c1c(Oc2cccc(-c3nc(C(O)c4ccccc4)c[nH]3)c2)ccc2[nH]ccc12]

A mixture of 5-(3-(4-(hydroxy(phenyl)methyl)-1H-imidazol-2-yl)phenoxy)-1H-indole-4-carboxylic acid (63 mg, 148 uM), HATU (112 mg, 0.3 mmol), triethylamine (0.061 mL, 0.45 mmol) and 2-methoxyethan-1-amine (16.6 mg, 1.5 eq) in DMF (3 mL) was stirred at room temperature for three hours, quenched with water and extracted with ethyl acetate. The combined organic extracts were dried over MgSO4, filtered and concentrated. The resulting orange oil was purified by silica gel column chromatography (80-100% ethyl acetate in heptane) to afford the title compound as an off-white solid (28 mg, 28%). MS m/z: 483 [M+H]⁺. LC-MS retention time: 0.66 minutes.

### Example 83

### 5-(3-(4-(Hydroxy(phenyl)methyl)-1H-imidazol-2-yl)phenoxy)-N-(3,3,3-trifluoro-2 hydroxypropyl)-1H-indole-4-carboxamide

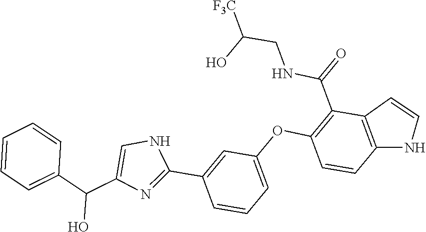 [SMILES: O=C(NCC(O)C(F)(F)F)c1c(Oc2cccc(-c3nc(C(O)c4ccccc4)c[nH]3)c2)ccc2[nH]ccc12]

### A. 5-(3-(4-(Hydroxymethyl)-1H-imidazol-2-yl)phenoxy)-1H-indole-4-carboxylic acid

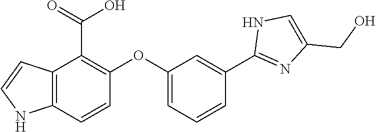 [SMILES: O=C(O)c1c(Oc2cccc(-c3nc(CO)c[nH]3)c2)ccc2[nH]ccc12]

To a mixture of methyl 5-(3-(4-(hydroxymethyl)-1H-imidazol-2-yl)phenoxy)-1H-indole-4-carboxylate (100 mg, 275 μmol) in THF (5 mL) was added potassium trimethylsilanolate (106 mg, 825 μmol). The reaction mixture was heated at 85° C. for two hours, cooled to room temperature and diluted with water and ether. The ether layer was discarded. The aqueous layer was acidified with 1N hydrochloric acid and extracted with ethyl acetate. The combined organic extracts were dried over MgSO₄, filtered and concentrated to afford the title compound as beige solid which was used without further purification. MS m/z: 350 [M+H]⁺.

### B. 5-(3-(4-(Hydroxymethyl)-1H-imidazol-2-yl)phenoxy)-N-(3,3,3-trifluoro-2-hydroxypropyl)-1H-indole-4-carboxamide

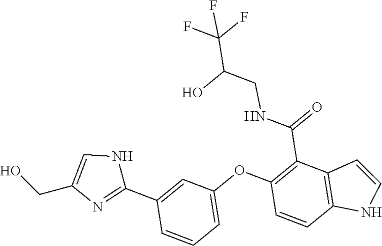 [SMILES: O=C(NCC(O)C(F)(F)F)c1c(Oc2cccc(-c3nc(CO)c[nH]3)c2)ccc2[nH]ccc12]

A mixture of 5-(3-(4-(hydroxymethyl)-1H-imidazol-2-yl)phenoxy)-1H-indole-4-carboxylic acid, 3-amino-1,1,1-trifluoropropan-2-ol (30 mg, 86 ummol), HATU (49 mg, 129 ummol) and triethylamine (0.035 mL, 258 ummol) in DMF (2 mL) was stirred at 40° C. for one hour. The solvent was evaporated and the residue was purified by flash chromatography (0-5% methanol in dichloromethane) to afford the title compound as a white solid (20 mg, 51%). MS m/z: 461 [M+H]⁺.

### C. 5-(3-(4-Formyl-1H-imidazol-2-yl)phenoxy)-N-(3,3,3-trifluoro-2-hydroxypropyl)-1H-indole-4-carboxamide

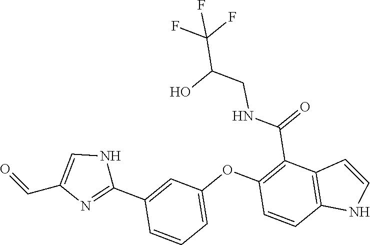 [SMILES: O=Cc1c[nH]c(-c2cccc(Oc3ccc4[nH]ccc4c3C(=O)NCC(O)C(F)(F)F)c2)n1]

To a solution of 5-(3-(4-(hydroxymethyl)-1H-imidazol-2-yl)phenoxy)-N-(3,3,3-trifluoro-2-hydroxypropyl)-1H-indole-4-carboxamide (20 mg, 43 μmol) in dichloromethane (2 mL) and a few drops of methanol was added manganese dioxide (38 mg, 430 μmol). The reaction mixture was stirred at room temperature for three hours, diluted with dichloromethane, filtered through a pad of Celite and concentrated to afford the title compound (19 mg, 95%), which was used without further purification. MS m/z: 459 [M+H]⁺.

### D. Example 83. 5-(3-(4-(Hydroxy(phenyl)methyl)-1H-imidazol-2-yl)phenoxy)-N-(3,3,3-trifluoro-2 hydroxypropyl)-1H-indole-4-carboxamide

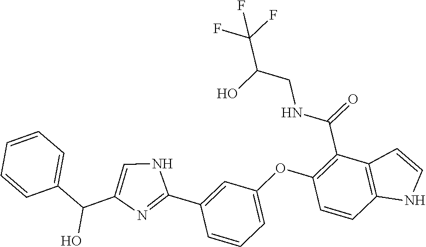 [SMILES: O=C(NCC(O)C(F)(F)F)c1c(Oc2cccc(-c3nc(C(O)c4ccccc4)c[nH]3)c2)ccc2[nH]ccc12]

To a stirred solution of 5-(3-(4-formyl-1H-imidazol-2-yl)phenoxy)-N-(3,3,3-trifluoro-2-hydroxypropyl)-1H-indole-4-carboxamide (20 mg, 44 μmol) in THF (3 mL) was added a 1M solution of phenyl magnesium bromide in THF (0.46 mL, 0.46 mmol) at 0° C. The reaction mixture was stirred at room temperature for one hour, diluted with THF (10 mL), quenched with saturated ammonium chloride and extracted with ethyl acetate. The combined organic extracts were dried over MgSO4, filtered and concentrated. The resulting residue was purified on silica gel column chromatography (10% methanol in dichloromethane) to afford the title compound as a white solid (3 mg, 13%). MS m/z: 537 [M+H]⁺. LC-MS retention time: 0.74 minutes.

### Biological Assays

### Example 84: Aggregation Analysis Using Differential Static Light Scattering (DSLS)

Purified recombinant NBD1 was produced using previously described methods (A. Schmidt, J. L. Mendoza, P. J. Thomas (2011) Biochemical and Biophysical Approaches to Probe CFTR Structure (365-376) M. D. Amaral, K. Kunzelmann (eds.), Cysic Fibrosis, Methods in Molecular Biology 741, Springer Science+Business Media). The effect of test compounds on thermal stability of NBD1 was evaluated by differential static light scattering (DSLS) using the Harbinger Stargazer-384 instrument (Epiphyte Three, Toronto, Canada). Test compounds were dissolved and diluted to desired concentrations in 100% DMSO. The compounds or DMSO controls (100 nL) were stamped into wells of a 385-well low volume optical plate (Corning Inc., Corning, NY) using the Echo 555 acoustic liquid handler (Labcyte Inc., San Jose, CA).

NBD1 protein was diluted to 0.2 mg/ml in S200 buffer (50 mM Tris-HCl, 150 mM NaCl, 5 mM MgCl₂, 2 mM ATP, 2 mM DTT, pH7.6) containing 1% glycerol. 10 uL of protein solution was aliquoted into the 384-well plate harboring the test compounds and 10 uL mineral oil was overlayed onto the protein solution, using the epMotion robotic liquid handler (Eppendorf North America, Hauppauge, NY). After placing into the Stargazer instrument, the plate was heated at 1° C. per minute to 70° C. Images were captured from 25° C. to 70° C. every 0.5° C. At the end of the experiment run, instrument software integrated image files and analyzed data automatically. A linear regression curve was generated for each well, representing the increase in light scattering over time. A temperature of aggregation (Tagg) was calculated based on the inflection point of the curve. To better compare data across experiments the average Tagg for DMSO control wells was calculated and subtracted from values for wells containing compounds to obtain a “ΔTagg” value. These ΔTagg values reflect stabilizing efficacy of the compounds.

### Example 85: TECC24 AUC Fold Over DMSO @ 10 μM

The effects of a test agent on CFTR-mediated transepithelial chloride transport is measured using TECC24 recording analysis. Test agents are solubilized in DMSO. Solubilized test agents are mixed with incubation medium containing DMEM/F12, Ultroser G (2%; Crescent Chemical, catalog #67042), Hyclone Fetal Clone 11(2%; GE Healthcare, catalog #SH30066.02), bovine brain extract (0.25%; Lonza, catalog #CC-4098), insulin (2.5 μg/mL), IL-13 (10 ng/mL), hydrocortisone (20 nM), transferrin (2.5 μg/mL), triiodothyronine (500 nM), ethanolamine (250 nM), epinephrine (1.5 μM), phosphoethanolamine (250 nM), and retinoic acid (10 nM). Primary human bronchial epithelial cells from a ΔF508 homozygous CF donor (CF-HBE cells; from University of North Carolina Cystic Fibrosis Tissue Procurement Center), grown on Transwell HTS 24-well cell culture inserts (Costar, catalog #3378), are exposed to test agents or controls dissolved in incubation medium. The CF-HBE cells are cultured at 36.5° C. for 48 hours before TECC24 recordings are performed in the presence or absence of test agent, a positive control or vehicle (DMSO).

Following incubation, the transwell cell culture inserts containing the test agent or control-treated CF-HBE cells are loaded onto a TECC24 apparatus (TECC v7 or MTECC v2; EP Design) to record the transepithelial voltage (VT) and resistance (TEER) using 4 AgCl electrodes per well configured in current-clamp mode. The apical and basolateral bath solutions both contain (in mM) 140 NaCl, 5 KCl, 2 CaCl₂, 1 MgCl₂, 10 Hepes, and 10 glucose (adjusted to pH 7.4 with NaOH). To inhibit basal Na+ absorption, the ENaC inhibitor benzamil (10 μM) is added to the bath. Then, the adenylate cyclase activator, forskolin (10 μM), is added to the bath to activate CFTR. The forskolin-stimulated C1-transport is halted by addition of CFTR inhibitor-172 (20 μM) to the bath at the end of the experiment to confirm specificity. VT and TEER recordings are digitally acquired at routine intervals using TECC or MTECC software (EP Design). VT and TEER are transformed into equivalent transpeithelial C1-current (IEQ), and the Area Under the Curve (AUC) of the IEQ timecourse between forskolin and CFTR inhibitor-172 addition is generated using Excel (Microsoft). Efficacy is expressed as the ratio of the test agent AUC divided by vehicle AUC. EC50s based on AUC are generated using the non-linear regression log(agonist) vs. response function in Prism software (Graphpad) with HillSlope fixed=1.

If a test agent increases the AUC of the forskolin-stimulated IEQ relative to vehicle in CF-HBE cells, and this increase is inhibited by CFTR inhibitor-172, then the test agent is considered a CFTR corrector.

Data for Compounds 1-83 are provided in Table 2 below.

TABLE 2

| | DSLS NBD1/3D |
|---|---|
| | Human Stability |
| | delta-T (° C.) |
| Example No | @ 100 (μM) |
| 1 | A |
| 2 | B |
| 3 | A |
| 4 | A |
| 5 | A |
| 6 | A |
| 7 | B |
| 8 | A |
| 9 | A |
| 10 | A |
| 11 | B |
| 12 | A |
| 13 | B |
| 14 | A |
| 15 | A |
| 16 | B |
| 17 | B |
| 18 | A |
| 19 | B |
| 20 | B |
| 21 | B |
| 22 | B |
| 23 | B |
| 24 | B |
| 25 | B |
| 26 | D |
| 27 | C |
| 28 | B |
| 29 | C |
| 30 | C |
| 31 | B |
| 32 | B |
| 33 | D |
| 34 | C |
| 35A | B |
| 35B | C |
| 36 | D |
| 37 | C |
| 38 | C |
| 39 | C |
| 40 | C |
| 41 | D |
| 42 | C |
| 43 | C |
| 44 | B |
| 45 | D |
| 46 | C |
| 47 | C |
| 48 | C |
| 49 | B |
| 50 | B |
| 51 | B |
| 52 | B |
| 53 | B |
| 54 | B |
| 55 | B |
| 56 | A |
| 57 | B |
| 58 | A |
| 59 | B |
| 60 | A |
| 61 | B |
| 62 | B |
| 63 | A |
| 63A | A |
| 63B | B |
| 64 | A |
| 65 | A |
| 66 | A |
| 67 | C |
| 68 | B |
| 69 | B |
| 70 | B |
| 71 | A |
| 72 | A |
| 73 | A |
| 74 | B |
| 75 | B |
| 76 | B |
| 77 | B |
| 76 | B |
| 79 | B |
| 80 | B |
| 81 | B |
| 82 | B |
| 83 | A |

*A: T ≥8° C.; B: 4-8° C.; C: ≤4° C.; D: Not Determined.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A compound of Formula (I): 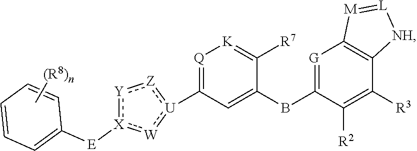
wherein
U is C;
W is N or NR^{a}, wherein R^{a} is H;
X is N or C;
Y is CR^{b}, wherein R^{b} is H, alkyl or carboxy, N, or NR^{c}, where R^{c} is alkyl;
Z is O, S, N or NR^{d}, where R^{d} is H;
B is —O—, —C(O)—, —S—, —S(O)—, —S(O)₂—, or —C(R¹⁰R¹¹)—, wherein R¹⁰ and R¹¹ are independently selected from H, hydroxy, and —(CH₂)pJ, wherein p=1-3, and J is —C(O)NH₂, —COOH, or —CN;
G is N or CR¹, wherein R¹ is H, halo, alkyl, alkenyl, amido, sulfinyl, sulfonyl and sulfonamido;
R² is H or halo;
R³ is H or halo;
L is N or CH;
M is N or CR⁹, wherein R⁹ is H, —CH₂COOH, —CH₂OH, or R⁹ taken together with R¹ and any intervening atoms forms an N-containing 6- or 7-membered heterocycle, optionally substituted with oxo;
Q is N or CR^{4c};
R^{4c} is selected from H or halo;
K is N or CH;
R⁷ is H or amino;
E is —S(O)—, —S(O)₂—, or —C(R⁵R⁶)—, wherein
R⁵ is selected from H, alkyl, hydroxy, alkoxy, carboxy, and amino;
R⁶ is H or alkyl; and
n=0-1;
R⁸ is halo, haloalkyl, or unsubstituted alkyl;
wherein each alkyl, alkenyl, amino, amido, and alkoxy is independently unsubstituted or substituted with one or more substituents selected from halo, hydroxy, amino, amido, alkyl, alkoxy, carboxy, heterocyclyl, and sulfonyl; wherein the alkyl substituent is optionally further substituted with one or more substituents selected from halo, hydroxy, amino, amido, carboxy, and sulfonamido.

2. The compound of claim 1, wherein the compound of Formula I is the compound of Formula I′

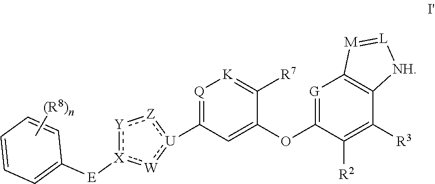

3. The compound of claim 1 or 2, wherein W is NH; X is C; Y is CH; and Z is N.

4. The compound of claim 1 or 2, wherein W is NH; X is C; Y is N; and Z is N.

5. The compound of claim 1 or 2, wherein W is N; X is C; Y is NH; and Z is N.

6. The compound of claim 1 or 2, wherein W is N; X is C; Y is CH; and Z is NH.

7. The compound of claim 1 or 2, wherein W is N; X is C; Y is CR^{b}; and Z is NH.

8. The compound of claim 1 or 2, wherein W is N; X is C; Y is C; and Z is N.

9. The compound of claim 1 or 2, wherein W is N; X is C; Y is N; and Z is NH.

10. The compound of claim 1 or 2, wherein W is N; X is N; Y is CH; and Z is N.

11. The compound of claim 1 or 2, wherein W is N; X is C; Y is CH; and Z is O.

12. The compound of claim 1 or 2, wherein W is N; X is C; Y is CH; and Z is S.

13. The compound of any one of claims 1-12, wherein L is N; M is CH; G is CR¹; R² is H; R³ is H; and R^{4c} is H.

14. The compound of any one of claims 1-12, wherein L is CH; G is N; M is CH; R² is H; R³ is H; R^{4c} is H.

15. The compound of any one of claims 1-12, wherein G is N; M is CCH₂OH; R² is H; R³ is H; R^{4c} is H.

16. The compound of any one of claims 1-12, wherein G is CR¹, and R¹ is selected from —COOH, —CH₂OH, —CH₂COOH, —CH₂NH₂, —CH₂NHMe, —CH₂NHEt, —CH₂NHiPr, —CH₂NHtBu, —CH₂NHCOMe, —CH₂CONH—SO₂Me, —CH₂CONH—CF₃, —CH₂CONH—SO₂-cyclopropyl, —SOMe, and —SO₂Me; R² is H; R³ is H; and R^{4c} is H.

17. The compound of any one of claims 1-12, wherein G is CR¹, and R¹ is selected from —CH₂CH₂COOH, —CH₂CH₂CONH₂, —CH₂CH₂CONHSO₂Me, and —CH₂CH₂CONHSO₂cyclopropyl; R² is H; R³ is H; and R^{4c} is H.

18. The compound of any one of claims 1-12, wherein G is CR¹, and R¹ is selected from C(O)NHR¹⁵; R² is H; R³ is H; R^{4c} is H; and
R¹⁵ is selected from H, —CH₂CH₂OH, —CH₂CH₂OMe, —CH₂CH₂NMe₂, —CH₂CH₂OCH₂CH₂OMe, —CH₂CH(OH)CH₂OH, —CH₂CH(OH)CH₂NEt₂, —CH₂CONHMe, —CH₂CMe₂OH, —CH₂CH(OH)CF₃, —CH₂CH₂SO₂Me, —CH₂-(3-oxetanyl)—CH₂OH, —CH₂-4-(2,2-dimethyl-1,3-dioxolanyl), —CH₂-thiazolyl, —OMe, —OCH₂CH₂OMe and

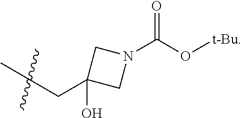 [SMILES: *CC1(O)CN(C(=O)OC(C)(C)C)C1]

19. The compound of any one of claims 1-12, wherein G is CR¹, and R¹ is selected from —CH(OH)CH₃, —COMe, —CONH₂, —CH₂OH, —SO₂Me, —SOCH₂CH₂OMe, —SO₂CH₂CH₂OMe, —SO₂CH₂CH₂NHMe 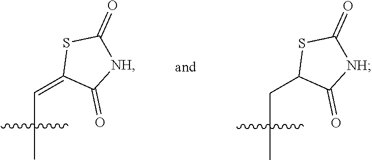 [SMILES: */C=C1/SC(=O)NC1=O ; *CC1SC(=O)NC1=O]
R² is F; R³ is H; and R^{4c} is H.

20. The compound of any one of claims 1-12, wherein G is CR¹, and R¹ is selected from Me, —CH₂COOH, —CH₂CH₂COOH, —CH₂CH₂SO₂NH₂, —SO₂Me, —SO₂NHMe, 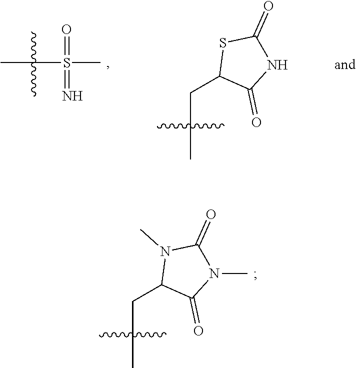
R² is F; R³ is H; and R^{4c} is F.

21. The compound of any one of claims 1-12, wherein L is CH; M is N; G is CH; R² is H; R³ is H; R^{4c} is H.

22. The compound of any one of claims 1-12, wherein L is CH; M is CH; G is CH; R² is H; R³ is H; R^{4c} is H.

23. The compound of any one of claims 1-12, wherein M is CR⁹, where R⁹ is selected from —CH₂COOH and —CH₂OH.

24. The compound of any one of claims 1-12, wherein M is CR⁹, where R⁹ taken together with R¹ and any intervening atoms forms an N-containing 6-membered heterocycle.

25. The compound of any one of claims 1-12, wherein M is CR⁹, where R⁹ taken together with R¹ and any intervening atoms forms an N-containing 7-membered heterocycle.

26. The compound of any one of claims 1-12, wherein M is CR⁹, where R⁹ taken together with R¹ and any intervening atoms forms an N-containing 6-membered heterocycle substituted with oxo.

27. The compound of any one of claims 1-12, wherein M is CR⁹, where R⁹ taken together with R¹ and any intervening atoms forms an N-containing 7-membered heterocycle substituted with oxo.

28. The compound of any one of claims 1-12, wherein M is CCH₂COOH; R¹ is Me; R² is F; R³ is H; and R^{4c} is F.

29. The compound of any one of claims 1-12, wherein M is CCH₂OH; R¹ is Me; R² is H; R³ is H; and G is N.

30. The compound of any one of claims 1-12, wherein R¹ is F; R² is F; R³ is H; and R^{4c} is H.

31. The compound of any one of claims 1-12, wherein R¹ is F; R² is H; R³ is H; and R^{4c} is H.

32. The compound of any one of claims 1-12, wherein R¹ is F; R² is F; R³ is H; and R^{4c} is F.

33. The compound of any one of claims 1-12, wherein R¹ is H; R² is F; R³ is H; and R^{4c} is H.

34. The compound of any one of claims 1-12, wherein R¹ is H; R² is H; R³ is F; and R^{4c} is H.

35. The compound of any one of claims 1-12, wherein R¹ is H; R² is H; R³ is H; and R^{4c} is H.

36. The compound of claim 1, wherein B is —O—; K is CH; Q is CR^{4c}; R^{4c} is H; R⁷ is H.

37. The compound of claim 1, wherein B is —O—; K is CH; Q is N; R⁷ is H.

38. The compound of claim 1, wherein B is —O—; K is CH; Q is N; R⁷ is NH₂.

39. The compound of claim 1, wherein B is —O—; Q is CR^{4c}; R^{4c} is F; R⁷ is H.

40. The compound of claim 1, wherein B is selected from —C(O)—, —S—, —S(O)—, —S(O)₂—.

41. The compound of claim 40, wherein p=1.

42. The compound of claim 40 or 41, wherein J is —C(O)NH₂, —COOH, or —CN.

43. The compound of any one of claims 1-42, wherein E is selected from —S(O)— and —S(O)₂—.

44. The compound of any one of claims 1-42, wherein E is —C(R⁵R⁶)—, R⁵ is selected from H and hydroxy; and R⁶ is H.

45. The compound of any one of claims 1-42, wherein E is —C(R⁵R⁶)—, R⁵ is selected from H and hydroxy; and R⁶ is alkyl.

46. The compound of any one of claims 1-42, wherein E is —C(R⁵R⁶)—, R⁵ is selected from alkoxy, carboxy, and amino; and R⁶ is H.

47. The compound of any one of claims 1-42, wherein E is —C(R⁵R⁶)—, R⁵ is selected from alkoxy, carboxy, and amino; and R⁶ is alkyl.

48. The compound of any one of claims 1-42, wherein E is —C(R⁵R⁶)—, R⁵ is selected from H, hydroxy, methyl, NH₂, CF₃, —COOH, —CH₂CH₂COOH, —OCH₂OH, —OCH₂COOH, —OCH₂CH₂NH₂, and —OCH₂CH(OH)CH₂OH; and R⁶ is selected from H and CH₃.

49. The compound of any one of claims 1-42, wherein E is —C(R⁵R⁶)—, and R⁵ and R⁶ are each H.

50. The compound of any one of claims 1-42, wherein E is —C(R⁵R⁶)—, R⁵ is hydroxy and R⁶ is H.

51. The compound of any one of claims 1-42, wherein E is —C(R⁵R⁶)—, R⁵ is hydroxy or methyl, and R⁶ is methyl.

52. The compound of any one of claims 1-42, wherein E is —C(R⁵R⁶)—, R⁵ is amino or methyl, and R⁶ is H.

53. The compound of any one of claims 1-42, wherein E is —C(R⁵R⁶)—, R⁵ and R⁶ are H, and R⁸ is halo.

54. The compound of any one of claims 1-52, wherein R⁸ is halo.

55. The compound of claim 1, wherein the compound of Formula I is a compound of Formula IA: 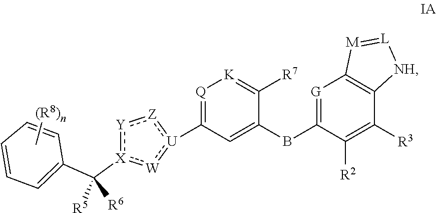
wherein
R⁵ is selected from H, alkyl, hydroxy, alkoxy, carboxy, and amino; and
R⁶ is H or alkyl.

56. The compound of claim 1, wherein the compound of Formula I is a compound of Formula IB: 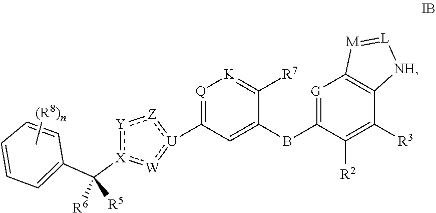
wherein
R⁵ is selected from H, alkyl, hydroxy, alkoxy, carboxy, and amino; and
R⁶ is H or alkyl.

57. A compound of Formula II: 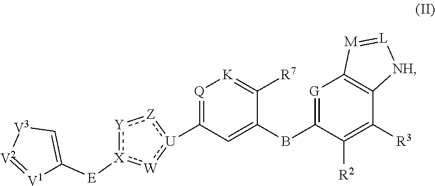
wherein
U is C;
W is N or NR^{a}, wherein R^{a} is H;
X is N or C;
Y is CR^{b}, wherein R^{b} is H, alkyl or carboxy, N, or NR^{c}, where R^{c} is alkyl;
Z is O, S, N or NR^{d}, where R^{d} is H;
B is —O—, —C(O)—, —S—, —S(O)—, —S(O)₂—, or —C(R¹⁰R¹¹)—, wherein R¹⁰ and R¹¹ are independently selected from H, hydroxy, and —(CH₂)pJ, wherein p=1-3, and J is —C(O)NH₂, —COOH, or —CN;
G is N or CR¹, wherein R¹ is H, halo, alkyl, alkenyl, amido, sulfinyl, sulfonyl and sulfonamido;
R² is H or halo;
R³ is H or halo;
L is N or CH;
M is N or CR⁹, wherein R⁹ is H, —CH₂COOH, —CH₂OH, or R⁹ taken together with R¹ and any intervening atoms forms an N-containing 6- or 7-membered heterocycle, optionally substituted with oxo;
Q is N or CR^{4c};
R^{4c} is selected from H or halo;
K is N or CH;
R⁷ is H or amino;
E is —S(O)—, —S(O)₂—, or —C(R⁵R⁶)—, wherein
R⁵ is selected from H, alkyl, hydroxy, alkoxy, carboxy, and amino;
R⁶ is H or alkyl;
V¹ is N or CH;
V² is N or CH;
V³ is NR¹² or CHR¹²; and
R¹² is H, unsubstituted alkyl, or haloalkyl;
wherein each alkyl, alkenyl, amino, amido, and alkoxy is independently unsubstituted or substituted with one or more substituents selected from halo, hydroxy, amino, amido, alkyl, alkoxy, carboxy, heterocyclyl, and sulfonyl; wherein the alkyl substituent is optionally further substituted with one or more substituents selected from halo, hydroxy, amino, amido, carboxy, and sulfonamido.

58. The compound of claim 57, wherein V¹ is CH; V² is N; V³ is NR¹²; and R¹² is unsubstituted alkyl.

59. The compound of claim 57 or 58, wherein V¹ is CH; V² is N; V³ is NR¹²; and R¹² is methyl.

60. The compound of any one of claims 57-59, wherein V¹ is CH; V² is N; V³ is NR¹²; R¹² is methyl; E is —C(R⁵R⁶)—; and R⁵ is hydroxy.

61. The compound of any one of claims 57-60, wherein V¹ is CH; V² is N; V³ is NR¹²; R¹² is methyl; E is —C(R⁵R⁶)—; R⁶ is H; and R⁵ is hydroxy.

62. The compound of any one of claims 1-56, wherein n=0.

63. The compound of any one of claims 1-56, wherein n=1.

64. A compound selected from any of the compounds as shown in Table 1.

65. The compound of any one of claims 1-64, wherein the compound is a CFTR stabilizer.

66. A pharmaceutical composition comprising a compound of any one of the preceding claims, and one or more pharmaceutically acceptable carriers or excipients.

67. The pharmaceutical composition of claim 66, further comprising one or more CFTR therapeutic agents.

68. A method of treating deficient CFTR activity in a cell, comprising contacting the cell with a compound of any one of claims 1-65, or the pharmaceutical composition of claim 66 or 67.

69. The method of claim 68, wherein contacting the cell occurs in a subject in need thereof, thereby treating a CFTR-mediated condition and/or disease.

70. The method of claim 69, wherein the disease or condition is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear plasy, Pick's disease, several polyglutamine neurological disorders, Huntington's, spinocerebullar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

71. The method of claim 69 or 70, wherein the disease or condition is selected from cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

72. The method of any one of claims 69-71, wherein the disease or condition is cystic fibrosis.

73. A method of treating cystic fibrosis in a subject, comprising administering to the subject a therapeutically effective amount of a compound of any one of claims 1-65, or the pharmaceutical composition of claim 66 or 67.

74. The method of claim 73, wherein the subject is human.

75. The method according to claim 73 or 74, wherein said subject is at risk of developing cystic fibrosis, and wherein said administering step is carried out prior to the onset of symptoms of cystic fibrosis in said subject.
